(19) United States
(12) Patent Application Publication
(10) Pub. No.: US 2025/0169734 A1
(43) Pub. Date: May 29, 2025

(54) METHODS, SYSTEMS, AND COMPUTER READABLE MEDIA FOR EARLY DETECTION OF A NEURODEVELOPMENTAL OR PSYCHIATRIC DISORDER USING SCALABLE COMPUTATIONAL BEHAVIORAL PHENOTYPING AND AUTOMATED MOTOR SKILLS ASSESSMENT

(71) Applicant: Duke University, Durham, NC (US)

(72) Inventors: Geraldine Dawson, Durham, NC (US); Guillermo Sapiro, Durham, NC (US)

(73) Assignee: Duke University, Durham, NC (US)

(21) Appl. No.: 18/759,804

(22) Filed: Jun. 28, 2024

Related U.S. Application Data
(60) Provisional application No. 63/523,803, filed on Jun. 28, 2023.
(60) Provisional application No. 63/523,761, filed on Jun. 28, 2023.

(51) Int. Cl. A61B 5/16; A61B 5/00; A61B 5/11; G16H 15/00; G16H 50/30

(52) U.S. Cl. CPC A61B 5/168; A61B 5/1124; A61B 5/4836; A61B 5/7267; A61B 5/7275; A61B 5/7435; A61B 5/7475; G16H 15/00; G16H 50/30

(57) ABSTRACT

The subject matter described herein includes methods, systems, and computer readable media for early detection of a neurodevelopmental or psychiatric disorder using scalable computational behavioral phenotyping. According to one method for early detection of a neurodevelopmental or psychiatric disorder using scalable computational behavioral phenotyping includes obtaining user related information, wherein the user related information includes metrics derived from a user interacting with one or more applications executing on at least one user device; generating, using the user related information and a machine learning based model, a user assessment report including a prediction value indicating a likelihood that the user has a neurodevelopmental or psychiatric (neurodevelopmental/psychiatric) disorder and a prediction confidence value computed using relative contributions of the metrics to the prediction value generated using the machine learning based model; and providing the user assessment report to a display or a data store.

### CROSS REFERENCE TO RELATED APPLICATIONS

This application claims benefit of and priority to U.S. Provisional Patent Application Ser. No. 63/523,761, filed Jun. 28, 2023, the contents of which is herein incorporated by reference in its entirety; and claims benefit of and priority to U.S. Provisional Patent Application Ser. No. 63/523,803, filed Jun. 28, 2023, the contents of which is herein incorporated by reference in its entirety.

### GOVERNMENT INTEREST

This invention was made with government support under grant nos. HD093074, MH121329, and MH120093 awarded by the National Institutes of Health (NIH). The government has certain rights in the invention.

### TECHNICAL FIELD

The subject matter described herein relates generally to autism detection and/or automated motor skills assessment. More particularly, the subject matter described herein includes methods, systems, and computer readable media for early detection of a neurodevelopmental or psychiatric disorder using scalable computational behavioral phenotyping and/or automated motor skills assessment.

### BACKGROUND

Neurodevelopmental or psychiatric (neurodevelopmental/psychiatric) disorders affect many people throughout the world. Current estimates indicate that 1 in 9 children may have or develop a neurodevelopmental and/or psychiatric disorder, such as an autism spectrum disorder (ASD), an anxiety disorder, or an attention deficient and hyperactivity disorder (ADHD). Research has shown that treatments for various behavioral disorders, including autism, can be more effective when diagnosed and treated early. Moreover, early intervention and consistent monitoring can be useful for tracking individual progress and may also be useful for understanding subjects in clinical trials. However, many children are not accurately screened and/or diagnosed as early as possible and/or do not receive adequate care after diagnosis. For example, the average age of autism diagnosis is close to 5 years old in the United States, yet autism may be diagnosed as early as 18 months. Current screening methods are less accurate when administered in real world settings, especially for girls and children of color. Current assessment techniques generally require trained clinicians and/or expensive equipment and can be very time intensive. Hence, current assessment techniques include barriers for early diagnosis and monitoring of many neurodevelopmental/psychiatric disorders.

### SUMMARY

This summary lists several embodiments of the presently disclosed subject matter, and in many cases lists variations and permutations of these embodiments. This summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this summary or not. To avoid excessive repetition, this summary does not list or suggest all possible combinations of such features.

The subject matter described herein includes methods, systems, and computer readable media for early detection of a neurodevelopmental or psychiatric (neurodevelopmental/psychiatric) disorder using scalable computational behavioral phenotyping. In some embodiments, a method for early detection of a neurodevelopmental or psychiatric disorder using scalable computational behavioral phenotyping occurs at a computing platform including at least one processor and memory. The method includes obtaining user related information, wherein the user related information includes metrics derived from a user interacting with one or more applications executing on at least one user device; generating, using the user related information and a machine learning based model, a user assessment report including a prediction value indicating a likelihood that the user has a neurodevelopmental or psychiatric (neurodevelopmental/psychiatric) disorder or risk for such a disorder and a prediction confidence value computed using relative contributions of the metrics to the prediction value generated using the machine learning based model; and providing the user assessment report to a display or a data store.

A system for early detection of a neurodevelopmental or psychiatric disorder using scalable computational behavioral phenotyping is also disclosed. In some embodiments, the system includes a computing platform including at least one processor and memory. In some embodiments, the computing platform is configured for: obtaining user related information, wherein the user related information includes metrics derived from a user interacting with one or more applications executing on at least one user device; generating, using the user related information and a machine learning based model, a user assessment report including a prediction value indicating a likelihood that the user has a neurodevelopmental or psychiatric (neurodevelopmental/psychiatric) disorder or risk for such a disorder and a prediction confidence value computed using relative contributions of the metrics to the prediction value generated using the machine learning based model; and providing the user assessment report to a display or a data store. The assessment report also includes individualized descriptions of the user's unique behavioral profile that can be used to guide treatment planning.

The subject matter described herein includes methods, systems, and computer readable media for automated motor skills assessment. According to one method for automated motor skills assessment includes obtaining touch input data associated with a user using a touchscreen while the user plays a video game involving touching visual elements that move; analyzing the touch input data to generate motor skills assessment information associated with the user, wherein the motor skills assessment information indicates multiple touch related motor skills metrics; determining, using the motor skills assessment information, that the user exhibits behavior indicative of a neurodevelopmental or psychiatric (neurodevelopmental/psychiatric) disorder; and providing, via a communications interface, the motor skills assessment information, a diagnosis, or related data.

A system for automated motor skills assessment is also disclosed. In some embodiments, the system includes a computing platform including at least one processor and memory, where the computing platform is configured for: obtaining touch input data associated with a user using a touchscreen while the user plays a video game involving touching visual elements that move; analyzing the touch input data to generate motor skills assessment information associated with the user, wherein the motor skills assessment information indicates multiple touch related motor skills metrics; determining, using the motor skills assessment information, that the user exhibits behavior indicative of a neurodevelopmental or psychiatric (neurodevelopmental/psychiatric) disorder; and providing, via a communications interface, the motor skills assessment information, a diagnosis, or related data.

The subject matter described herein may be implemented in software in combination with hardware and/or firmware. For example, the subject matter described herein may be implemented in software executed by a processor (e.g., a hardware-based processor). In one example implementation, the subject matter described herein may be implemented using a non-transitory computer readable medium having stored thereon computer executable instructions that when executed by the processor of a computer control the computer to perform steps. Example computer readable media suitable for implementing aspects or portions of the subject matter described herein include non-transitory devices, such as disk memory devices, chip memory devices, programmable logic devices, such as field programmable gate arrays, and application specific integrated circuits. In addition, a computer readable medium that implements the subject matter described herein may be located on a single device or computing platform or may be distributed across multiple devices or computing platforms.

As used herein, the term “node” refers to a physical computing platform including one or more processors and memory.

As used herein, the terms “function” or “module” refer to software in combination with hardware and/or firmware for implementing features described herein. In some embodiments, a module may include a field-programmable gate array (FPGA), an application-specific integrated circuit (ASIC), or a processor.

Although some of the aspects of the subject matter disclosed herein have been stated hereinabove and are achieved in whole or in part by the presently disclosed subject matter, other aspects will become evident as the description proceeds when taken in connection with the accompanying drawings as best described hereinbelow.

### BRIEF DESCRIPTION OF THE DRAWINGS

The patent or application file contains at least one drawing executed in color. Copies of this patent or patent application publication with color drawings will be provided by the Office upon request and payment of the necessary fee.

The subject matter described herein will now be explained with reference to the accompanying drawings of which:

FIG. 1 is a diagram illustrating an example computing platform for early detection of a neurodevelopmental or psychiatric (neurodevelopmental/psychiatric) disorder using scalable computational behavioral phenotyping;

FIGS. 2A-2F are diagrams illustrating aspects of a workflow and output for detection of a neurodevelopmental/psychiatric disorder;

FIGS. 3A-3C illustrate example statistics associated with a study involving a trained machine learning based prediction or diagnostic model and examples of digital phenotypic profiles generated for unique users;

FIG. 4 is a diagram illustrating an example process for early detection of a neurodevelopmental/psychiatric disorder using scalable computational behavioral phenotyping;

FIG. 5 is a diagram illustrating an example computing platform for automated motor skills assessment.

FIG. 6 is a diagram illustrating an example process for automated motor skills assessment.

FIG. 7 is a series of distributions for each of the app variables. Distributions are shown for all autistic (orange) and neurotypical (blue) participants and for one neurotypical (red) and one autistic (purple) participant who were correctly classified. The distributions represent the actual empirical distributions for the autistic (N=49) and neurotypical (N=328) study cohorts.

FIGS. 8A-8C. SenseToKnow app administration and movies. FIG. 8A is an illustrative example of the app administration, a toddler watches a set of developmentally appropriate movies on a tablet, which are strategically designed to elicit early signs of a developmental disorder, including autism. In FIG. 8B, participants play a “bubble popping” game After watching the movies. FIG. 8C is a series of illustrations of the movies presented (in order), from left to right. The movies are referred to as: Floating Bubbles, Dog in Grass, Spinning Top, Mechanical Puppy, Blowing Bubbles, Rhymes and Toys, Make Me Laugh, Playing with Blocks, and Fun at the Park. Around each image representing the movies, a green/yellow box indicates if the movies present mainly social or non-social content. Movies are presented in English or Spanish and include actors of diverse ethnic/racial backgrounds.

FIG. 9. App variables pairwise correlation coefficients. “W,” “M,” and “S” denote Weak, Medium, and Strong associations, respectively. An association between two variables was considered weak if their Spearman rho correlation coefficient was higher than 0.3 in absolute value, 0.5 for a medium association, and 0.5 for a strong association. *: p-value<0.05; **: p-value<0.01; ***: p-value<0.001.

FIG. 10. Rate of missing data per app variables. For each variable, we computed the number of missing data over the sample size. As we can observe, the rate of missingness is relatively low, with a higher percentage in the case of the average delay when responding to the name calls. This is expected since participants who did not respond to the name calls miss this variable.

FIG. 11. Distribution of the prediction confidence score for the autistic and neurotypical groups. Participants having a prediction confidence score closer to 0 or 1 correspond to app variables either consistently related to neurotypical or autistic behavioral phenotypes.

FIG. 12: Sample of one of the XGBoost optimized trees. The final leaf score attributed to a participant on this tree depends on the value of their app variables. The final prediction is computed averaging the leaf scores of the 100 estimators.

FIG. 13A is an illustration of the computation of the variables contributions for present and missing app variables. FIG. 13B is a bar graph of normalized variables contribution for discriminating autistic from neurotypical participants, including the contribution of missing variables. Note that only the contributions of available variables (in dark blue) are used to compute the variables importance used in the computation of the quality score.

FIGS. 14A-14C: Illustration of the different steps to compute the quality score. (FIG. 14A) Computation of the confidence score for each app variable. This score accounts for how many times the measurement was available and resulted in a confidence score between 0 and 1. (FIG. 14B) Computation of the app variables importance. These scores are normalized and represent the average contribution of each app variable to the model performances. See FIG. 3B. Note that (i) these scores are global (as computed from all participants' SHAP values) and fixed to compute the quality score of all participants and (ii) missing data were discarded following the methodology explained in FIG. 13 to estimate the true importance of each app variable when they were available. (FIG. 14C) Computation of the quality score as a weighted sum of the confidence score by the variables importance.

FIG. 15. Distribution of the quality score of the analyzed cohort. A quality score close to 1 implies an administration with all app variables computed, while a quality score close to 0 implies that none of the app variables were collected during the assessment.

FIG. 16. Importance plots of the app variables to the model's prediction. The x-axis represents the variables range, with ticks on the left representing the missing variables. The y-axis represents the normalized SHAP values of that variable. Each point corresponds to the SHAP value of the corresponding variable for each of the participants from the neurotypical and autistic groups. SHAP values of participants #1 and #2, presented in the manuscript, are shown with large purple and red dots, and participants #3, #4, and #5, presented in EXAMPLE 2, are reported in grey, green, and sky-blue dots, respectively.

FIGS. 17A-17C: Additional illustrative digital phenotypes. (FIG. 17A) An autistic girl who did not receive a commonly used screening questionnaire, the M-CHAT-R/F. Her digital phenotype shows a mix of autistic and neurotypical-related variables, as illustrated in her SHAP values and prediction confidence score of 0.48. (FIG. 17B) App variables contributions of a misclassified neurotypical participant, whose digital phenotype was typically associated with autistic behavioral patterns. (FIG. 17C) App variables of a misclassified autistic participant, whose digital phenotype was typically associated with neurotypical patterns. Note that even misclassifications are provided with detailed explanations by the proposed framework. SHAP values of these participants are reported in FIG. 16 with grey, green, and sky-blue points.

FIGS. 18A-18D. P-values and associated effect sizes for group comparisons of the touch-related features for autistic versus neurotypical participants in study 1 (FIGS. 18A and 18B), and autistic participants with and without co-occurring ADHD in study 2 (FIGS. 18C and 18D). (1) number of touches; (2) number of pops; (3) bubble popping rate; (4) double touch rate; (5) screen exploratory percentage; (6) number of targeted; (7) number of transitions; (8) repeat percentage; (9) touch duration; (10) touch length of the touch motion; (11) touch velocity; (12) applied force; (13) distance to the center; (14) popping accuracy; (15) average variation of the popping accuracy; (16d) variability of the average popping accuracy; (16e) variability of the maximum popping accuracy; (17) number of touches per target; (18) touch frequency; (19) time spent on a targeted bubble. a Mean; b Median; c Standard deviation; *p<0.05; **p<0.01; ***p<0.001; *4:p<0.00001; *5:p<0.000001. P-values were computed using a one-way ANCOVA. Red dotted line indicates statistical significance at level 5%. P-values were corrected using Benjamini-Hochberg procedure to control for FDR. Red, orange, and green dotted lines indicate standard levels associated with a low (η²=0.01), middle (η²=0.04), and large (η²=0.14) effect size.

FIGS. 19A-19F. Group comparisons of distributions of several touch-related features for autistic versus neurotypical participants in study 1. These motor-related features show statistically significant differences between the groups (except for the number of touches). The extracted features presented here are detailed in the features extraction section. P-values were computed using a one-way ANCOVA, and corrected using Benjamini-Hochberg procedure to control for FDR. Effect sizes are denoted as η². The line within the boxplot represents the median, the box represents the interquartile range, and the whiskers show extreme values. Scatter points show feature values for each participant.

FIGS. 20A-20K. Boxplot of the distribution of a few touch-related features for autistic versus neurotypical children, for the study 1 sample. Analysis was performed matching participants' age and experience across the neurotypical and autistic groups. These motor-related features show statistically significant differences between autistic and neurotypical toddlers (except for the number of touches). The extracted features presented here are detailed in the features extraction section. P-values were corrected using Benjamini-Hochberg procedure to control for FDR. Effect sizes are denoted as h!. The line within the boxplot represents the median, the box represents the interquartile range, and the whiskers show extreme values. Scatter points show feature values for each participant.

FIGS. 21A-21F. Group comparisons of distributions of several touch-related features for autistic versus autistic+ADHD participants in study 2. These motor-related features show statistically significant differences between the groups (except for the number of touches). The extracted features presented here are detailed in the features extraction section. P-values were computed using a one-way ANCOVA, and corrected using Benjamini-Hochberg procedure to control for FDR. Effect sizes are denoted as η2. The line within the boxplot represents the median, the box represents the interquartile range, and the whiskers show extreme values. Scatter points show feature values for each participant.

FIG. 22A-22G. Boxplot of the distribution of a few touch-related features for autistic children and those co-occurring ADHD, for the study 2 sample. Analysis was performed matching participants' age, IQ, and experience across the autistic participants with and without ADHD. These motor-related features show statistically significant differences between autistic participants with and without ADHD (except for the number of touches). The extracted features presented here are detailed in the features extraction section. P-values were corrected using Benjamini-Hochberg procedure to control for FDR. Effect sizes are denoted as η². The line within the boxplot represents the median, the box represents the interquartile range, and the whiskers show extreme values. Scatter points show feature values for each participant.

FIGS. 23A and 23B. Receiver operating characteristic (ROC) curves and areas under the curve (AUC). ROCs and AUCs were obtained using logistic regression classifiers trained on a single, two, and three features, when differentiating autistic and neurotypical toddlers in study 1 (left) and autistic children aged 3-10 years with and without co-occurring ADHD in study 2 (right) samples. In both studies, level of group discrimination improves when adding features to the model. Confidence intervals were computed with the Hanley and McNeil method at 95% level. F1: Average length [mm]; F2: Average touch duration [s]; F3: Average time spent [s]; F4: Average distance to the center [mm]; F5: Number of targets]; F6: Screen exploratory percentage.

FIGS. 24A-24G. Correlations between computed motor-related variables (columns) and clinical measures (rows) for the study 1 sample. The height of the bar indicates the value of the partial correlation between a specific game variable and a clinical measure. (1) number of touches; (2) number of pops; (3) bubble popping rate; (4) double touch rate; (5) screen exploratory percentage; (6) number of targeted; (7) number of transitions; (8) repeat percentage; (9) touch duration; (10) touch length of the touch motion; (11) touch velocity; (12) applied force; (13) distance to the center; (14) popping accuracy; (15) average variation of the popping accuracy; (16d) variability of the average popping accuracy; (16e) variability of the maximum popping accuracy; (17) number of touches per target; (18) touch frequency; (19) time spent on a targeted bubble. a Mean; b Median; c Standard deviation; *p<0.05; **p<0.01; ***p<0.001. P-values were computed using a Student's t-test. Red dotted line indicates level of correlation of 0.3 and −0.3. MSEL Mullen Scales of Early Learning, ELC Early Learning Composite Score, EL Expressive Language T-Score, FM Fine Motor T-Score, VR Visual Reception T-Score, ADOS-2 Autism Diagnostic Observation Schedule—Second Edition.

FIGS. 25A-25H. Correlations between computed motor-related variables (columns) and clinical measures (rows), for the study 2 sample. The height of the bar indicates the value of the partial correlation between a specific game variable and a clinical measure. (1) number of touches; (2) number of pops; (3) bubble popping rate; (4) double touch rate; (5) screen exploratory percentage; (6) number of targeted; (7) number of transitions; (8) repeat percentage; (9) touch duration; (10) touch length of the touch motion; (11) touch velocity; (12) applied force; (13) distance to the center; (14) popping accuracy; (15) average variation of the popping accuracy; (16d) variability of the average popping accuracy; (16e) variability of the maximum popping accuracy; (17) number of touches per target; (18) touch frequency; (19) time spent on a targeted bubble. a Mean; b Median; c Standard deviation; *P<0.05; **P<0.01; ***P<0.001. Spearman's rho correlation was used to assess the relationship between motor features and clinical variables, with statistical significance computed using a Student's t-distribution. Red dotted line indicates level of correlation of 0.3 and −0.3. ADOS-2 Autism Diagnostic Observation Schedule—Second Edition, ADHD-RS Attention Deficit/Hyperactivity Disorder Rating Scale, DAS Differential Abilities Scale, GCA General Conceptual Ability, NVC Non-Verbal Composite.

FIGS. 26A-26I. Illustration of the bubble popping game and the touch-based features extracted. This game is composed of 5 vertical tracks with bubbles appearing from the bottom and moving upwards. Any time a bubble is touched, the bubble pops, making a distinct popping sound releasing a cartoon animal character inside the bubble.

When the bubble is popped, it appears again (same cartoon character) from the bottom of the same lane, otherwise a random one appears after the bubble exits the screen from the top. FIGS. 26A-26I graphically represent many of the touch-based features extracted from the game (see Methods).

FIG. 27A. Example of the Z-acceleration (orthogonal direction of the screen) of the iPad during the game, with duration of the child's touches represented. FIG. 27B. Example of the computed iPad energies. To compute a proxy for the force engaged by the child when touching the screen, we integrated the acceleration signal—indicative of the device's dynamical response to a touch—over the duration of a touch (grey shades), and then sum over the X, Y and Z components, as explained in the Algorithms 1 and 2.

FIG. 28A. Illustration of the popping accuracy assignment for all the sample points of a touch. FIGS. 28B-28D. Popping accuracy evolution for three different participants. The popping accuracy provides information about the evolution of the accuracy of a child while their finger is touching the screen. FIG. 28A. Each sample point of a child's touch was assigned a score between 0 and 1 reflecting its closeness to the bubble. FIG. 28B. This participant showed high popping accuracy across their touches, low intra-touches variability (average variation of the popping accuracy), and low inter-touches variability (variability of the average popping accuracy). FIG. 28C. This participant showed medium popping accuracy, low intra-touches variability, but high inter-touches variability. FIG. 28D. This participant showed medium popping accuracy, high intra-touches variability, and low inter-touches variability.

FIG. 29A. Example of a chronogram of gameplay events. FIG. 29B. Diagram depicting how a touch was assigned to a bubble. FIG. 29A. The grouped touches correspond to several touches intended to touch the same bubble. We assumed that a touch was intended to touch a specific bubble if the distance between the edge of that bubble and the touch onset location was less than 3.71 cm, corresponding to 2R. Sub-figure FIG. 29B illustrates how we made the association between a touch and a bubble.

FIGS. 30A-30F. Movies used in this study: The social movies were (FIG. 30A) Blowing Bubbles, (FIG. 30B) Spinning Top, (FIG. 30C) Rhymes and Toys, and (FIG. 30D) Make Me Laugh; the non-social movies were, (FIG. 30E) Mechanical Puppy, and (FIG. 30F) Dog in Grass Right-Right-Left.

FIGS. 31A and 31B. Landmarks and associated features, representing (FIG. 31A) the 49 landmark points and (FIG. 31B) an example representing the time-series of extracted features such as headpose angles, landmarks' dynamics associated with eyebrows and mouth regions, and affect probabilities for a 20 second window of a randomly chosen TD participant. The dotted circles with red colored landmark points (in FIG. 31A) were used for the preprocessing (face transform to canonical form). The blue dots represent landmarks for the eyebrows and the green ones were for mouth. The red colored masks (in FIG. 31B) exemplify filtered-out intervals in the time-series.

FIGS. 32A and 32B. Schematic of measuring multiscale entropy (MSE) using sample entropy (SampEn) with missing values. (FIG. 32A) Example of generating coarse-grained time-series data for three different scales. (FIG. 32B) Example of calculating SampEn with m ¼ 2. Template vectors of m and m [symbol] 1 were defined from a time-series signal with varying amplitude having data points across time (columns, N ¼ 28) lying on a specific tolerance (r, represented as equally spaced dotted-lines/rows). The dotted circles indicate that the specific data point was missing in the original time-series. The negative natural logarithm was computed for the ratio between the number of matching (or repeating) templates associated with the m and m [symbol] 1 dimensional vectors. For example, in the 2-dimensional (m¼2) vectors case, the template vectors denoted as · was repeated 2 times, # was repeated 3 times, * was repeated 4 times, and so on. In total, the repeated vector sequences was Cm¼16. Similarly, for the m [symbol] 1-component vectors the number of repeated vector sequences was Cm [symbol] ¼5. Then the SampEn for this example was —In(5/16). Please note that the embedding vectors having missing data points marked with a red rectangular area were not considered while estimating Cm and Cm [symbol] 1. Instead, if we would have just concatenated the data points, the template 16 or 17 (without considering the missing value) would have matched with 25 and 26, thereby (artificially) increasing Cm without necessarily increasing Cm [symbol] 1, or vice versa, leading to inaccurate results.

FIG. 33. Results of the MSE for all the social and non-social movies with significant differences (p-value) between the ASD and TD groups, represented across the scales (1-30). The number of participants in the ASD and TD group varies across the movies since we have considered only individuals having at least 40% of valid data per each movie. Symbols: triangle: p<0.0001, +: p<0.001, circles: p<0.01 and ⋆: p<0.05.

FIG. 34. Comparative analysis of integrated entropy between the ASD and TD groups for all the social and non-social movies. Symbols: triangle—p<0.00001 and circle—p<0.01. The numbers inside the figure represent the effect size r. BB=Blowing Bubbles, MML=Make Me Laugh, ST=Spinning Top, RAT=Rhymes and Toys, RRL=Dog in Grass RRL, and Mpuppy=Mechanical Puppy.

FIG. 35. ROC curves for each of the social movies. The AUC of the ROC was higher while the classifier was trained with only integrated entropy compared to either of PositiveEnergy0 or the combination of both (except for Spinning Top) the measures.

FIG. 36. Images of movies with high social content, and movies with low social content (nonsocial).

FIG. 37. Plot of facial landmarks consisting of 2D-positional coordinates.

FIG. 38. Time-series plots of the head movement rate per 1/3 s for each of the movies.

FIG. 39. Displays of the MSE of the head movements across 1-30 scales.

FIG. 40. Testing for individual movies, Mean_accelHM and Integrated_entropyHM distinguished the autistic and neurotypical groups.

### DETAILED DESCRIPTION

The subject matter described herein discloses methods, systems, and computer readable media for early detection of a neurodevelopmental or psychiatric disorder using scalable computational behavioral phenotyping and/or automated motor skills assessment. Autism is a neurodevelopmental condition associated with challenges in socialization and communication. Early detection of autism ensures timely access to intervention. Autism screening questionnaires have low accuracy when used in real world settings, such as primary care.

Behavioral signs of autism emerge between 9-18 months and include reduced attention to people, lack of response to name, differences in facial expressions, and motor delays. Commonly, children are screened for autism at the 18-24-month well-child visit using a parent questionnaire, which has shown to have lower accuracy in primary care settings, particularly for girls and children of color. There is a need for objective, scalable screening tools to increase the accuracy of autism screening with a goal of reducing disparities in access to early intervention and improving outcomes.

Eye tracking of social attention has been investigated as an objective early autism biomarker. An eye-tracking measure of social attention evaluated in 1,863 12-48-month-old children showed strong specificity (98.0%), but poor sensitivity (17.0%). The complex presentation of autism may be better captured by quantifying multiple autism-related behaviors. To this end, we developed an app which is administered on a tablet or smartphone and displays brief, strategically designed stimuli while the child's behavioral responses are recorded via the device's frontal camera and quantified via computer vision analysis (CVA) and machine learning (ML). Using the app, various computational approaches to measure individual autism-related behaviors, including social attention, facial expressions and dynamics, head movements, response to name, blink rate, and motor skills, can be utilized.

The subject matter described herein includes methods, systems, methods, or aspects related to using ML to create a novel algorithm that combines multiple behaviors and assesses the feasibility and accuracy of the app for fully automatic autism detection. For example, an algorithm, an app, a ML based model, or a related module in accordance with various aspects of the subject matter described herein can utilize novel methods for generating individualized user assessment reports. In this example, a user assessment report may quantify app administration quality (e.g., a quality score) and quantify the confidence of autism prediction (e.g., a prediction confidence score) for a given user (e.g., a child between 17 and 36 months old), and for explaining the user's unique digital phenotype profile.

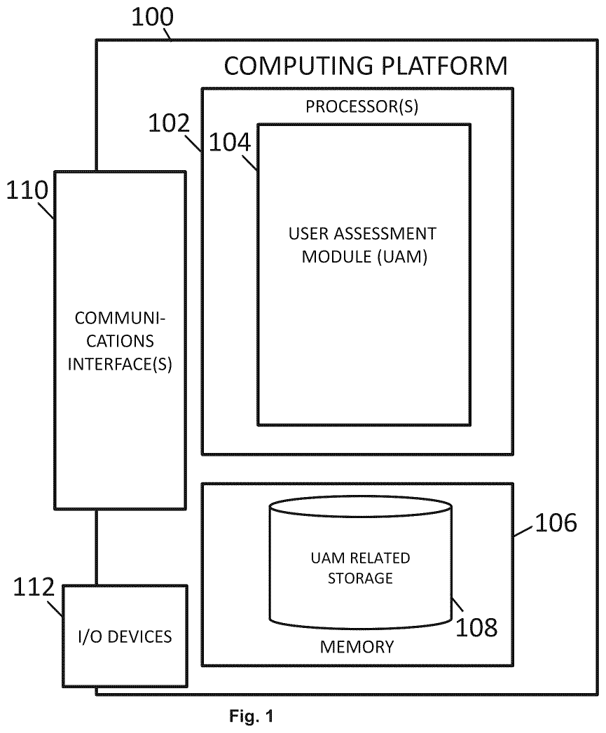

The subject matter described herein includes results of a study assessing the accuracy of an autism screening digital application (e.g., a mobile app) administered during a pediatric well-child visit to 475 17-36-month-old children, 49 diagnosed with autism and 98 with developmental delay without autism. In the study, the app displayed stimuli designed for eliciting behavioral signs of autism, which were quantified using computer vision analysis (CVA) and machine learning (ML). In particular, up to twenty-three digital phenotypes (e.g., behavioral traits observed from user interaction with a digital app) based on CVA or touch quantified social attention, facial expressions and dynamics, blink rates, head movements, response to name, and motor skills (see FIG. 1 and EXAMPLE 2 below) were obtained or derived in the study. Quality scores reflecting the amount of available app variables weighted by their predictive power were high (median score=93.9%, Q1-Q3 [90.0%-98.4%]), with no group differences. A prediction confidence score for accurately classifying an individual child indicated that, at the 20% threshold, 311/377 administrations were rated high confidence (see EXAMPLE 2 below).

The subject matter described herein includes an ML algorithm (e.g., a trained model) for receiving as input a digital phenotype profile (e.g., observable behavioral traits represented by metrics observed or derived from user interaction with a digital app executing on a user device) and outputting diagnostic or predictive information. In a study described herein, the ML algorithm combining app derived traits (also referred to as app variables or app features) showed high diagnostic accuracy: area under the receiver operating characteristic curve (AUC)=0.90, sensitivity 87.8%, and specificity 80.8% distinguishing autism versus neurotypical children; AUC=0.86, sensitivity 81.6%, and specificity 80.5% distinguishing autism versus non-autism. Results demonstrate that digital phenotyping is an objective, scalable approach to autism screening in real-world settings.

Further, by providing techniques, mechanisms, and/or methods for early detection of a neurodevelopmental or psychiatric disorder using scalable computational behavioral phenotyping and automated motor skills assessment, diagnosis and/or treatment for various neurodevelopmental/psychiatric disorders (e.g., an autism spectrum disorder (ASD), an anxiety disorder, or an attention deficient and hyperactivity disorder (ADHD)) may be performed quickly and efficiently. Moreover, by providing automated user and motor skills assessments using a camera, a touchscreen, and/or software executing on mobile devices or other relatively inexpensive devices, cost barriers associated with diagnosis and/or treatment of neurodevelopmental/psychiatric disorders may be alleviated. Furthermore, using aspects of the present subject matter, diagnosis and/or treatment for various neurodevelopmental/psychiatric disorders in young children (e.g., ages 1-5) may be facilitated and/or improved over conventional methods, thereby allowing treatments, strategies, and/or intervention methods to be implemented more broadly and earlier than previously possible with conventional methods.

Additional details and example methods, mechanisms, techniques, and/or systems for early detection of autism or related aspects are further described in the EXAMPLES provided herein.

FIG. 1 is a diagram illustrating an example computing platform 100 for early detection of a neurodevelopmental or psychiatric (neurodevelopmental/psychiatric) disorder or risk for such a disorder using scalable computational behavioral phenotyping.

Computing Platform 100 may be any suitable entity (e.g., a mobile device or a server) configurable for generating user assessments using scalable computational behavioral phenotyping. For example, computer platform 100 may include a memory and at least one processor for executing a module (e.g., an app or other software) for early detection of neurodevelopmental or psychiatric (neurodevelopmental/psychiatric) disorder or risk for such a disorder using scalable computational behavioral phenotyping. In this example, computer platform 100 may also include a user interface (e.g., a display or a touchscreen) for providing a video or a video game containing stimuli designed or usable to identify neurodevelopmental or psychiatric (neurodevelopmental/psychiatric) disorder or risk for such a disorder in the user (e.g., a child, an adult, etc.) and a camera (e.g., a video camera) or other sensor(s) (e.g., touchscreen sensor(s)) for capturing user responses or behaviors (e.g., eye gaze, eye movements, visual and/or hand motor skills data (e.g., eye and/or hand movements during gameplay), facial expressions, head poses, head movements, eyebrow and mouth movements, responses to video-based stimuli, attention indicators to various stimuli, and/or responses to name being called). Continuing with this example, the module executing at computing platform 100 may generate and use metrics from the captured user interaction or related data in a prediction or diagnostic model (e.g., a trained machine learning based model that utilizes a multiple tree-based extreme gradient-boosting (XGBoost) algorithm). For example, the prediction model may be used in determining user assessment information and/or a related diagnosis (e.g., a diagnosis of a neurodevelopmental/psychiatric disorder or a related metric, such as a decimal value between 0 and 1 indicating the likelihood of a user having a particular neurodevelopmental/psychiatric disorder). See Chen et al. titled “XGBoost: A Scalable Tree Boosting System” (Proceedings of 22nd ACM SIGKDD International Conference on Knowledge Discovery and Data Mining 2016: 785-94) for additional or example methodological details.

In some embodiments, computer platform 100 or a related module may include functionality for generating a user assessment report using a machine learning based model and a digital phenotype profile (e.g., data indicating behavioral traits obtained from or derived from user interaction with one or more apps executing on computer platform 100 or another device). In such embodiments, the user assessment report may include a prediction value related to a diagnosis (e.g., indicating the likelihood a user has a neurodevelopmental or psychiatric (neurodevelopmental/psychiatric) disorder or risk for such a disorder) generated by the model along with a prediction confidence value indicating a confidence or likelihood that the prediction value is accurate and a quality value indicating whether an assessment should be readministered (e.g., because the metrics (e.g., app-derived features) are poor quality or insufficient for an accurate assessment).

Computing platform 100 may include processor(s) 102. Processor(s) 102 may represent any suitable entity or entities (e.g., one or more hardware-based processor) for processing information and executing instructions or operations. Each of processor(s) 102 may be any type of processor, such as a central processor unit (CPU), a microprocessor, a multi-core processor, and the like. Computing platform 100 may further include a memory 106 for storing information and instructions to be executed by processor(s) 102.

In some embodiments, memory 106 can comprise one or more of random access memory (RAM), read only memory (ROM), static storage such as a magnetic or optical disk, or any other type of machine or non-transitory computer-readable medium. Computing platform 100 may further include one or more communications interface(s) 110, such as a network interface card or a communications device, configured to provide communications access to various entities (e.g., other computing platforms). In some embodiments, one or more communications interface(s) 110 may include a user interface configured for allowing a user (e.g., a diagnostic subject for assessment or an assessment operator) to interact with computing platform 100 or related entities. For example, a user interface may include a graphical user interface (GUI) for providing a questionnaire to user and/or for receiving input from the user and/or for displaying region-based stimuli to a user. In some embodiments, memory 106 may be utilized to store a user assessment module (UAM) 104, or software therein, and a UAM related storage 108.

UAM 104 may be any suitable entity (e.g., software executing on one or more processors) for performing one or more aspects associated with user assessment. In some embodiments, UAM 104 may be configured for early detection of a neurodevelopmental or psychiatric (neurodevelopmental/psychiatric) disorder or risk for such a disorder using scalable computational behavioral phenotyping. For example, UAM 104 may be configured for obtaining user related information, wherein the user related information includes metrics derived from a user interacting with one or more applications executing on at least one user device; generating, using the user related information and a machine learning based model, a user assessment report including a prediction value indicating a likelihood that the user has a neurodevelopmental or psychiatric (neurodevelopmental/psychiatric) disorder or risk for such a disorder and a prediction confidence value computed using relative contributions of the metrics to the prediction value generated using the machine learning based model; and providing the user assessment report to a display or a data store.

In some embodiments, UAM 104 or another entity may provide a diagnostic application (e.g., an autism screening digital application) for users (e.g., small children) that provide various stimuli in various forms (e.g., videos, games, audio, etc.). In such embodiments, UAM 104 or another entity may capture user responses and/or related data (e.g., recordings of the user and environment, touchscreen data, etc.) and may then generate or derive metrics from the user interaction (e.g., using various algorithms, techniques, methods) and use the metrics as input into a machine learning based algorithm (e.g., an XGBoost model) for determining a prediction value. In some embodiments, UAM 104 or another entity may use various techniques or algorithms (e.g., SHAP analysis, a local interpretable model-agnostic explanations (LIME) approach, a permutation importance approach, a feature importance approach, etc.) and related data to determine user-specific model interpretability data, e.g., a prediction confidence value and a quality value.

In some embodiments, computing platform 100 and/or UAM 104 may be communicatively coupled to one or more input or output (I/O) device(s), e.g., a camera, a touchscreen, a mouse, a keyboard, an input sensor, a display, etc. I/O device(s) 112 may represent any suitable entity (e.g., a camera sensor or camera chip in a smartphone) for providing data to a user (e.g., a display) or for obtaining data from or about the user (e.g., a camera for recording visual images or audio and/or a touchscreen for recording touch input). For example, I/O device(s) 112 may include a two-dimensional camera, a three dimensional camera, a heat-sensor camera, a touchscreen, touch sensors, etc. In some embodiments, I/O device(s) 112 may be usable for recording a user and user input during a user assessment (e.g., while the user is watching a video containing region-based stimuli or playing a video game).

In some embodiments, UAM 104 or another entity may quantify multiple digital phenotypes, e.g., behavioral traits or attributes, associated with a user (e.g., an ASD assessment subject). For example, UAM 104 or another entity may measure 19 CVA-based and 4 touch-based traits. Example digital phenotypes and related methods are discussed below. Additional information regarding the computation of these variables, missing data rate, and their pairwise correlation coefficients are further discussed EXAMPLE 2 below.

Facing forward: During social and non-social videos, UAM 104 or another entity may compute the average percentage of time that a user faced the screen. In some embodiments, filtering-in frames representing when a user is facing forward may be determined using three rules: eyes were open, estimated gaze was at or close to the screen area, and the face was relatively steady. For example, a digital phenotype or metric “Facing Forward” indicating the average percentage of time that a user faced the screen may be used as a proxy for the user's attention to the videos. See Chang et al. titled “Computational Methods to Measure Patterns of Gaze in Toddlers With Autism Spectrum Disorder” (JAMA Pediatr 2021; 175(8): 827-36) for additional or example methodological details.

Social attention: UAM 104 or another entity may display two videos featuring clearly separable social and non-social stimuli on each side of the screen, designed to capture social/non-social attentional preference. For example, a digital phenotype or metric “Gaze Percent Social” may be defined as the percentage of time the user gazed at the social half of the screen, and the “Gaze Silhouette Score” may reflect how concentrated versus spread out the detected gaze clusters were. See the Chang et al. reference for additional or example methodological details.

Attention to speech: UAM 104 or another entity may display a video with two actors, one on each side of the screen, taking turns in a conversation (see Video 1). For example, a digital phenotype or metric may be defined as the correlation between a user's gaze patterns and the alternating conversation. See the Chang et al. reference for additional methodological details.

Blink rate: During social and non-social videos, UAM 104 or another entity may compute a blink rate for a user. For example, a digital phenotype or metric “Blink Rate” may be used as a proxy to indicate attentional engagement and may be computed using CVA involving a recording of the user's eyes. See Babu et al. (2023) Blink rate and facial orientation reveal distinctive patterns of attentional engagement in autistic toddlers: a digital phenotyping approach. Scientific Reports 13(1): 7158 for methodological details

Facial dynamics complexity: UAM 104 or another entity may compute or estimate the complexity of facial landmarks' dynamics, e.g., by estimating the eyebrows and mouth regions of a user's face using multiscale entropy. For example, a digital phenotype or metric “Mouth Complexity” may be computed for indicating the average complexity of the mouth region during social and non-social videos and another digital phenotype or metric “Eyebrows Complexity” may be computed for indicating the average complexity of the eyebrows region during social and non-social videos. See Krishnappa Babu et al. titled “Exploring complexity of facial dynamics in autism spectrum disorder” (IEEE Trans Affect Comput 2021) for additional or example methodological details.

Head movement: UAM 104 or another entity may compute the rate of head movement (computed from a time series of detected facial landmarks) for social and non-social videos. For example, a digital phenotype or metric “Head Movement” may indicate the average head movement of a user during a video. In some embodiments, complexity and acceleration of head movements may be computed for both social stimuli and non-social stimuli using multiscale entropy and derivative of the time series, respectively. See Krishnappa Babu et al. titled “Complexity analysis of head movements in autistic toddlers” (J Child Psychol Psychiatry 2023; 64(1): 156-66) for additional or example methodological details.

Response to name: UAM 104 or another entity may perform automatic detection of a user's name being called and the user's response to their name, e.g., by using a recording and audio analysis to detect the name being called and CVA techniques and facial landmarks to detect head turns. For example, a digital phenotype or metric “Response to Name Proportion” indicating the proportion of times a user oriented to their name being called, and another digital phenotype or metric “Response to Name Delay” indicating the average delay (in seconds) between the offset of the name call and the head turn. See Perochon et al. titled “A scalable computational approach to assessing response to name in toddlers with autism” (J Child Psychol Psychiatry 2021; 62(9): 1120-31) for additional or example methodological details.

Touch-based visual-motor skills: UAM 104 or another entity may use touch and device kinetic information provided by a touchscreen or other sensors when a user's plays a video game, e.g., a bubble popping game, to quantify touch-based visual motor skills. For example, a digital phenotype or metric “Touch Popping Rate” associated with a bubble popping game may indicate the ratio of popped bubbles over the number of touches; another digital phenotype or metric “Touch Error Variation” associated with the bubble popping game may indicate the standard deviation of the distance between a user's finger position when touching the screen and the center of the closest bubble; another digital phenotype or metric “Touch Average Length” associated with the bubble popping game may indicate the average length of a user's finger trajectory on the screen, and another digital phenotype or metric “Touch Average Applied Force” associated with the bubble popping game may indicate the average estimated force applied on the screen when touching it. See Perochon et al. titled “A tablet-based game for the assessment of visual motor skills in autistic children” (NPJ Digit Med 2023; 6(1): 17) for additional or example methodological details.

In some embodiments, UAM 104 or a related entity (e.g., a medical provider) may administer to a user a therapy or therapies for treating a neurodevelopmental/psychiatric disorder. For example, after performing a user assessment and/or a related diagnosis of a neurodevelopmental/psychiatric disorder, UAM 104 may provide one or more training programs for treating or improving attention, social interaction skills, or motor skills in a user. In this example, the one or more training programs may be based on a number of factors, including user related factors, such as age, name, knowledge, skills, sex, medical history, and/or other information.

In some embodiments, UAM 104 may determine and/or provide user assessment information, a diagnosis, and/or related information (e.g., follow-up information and/or progress information) to one or more entities, such as a user, a system operator, a medical records system, a healthcare provider, a caregiver of the user, or any combination thereof. For example, user assessment information, a diagnosis, and/or related information may be provided via a phone call, a social networking message (e.g., Facebook or Twitter), an email, or a text message. In another example, user assessment information may be provided via an app and/or communications interface(s) 110. When provided via an app, user assessment information may include progress information associated with a user. For example, progress information associated with a user may indicate (e.g., to a caregiver or physician) whether certain therapies and/or strategies are improving or alleviating symptoms associated with a particular neurodevelopmental/psychiatric disorder. In another example, progress information may include aggregated information associated with multiple videos and/or assessment sessions.

Memory 106 may be any suitable entity or entities (e.g., non-transitory computer readable media) for storing various information. Memory 106 may include UAM related storage 108. UAM related storage 108 may be any suitable entity (e.g., a database embodied or stored in computer readable media) storing user data, stimuli (e.g., digital content, games, videos, or video segments), recorded or captured responses, and/or predetermined information. For example, UAM related storage 108 may include machine learning algorithms, algorithms for statistical analysis, SHAP analysis, and/or report generation logic. UAM related storage 108 may also include user data, such as age, name, knowledge, skills, sex, and/or medical history. UAM related storage 108 may also include predetermined information, including information gathered by clinical studies, patient and/or caregiver surveys, and/or doctor assessments.

In some embodiments, predetermined information may include information for analyzing responses; information for determining based responses; information for determining assessment thresholds; coping strategies; recommendations (e.g., for a caregiver or a child); treatment and/or related therapies, information for generating or selecting games, videos, video segments, digital content, or related stimuli usable for a user assessment; and/or other information.

In some embodiments, UAM related storage 108 or another entity may maintain associations between relevant health information and a given user or a given population (e.g., users with similar characteristics and/or within a similar geographical location). For example, users associated with different conditions and/or age groups may be associated with different recommendations, base responses, and/or assessment thresholds for indicating whether user responses are indicative of neurodevelopmental/psychiatric disorders.

In some embodiments, UAM related storage 108 may be accessible by UAM 104 and/or other modules of computing platform 100 and may be located externally to or integrated with UAM 104 and/or computing platform 100. For example, UAM related storage 108 may be stored at a server located remotely from a mobile device containing UAM 104 but still accessible by UAM 104. In another example, UAM related storage 108 may be distributed or separated across multiple nodes.

It will be appreciated that the above described modules or entities are for illustrative purposes and that features or portions of features described herein may be performed by different and/or additional modules, components, or nodes. For example, aspects of user assessment described herein may be performed by UAM 104, computing platform 100, and/or other modules or nodes.

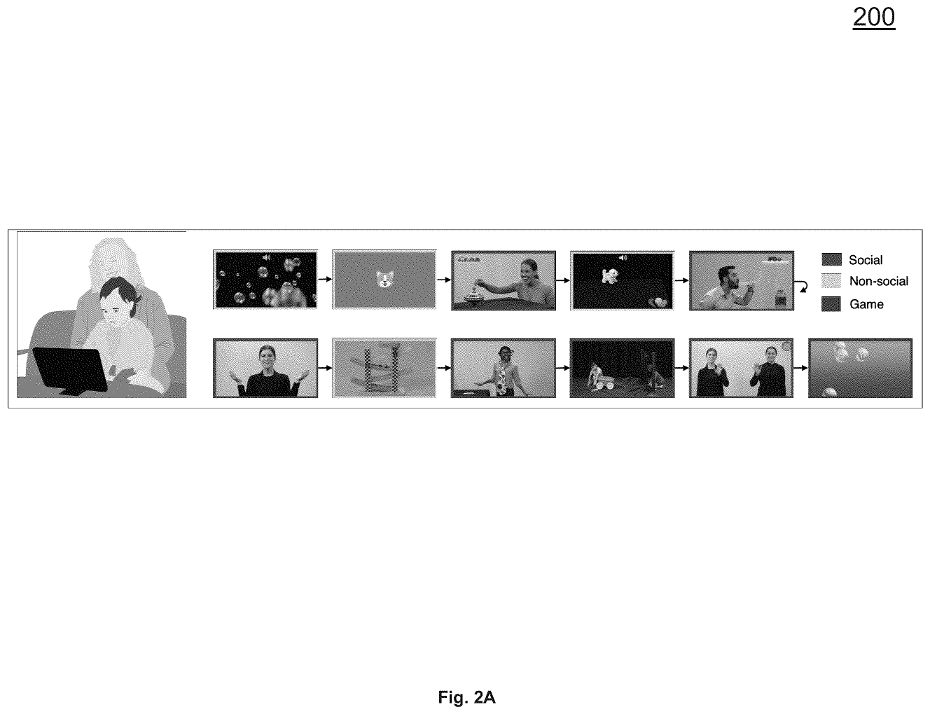

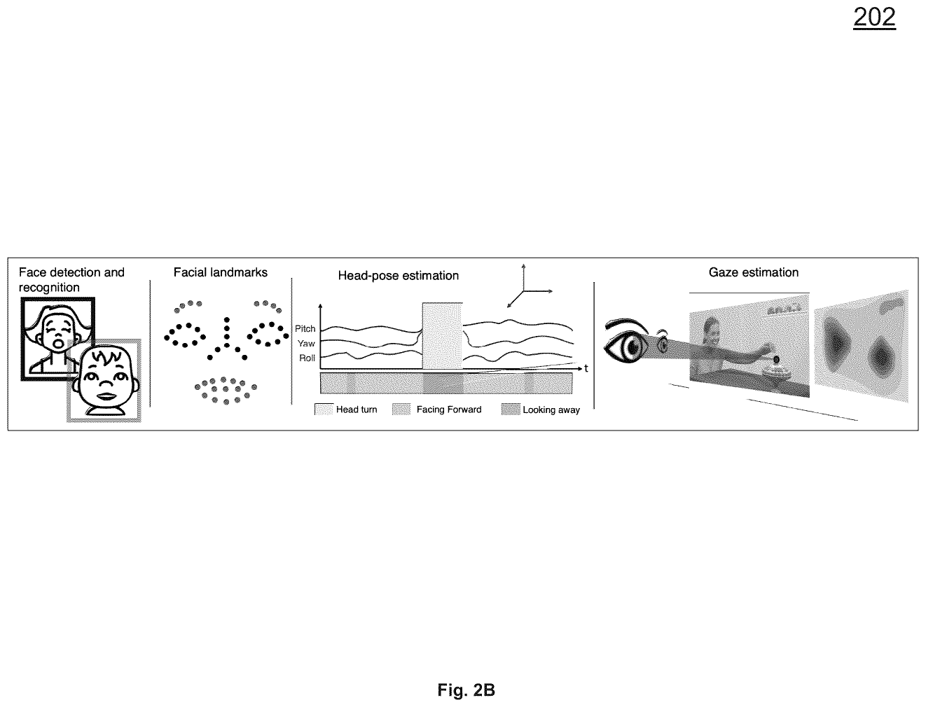

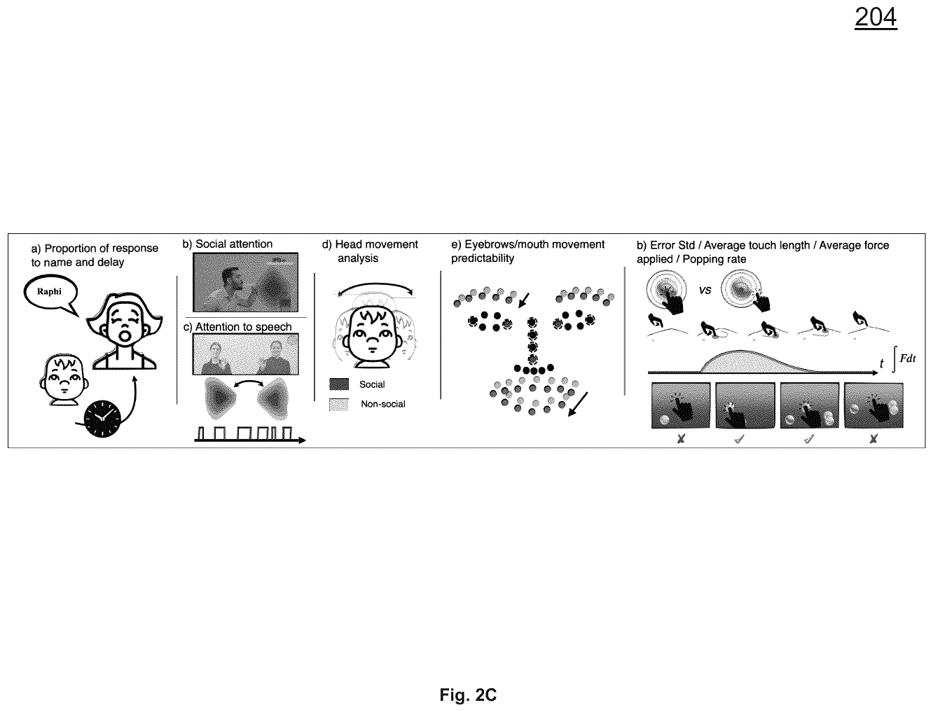

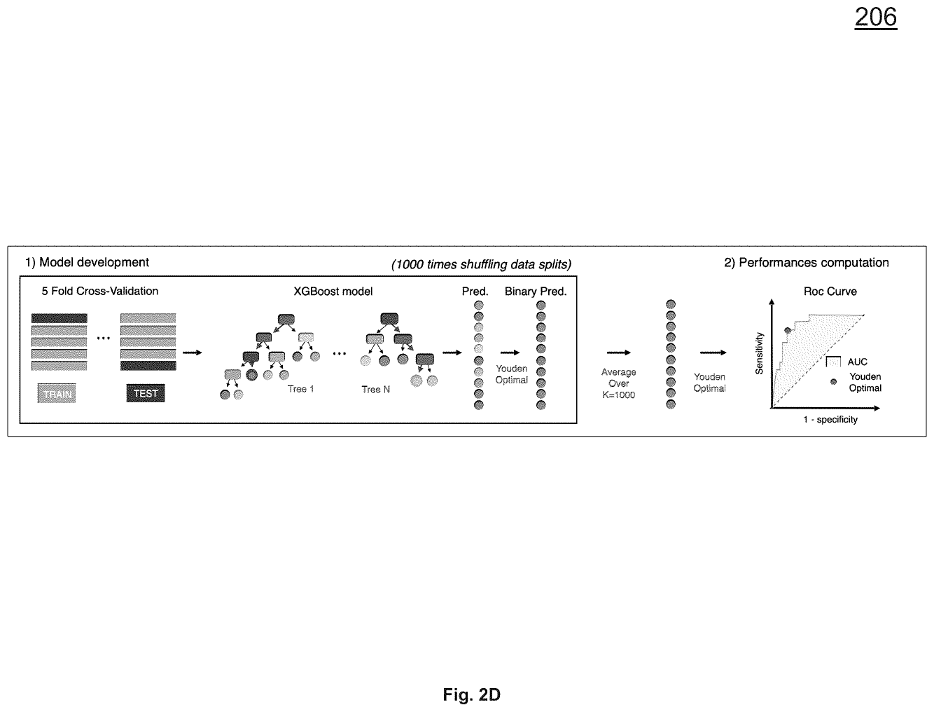

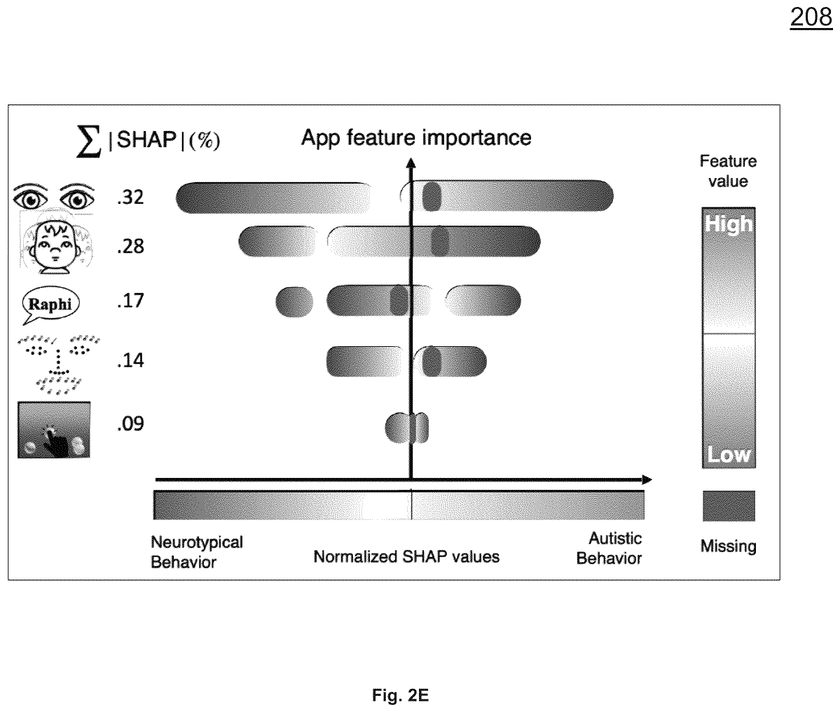

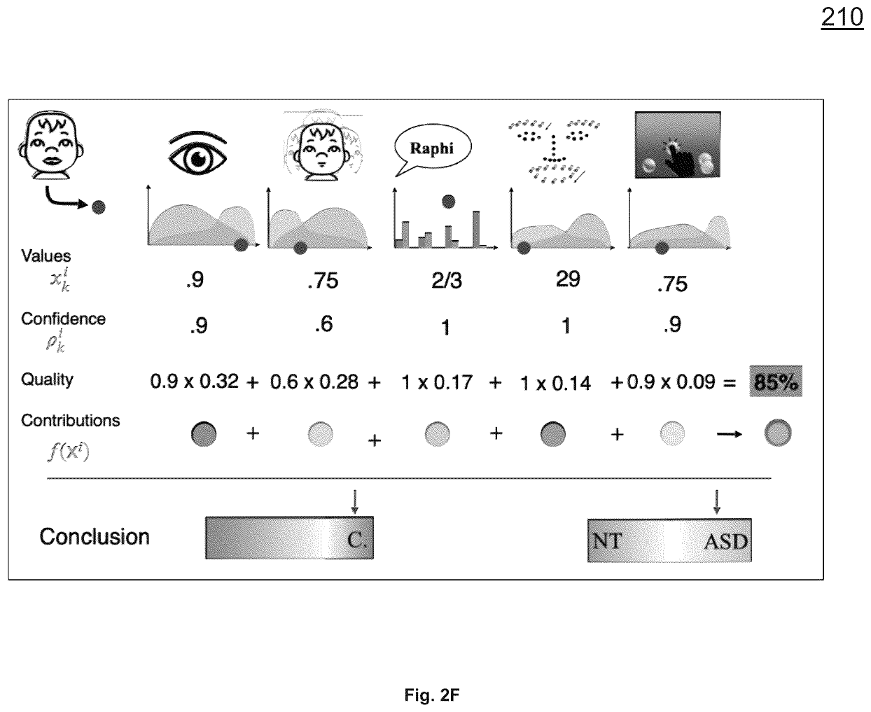

FIGS. 2A-2E are diagrams illustrating aspects of a workflow and output for detecting a neurodevelopmental or psychiatric (neurodevelopmental/psychiatric) disorder or risk for such a disorder. In some embodiments, the workflow represented by FIGS. 2A-2E may involve or utilize UAM 104 or another entity may include an autism screening digital application (e.g., a mobile app) that executes on computing platform 100 or a user device, e.g., a tablet computer. In such embodiments, UAM 104 or another entity FIG. 2A depicts a diagram 200 representing aspects of a data collection environment involving a diagnostic app and strategically-designed, developmentally appropriate stimuli (e.g., digital content) presented by the app. For example, UAM 104 or a related entity may provide or present a video or videos with social and non-social segments and a game for eliciting user interactions. In this example, the videos and games may presented to a user (e.g., a toddler or child sitting with a caregiver) via a display or a touchscreen associated with a user device or computing platform 100. The user's responses and behaviors are recorded using sensors on the device, such as the device's embedded camera or kinetic sensors.

FIG. 2B depicts a diagram 202 representing aspects of feature extraction. For example, UAM 104 or a related entity may analyze captured data (e.g., video recordings) to detect faces and then extract data or related metrics. In this example, feature extraction may involve extracting or obtaining information regarding facial landmarks, head pose estimations, and gaze coordinates, e.g., from one or more video frames using CVA or other techniques.

FIG. 2C depicts a diagram 204 representing aspects of automatic computation of digital phenotypes (e.g., traits, or related metrics) associated with a user. For example, UAM 104 or a related entity may generate or derive metrics representing behavioral traits or features observed or derived from user interaction with a digital app. In this example, digital phenotypes or related metrics may be related to proportion of response to user's name and related delay; social attention; attention to speech, head movement associated with social and non-social stimuli; eyebrows and/or mouth movement; visual motor skills (e.g., touch based skills); hand motor skills; or combinations thereof.

FIG. 2D depicts a diagram 206 representing aspects of model training and performance evaluation. For example, UAM 104 or a related entity may generate or derive metrics representing behavioral traits or features observed or derived from user interaction with a digital app. In this example, digital phenotypes or related metrics may be related to proportion of response to user's name and related delay; social attention; attention to speech, head movement associated with social and non-social stimuli; eyebrows and/or mouth movement; visual motor skills (e.g., touchscreen based skills); hand motor skills; or combinations thereof.

FIG. 2E depicts a diagram 208 representing aspects of model interpretability using SHAP values analysis. For example, UAM 104 or a related entity may generate normalized SHAP interaction values for various app-derived features (e.g., gaze, head movement, name response, eyebrows/mouth movements, visual motor skills (e.g., touchscreen based skills)) where the normalized SHAP interaction values indicate the relative importance to a trained model's prediction or diagnostic value.

FIG. 2F depicts a diagram 210 representing aspects of Report of an individualized app administration showing the participant's unique digital phenotype profile, prediction confidence value, and assessment of the quality of the app administration. For example, UAM 104 or a related entity may generate a user assessment report for indicating what app-derived features and how much of those app-derived features contributed to a user's diagnostic score (e.g., a prediction value) generated by trained model.

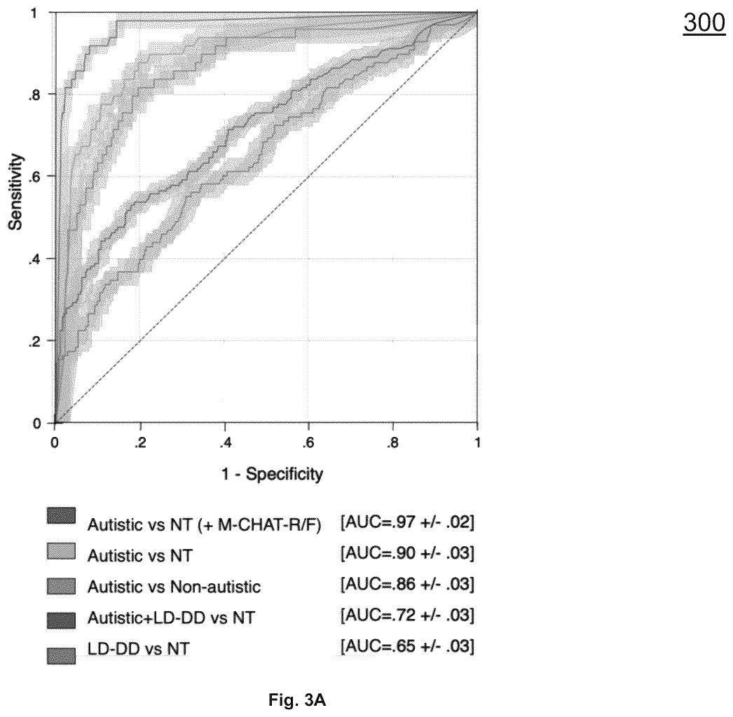

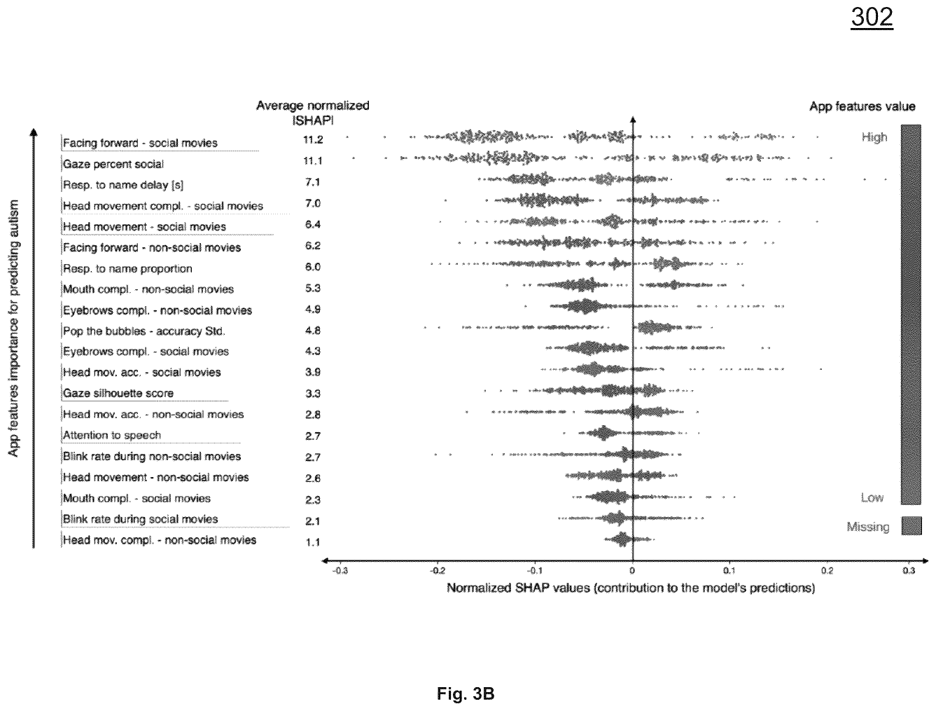

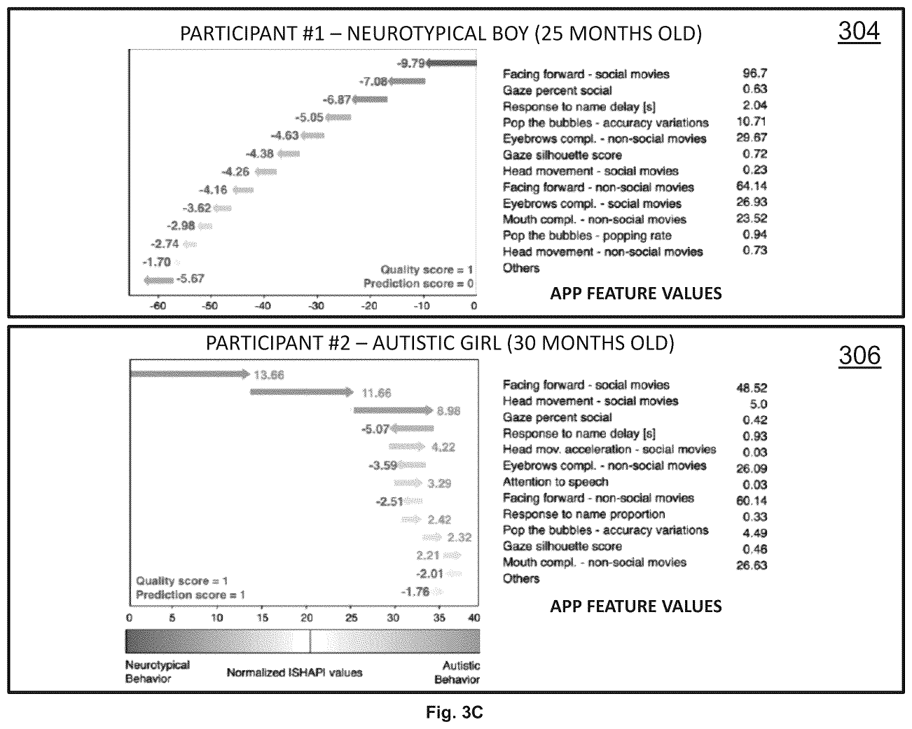

FIGS. 3A-3C illustrate example statistics associated with a study involving a trained machine learning based prediction or diagnostic model. The trained model (e.g., a 1000 decision tree-based XGBoost model) may be usable to classify diagnostic groups. In some embodiments, an XGBoost model may be trained using 5-fold cross-validation where the data is shuffled to compute individual intermediary binary predictions and SHapley Additive exPlanations (SHAP) value statistics (metrics mean and standard deviation).

In the study associated with FIGS. 3A-3C, final prediction confidence scores, between 0 and 1, were computed averaging the K predictions (see EXAMPLE 2 herein below for additional details). Missing data were encoded with a value out of the range of the app variables, such that the optimization of the decision trees considered the missing data as information. Overfitting was controlled using a tree maximum depth of 3, subsampling app variables at a rate of 80%, and using regularization parameters during the optimization process. Diagnostic group imbalance was addressed by weighting training instances by the imbalance ratio. Details regarding the algorithm and hyperparameters are provided in EXAMPLE 2 herein below for additional details. The contribution of the app variables to individual predictions was assessed by the SHAP values, computed for each child using all other data to train the model and normalized such that the features' contributions to the individual predictions range from 0 to 1. See EXAMPLE 2 herein below for additional details.

FIG. 3A depicts a specificity and sensitivity diagram 300 illustrating the area under the receiver operating characteristic curve (AUC) values for classification of autism versus other groups, including results combining app results with the Modified Checklist for Autism in Toddlers-Revised with Follow-up (M-CHAT-R/F), a commonly used screening questionnaire. In FIG. 3A, the acronym “NT” represents Neurotypical and the acronym “DD-LD” represents Developmental Delay and/or Language Delay

Based on the Youden Index, an algorithm integrating all app variables showed a high level of accuracy for classification of autism versus neurotypical development with AUC=0.90, CI [0.87-0.93], sensitivity 87.8% (SD=4.9), and specificity 80.8% (SD=2.3). Restricting to administrations with high prediction confidence, the AUC increased to 0.93 (CI [0.89-0.96]). Extended Data Table 2, EXAMPLE 1, and EXAMPLE 2 show all performance results based on individual and combined app variables. Classification of autism versus non-autism (DD-LD combined with neurotypical) also showed strong accuracy: AUC=0.86 (CI [0.83-0.90]), sensitivity 81.6% (SD=5.4), and specificity 80.5% (SD=1.8). Moreover, accuracy for predicting autism remained high when stratifying groups by sex, race, ethnicity, and age (see Extended Data Table 3, EXAMPLE 1, and EXAMPLE 2).

Nine autistic children scoring negative on the M-CHAT-R/F were correctly classified by the app as autistic as determined by expert evaluation. Among 40 children screening positive on the M-CHAT-R/F, there were 2 classified neurotypical based on expert evaluation and both were correctly classified by the app. Combining the app algorithm with the M-CHAT-R/F further increased classification performance to AUC=0.97 (CI [0.96-0.98]), specificity=91.8% (SD=4.5), and sensitivity=92.1% (SD=1.6).

To address model interpretability, SHAP values may be used to examine the relative contributions of the app variables to the model's prediction and disambiguate the contribution of each feature from their missingness (see EXAMPLE 2 for additional details).

FIG. 3B illustrates the ordered normalized importance of the app variables for the overall model. In particular, FIG. 3B depicts a normalized SHAP value analysis plot diagram 302 showing the app variables importance for an individualized prediction. The x-axis represents the features contribution to the prediction, with positive values associated with an increase in autism prediction. The y-axis represents the app variables in descending order of importance. The blue-red gradient color spans the app variables relevance for the score, from low to high values, with grey samples associated with missing variable. For each app variable, a point represents the normalized SHAP value of a participant.

Referring to FIG. 3B, facing forward during social movies was the strongest predictor (Mean|SHAP|=11.2% (SD=6.0%)), followed by percent of time gazing at social stimuli (Mean|SHAP|=11.1% (SD=5.7%)), and delay in response to a name call (Mean|SHAP|=7.1% (SD=4.9%)). The SHAP values as a function of the app variable values are provided in EXAMPLE 2 below.

SHAP interaction values indicated that interactions between predictors were significant contributors to the model; average contribution of app variables alone was 64.6% (SD=3.4%) and 35.4% (SD=3.4%) for the feature interactions. Analysis of the missing data SHAP values revealed that missing variables were contributing to 5.2% (SD=13.2%) of the model predictions. See EXAMPLE 2 below for additional details. Analysis of the individual SHAP values revealed individual behavioral patterns that explained the model's prediction for each participant.

FIG. 3C depicts two example user assessment reports 304 and 306 illustrating how positive or negative contributions of app variables (e.g., app features) to the predictions can be used to deliver intelligible explanations about the user's app administration and diagnostic prediction, highlight individualized behavioral patterns associated with autism or neurotypical development, and identify misclassified digital profile patterns. This information can be used to inform individualized assessment and therapeutic strategies.

Results of the study depicted in FIGS. 3A-3C demonstrated high accuracy of an ML and CVA-based algorithm using multiple autism-related digital phenotypes assessed via a mobile app (e.g., an autism screening digital application) administered on a tablet in primary care settings for identification of autism in a large sample of toddler-age children. The app captured multiple early signs associated with autism and was robust to missing data. ML allowed optimization of the prediction algorithm based on weighting different behavioral variables and their interactions. High levels of usability and diagnostic accuracy for classification of autism and neurotypical development and autism versus non-autism (neurotypical and other developmental/language delay) were demonstrated. Accuracy for detecting autism did not significantly differ based on the child's age, sex, race, or ethnicity.

In some embodiments, methods for automatic assessment of the quality of the app administration and prediction confidence scores to facilitate the use of a mobile app in real world settings are described herein. For example, UAM 104 may provide an app usable for providing interactive content and can capture user interaction data with the app. In this example, UAM 104 may also use the data to generate metrics or related data that can be inputted into a trained machine learning based model to output a prediction or a diagnostic value. Continuing with this example, UAM 104 may include logic for assessing quality of the app administration and prediction confidence scores using SHAP analysis and then generate a user assessment comprising a quality score and a prediction confidence score. For example, using SHAP analyses, the app output can provide interpretable information regarding which behavioral features are contributing to the overall prediction model and each child's diagnostic prediction. The latter information could be used prescriptively to identify areas in which behavioral intervention should be targeted.

In some embodiments, a generated quality score may indicate whether the app assessment should be readministered. In this example, the quality score can be included with a prediction confidence score which can inform a provider about the degree of certainty regarding the likelihood a child will be diagnosed with autism. Children with uncertain values could be followed to determine whether autism signs become more pronounced, whereas children with high confidence values could be prioritized for referral or begin intervention while waiting for an evaluation.

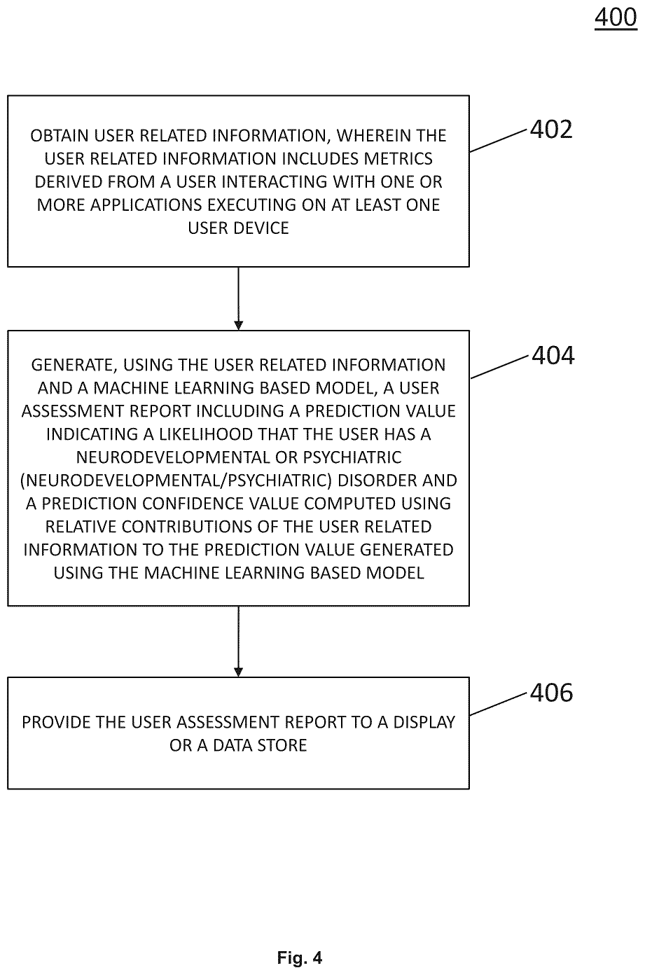

FIG. 4 is a diagram illustrating an example process 400 for early detection of a neurodevelopmental or psychiatric (neurodevelopmental/psychiatric) disorder or risk for such a disorder using scalable computational behavioral phenotyping. In some embodiments, process 400 described herein, or portions thereof, may be performed at or by computing platform 100, UAM 104, and/or another module or node. For example, computing platform 100 may be a mobile device, a computer, or other equipment (e.g., a computerized chair or room) and UAM 104 may include or provide an application running or executing on computing platform 100. In some embodiments, process 400 may include steps 402-406.

In step 402, user related information may be obtained. In some embodiments, user related information may include metrics derived from a user interacting with one or more applications executing on at least one user device. For example, user related information may include a digital phenotype profile or related information indicating a user's behavioral traits or characteristics.

In step 404, a user assessment report may be generated using the user related information and a machine learning based model. In some embodiments, a user assessment report may include a prediction value indicating a likelihood that the user has a neurodevelopmental or psychiatric (neurodevelopmental/psychiatric) disorder or risk for such a disorder and a prediction confidence value computed using relative contributions of the metrics to the prediction value. For example, a user assessment report may be a customized report for indicating what user metrics (e.g., captured application variable data and/or scores) contributed to a user's diagnostic score (e.g., a prediction value) generated by a machine learning based model.

In step 406, the user assessment report may be provided to a display or a data store. For example, a user assessment report may be generated using UAM 104 or a machine learning based model therein and provided to a user (e.g., a subject, a parent, or a medical provider), e.g., via a display device and/or communications interface(s) 110 (e.g., a GUI). In another example, a user assessment report may be generated and stored in memory 106 or UAM related storage 108.

In some embodiments, process 400 may also include administering to the user a therapy for treating the neurodevelopmental/psychiatric disorder. For example, UAM 104 executing on a smartphone may display one or more therapeutic videos for improving a user's attention span for various types of stimuli (e.g., social stimuli), including coaching in strategies that caregivers can use at home to promote social and language skills. In another example, UAM 104 executing on a smartphone may provide interactive content or recommendations to improve a user's social interaction skills and/or motor skills. Another example may involve recommendations for coaching caregivers that promote specific skills, such as learning, communication, and social interaction.

In some embodiments, a user assessment report may include an assessment administration quality value. For example, an assessment administration quality value indicates whether a user assessment should be readministered or retaken. In another example, an assessment administration quality value may be computed based on user metrics (e.g., derived from user interaction with a diagnostic application) weighted by their relative contributions to the prediction value.

In some embodiments, computing a prediction confidence value may include performing a model interpretability analysis (e.g., a SHAP analysis, a LIME analysis, a permutation importance analysis, a feature importance analysis, etc.) involving metrics and a machine learning based model. For example, using SHAP or LIME analysis, UAM 104 or another entity may explain or interpret how various app variables (e.g., app-derived metrics) affect the model's behavior or output. In this example, using normalized SHAP interaction values, UAM 104 or another entity may identify the relative importance of each potential app variable to the model's output, e.g., at an overall level or population level. Continuing with this example, UAM 104 or another entity may also use normalized SHAP interaction values for the actual app variables obtained for a particular user (e.g., the actual app variables may be a subset of the potential app variables that can be obtained or derived) to determine how those particular app variables affected the user's particular prediction value generated by the model.

In some embodiments, performing a model interpretability analysis may include generating normalized SHAP interaction values associated with app-derived metrics and using the normalized interaction values in generating a user-specific prediction profile indicating how the user's metrics affected the user's diagnosis or prediction value. For example, SHAP value analysis may provide information about the relative contribution of each of the potential app-derived metrics to the prediction output (e.g., ASD or neurotypical) of a model (e.g., at a population level) and may also provide information usable when generating a user's unique profile indicating what specific metrics (e.g., the user's digital phenotype profile) and to what extent these metrics contributed to the user's diagnosis or prediction value. These metrics can be used for treatment planning and to monitor progress in treatment.

In some embodiments, a machine learning based model may include an XGBoost algorithm. For example, a trained XGBoost model may comprise or utilize multiple decision trees (e.g., 1,000 trees) that are trained using 5-fold cross-validation where the data is shuffled to compute individual intermediary binary predictions.

In some embodiments, obtaining user related information may include providing stimuli (e.g., interactive digital content, videos, video games, etc. for eliciting behavioral responses) to the user via a display, capturing user interaction data (e.g., recording of user face, eye, and/or posture, touchscreen input data, user selections, etc.) using one or more input devices (e.g., I/O device(s) 112), and generating metrics related to facial orientation, attention, social attention, facial expressions, head movements, eye movements, gaze, eyebrow movements, mouth movements, user responses to name, blink rate, hand motor skills, visual motor skills, or any combinations thereof.

In some embodiments, a neurodevelopmental/psychiatric disorder may an ASD, an ADHD, or an anxiety disorder diagnosis.

In some embodiments, user assessment and actionable guidance information or related data may be provided to a user, a medical records system, a service provider, a healthcare provider, a system operator, a caregiver of the user, or any combination thereof. For example, e.g., where information is provided to a clinician or a medical professional, a user assessment may include stimuli used in a test, recording of the user during the test, test results, and/or other technical or clinical information, such as recommendations for further assessment or treatment planning. In another example, e.g., where information is provided to a parent, a user assessment may include a metric associated with an easy to understand scale (e.g., 0-100%) for indicating the likelihood of a user (e.g., a child) having a particular neurodevelopmental/psychiatric disorder and useful suggestions for improving one or more related symptoms associated with neurodevelopmental/psychiatric disorder.

In some embodiments, computing platform 100 may include a mobile device, a smartphone, a tablet computer, a laptop computer, a computer, a user assessment device, or a medical device.

It will be appreciated that process 400 is for illustrative purposes and that different and/or additional actions may be used. It will also be appreciated that various actions described herein may occur in a different order or sequence.

It should be noted that computing platform 100, UAM 104, and/or functionality described herein may constitute a special purpose computing device. Further, computing platform 100, UAM 104, and/or functionality described herein can improve the technological field of diagnosing and treating various neurodevelopmental/psychiatric disorders by providing mechanisms for early detection of neurodevelopmental or psychiatric (neurodevelopmental/psychiatric) disorder or risk for such a disorder using scalable computational behavioral phenotyping and/or for providing a user assessment report indicating a prediction value and a prediction confidence value. Moreover, such mechanisms can alleviate various barriers, including costs, equipment, and human expertise, associated with conventional (e.g., clinical) methods of diagnosis and treatment of neurodevelopmental/psychiatric disorders.

The subject matter described herein for early detection of a neurodevelopmental or psychiatric (neurodevelopmental/psychiatric) disorder or risk for such a disorder using scalable computational behavioral phenotyping improves the functionality of user assessment devices and equipment by providing mechanisms (e.g., a user assessment algorithm or a machine learning based algorithm) that generates a user assessment regarding the likelihood of an ASD diagnosis using user related information (e.g., a digital phenotype profile comprising data obtained or derived from user interaction with one or more applications executing on a user device).

It should also be noted that computing platform 100 that implements subject matter described herein may comprise a special purpose computing device usable for various aspects of user assessments, including obtaining metrics associated with a user's digital phenotype profile, generating and using a diagnostic or prediction model using the metrics or other data to output a prediction or a diagnosis associated with the user; and generating a user assessment report including a prediction value indicating a likelihood that the user has neurodevelopmental or psychiatric (neurodevelopmental/psychiatric) disorder or risk for such a disorder and a prediction confidence value and/or an assessment administration quality value. A prediction confidence value can be used to prioritize assessment and therapy services in the context of long wait-lists for such services.

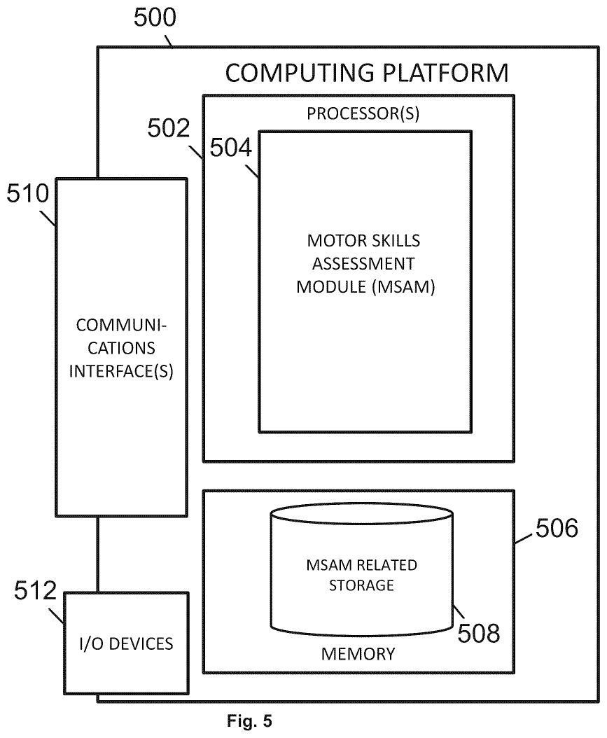

FIG. 5 is a diagram illustrating an example computing platform 500 for automated motor skills assessment. Computing platform 500 may be any suitable entity (e.g., a mobile device or a server) configurable for automated motor skills assessments. For example, computer platform 500 may include a memory and at least one processor for executing a module (e.g., an app or other software) for automated motor skills assessment. In this example, computer platform 500 may also include a user interface (e.g., a display or a touchscreen) for providing a video game containing stimuli designed or usable to identify a neurodevelopmental/psychiatric disorder in the player (e.g., a child, an adult, etc.) and a camera (e.g., a video camera) or other sensor(s) (e.g., touchscreen sensor(s)) for capturing user responses or behaviors, e.g., touch input data, motor skills data (e.g., eye and/or hand movements during gameplay), etc. Continuing with this example, the captured user responses or behaviors may be analyzed to generate related metrics, motor skills assessment information, or a diagnosis of a neurodevelopmental/psychiatric disorder.

Computing platform 500 may include processor(s) 502. Processor(s) 502 may represent any suitable entity or entities (e.g., one or more hardware-based processor) for processing information and executing instructions or operations. Each of processor(s) 502 may be any type of processor, such as a central processor unit (CPU), a microprocessor, a multi-core processor, and the like. Computing platform 500 may further include a memory 506 for storing information and instructions to be executed by processor(s) 502.

In some embodiments, memory 506 can comprise one or more of random access memory (RAM), read only memory (ROM), static storage such as a magnetic or optical disk, or any other type of machine or non-transitory computer-readable medium. Computing platform 500 may further include one or more communications interface(s) 510, such as a network interface card or a communications device, configured to provide communications access to various entities (e.g., other computing platforms). In some embodiments, one or more communications interface(s) 510 may include a user interface configured for allowing a user (e.g., a diagnostic subject for assessment or an assessment operator) to interact with computing platform 500 or related entities. For example, a user interface may include a graphical user interface (GUI) for providing an interactive game or content to the user. In some embodiments, memory 506 may be utilized to store a motor skills assessment module (MSAM) 504, or software therein, and a MSAM related storage 508.

MSAM 504 may be any suitable entity (e.g., software executing on one or more processors) for performing one or more aspects associated with automated motor skills assessments. For example, MSAM 504 may be configured for obtaining touch input data associated with a user using a touchscreen while the user plays a video game involving touching visual elements that move; analyzing the touch input data to generate motor skills assessment information associated with the user, wherein the motor skills assessment information indicates multiple touch related motor skills metrics; determining, using the motor skills assessment information, that the user exhibits behavior indicative of a neurodevelopmental or psychiatric (neurodevelopmental/psychiatric) disorder; and providing, via a communications interface, the motor skills assessment information, a diagnosis, or related data.

In some embodiments, MSAM 504 or another entity may provide a diagnostic application (e.g., an autism screening digital application) for users (e.g., small children) that provide various stimuli in various forms (e.g., videos, games, audio, etc.). In such embodiments, MSAM 504 or another entity may capture user responses and/or related data (e.g., recordings of the user and environment, touchscreen data, etc.) and may then generate or derive metrics from the user interaction (e.g., using various algorithms, techniques, methods) and use the metrics (e.g., as input to a trained machine learning based model) to perform a motor skills assessment or a diagnosis of a neurodevelopmental/psychiatric disorder.

In some embodiments, computing platform 500 and/or MSAM 504 may be communicatively coupled to one or more input or output (I/O) device(s), e.g., a camera, a touchscreen, a mouse, a keyboard, an input sensor, a display, etc. I/O device(s) 512 may represent any suitable entity (e.g., a camera sensor or camera chip in a smartphone) for providing data to a user (e.g., a display) or for obtaining data from or about the user (e.g., a camera for recording visual images or audio and/or a touchscreen for recording touch input). For example, I/O device(s) 512 may include a two-dimensional camera, a three dimensional camera, a heat-sensor camera, a touchscreen, touch sensors, etc. In some embodiments, I/O device(s) 512 may be usable for recording a user and user input during a motor skills assessment (e.g., while the user is playing a video game).

MSAM 504 or another entity may use touch and device kinetic information provided by a touchscreen or other sensors when a user's plays a video game, e.g., a bubble popping game, to quantify touch-based visual motor skills. For example, a digital phenotype or metric “Touch Popping Rate” associated with a bubble popping game may indicate the ratio of popped bubbles over the number of touches; another digital phenotype or metric “Touch Error Variation” associated with the bubble popping game may indicate the standard deviation of the distance between a user's finger position when touching the screen and the center of the closest bubble; another digital phenotype or metric “Touch Average Length” associated with the bubble popping game may indicate the average length of a user's finger trajectory on the screen, and another digital phenotype or metric “Touch Average Applied Force” associated with the bubble popping game may indicate the average estimated force applied on the screen when touching it. See Perochon et al. titled “A tablet-based game for the assessment of visual motor skills in autistic children” (NPJ Digit Med 2023; 6(1): 17) for additional or example methodological details; the disclosure of which is part of the instant specification and is incorporated herein by reference in its entirety.

In some embodiments, MSAM 504 or a related entity (e.g., a medical provider) may administer to a user a therapy or therapies for treating a neurodevelopmental/psychiatric disorder. For example, after performing a motor skills assessment and/or a related diagnosis of a neurodevelopmental/psychiatric disorder, MSAM 504 may provide recommendations or one or more training programs for treating or improving the motor skills of a user. In this example, the recommendations or training programs may be based on a number of factors, including user related factors, such as age, name, knowledge, skills, sex, medical history, and/or other information.

In some embodiments, MSAM 504 may determine and/or provide motor skills assessment information, a diagnosis, and/or related information (e.g., follow-up information and/or progress information) to one or more entities, such as a user, a system operator, a medical records system, a healthcare provider, a caregiver of the user, or any combination thereof. For example, motor skills assessment information, screening results, and/or related information may be provided via a phone call, a social networking message (e.g., Facebook or Twitter), an email, or a text message. In another example, motor skills assessment information may be provided via an app and/or communications interface(s) 510. When provided via an app, motor skills assessment information may include progress information associated with a user. For example, progress information associated with a user may indicate (e.g., to a caregiver or physician) whether certain therapies and/or strategies are improving or alleviating symptoms associated with a particular neurodevelopmental/psychiatric disorder. In another example, progress information may include aggregated information associated with multiple videos and/or assessment sessions.

Memory 506 may be any suitable entity or entities (e.g., non-transitory computer readable media) for storing various information. Memory 506 may include MSAM related storage 508. MSAM related storage 508 may be any suitable entity (e.g., a database embodied or stored in computer readable media) storing user data, stimuli (e.g., digital content, games, etc.), recorded or captured user input, and/or predetermined information. For example, MSAM related storage 508 may include machine learning algorithms, algorithms for statistical analysis, and/or report generation logic. MSAM related storage 508 may also include user data, such as age, name, knowledge, skills, sex, and/or medical history. MSAM related storage 508 may also include predetermined information, including information gathered by clinical studies, patient and/or caregiver surveys, and/or doctor assessments.

In some embodiments, predetermined information may include information for analyzing responses; information for determining based responses; information for determining assessment thresholds; coping strategies; recommendations (e.g., for a caregiver or a child); treatment and/or related therapies, information for generating or selecting games, digital content, or related stimuli usable for performing a motor skills assessment; and/or other information.

In some embodiments, MSAM related storage 508 or another entity may maintain associations between relevant health information and a given user or a given population (e.g., users with a same condition, users with similar characteristics and/or within a similar geographical location). For example, users associated with different conditions and/or age groups may be associated with different recommendations, base responses, and/or assessment thresholds for indicating whether user responses are indicative of neurodevelopmental/psychiatric disorders.

In some embodiments, MSAM related storage 508 may be accessible by MSAM 504 and/or other modules of computing platform 500 and may be located externally to or integrated with MSAM 504 and/or computing platform 500. For example, MSAM related storage 508 may be stored at a server located remotely from a mobile device containing MSAM 504 but still accessible by MSAM 504. In another example, MSAM related storage 508 may be distributed or separated across multiple nodes.

It will be appreciated that the above described modules or entities are for illustrative purposes and that features or portions of features described herein may be performed by different and/or additional modules, components, or nodes. For example, aspects of motor skills assessment described herein may be performed by MSAM 504, computing platform 500, and/or other modules or nodes.

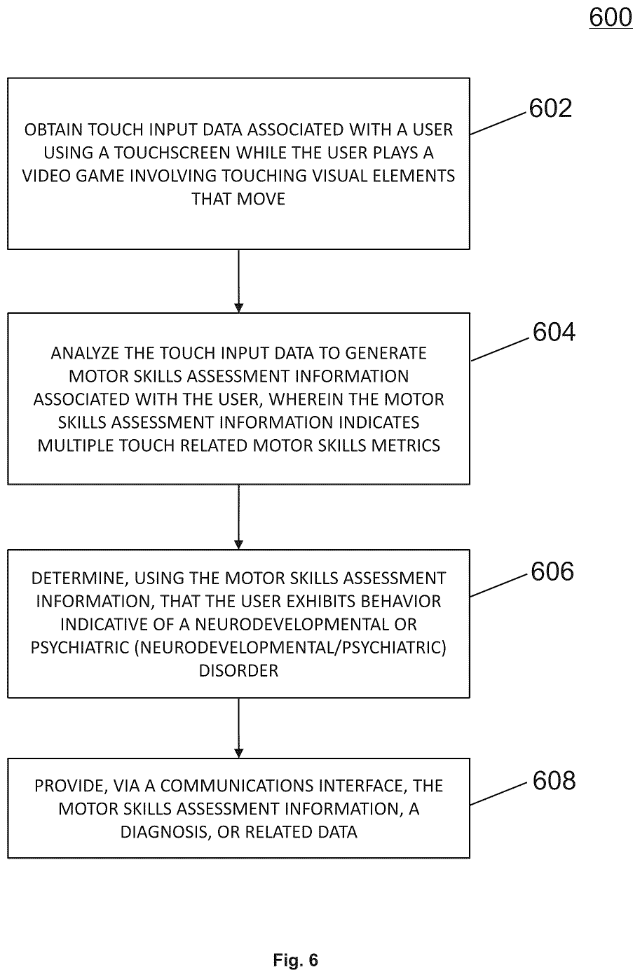

FIG. 6 is a diagram illustrating an example process 600 for automated motor skills assessment. In some embodiments, process 600 described herein, or portions thereof, may be performed at or by computing platform 500, MSAM 504, and/or another module or node. For example, computing platform 500 may be a mobile device, a tablet computer, a laptop computer, or other equipment (e.g., an augmented reality system) and MSAM 504 may include or provide an application running or executing on computing platform 500. In some embodiments, process 600 may include steps 602-608.

In step 602, touch input data associated with a user using a touchscreen may be obtained while the user plays a video game involving touching visual elements that move.

In step 604, the touch input data may be analyzed to generate motor skills assessment information associated with the user, where the motor skills assessment information indicates multiple touch related motor skills metrics.

In step 606, it may be determined, using the motor skills assessment information, that the user exhibits behavior indicative of a neurodevelopmental or psychiatric (neurodevelopmental/psychiatric) disorder.

In step 608, the motor skills assessment information, a diagnosis, or related data may be provided via a communications interface.

In some embodiments, process 600 may also include administering to the user a therapy for treating the neurodevelopmental/psychiatric disorder. In some embodiments, an administered therapy may include recommendations for improving motor skills or an interactive or dynamic game or digital content for improving motor skills over time. For example, MSAM 504 executing on a smartphone may provide a video game (e.g., similar to the video game used in a motor skills assessment or one configured based on the user's assessment score) to improve motor skills. In this example, MSAM 504 or another entity may monitor changes in motor skills over time (e.g., by monitoring game progress), and may use the gathered information to inform therapeutic strategies to improve motor skills (e.g., by changing game parameters, a skill level, or other settings over time). In another example, MSAM 504 executing on a smartphone may provide interactive content or recommendations to improve a user's social interaction skills and/or motor skills.

In some embodiments, the touch related motor skills metrics may relate to number of touches, number of misses, number of pops, a popping rate, touch duration, applied force, length of touch motion, number of touch per target, time spent targeting a visual element, touch frequency, touch velocity, popping accuracy, repeat percentage, distance to the center of a visual element, or number of transitions.

In some embodiments, determining, using the motor skills assessment information, that a user exhibits behavior indicative of a neurodevelopmental/psychiatric disorder may include comparing the motor skills assessment information to information from a population having the neurodevelopmental/psychiatric disorder.

In some embodiments, determining, using the motor skills assessment information, that a user exhibits behavior indicative of a neurodevelopmental/psychiatric disorder may include using the motor skills assessment information as input for a trained machine learning algorithm or model that outputs diagnostic or predictive information regarding the likelihood of the user having the neurodevelopmental/psychiatric disorder.

In some embodiments, a trained machine learning algorithm or model may take as input metrics related to digital phenotyping involving the user, e.g., metrics relate to gaze patterns, social attention, facial expressions, facial dynamics, or postural control.

In some embodiments, a neurodevelopmental/psychiatric disorder may an ASD, language delay, motor delay, intellectual or developmental delay, ADHD, an anxiety disorder diagnosis, or any combination thereof.

In some embodiments, motor skills assessment information or related data may be provided to a user, a medical records system, a service provider, a healthcare provider, a system operator, a caregiver of the user, or any combination thereof. For example, e.g., where information is provided to a clinician or a medical professional, motor skills assessment information may include stimuli used in a test, captured touch input data during the test, test results, and/or other technical or clinical information. In another example, e.g., where information is provided to a parent, a motor skills assessment report may include a metric associated with an easy to understand scale (e.g., 0-100%) for indicating the likelihood of a user (e.g., a child) having a particular neurodevelopmental/psychiatric disorder and useful suggestions for improving one or more related symptoms associated with neurodevelopmental/psychiatric disorder.

In some embodiments, computing platform 500 may include a mobile device, a smartphone, a tablet computer, a laptop computer, a computer, a motor skills assessment device, or a medical device.

It will be appreciated that process 600 is for illustrative purposes and that different and/or additional actions may be used. It will also be appreciated that various actions described herein may occur in a different order or sequence.

It should be noted that computing platform 500, MSAM 504, and/or functionality described herein may constitute a special purpose computing device. Further, computing platform 500, MSAM 504, and/or functionality described herein can improve the technological field of diagnosing and treating various neurodevelopmental/psychiatric disorders by providing mechanisms for automated motor skills assessment using touch input data and related metrics (e.g., obtained or derived from user interaction with a touchscreen during a video game). Moreover, such mechanisms can alleviate many barriers, including costs, equipment, and human expertise, associated with conventional (e.g., clinical) methods of diagnosis and treatment of neurodevelopmental/psychiatric disorders.

The subject matter described herein for automated motor skills assessment improves the functionality of motor skills assessment devices and equipment by providing mechanisms that analyze a user's touch input data obtained or derived from a touchscreen to generate touch related motor skills metrics, use the metrics to determine motor skills assessment information for the user, and determine, using the motor skills assessment information, that the user exhibits or does not exhibit behavior indicative of a neurodevelopmental/psychiatric disorder. It should also be noted that computing platform 500 that implements subject matter described herein may comprise a special purpose computing device usable for various aspects of motor skills assessments, including videos containing region-based stimuli and/or gaze analysis.

Additional details and example methods, mechanisms, techniques, and/or systems for early detection of autism or related aspects are further described in the Examples herein below entitled “A tablet-based game for the assessment of visual motor skills in autistic children,” “Exploring Complexity of Facial Dynamics in Autism Spectrum Disorder,” “Complexity analysis of head movements in autistic toddlers,” and “Blink rate and facial orientation reveal distinctive patterns of attentional engagement in autistic toddlers: a digital phenotyping approach,” and additional Examples.

### EXAMPLES

The presently disclosed subject matter will be now be described more fully hereinafter with reference to the accompanying EXAMPLES, in which representative embodiments of the presently disclosed subject matter are shown. The presently disclosed subject matter can, however, be embodied in different forms and should not be construed as limited to the embodiments set forth herein. Rather, these embodiments are provided so that this disclosure will be thorough and complete, and will fully convey the scope of the presently disclosed subject matter to those skilled in the art.

### Example 1

### Early Detection of Autism in Primary Care Based on Scalable Computational Behavioral Phenotyping

Autism is a neurodevelopmental condition associated with challenges in socialization and communication. Early detection of autism ensures timely access to intervention. Autism screening questionnaires have lower accuracy when used in primary care. Here we report the results of a prospective study assessing the accuracy of an autism screening digital application (app) administered during a pediatric well-child visit to 475 17-36-month-old children, 49 diagnosed with autism and 98 with developmental delay without autism. The app displayed stimuli that elicited behavioral signs of autism, which were quantified using computer vision and machine learning. An algorithm combining multiple digital phenotypes showed high diagnostic accuracy: Area under the receiver operating characteristic curve (AUC)=0.90, sensitivity 87.8%, and specificity 80.8% distinguishing autism versus neurotypical children; AUC=0.86, sensitivity 81.6%, and specificity 80.5% distinguishing autism versus non-autism. Results demonstrate that digital phenotyping is an objective, scalable approach to autism screening in real-world settings.

Autism spectrum disorder (henceforth “autism”) is a neurodevelopmental condition associated with qualitative challenges in social communication abilities and the presence of restricted and repetitive behaviors. Autism signs emerge between 9-18 months and include reduced attention to people, lack of response to name, differences in affective engagement and expressions, and motor delays, among other features.¹ Commonly, children are screened for autism at their 18-24 well-child visits using a parent questionnaire, the Modified Checklist for Autism in Toddlers-Revised with Follow Up (M-CHAT-R/F).² The M-CHAT-R/F has been shown to have higher accuracy in research settings³ compared to real-world settings, such as primary care, particularly for girls and children of color.⁴⁻⁷ This is, in part, due to low rates of completion of the follow-up interview by pediatricians.⁸ A study of >25,000 children screened in primary care found that the M-CHAT/F's specificity was high (95.0%) but sensitivity was poor (39.0%).⁶ Thus, there is a need for accurate, objective, and scalable autism screening tools to increase the accuracy of autism screening and reduce disparities in access to early diagnosis and intervention, which can improve outcomes.⁹

A promising screening approach is the use of eye-tracking technology to measure children's attentional preferences for social versus non-social stimuli.¹⁰ Autism is characterized by reduced spontaneous visual attention to social stimuli.¹⁰ Studies of preschool and school-age children using machine learning (ML) of eye-tracking data reported encouraging findings for the use of eye tracking for distinguishing autistic and neurotypical children.^{11,12} However, because autism has a heterogeneous presentation involving multiple behavioral signs, eye-tracking tests alone may be insufficient as an autism screening tool. An eye-tracking screening measure of social attention evaluated in 1,863 12-48-month-old children had strong specificity (98.0%) but poor sensitivity (17.0%). The authors conclude that the eye-tracking task is useful for detecting a subtype of autism.¹³

By quantifying multiple autism-related behaviors, it may be possible to better capture the complex presentation of autism reflected in current diagnostic assessments. Digital phenotyping can detect differences between autistic and neurotypical children in social attention, head movements, facial expressions, and motor behaviors.¹⁴⁻¹⁸ We developed an application (app), SenseToKnow, which is administered on a tablet and displays brief, strategically designed movies while the child's behavioral responses are recorded via the frontal camera embedded in the device and quantified via computer vision analysis (CVA) and ML. The app elicits and measures a wide range of autism-related behaviors, including social attention, facial expressions and dynamics, head movements, response to name, blink rate, and motor skills (see FIGS. 2A-2F).¹⁹⁻²⁵ In the present study, the app was administered during a pediatric primary care well-child visit to 475 17-36-month-old toddlers, 49 of whom were subsequently diagnosed with autism and 98 with developmental or language delay without autism (see Table 1 for demographic and clinical characteristics). The app used ML to integrate 23 digital phenotypes into a combined algorithm and demonstrated that the algorithm can classify autistic versus non-autistic toddlers with a high degree of accuracy.

FIGS. 2A to 2F show a presentation of the SenseToKnow app workflow from the data collection to the fully automatic individualized and interpretable predictions. In FIG. 2A, video and touch data are recorded via SenseToKnow application, which displays brief movies and a bubble popping game (see also EXAMPLE 2). In FIG. 2B, faces are automatically detected using CVA and child's face is identified and validated using sparse semi-automatic human annotations. 49 facial landmarks, head pose, and gaze coordinates are extracted for every frame using CVA. In FIG. 2C, shown is automatic computation of multiple digital behavioral phenotypes. In FIG. 2D, shown is training of K=1000 XGBoost classifier from multiple phenotypes using 5-fold cross-validation and overall performance evaluation, and estimation of the final prediction confidence score based on Youden optimality index. In FIG. 2E, show is analysis of the model interpretability using SHAP values analysis, showing features' values in blue/red, and direction of their contributions to the model prediction in blue/orange. FIG. 2F shows a report of an individualized app administration summary showing the participants' unique digital phenotype (red dot on the graphs) along with group-wise distributions (autism in orange and neurotypical in blue), confidence and quality scores, and the personalized app variables contributions summary.

FIGS. 3A to 3C show accuracy metrics and normalized SHAP value analysis. In FIG. 3A, The Receiver Operating Characteristic Curves (ROC) illustrate the model performances when classifying different diagnosis groups, using all app variables. Sample N=475; 49 diagnosed with autism, 98 diagnosed with developmental or language delay without autism. The final score of the M-CHAT-R/F is used when available (N=374/377). 95% confidence intervals (CI) are computed by the Hanley McNeil method. AUC denotes the area under the ROC curve. In FIG. 3C, app administration reports for a 25-month-old neurotypical boy and a 30-month-old autistic girl, both correctly classified, including each child's app quality score, confidence score, and the contributions of each app variable to the child's individualized prediction. In FIG. 3B normalized SHAP value analysis showing the app variables importance for the prediction of autism. The x-axis represents the features contribution to the final prediction, with positive (respectively, negative) values associated with an increase in likelihood of autism (respectively, neurotypical). The y-axis represents the app variables in descending order of importance. The blue-red gradient color spans the app variables relevance for the score, from low to high values, with grey samples associated with missing variable. For each app variable, a point represents the normalized SHAP value of a participant. NT—Neurotypical; DD-LD—Developmental Delay and/or Language Delay.

### Results

Quality and prediction confidence scores. Quality scores were automatically computed for each app administration, which reflected the amount of available app variables weighted by their predictive power. Quality scores were found to be high (median score=93.9%, Q1-Q3 [90.0%-98.4%]), with no diagnostic group differences. A prediction confidence score for accurately classifying an individual child was also calculated. At the 20% threshold, 311/377 administrations were rated high confidence (see also EXAMPLE 2 for details).

Diagnostic accuracy of SenseToKnow for the detection of autism. Using all app variables, we trained a model comprised of K=1000 tree-based extreme gradient-boosting algorithms (XGBoost) to classify diagnostic groups.²⁶ FIG. 3A displays the AUC results for classification of autism versus each of the other groups (neurotypical, non-autism, DD-LD), including accuracy based on the combination of the app results with the M-CHAT-R/F², which was administered as a study measure.

Based on the Youden Index,²⁷ an algorithm integrating all app variables showed a high level of accuracy for classification of autism versus neurotypical development with AUC=0.90, CI [0.87-0.93], sensitivity 87.8% (SD=4.9), and specificity 80.8% (SD=2.3). Restricting administrations to those with high prediction confidence, the AUC increased to 0.93 (CI [0.89-0.96]). Table 2 shows performance results for autism versus neurotypical group classification based on individual and combined app variables. Classification of autism versus non-autism (DD-LD combined with neurotypical) also showed strong accuracy: AUC=0.86 (CI [0.83-0.90]), sensitivity 81.6% (SD=5.4), and specificity 80.5% (SD=1.8).

Nine autistic children who scored negative on the M-CHAT-R/F were correctly classified by the app as autistic as determined by expert evaluation. Among 40 children screening positive on the M-CHAT-R/F, there were 2 classified neurotypical based on expert evaluation and both were correctly classified by the app. Combining the app algorithm with the M-CHAT-R/F further increased classification performance to AUC=0.97 (CI [0.96-0.98]), specificity=91.8% (SD=4.5), and sensitivity=92.1% (SD=1.6).

Diagnostic accuracy of SenseToKnow for subgroups based on sex, race, and ethnicity. Classification performance of the app based on AUCs remained consistent when stratifying groups by sex (AUC for girls=89.1(CI [82.6-95.6]), and for boys AUC=89.6 (CI [86.2-93.0])), as well as race, ethnicity, and age. Table 3 provides exhaustive results for all subgroups. However, confidence intervals were larger due to smaller sample sizes for subgroups.

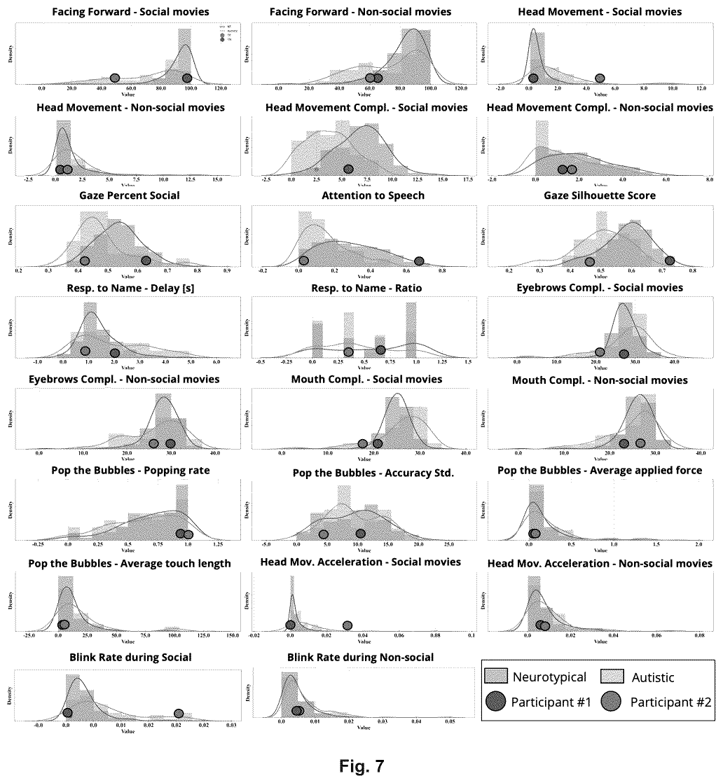

Model interpretability. Distributions for each app variable for autistic and neurotypical participants are shown in FIG. 7. To address model interpretability, we used SHapley Additive exPlanations (SHAP) values²⁸ for each child to examine the relative contributions of the app variables to the model's prediction and disambiguate the contribution of each feature from their missingness (see FIGS. 3B and 3C and EXAMPLE 2 for details). FIG. 3C illustrates the ordered normalized importance of the app variables for the overall model. Facing forward during social movies was the strongest predictor (Mean|SHAP|=11.2% (SD=6.0%)), followed by percent of time gazing at social stimuli (Mean|SHAP|=11.1% (SD=5.7%)), and delay in response to a name call (Mean|SHAP|=7.1% (SD=4.9%)). The SHAP values as a function of the app variable values are provided in EXAMPLE 2.

SHAP interaction values indicated that interactions between predictors were significant contributors to the model; average contribution of app variables alone was 64.6% (SD=3.4%) and 35.4% (SD=3.4%) for the feature interactions. Analysis of the missing data SHAP values revealed that missing variables were contributing to 5.2% (SD=13.2%) of the model predictions. See EXAMPLE 2 for details.

Individualized interpretability. Analysis of the individual SHAP values revealed individual behavioral patterns that explained the model's prediction for each participant. FIG. 3C shows individual cases illustrating how the positive or negative contributions of the app variables to the predictions can be used to (i) deliver intelligible explanations about the child's app administration and diagnostic prediction, (ii) highlight individualized behavioral patterns associated with autism or neurotypical development, and (iii) identify misclassified digital profile patterns. Additional examples including illustrative videos are described in EXAMPLE 2.

### Discussion of Example 1

When used in primary care, the accuracy of autism screening parent questionnaires has been found to be lower than in research contexts, especially for children of color and girls, which can increase disparities in access to early diagnosis and intervention. Studies using eye-tracking of social attention alone as an objective, quantitative index of autism have reported inadequate sensitivity, perhaps because assessments based on only one autism feature (differences in social attention) do not adequately capture the complex and heterogenous clinical presentation of autism.

We evaluated the accuracy of an ML and CVA-based algorithm using multiple autism-related digital phenotypes assessed via a mobile app (SenseToKnow) administered on a tablet in primary care settings for identification of autism in a large sample of toddler-age children, the age at which screening is routinely conducted. The app captured the wide range of early signs associated with autism, including differences in social attention, facial expressions, head movements, response to name, blink rates, and motor skills, and was robust to missing data. ML allowed optimization of the prediction algorithm based on weighting different behavioral variables and their interactions. We demonstrated high levels of usability and diagnostic accuracy for classification of autistic versus neurotypical children and autistic versus non-autistic children (neurotypical and other developmental/language delays). The accuracy of SenseToKnow for detecting autism did not differ based on the child's sex, race, or ethnicity, suggesting that an objective digital screening approach that relies on direct quantitative observations of multiple behaviors may improve autism screening in diverse populations.

We developed methods for automatic assessment of the quality of the app administration and prediction confidence scores, both of which could facilitate the use of SenseToKnow in real world settings. The quality score provides a simple, actionable means of determining whether the app should be re-administered. This can be combined with a prediction confidence score which can inform a provider about the degree of certainty regarding the likelihood a child will be diagnosed with autism. Children with uncertain values could be followed to determine whether autism signs become more pronounced, whereas children with high confidence values could be prioritized for referral or begin intervention while the parent waits for their child to be evaluated. Using SHAP analyses, the app output provides interpretable information regarding which behavioral features are contributing to the diagnostic prediction for an individual child. Such information could be used prescriptively to identify areas in which behavioral intervention should be targeted. Notably, the app quantifies autism signs related to social attention, facial expressions, response to language cues, and motor skills, but does not capture behaviors in the restricted and repetitive behavior domain.

In the context of an overall pathway for autism diagnosis, our vision is that autism screening in primary care should be based on integrating multiple sources of information, including screening questionnaires based on parent report questionnaires and digital screening based on direct behavioral observation. Recent work suggests that ML analysis of a child's healthcare utilization patterns using data passively derived from the electronic health record could also be useful for early autism prediction.²⁹ Results of the present study support this multimodal screening approach. A large study conducted in primary care found that the PPV of the M-CHAT/F was 14.6% and was lower for girls and children of color.⁶ In comparison, the PPV of the app in the present study was 40.6%, and the app performed similarly across children of different sex, race, and ethnicity. Furthermore, combining the M-CHAT-R/F with digital screening resulted in an increased PPV of 63.4%. Thus, our results suggest that a digital phenotyping approach will improve the accuracy of autism screening.

A possible limitation of the present study includes possible validation bias given that it was not feasible to conduct a comprehensive diagnostic evaluation on participants considered neurotypical. This was mitigated by the fact that diagnosticians were naïve with respect to the app results. The percentage of autism versus non-autism cases in this study is higher than in the general population, raising the potential for sampling bias. It is possible that parents who had developmental concerns about their child were more likely to enroll the child in the study. Although prevalence bias is addressed statistically by calibrating the performance metrics to the population prevalence of autism, this remains a possible limitation of the study. Accuracy assessments potentially could have been inflated due to differences in language abilities between the autism and developmental delay groups, although the two groups had similar nonverbal abilities. Future studies evaluate the app's performance in an independent sample with children of different ages and language and cognitive abilities. Strengths of this study include the sample diversity, evaluation of the app in a real-world setting at an age at which autism screening is routinely conducted, and following children through age 4 years to determine final diagnosis.

We conclude that quantitative, objective, and scalable digital phenotyping offers promise in increasing the accuracy of autism screening and reducing disparities in access to diagnosis and intervention, complementing existing autism screening questionnaires. While we believe that this study represents a significant step forward in developing improved autism screening tools, accurate use of these screening tools requires training and systematic implementation by primary providers, and a positive screen must then be linked to appropriate referrals and services. Each of these touch points along the clinical care pathway contributes to the quality of early autism identification and can impact timely access to interventions and services than can influence long-term outcomes.

### Methods

Study cohort. The study was conducted from December 2018 to March 2020 (Pro00085434). Participants were 475 children, 17-36 months, who were consecutively enrolled at one of four Duke University Health System (DUHS) pediatric primary care clinics during their well-child visit. Inclusion criteria were age 16-38 months, not ill, and caregiver's language was English or Spanish. Exclusion criteria were sensory or motor impairment that precluded sitting or viewing the app, unavailable clinical data, and child too upset at their well-child visit. Table 1 describes sample demographic and clinical characteristics.

754 participants were approached and invited to participate, 214 declined participation and 475 (93% of enrolled participants) completed study measures. All parents or legal guardians provided written informed consent, and the study protocol was approved by the Duke University Health System Institutional Review Board.

Diagnostic classification. Children were administered the M-CHAT-R/F,² a parent survey querying different autism signs. Children with a final M-CHAT-R/F score of >2 or whose parents and/or provider expressed any developmental concern were provided a gold standard autism diagnostic evaluation based on the Autism Diagnostic Observation Schedule-Second Edition (ADOS-2),²⁹ DSM-5 criteria checklist, and Mullen Scales of Early Learning,³⁰ conducted by a licensed, research-reliable psychologist who was blind with respect to app results. Mean duration between app screening and evaluation=3.5 months, which is a similar or shorter duration compared to real-world settings. Diagnosis of autism spectrum disorder required meeting full DSM-5 diagnostic criteria. Diagnosis of developmental or language delay without autism (DD-LD) was defined as failing the M-CHAT-R/F and/or having provider or parent concerns and having been administered the ADOS-2 and Mullen Scales and determined by the psychologist not to meet diagnostic criteria for autism and exhibiting developmental and/or language delay based on the Mullen Scales (scoring>9 points below the mean on at least one Mullen Scales subscale; SD=10).

In addition, each participant's DUHS electronic health record (EHR) was monitored through age 4 years to confirm whether the child subsequently received a diagnosis of either autism spectrum disorder or DD-LD. Following validated methods used by Guthrie et al., children were classified as autistic or DD-LD based on their EHR record if an ICD-9/10 diagnostic code for autism spectrum disorder or DD-LD (without autism) appeared more than once or was provided by an autism specialty clinic.⁶ If a child did not have an elevated M-CHAT-R/F score, no developmental concerns were raised by the provider or parents, and there were no autism or DD-LD diagnostic codes in the EHR through age four, they were considered neurotypical. There were 2 children classified as neurotypical who scored positive on the M-CHAT-R/F who were considered neurotypical based on expert diagnostic evaluation and had no autism or DD-LD EHR diagnostic codes.

Based on these procedures, 49 children were diagnosed with autism spectrum disorder (6 based on EHR only), 98 children were diagnosed DD-LD without autism (78 based on EHR only), and 328 children were considered neurotypical. Diagnosis of autism or developmental delay was made naïve to app results.

SenseToKnow app. Parents held their child on their lap while brief, engaging movies were presented on an iPad set on a tripod approximately 60 cm away from the child. Parents were asked to refrain from talking during the movies. The frontal camera embedded in the device recorded the child's behavior at resolutions of 1280×720, 30 frames per second. While children were watching the movies, their name was called three times by an examiner standing behind them at pre-defined timestamps. The children then participated in a “Bubble Popping” game using their finger to pop a set of colored bubbles that moved continuously across the screen. App completion took <10 minutes. English and Spanish versions were shown. Additional details can be found in EXAMPLE 2.

App variables. CVA and ML was used to quantify multiple digital phenotypes. Detailed information regarding the identification and recognition of the child's face and the estimation of the frame-wise facial landmarks, head pose, and gaze has been described previously.¹⁹ Several CVA-based and touch-based behavioral variables were computed, described next.

Facing forward. During the social and non-social movies, we computed the average percentage of time the children faced the screen, filtering-in frames using three rules: eyes were open, estimated gaze was at or close to the screen area, and the face was relatively steady, referred to as Facing Forward. This variable was used as a proxy for the child's attention to the movies. See Chang et al. for methodological details.¹⁹

Social attention. The app includes two movies featuring clearly separable social and non-social stimuli on each side of the screen, designed to capture social/non-social attentional preference. The variable Gaze Percent Social was defined as the percentage of time the child gazed at the social half of the screen, and the Gaze Silhouette Score reflected how concentrated versus spread out the gaze clusters were. See Chang et al. for methodological details.¹⁹

Attention to speech. One of the movies features two actors, one on each side of the screen, taking turns in a conversation. We computed the correlation between the child's gaze patterns and the alternating conversation, defined as the Gaze Speech Correlation variable. See Chang et al. for methodological details.¹⁹

Facial dynamics complexity. The complexity of the facial landmarks' dynamics was estimated for the eyebrows and mouth regions of the child's face using multiscale entropy. We computed the average complexity of the mouth and eyebrows regions during social and non-social movies, referred to as the Mouth Complexity and Eyebrows Complexity. See Krishnappa Babu et al. for methodological details.²⁰

Head movement. We evaluated the rate of head movement (computed from the time series of the facial landmarks) for social and non-social movies. Average head movement was referred to as Head Movement. Complexity and acceleration of the head movements were computed for both types of stimuli using multiscale entropy and derivative of the time series, respectively. See Krishnappa Babu et al. for methodological details.²²

Response to name. Based on automatic detection of the name calls and the child's response to their name by turning their head computed from the facial landmarks, we defined two CVA-based variables: Response to Name Proportion, representing the proportion of times the child oriented to the name call, and Response to Name Delay, the average delay (in seconds) between the offset of the name call and head turn. See Perochon et al. for methodological details.²³

Blink rate. During the social and non-social movies, CVA was used to extract the blink rates as indices of attentional engagement, referred to as Blink Rate. See Krishnappa Babu et al. for methodological details.²⁴

Touch-based visual-motor skills. Using the touch and device kinetic information provided by the device sensors when the child played the bubble popping game (see EXAMPLE 2), we defined Touch Popping Rate as the ratio of popped bubbles over the number of touches, Touch Error Variation as the standard deviation of the distance between the child's finger position when touching the screen and the center of the closest bubble, Touch Average Length as the average length of the child's finger trajectory on the screen, and Touch Average Applied Force as the average estimated force applied on the screen when touching it. See Perochon et al. for methodological details.²⁵

In total, we measured 23 app-derived variables, comprised of 19 CVA-based and 4 touch-based variables. Additional information regarding the computation of these variables, missing data rate, and their pairwise correlation coefficients is detailed in EXAMPLE 2.

Statistical analysis. Using the app variables, we trained a model comprised of K=1000 tree-based extreme gradient-boosting algorithms (XGBoost) to differentiate diagnostic groups.²⁶ For each XGBoost model, 5-fold cross-validation was used while shuffling the data to compute individual intermediary binary predictions and SHapley Additive exPlanations (SHAP) value statistics (metrics mean and standard deviation).²⁸ The final prediction confidence scores, between 0 and 1, were computed averaging the K predictions (see EXAMPLE 2). We implemented a five-fold nested cross validation stratified by diagnosis group to separate the data used for training the algorithm and the evaluation on unseen data.³² Missing data were encoded with a value out of the range of the app variables, such that the optimization of the decision trees considered the missing data as information. Overfitting was controlled using a tree maximum depth of 3, subsampling app variables at a rate of 80%, and using regularization parameters during the optimization process. Diagnostic group imbalance was addressed by weighting training instances by the imbalance ratio. Details regarding the algorithm and hyperparameters are provided in the EXAMPLE 2. The contribution of the app variables to individual predictions was assessed by the SHAP values, computed for each child using all other data to train the model and normalized such that the features' contributions to the individual predictions range from 0 to 1. See details in EXAMPLE 2.

A quality score was computed based on the amount of available app variables weighted by their predictive power (measured as their relative importance to the model; see details in EXAMPLE 2).

The prediction confidence score quantified the confidence in the model's prediction and was used to analyze the varying performance of the app when removing app administrations rated uncertain based on different thresholds (5%, 10%, 15%, and 20%; see details in EXAMPLE 2). Performance was evaluated using the receiver operator characteristic curve (ROC) area under the curve (AUC), with 95% confidence intervals (CI) computed using the Hanley McNeil method.³² Unless otherwise mentioned, sensitivity, specificity, positive predictive value (PPV), and negative predictive value (NPV) were defined using the operating point of the ROC that optimized the Youden index, with an equal weight given to sensitivity and specificity.¹³ Given that the study sample autism prevalence

$$( {π}_{study} = \frac{49}{328} \approx 14.9 \% )$$

differs from the general population in which the screening tool would be used (πₚₒₚᵤₐₜᵢₒₙ≈2%), we also report the adjusted PPV and NPV to provide a more accurate estimation of the app performance as a screening tool deployed at scale in practice (see details in EXAMPLE 2). Statistics were calculated in Python V.3.8.10, using SciPy low-level functions V.1.7.3, XGBoost and SHAP official implementations V.1.5.2 and V.0.40.0, respectively.

TABLE 1
Study Sample Demographic and Clinical Characteristics

| | Neurotypical | | DD-LD ^{a} |
|---|---|---|---|
| | (N = 328) | Autism (N = 49) | (N = 98) |
| Age (in months) - Mean (SD) | 20.4 (3.0) | 24.2 (4.6) | 21.2 (3.55) |
| Sex - (%) | | | |
| Boys | 170 (51.8) | 38 (77.5) | 61 (62.0) |
| Girls | 158 (48.2) | 11 (22.5) | 37 (38.0) |
| Ethnicity - (%) | | | |
| Not Hispanic/Latino | 306 (93.3) | 36 (73.4) | 83 (84.7) |
| Hispanic/Latino | 22 (6.7) | 13 (26.6) | 15 (15.3) |
| Race - (%) | | | |
| Unknown/Declined | 0 (0.0) | 0 (0.0) | 1 (1.0) |
| American Indian/Alaskan Native | 1 (0.3) | 3 (6.1) | 0 (0.0) |
| Asian | 6 (1.8) | 1 (2.0) | 0 (0.0) |
| Black or African American | 28 (8.5) | 11 (22.4) | 15 (15.3) |
| White/Caucasian | 255 (77.7) | 23 (46.9) | 69 (70.4) |
| More than one race | 32 (9.9) | 7 (14.3) | 8 (8.2) |
| Other | 6 (1.8) | 4 (8.3) | 5 (5.1) |
| Highest Level of Education - (%) | | | |
| Unknown/Not Reported | 2 (0.6) | 0 (0.0) | 0 (0.0) |
| Without High School Diploma | 1 (0.3) | 4 (8.2) | 5 (5.1) |
| High School Diploma or Equivalent | 12 (3.6) | 8 (16.3) | 8 (8.2) |
| Some College Education | 32 (9.8) | 10 (20.4) | 11 (11.2) |
| 4-Year College Degree or More | 281 (85.7) | 27 (55.1) | 74 (75.5) |
| M-CHAT-R/F ^{b} -Total (#) | | | |
| Unknown/Not Reported | 1 (0.3) | 2 (4.0) | 0 (0.0) |
| Positive | 2 (0.6) | 38 (77.5) | 18 (18.4) |
| Negative | 325 (99.1) | 9 (18.5) | 80 (81.6) |
| ADOS ^{c} Calibrated Severity Score (CSS) | | | |
| Unknown/Not Reported - Total (%) | N/A | 6 (12.2) | 85 (86.7) |
| Restricted/Repetitive Behavior CSS | N/A | 7.76 (1.64) | 5.23 (1.42) |
| Social Affect CSS | N/A | 6.97 (1.71) | 3.77 (1.69) |
| Total CSS | N/A | 7.41 (1.79) | 3.69 (1.32) |
| Mullen Scales of Early Learning | | | |
| Unknown/Not Reported Total (%) | N/A | 6 (12.2) | 82 (100.0) |
| Early Learning Composite Score | N/A | 63.6 (10.12) | 73.85 (15.30) |
| Expressive Language T-Score | N/A | 28.34 (7.56) | 35.23 (10.00) |
| Receptive Language T-Score | N/A | 23.37 (5.60) | 32.46 (12.94) |
| Fine Motor T-Score | N/A | 34.24 (10.06) | 39.30 (6.60) |
| Visual Reception T-Score | N/A | 33.42 (10.60) | 36.30 (12.03) |

^{a} DD-LD: developmental delay and/or language delay.
^{b} M-CHAT-R/F: Modified Checklist for Autism in Toddlers, Revised with Follow Up.
^{c} ADOS-2: Autism Diagnostic Observation Schedule - Second Edition.

TABLE 2
App performance based on individual and combined app variables.

| | AUROC | | | | |
|---|---|---|---|---|---|
| | (95% CI) | Sensitivity | Specificity | PPV ^{a} | NPV ^{a} |
| All app variables | 89.9 (3.0) | 87.8 (4.9) | 80.8 (2.3) | 40.6 (8.8) | 97.8 (99.7) |
| Facing forward | 83.8 (3.7) | 87.8 (4.4) | 65.9 (2.6) | 27.7 (5.2) | 97.3 (99.6) |
| Gaze ^{b} | 77.6 (4.0) | 63.3 (7.7) | 85.4 (1.8) | 39.2 (8.4) | 94.0 (99.1) |
| Facial dynamics complexity | 75.9 (4.2) | 63.3 (6.5) | 82.9 (2.3) | 35.6 (7.3) | 93.8 (99.1) |
| Head movements | 86.4 (3.4) | 87.8 (4.1) | 74.4 (2.4) | 33.9 (6.8) | 97.6 (99.7) |
| Response to name | 65.8 (4.4) | 83.7 (5.1) | 46.6 (2.4) | 19.0 (3.2) | 95.0 (99.3) |
| Touch-based (game) | 57.6 (4.5) | 79.6 (5.2) | 39.0 (2.5) | 16.3 (2.7) | 92.8 (8.9) |
| All app variables + M- | 96.6 (1.8) | 91.8 (4.5) | 92.1 (1.6) | 63.4 (19.7) | 98.7 (99.8) |
| CHAT-R/F Score ^{c} | | | | | |

Results represent performance of the XGBoost model trained to classify autistic and neurotypical groups based on individual and combined app variables (digital phenotypes).
^{a} PPV and NPV values adjusted for population prevalence (see EXAMPLE 2)
^{b} Gaze silhouette score, gaze speech correlation, and gaze percent social
^{c} Modified Checklist for Autism in Toddlers - Revised with Follow-up final score

TABLE 3
App performance stratified by sex, race, ethnicity, age, quality score, prediction confidence threshold

| | | | NT | | Not | AUC (%) | Sensitivity | Specificity | PPV | NPV |
|---|---|---|---|---|---|---|---|---|---|---|
| | Group | N | Autism | Correct | Correct | (95% CI) | (STD) | (STD) | (Adjusted) | (Adjusted) |
| Sex | Boys | 196 | 158 | 123 | 35 | 89.6 | 86.8 | 77.8 | 48.5 | 96.1 |
| | | | 38 | 33 | 5 | (3.4) | (5.3) | (3.2) | (7.7) | (99.6) |
| | Girls | 181 | 170 | 142 | 28 | 89.1 | 90.9 | 83.5 | 26.3 | 99.3 |
| | | | 11 | 10 | 1 | (6.5) | (9.1) | (2.9) | (10.5) | (99.8) |
| Race | White | 278 | 255 | 211 | 44 | 86.9 | 82.6 | 82.7 | 30.2 | 98.1 |
| | | | 23 | 19 | 4 | (4.9) | (7.8) | (2.4) | (9.2) | (99.5) |
| | Black | 39 | 28 | 15 | 13 | 81.2 | 90.9 | 53.6 | 43.5 | 93.8 |
| | | | 11 | 10 | 1 | (8.5) | (9.0) | (9.5) | (4.0) | (99.6) |
| | Other | 60 | 45 | 39 | 6 | 97.6 | 93.3 | 86.7 | 70.0 | 97.5 |
| | | | 15 | 14 | 1 | (2.8) | (7.2) | (4.6) | (12.9) | (99.8) |
| Ethnicity | Not | 342 | 306 | 245 | 61 | 87.8 | 86.1 | 80.1 | 33.7 | 98.0 |
| | Hispanic/ | | 36 | 31 | 5 | (3.8) | (5.7) | (2.3) | (8.4) | (99.8) |
| | Latino | | | | | | | | | |
| | Hispanic/ | 35 | 22 | 20 | 2 | 95.3 | 92.3 | 90.9 | 85.7 | 95.2 |
| | Latino | | 13 | 12 | 1 | (4.3) | (7.1) | (6.2) | (17.7) | (99.8) |
| Age | 17-18.5 | 164 | 159 | 125 | 34 | 94.5 | 1.00 | 78.6 | 12.8 | 1.0 |
| (months) | | | 5 | 5 | 0 | (7.1) | (0.0) | (2.8) | (9.0) | (1.0) |
| | 18.5-24 | 104 | 86 | 72 | 14 | 89.5 | 83.3 | 83.7 | 51.7 | 96.0 |
| | | | 18 | 15 | 3 | (5.1) | (9.5) | (4.7) | (9.8) | (99.6) |
| | 24-36 | 109 | 83 | 68 | 15 | 90.1 | 88.5 | 81.9 | 40.6 | 97.8 |
| | | | 26 | 23 | 3 | (4.2) | (6.0) | (4.3) | (8.8) | (99.7) |
| Quality | Higher | 349 | 310 | 259 | 51 | 89.6 | 84.6 | 83.5 | 39.3 | 97.7 |
| Score | than 75% | | 39 | 33 | 6 | (3.4) | (5.0) | (2.1) | (9.8) | (99.6) |
| | Lower | 28 | 18 | 6 | 12 | 76.1 | 1.0 | 33.3 | 45.5 | 1.0 |
| | than 75% | | 10 | 10 | 0 | (10.0) | (0.0) | (12.3) | (3.1) | (1.0) |
| Prediction | Threshold | 251 | 216 | 201 | 15 | 92.6 | 91.4 | 93.1 | 68.1 | 98.5 |
| Confidence | 5% | | 35 | 32 | 3 | (3.1) | (4.4) | (1.6) | (21.9) | (99.8) |
| Thresholds | Threshold | 279 | 243 | 219 | 24 | 92.4 | 88.9 | 90.1 | 57.1 | 98.2 |
| | 10% | | 36 | 32 | 4 | (3.0) | (4.9) | (2.1) | (16.0) | (99.7) |
| | Threshold | 297 | 258 | 228 | 30 | 92.0 | 89.7 | 88.4 | 53.8 | 98.3 |
| | 15% | | 39 | 35 | 4 | (3.0) | (5.1) | (2.0) | (14.1) | (99.7) |
| | Threshold | 311 | 270 | 238 | 32 | 91.6 | 87.8 | 88.1 | 52.9 | 97.9 |
| | 20% | | 41 | 36 | 5 | (3.0) | (5.4) | (1.7) | (13.6) | (99.7) |
| Diagnostic | Autistic | 475 | 426^{a} | 343 | 83 | 86.4 | 81.6 | 80.5 | 32.5 | 97.4 |
| groups | vs non- | | 49^{b} | 40 | 9 | (3.4) | (5.4) | (1.8) | (8.2) | (99.5) |
| | Autistic | | | | | | | | | |
| | Autistic + | 475 | 328^{c} | 267 | 61 | 71.7 | 53.7 | 81.4 | 56.4 | 79.7 |
| | DD-LD vs | | 147^{d} | 79 | 68 | (2.7) | (3.9) | (2.1) | (5.8) | (98.8) |
| | NT | | | | | | | | | |
| | DD-LD | 426 | 328^{c} | 227 | 101 | 65.1 | 55.1 | 69.2 | 34.8 | 83.8 |
| | vs NT | | 98^{e} | 54 | 44 | (3.3) | (5.2) | (2.6) | (3.7) | (98.6) |

The operating point (or positivity threshold) corresponds to the one maximizing the Youden Index. PPV and NPV values were adjusted for population prevalence (see Supplementary Information). Stratification by diagnosis group refers to neurotypical (NT; first row) and autistic (second row) except for the Diagnostic groups category;
^{a}Non-autistic group (neurotypical + DD-LD);
^{b}Autistic;
^{c}Neurotypical (NT);
^{d}Autistic + DD-LD;
^{e}DD-LD. Correct: number of correct diagnosis predictions; Not Correct: number of incorrect predictions.

### REFERENCES FOR EXAMPLE 1

The disclosure of each of the following references is incorporated herein by reference in its entirety to the extent that it is not inconsistent herewith and to the extent that it supplements, explains, provides a background for, or teaches methods, techniques, and/or systems employed herein. The numbers below correspond to the superscripted numbers in EXAMPLE 1.
1. Dawson et al. Prediction of autism in infants: progress and challenges. Lancet Neurol 2023; 22(3):244-254.
2. Robins et al. Validation of the modified checklist for Autism in toddlers, revised with follow-up (M-CHAT-R/F). Pediatrics 2014; 133(1): 37-45.
3. Wieckowski et al. Sensitivity and Specificity of the Modified Checklist for Autism in Toddlers (Original and Revised): A Systematic Review and Meta-analysis. JAMA Pediatr 2023; Feb 20: e225975.
4. Scarpa et al. The Modified Checklist for Autism in Toddlers: Reliability in a diverse rural American sample. J Autism Dev Disord 2013; 43(10): 2269-79.
5. Donohue et al. Race influences parent report of concerns about symptoms of autism spectrum disorder. Autism 2019; 23(1): 100-11.
6. Guthrie et al. Accuracy of Autism Screening in a Large Pediatric Network. Pediatrics 2019; 144(4).
7. Carbone et al. Primary Care Autism Screening and Later Autism Diagnosis. Pediatrics 2020; 146(2): e20192314.
8. Wallis et al. Adherence to screening and referral guidelines for autism spectrum disorder in toddlers in pediatric primary care. PLoS One 2020; 15(5):e0232335.
9. Franz et al. Early intervention for very young children with or at high likelihood for autism spectrum disorder: An overview of reviews. Dev Med Child Neurol 2022; 64(9): 1063-76.
10. Shic et al. The autism biomarkers consortium for clinical trials: evaluation of a battery of candidate eye-tracking biomarkers for use in autism clinical trials. Mol Autism 2022; 13(1): 15.
11. Wei et al. Machine learning based on eye-tracking data to identify autism spectrum disorder: A systematic review and meta-analysis. J Biomed Inform 2023; 137: 104254.
12. Minissi et al. Assessment of the Autism Spectrum Disorder Based on Machine Learning and Social Visual Attention: A Systematic Review. J Autism Dev Disord 2022; 52(5): 2187-202.
13. Wen et al. Large scale validation of an early-age eye-tracking biomarker of an autism spectrum disorder subtype. Sci Rep 2022; 12(1): 4253.
14. Martin et al. Objective measurement of head movement differences in children with and without autism spectrum disorder. Mol Autism 2018; 9: 14.
15. Alvari et al. Is Smiling the Key?Machine Learning Analytics Detect Subtle Patterns in Micro-Expressions of Infants with ASD. J Clin Med 2021; 10(8).
16. Deveau et al. Machine learning models using mobile game play accurately classify children with autism. Intell Based Med 2022; 6: 100057.
17. Simeoli et al. Using Technology to Identify Children With Autism Through Motor Abnormalities. Front Psychol 2021; 12: 635696.
18. Anzulewicz et al. Toward the Autism Motor Signature: Gesture patterns during smart tablet gameplay identify children with autism. Sci Rep 2016; 6: 31107.
19. Chang et al. Computational Methods to Measure Patterns of Gaze in Toddlers With Autism Spectrum Disorder. JAMA Pediatr 2021; 175(8): 827-36.
20. Krishnappa Babu et al. Exploring complexity of facial dynamics in autism spectrum disorder. IEEE Trans Affect Comput 2021.
21. Carpenter et al. Digital Behavioral Phenotyping Detects Atypical Pattern of Facial Expression in Toddlers with Autism. Autism Res 2021; 14(3): 488-99.
22. Krishnappa Babu et al. Complexity analysis of head movements in autistic toddlers. J Child Psychol Psychiatry 2023; 64(1): 156-66.
23. Perochon et al. A scalable computational approach to assessing response to name in toddlers with autism. J Child Psychol Psychiatry 2021; 62(9): 1120-31.
24. Krishnappa Babu et al. Blink rate and facial orientation reveal distinctive patterns of attentional engagement in autistic toddlers: a digital phenotyping approach. Scientific Reports 2023, 13(1): 7158.
25. Perochon et al. A tablet-based game for the assessment of visual motor skills in autistic children. NPJ Digit Med 2023; 6(1): 17.
26. Chen & Guestrin. XGBoost: A Scalable Tree Boosting System. Proceedings of 22nd ACM SIGKDD International Conference on Knowledge Discovery and Data Mining 2016: 785-94.
27. Perkins & Schisterman. The Youden Index and the optimal cut-point corrected for measurement error. Biom J 2005; 47(4): 428-41.
28. Scott & Su-In. A unified approach to interpreting model predictions. Proceedings of 31st International Conference on Neural Information Processing Systems 2017: 4768-77.
29. Engelhard et al. Predictive Value of Early Autism Detection Models Based on Electronic Health Record Data Collected Before Age 1 Year. JAMA Network Open. 2023; 6(2): e2254303.
30. Lord et al. Autism diagnostic observation schedule: a standardized observation of communicative and social behavior. J Autism Dev Disord 1989; 19(2): 185-212.
31. Bishop et al. Convergent validity of the Mullen Scales of Early Learning and the differential ability scales in children with autism spectrum disorders. Am J Intellect Dev Disabil 2011; 116(5): 331-43.
32. Vabalas et al. Machine learning algorithm validation with a limited sample size. Plos One 14, e0224365 (2019).
33. Hanley & McNeil. The meaning and use of the area under a receiver operating characteristic (ROC) curve. Radiology 1982; 143(1): 29-36.

### Example 2

### Additional Information for Early Detection of Autism in Primary Care Based on Scalable Computational

### Behavioral Phenotyping

### Table of Contents

1. Glossary
2. Description of the app (movies and game)
3. Additional information about app variable computation
4. Additional information about app variables statistics
5. Computation of the prediction confidence score
6. Extreme Gradient Boosting (XGBoost) algorithm implementation
7. SHapley Additive exPlanations (SHAP) computation
8. Computation of the quality score
9. Adjusted/calibrated PPV and NPV scores
10. Exhaustive performances for all operating points
11. Model interpretability using SHAP values analysis
12. Additional illustrative examples

### 1. Glossary

ADOS-2. Autism Diagnostic Observation Schedule—Second Edition
App. Application, a type of software that can be installed and used on a computer, tablet, or smartphone
AUC. Area under the receiver operating characteristic (ROC) curve
CVA. Computer vision analysis is used to extract meaningful information from the toddler's videos, such as the detection of faces, the extraction of facial landmarks and head pose, etc.
DD-LD. Developmentally delayed or language delayed
DSM-5. American Psychiatric Association Diagnostic and Statistical Manual of Mental Disorders—5^{th} Edition
DUHS. Duke University Health System
EHR. Electronic Health Record
ICD-9/10. International Classification of Diseases, Ninth and Tenth Revisions
Interpretable machine learning. This refers to methods and models that make the behavioral phenotypes and predictions of machine learning algorithms understandable to humans. See Molnar C. Interpretable Machine Learning: A Guide for Making Black Box Models Explainable (2nd Edition). Munich: Independently published, 2022. In this work, this translates to the ability of our approach to determine the contributions of the app (behavioral) variables (and corresponding behaviors) to the individualized predictions.
IRB. Institutional Review Board
M-CHAT-R/F. Modified Checklist for Autism in Toddlers with Follow Up
NPV. Negative Predictive Value. Likelihood a person who has a negative test result does not have the condition being tested.
PPV. Positive Predictive Value. Likelihood a person with a positive test has the condition being tested.
ROC. Receiver Operating Characteristic Curve Sensitivity. The ability of a test to correctly identify persons with a condition.
SHapley Additive exPlanations (SHAP) values. SHAP values measure the contribution of the app variables to the final prediction. It measures the impact of having a certain value for a given variable in comparison to the prediction we would be making if that variable took a baseline value. See Scott ML, Su-In L. A unified approach to interpreting model predictions. Proceedings of the 31st International Conference on Neural Information Processing Systems 2017: 4768-77.
Specificity. The ability of a test to correctly identify persons without a condition.
XGBoost. EXtreme Gradient Boosting algorithm is a popular model based on several decision-trees whose node variables and split decisions are optimized using gradient statistics of a loss function. It constructs multiple graphs that examine the app variables under various sequential “if” statements. The algorithm progressively adds more “if” conditions to the decision tree to improve the predictions of the overall model. See Chen T, Guestrin C. XGBoost: A Scalable Tree Boosting System. Proceedings of the 22nd ACM SIGKDD International Conference on Knowledge Discovery and Data Mining 2016: 785-94.
Youden Index. Captures the performance of a dichotomous diagnostic test's ability to balance sensitivity and specificity. See Perkins NJ, Schisterman E F. The Youden Index and the optimal cut-point corrected for measurement error. Biom J 2005; 47(4): 428-41.

### 2. Description of the SenseToKnow App (Movies and Game)

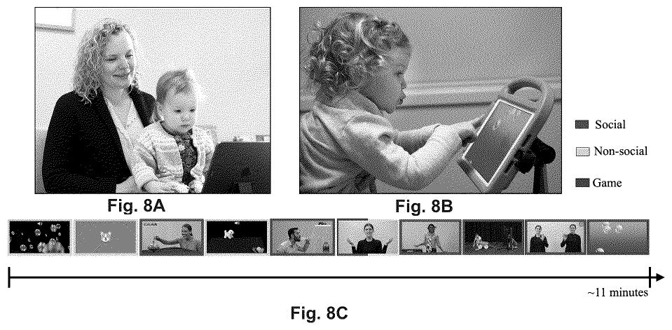

The stimuli (brief movies) and game used in the SenseToKnow app are as follows and are illustrated in FIG. 8. We note that there are images of the faces of individuals in the figures and videos. Consent has been given by these individuals (or their parents/guardians) for these images to be published in this manuscript.
Floating Bubbles. 35-second movie in which bubbles float upward producing a natural bubble sound.
Spinning Top. 53-second movie including an actress interacting with toys with her face and body on the right portion of the screen and the toys she interacts with on the left portion of the screen.
Mechanical Puppy. 25-second movie of a mechanical puppy toy that barks and wags its tail while it walks toward a set of toy fruit.
Blowing Bubbles. 64-second movie. Similar to Spinning Top, this movie displays an actor on one side of the frame (left) blowing bubbles toward the opposite side of the frame (right).
Rhymes and Toys. 49-second movie consisting of two separate parts: (1) a woman reciting nursery rhymes with gestures for 30 seconds and (2) dynamic toys with sound for 19 seconds.
Make Me Laugh. 56-second movie in which an actress wearing a polka-dotted clown tie engages in various actions designed to elicit smiles and laughter.
Dog in the Grass. 45-second movie in which a cartoon brown puppy appears in the corners of the screen and in the left and right sides of the screen, each time with a barking noise.
Playing with Blocks. 71-second movie in which two child actors (a boy and a girl) are interacting and playing with toys.
Fun at the Park. 51-second movie in which two women (one on each side of the frame) have a conversation. They make no gestures, and the conversation has a natural turn-taking flow.
Pop the Bubbles game. In this animated 40-second game, clear bubbles appear from the bottom of the screen (moving upward). Each bubble contains a marine animal inside, which can float away if the bubble is popped (by touching the screen where the bubble is). The game includes playful background music, and any time a bubble is popped, a distinct popping sound is heard. The animal is then fully seen, and it spins around before floating off the screen. An example video is of a child playing the game.

### 3. Additional Information about App Variable Computation

Videos were first analyzed using face detection and face recognition algorithms, which ensured the facial information of the target participant was analyzed while the facial information of others in the room was ignored. Human supervision was used to validate the outcome of the facial recognition procedure if the algorithm confidence was below a predefined threshold. On average, less than 10 frames per video required manual input. We extracted 49 facial landmark points consisting of 2D-positional coordinates that were time-synchronized with the movies. Using the facial landmarks, for each frame we computed the child's head pose angles relative to the tablet's frontal camera such as θ_{yaw} (left-right), θ_{pitch} (up-down), and θᵣₒₗₗ (tilting left-right) as described in Hashemi et. al. The participant's gaze information (projected onto the device screen) was extracted using an automatic gaze estimation algorithm based on a pre-trained deep neural network. This information was leveraged to extract relevant CVA-based behavioral phenotypes. See the following for more information: Baltrusaitis et al. Openface 2.0: Facial behavior analysis toolkit. IEEE International Conference on Automatic Face & Gesture Recognition 2018: 59-66.; King. Dlib-ml: A machine learning toolkit. J Machine Learning Research 2009; 10: 1755-8.; De la Torre et al. IntraFace. IEEE Int Conf Automatic Face Gesture Recognition Workshops 2015; 1.; Hashemi et al. Computer Vision Analysis for Quantification of Autism Risk Behaviors. IEEE Transactions on Affective Computing 2021; 12(1): 215-26; Krafka et al. Eye tracking for everyone. Proceedings of the IEEE Conference on Computer Vision and Pattern Recognition 2016: 2176-84.

Response to name. While children were watching the movies, their name was called three times by an examiner standing behind them. The analysis of the video audio allowed us to automatically detect the instant in which the participant's name was called, while the dynamics of the head orientation (pitch, yaw, and roll) were used to automatically detect if and when the child responded (oriented) toward the person calling their name. Computed vision and audio algorithms were originally proposed in Campbell and colleagues and improved by Perochon and colleagues. Both studies found that autistic toddlers oriented to their name less frequently, and those who oriented had a longer latency/delay in doing so. In the present work, we followed the implementation details described in Perochon and colleagues. Based on the automatic detection of the name call and the head turn events, we defined two CVA-based variables: the response to name ratio representing the proportion of times the participant oriented to their name call, and the response to name delay, which reflected the average delay (in seconds) between the beginning of the name call and the beginning of the head turn. See Campbell et al. Computer vision analysis captures atypical attention in toddlers with autism. Autism 2019; 23(3): 619-28 and Perochon et al. A scalable computational approach to assessing response to name in toddlers with autism. J Child Psychol Psychiatry 2021; 62(9): 1120-31 for more information.

Social attention. For the movies Spinning Top and Blowing Bubbles, we computed the percent of time the child gazed toward the social stimulus. The average across the two movies was represented by the variable Gaze Percent Social. Chang and colleagues reported that autistic toddlers spent more time gazing at the non-social stimulus compared to the social stimulus. See Chang et al. Computational Methods to Measure Patterns of Gaze in Toddlers With Autism Spectrum Disorder. JAMA Pediatr 2021; 175(8): 827-36.

During the Fun at the Park movie, in which two women take turns in a conversation, we evaluated the correlation of the gaze left-right patterns with the patterns of speech, reflected in the variable Gaze Speech Correlation. Chang and colleagues reported that the autistic toddlers' gaze patterns were less coordinated with the flow of conversational speech. See Chang et al., 2021.

For both the social attention (Gaze Percent Social) and attention to speech variables (Gaze Speech Correlation), gaze information was first estimated from the facial and eyes appearance using a pretrained deep neural network model. After this initial estimation, an attention proxy was considered by combining the following automatically computed rules: the child's eyes are open, the estimated gaze is within the screen region of interest, the face head pose is oriented toward the screen, and excluding periods in which the head is moving quickly (e.g., during head turns). All these steps were fully automatic and based on CVA; implementation details are provided by Chang and colleagues. Restricting the gaze information to the intervals in which the children attended to the movies (measured by our CVA-based proxy), we computed three behavioral variables. See Krafka et al. Eye tracking for everyone. Proceedings of the IEEE Conference on Computer Vision and Pattern Recognition 2016: 2176-84 and Chang et al., 2021.

Gaze silhouette score. During the social attention and attention to speech movies (Spinning Top, Blowing Bubbles, and Fun at the Park, respectively), it would typically be expected that children would alternate their gaze between the relevant social and non-social stimuli located on the right and left sides of the screen. As described by Chang et al., 2021, a clustering methodology can be used to automatically and robustly evaluate when participants are looking distinctly toward the right and left portions of the screen where relevant social and non-social stimuli are located (versus looking in a less structured/focused pattern), since the concentration of clusters can be mathematically assessed using the silhouette score. See Rousseeuw. Silhouettes: a graphical aid to the interpretation and validation of cluster analysis. Journal of Computational and Applied Mathematics 1987; 20: 53-65. Chang and colleagues found that autistic toddlers showed less coherent (clustered) gaze patterns. Using this approach, we defined the Gaze Silhouette Score as the average silhouette score over the three movies. This measure quantified how concentrated (focused gaze) or spread the gaze distribution was throughout these three movies.

Head movement. Using the facial landmarks associated with the corner of the eyes and the tip of the nose, we assessed the participant's head movement while they attended to the movies. The proxy for attention defined above was used to select the frames of interest, and the variation in the distance between the eyes was used to add invariance with respect to the distance to the screen. We computed the average head movement across each stimulus according to previously established studies. See Dawson et al. Atypical postural control can be detected via computer vision analysis in toddlers with autism spectrum disorder. Sci Rep 2018; 8(1): 17008 and Krishnappa Babu et al. Complexity analysis of head movements in autistic toddlers. J Child Psychol Psychiatry 2023; 64(1): 156-66. Dawson and colleagues reported that autistic toddlers showed more frequent head movement while watching the movies, accentuated during movies of high social content.¹⁸ We compared the rate of head movement across movies that contain a high level of social content (Spinning Top, Blowing Bubbles, Make Me Laugh, Playing with Blocks, and Fun at the Park) and those that contain primarily toys and animated objects (Floating Bubbles, Dog in Grass, and Mechanical Puppy). The average head movement across these two sets of movies were referred to as Head Movement during social and non-social stimuli.

Facing forward. A child's orientation towards the screen, i.e., ‘facing forward’ during any given frame was defined using their (i) head pose angle, (ii) eye gaze, and (iii) rapidity in head movement. The child's head pose |θ_{yaw}|25° was used, acting as a proxy for attentional focus on the screen, which is supported by the central bias theory for gaze estimation. See Li et al. Learning to predict gaze in egocentric video. Proceedings of the IEEE International Conference on Computer Vision 2013: 3216-23 and Mannan et al. Automatic control of saccadic eye movements made in visual inspection of briefly presented 2-D images. Spat Vis 1995; 9(3): 363-86. Then, for each frame we checked if the estimated gaze of the participant was on the tablet's screen and their eyes were open. Finally, we excluded the frames where the head was moving rapidly (this can lead to errors in the CVA). To this end, we first performed smoothing of the head pose signal θ_{yaw}, obtaining θ_{yaw}′. The head was considered to be moving rapidly if at any point θ_{yaw}′ of the current frame was >150% of the previous frame. Finally, the Facing Forward variable was estimated as a percentage of frames ‘facing forward’ out of the number of frames for each movie (ranging between 0 and 100). Details on the algorithm are presented in the supplementary materials of Chang et al., 2021.

Blink rate. Using CVA, we estimated the participant's number of blinks while they were watching each of the presented movies, as described next. OpenFace, a facial analysis toolkit that offered facial action units on a frame-by-frame basis, was used. These action units are based on the standard facial action coding system per Ekman. For the blinking action, we used action unit 45 (AU45) to estimate the child's blinks. A smoothing of the AU45 time-series signal was performed, followed by detecting the number of peaks, which are associated with blink actions. To obtain the blink rate, we normalized the number of blinks with respect to the number of valid frames. The valid frames were defined as frames during which the child was (i) facing forward (as defined above) and (ii) the confidence outcome of the OpenFace was at or above the recommended threshold (i.e., 0.75). For more details, see Krishnappa Babu et al. Blink rate and facial orientation reveal distinctive patterns of attentional engagement in autistic toddlers: a digital phenotyping approach. Scientific Reports 2023, 13(1): 7158.

Touch-based (visual motor skills) variables. We collected the tablet touch and device kinetic information while participants played the bubble popping game. In a recent study, we reported findings related to several touch-based variables. See Perochon et al. A tablet-based game for the assessment of visual motor skills in autistic children. NPJ Digit Med 2023; 6(1): 17. Based on the results of this previous study, we further evaluated four touch-based variables explored in the present study: the Touch Popping Rate (TPR), the Touch Average Length (TAL), the Touch Average Applied Force (TAAF), and the Touch Error Std (TES). The TPR is defined as the number of popped bubbles over the number of total touches to the screen, and it provides a notion of the accuracy and overall performance during the game. The TAL evaluated the average touch length, meaning the average length of the trajectory of the child's finger while it is in contact with the screen. The TAAF measures the average force associated with each individual touch, which can be estimated from the data collected by the tablet sensors. Finally, the TES is defined as the standard deviation of the distance between the child's finger position when hitting the screen and the center of the closest bubble. See also Video 2 showing a child playing the game.

### 4. Additional Information about App Variables Statistics

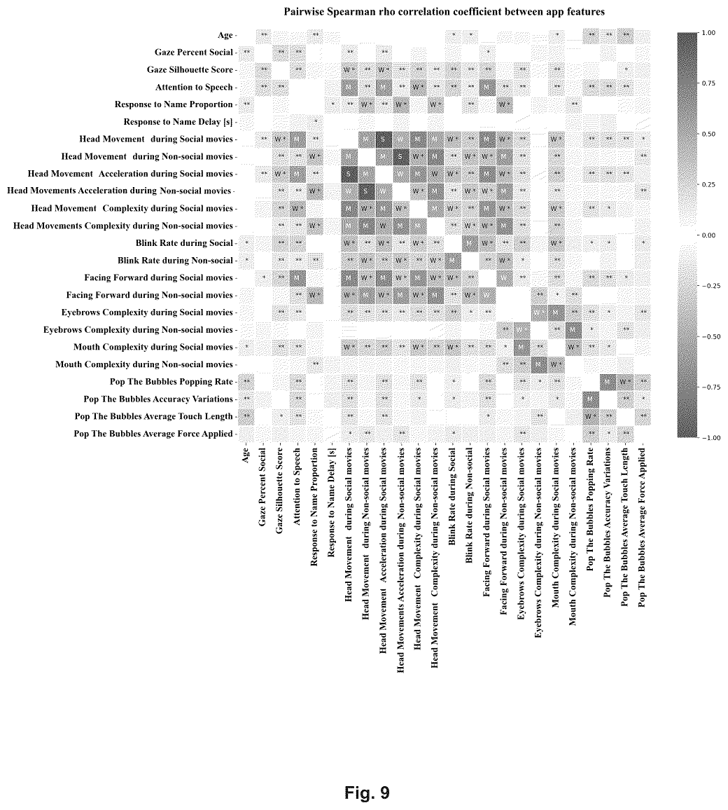

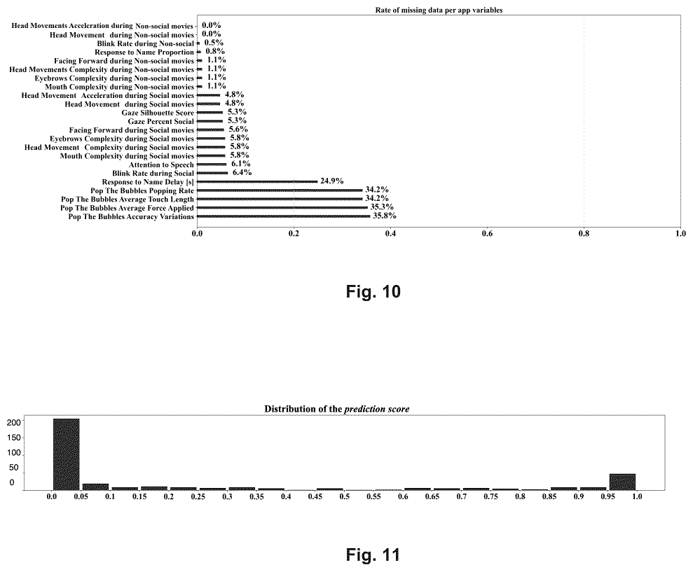

The app variables pairwise correlation coefficients are shown in FIG. 9 and the rate of missing data in FIG. 10.

### 5. Computation of the Prediction Confidence Score

We now explain the computation of the prediction confidence score, which is used to compute the model performances and assess the certainty of our approach to predict one of the diagnosis groups. The heterogeneity of the autistic condition implies that some autistic toddlers may show more complex behavioral patterns not primarily captured by the app variables, or only by a subset of them. The same holds for non-autistic participants who may exhibit behavioral patterns typically associated with autism (e.g., nonresponding to their name, or responding verbally instead). From a data science perspective, these challenging cases may be represented in ambiguous regions of the app variables space, as their variables might have a mix of autistic and neurotypical-related values. Therefore, the decision boundaries associated with these regions of the variable space may fluctuate when training the algorithm over different splits of the dataset, which we used to reveal the difficult cases. We counted the proportion of positive and negative predictions of each participant, over the K=1000 experiments. The distribution of these averaged prediction for each participant (which we called prediction confidence score; see FIG. 11) shows participants with consistent neurotypical predictions (prediction confidence score close to 0; at the extreme left of the figure), and with consistent autistic predictions (prediction confidence score close to 1; at the extreme right of the figure). The cases in between are considered more difficult since their prediction fluctuated between the two groups over the different training of the algorithm. We considered conclusive the administrations whose predictions were the same in at least 80% of the cases (either positive or negative predictions) and inconclusive otherwise. Interestingly, as illustrated in FIG. 11, the prediction confidence score can be related to the SHAP values of the participants. Indeed, conclusive administrations of the app have app variables contributions to the prediction that point to the same direction (either towards a positive or negative prediction), while inconclusive administrations show a mix of positive and negative contributions of the app variables.

### 6. Extreme Gradient Boosting (XGBoost) Algorithm Implementation

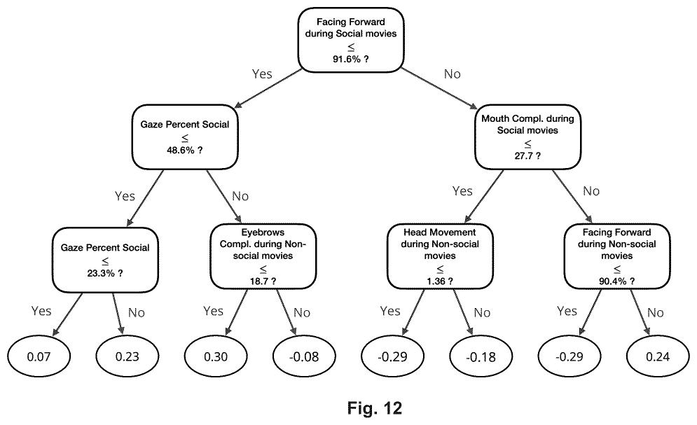

We used standard implementation of XGBoost as provided by the authors. We used all default parameters of the algorithms, except the ones in bold that we changed to account for the relatively small sample size and the class imbalance, and to prevent overfitting. n_estimators=100; max_depth=3 (default is 6, prompt to overfitting in this setting); objective=“binary:logistic”; booster=“gbtree”; tree_method=“exact” instead of “auto” since the sample size is relatively small; colsample_bytree=0.8 instead of 0.5 due to the relatively small sample size; subsample=1; colsumbsample=0.8 instead of 0.5 due to the relatively small sample size; learning_rate=0.15 instead of 0.3; gamma=0.1 instead of 0 to prevent overfitting, as this is a regularization parameter; reg_lambda=0.1; alpha=0. FIG. 12 illustrates one of the estimators of the trained model.

### 7. SHapley Additive exPlanations (SHAP) Computation

The Shapley Additive exPlanations values, originated in the cooperative game theory field, is a state-of-the-art method employed to shed lights into “black box” machine learning algorithms. This framework benefits from strong theoretical guarantees to explain the contribution of each input variable to the final prediction, accounting and estimating the contributions of the variable's interactions. See Molnar, Chapter 9.6 for a gentle introduction to the theoretical aspects of SHAP values.

In this work, the SHAP values were computed and stored for each sample of the test sets when performing cross-validation, i.e., training a different model every time with the rest of the data. Therefore, we needed to normalize the SHAP values first to compare them across different splits. The normalized contribution of the app variable was denoted as k (k∈[1,K]), for an individual i (i∈[1,N]), is

$${ϕ}_{k , Normalized}^{i} = \frac{{ϕ}_{k}^{i}}{{\sum }_{k = 1}^{K} \vert {ϕ}_{k}^{i} \vert} ∈ [ - 1 , 1 ] .$$

We conserved the sign of the SHAP values as it indicates the direction of the contribution, either toward autistic or neurotypical-related behavioral patterns.

In the learning algorithm used, being robust to missing values, an individual i may have a missing value for variable k, which will be used by the algorithm to compute a diagnosis prediction. In this case, the contribution (i.e., a SHAP value) of the missing data to the final prediction, still denoted as ϕₖⁱ, accounts for the contribution of this variable being missing.

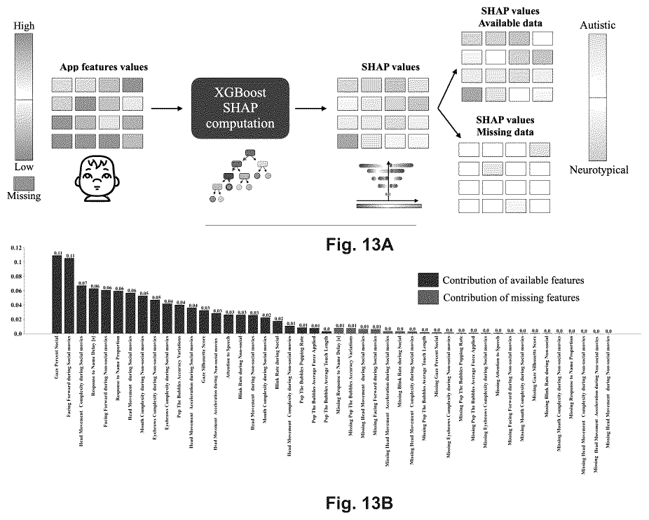

In order to disambiguate the contribution of actual variables from their missingness, we set to 0 the SHAP value associated with variable k for that sample and defined as ϕ_{Z}_{ₖ}ⁱ the contribution of having variable k missing for that sample. This is illustrated in FIG. 13.

This process leads to 2NK SHAP values for the study cohort, used to compute:
The importance of variable k to the model as the average contribution of that variable:

$${ϕ}_{k} = \frac{1}{N} {\sum }_{i = 1}^{N} \vert {ϕ}_{k}^{i} \vert ∈ [ 0 , 1 ] .$$

These contributions are represented in dark blue in FIG. 13 (panel b).
The importance of the missingness of variable k to the model, measured as the average contribution of the missingness of that variable:

$${ϕ}_{{Z}_{k}} = \frac{1}{N} {\sum }_{i = 1}^{N} \vert {ϕ}_{{Z}_{k}}^{i} \vert ∈ [ 0 , 1 ] .$$

These contributions are represented in sky blue in FIG. 13B.

### 8. Computation of the Quality Score

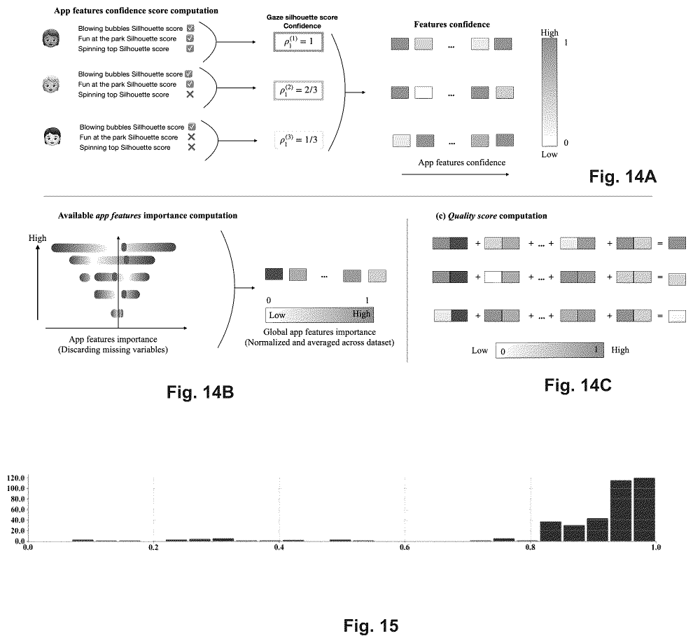

We now explain how the quality score is computed for each app administration, based on the amount of available information computed using the app data, weighted by the predictive ability (or variables importance) of each of the app variables. This score, between 0 and 1, quantifies the potential for the collected data on the participant to lead to a meaningful prediction of autism. FIG. 14 illustrates the intuition behind the computation of the confidence score of the app variables, the variables importance, and the quality score of an individual app administration.

Computation of the app variables confidence (prediction confidence score). Given the set of app variables (xₖⁱ)_{k∈[1,K]} for a participant i, we first compute a measure of confidence (or certainty) of each app variable, denoted by (ρₖⁱ)_{k∈[1,K]}. The intuition behind the computation of these confidence scores follows the weak law of large numbers which states that the average of a sufficiently large number of observations will be close to the expected value of the measure. We describe next the computation of the app variables confidence scores ρ.
As illustrated in FIG. 14, some app variables are computed as aggregates of several measurements. For instance, the Gaze Percent Social variable is the average percentage the participants spent looking at the social part of two of the presented movies. The confidence pₖⁱ of an aggregated variable k for participant i is the ratio of available measurements computed for participant i over the maximum number of measurements to compute that variable. Reasons for missing a variable for a movie include (i) the child did not attend to enough of the movie to trust the computation of that measurement, (ii) the movie was not presented to the participant due to technical issues, or (iii) the administration of the app was interrupted.
For the two variables related to the participant's response when their name is called, namely the proportion of response and the average delay when responding, the confidence was the proportion of valid name call experiments. Since their name was called a maximum of three times, the confidence score ranges from 0/3 to 3/3.
For the variables collected during the bubble popping game, we used as a measure of confidence the number of times of the participant touched the screen. The confidence is proportional to the number of touches when it is below or equal to 15, with 1 for higher number of touches and 0 otherwise.
The confidence of a missing variable is set to 0.

Computation of the app variables predictive power. When assessing the quality of the administration, one might want to put more weight in variables that contribute the most to the predictive performances of the model. Therefore, to compute the quality score of an administration, we used the normalized app variables importance (G(Xₖ))_{k∈[1,K]}to weight the app variables. Note that for computing the predictive power of the app variables, we used only the SHAP values of available variables, setting to 0 the SHAP values of missing variables.

Computation of the app administration quality score. After we compute for each administration i the confidence score (ρₖⁱ)_{k∈[1,K]}of each app variable (xₖⁱ)_{k∈[1,K]} and gain an idea of their expected predictive power (E_{X}[G(Xₖ)])_{k∈[1,K]}, the quality score is computed as:

$$Quality Score ( {x}^{i} ) = {\sum }_{k = 0}^{K} {E}_{X} [ G ( {X}_{k} ) ] {ρ}_{k}^{i} .$$

When all variables are missing, (ρₖⁱ)_{k∈[1,K]}=(0, . . . , 0), the score is equal to 0, and when all the app variables were measured with the maximum amount of information, (ρₖⁱ)_{k∈[1,K]}=(1, . . . , 1), then the quality score is equal to the sum of normalized variables contributions, which is equal to 1.

FIG. 15 shows the distribution of the quality score.

### 9. Adjusted/Calibrated PPV and NPV Scores

The prevalence of autism in the cohort analyzed in this study, as in many studies in the field, differs from the reported prevalence of autism in the broader population. While the 2018 prevalence of autism in the United States is of one over forty-four

$$( {π}_{population} = \frac{1}{44} \approx 2.3 \% ) ,$$

the analyzed cohort in this study is composed of 49 autistic participants and 328 non-autistic participants

$$( {π}_{population} = \frac{49}{328} \approx 14.9 \% ) .$$

Some screening tool performance metrics, such as the specificity, sensitivity, or the Area Under the Roc Curve (AUROC), are invariant to such prevalence differences, as their values don't depend on the group ratio (e.g., the sensitivity only depends on the measurement tool performance on the autistic group; the specificity only depends on the measurement tool performance on the non-autistic group). Therefore, providing an unbiased sampling of the population and a large enough sample size, the reported prevalence-invariant metrics should provide a good estimate of what would be the value of those metrics if the tool were implemented in the general population.

However, precision-based performance measures, such as the precision (or Predictive Positive Value; PPV), the Negative Predictive Value (NPV), or the FR scores depend on the autism prevalence in the analyzed cohort. Thus, these measures provide inaccurate estimates of the expected performance when the measurement tool is deployed outside of research settings.

Therefore, we now report the expected performance we would have if the autism prevalence in this study were the one in the population, following the procedure detailed in Siblini et al. Master Your Metrics with Calibration. In: Berthold et al. (eds) Advances in Intelligent Data Analysis XVIII. Cham: Springer International Publishing; 2020. p. 457-69.

For a reference prevalence, πₚₒₚᵤₗₐₜᵢₒₙ, and a study prevalence of π_{study}, the corrected PPV (or precision), corrected NPV, and F_{β} are:

$${PPV}_{C} = \frac{TP}{TP + \frac{{π}_{study} ( 1 - {π}_{population} )}{{π}_{population} ( 1 - {π}_{study} )} FP} , {F}_{β , C} = ( 1 + {β}^{2} ) \frac{{Precision}_{C} \cdot Sensitivity}{{β}^{2} Sensitivity + {Precision}_{C}} ,$$

and

TABLE S1
Table showing the sensitivity, specificity, and (corrected) Positive
Predictive Values (PPV) and Negative Predictive Values (NPV) for different
operating points of the Receiver Operating Curve. All app variables were used in
these analyses, and the model is trained to differentiate autistic (N = 49) from
neurotypical (N = 328) participants. The red and green rows correspond to the operating
point that optimizes the Youden and F₂ measures, respectively.

| | | Sensi- | Specifi- | | | PPV | NPV |
|---|---|---|---|---|---|---|---|
| Index | | tivity | city | | | Adjusted | Adjusted |
| threshold | Threshold | (%) | (%) | PPV (%) | NPV (%) | (%) | (%) |
| 1 | 1.0 | 34.7 | 98.5 | 77.3 | 32.6 | 91.0 | 98.6 |
| 2 | 0.999 | 34.7 | 97.9 | 70.8 | 25.7 | 90.9 | 98.6 |
| 3 | 0.998 | 36.7 | 97.3 | 66.7 | 22.1 | 91.1 | 98.6 |
| 4 | 0.997 | 40.8 | 97.0 | 66.7 | 22.1 | 91.6 | 98.7 |
| 5 | 0.996 | 46.9 | 97.0 | 69.7 | 24.6 | 92.4 | 98.9 |
| 6 | 0.989 | 51.0 | 97.0 | 71.4 | 26.2 | 93.0 | 98.9 |
| 7 | 0.986 | 53.1 | 96.3 | 68.4 | 23.6 | 93.2 | 99.0 |
| 8 | 0.961 | 61.2 | 96.3 | 71.4 | 26.2 | 94.3 | 99.2 |
| 9 | 0.957 | 61.2 | 96.0 | 69.8 | 24.7 | 94.3 | 99.1 |
| 10 | 0.954 | 65.3 | 96.0 | 71.1 | 25.9 | 94.9 | 99.2 |
| 11 | 0.951 | 65.3 | 95.4 | 68.1 | 23.3 | 94.8 | 99.2 |
| 12 | 0.935 | 65.3 | 94.2 | 62.7 | 19.3 | 94.8 | 99.2 |
| 13 | 0.932 | 65.3 | 93.6 | 60.4 | 17.8 | 94.8 | 99.2 |
| 14 | 0.899 | 65.3 | 92.4 | 56.1 | 15.4 | 94.7 | 99.2 |
| 15 | 0.898 | 67.3 | 92.1 | 55.9 | 15.3 | 95.0 | 99.3 |
| 16 | 0.895 | 69.4 | 92.1 | 56.7 | 15.7 | 95.3 | 99.3 |
| 17 | 0.879 | 69.4 | 91.2 | 54.0 | 14.3 | 95.2 | 99.3 |
| 18 | 0.858 | 71.4 | 91.2 | 54.7 | 14.6 | 95.5 | 99.3 |
| 19 | 0.848 | 71.4 | 90.5 | 53.0 | 13.8 | 95.5 | 99.3 |
| 20 | 0.834 | 73.5 | 90.5 | 53.7 | 14.2 | 95.8 | 99.4 |
| 21 | 0.777 | 73.5 | 89.9 | 52.2 | 13.4 | 95.8 | 99.4 |
| 22 | 0.775 | 75.5 | 89.9 | 52.9 | 13.8 | 96.1 | 99.4 |
| 23 | 0.765 | 75.5 | 89.3 | 51.4 | 13.1 | 96.1 | 99.4 |
| 24 | 0.755 | 77.6 | 89.3 | 52.1 | 13.4 | 96.4 | 99.5 |
| 25 | 0.657 | 77.6 | 86.3 | 45.8 | 10.7 | 96.3 | 99.5 |
| 26 | 0.654 | 79.6 | 86.3 | 46.4 | 11.0 | 96.6 | 99.5 |
| 27 | 0.653 | 79.6 | 86.0 | 45.9 | 10.8 | 96.6 | 99.5 |
| 28 | 0.65 | 81.6 | 86.0 | 46.5 | 11.0 | 96.9 | 99.5 |
| 29 | 0.607 | 81.6 | 83.8 | 43.0 | 9.7 | 96.8 | 99.5 |
| 30 | 0.579 | 83.7 | 83.8 | 43.6 | 9.9 | 97.2 | 99.6 |
| 31 | 0.471 | 83.7 | 82.0 | 41.0 | 9.0 | 97.1 | 99.6 |
| 32 | 0.466 | 85.7 | 82.0 | 41.6 | 9.2 | 97.5 | 99.6 |
| 33 (F² | 0.382 | 85.7 | 80.8 | 40.0 | 8.7 | 97.4 | 99.6 |
| optimal) | | | | | | | |
| 34 (Youden | 0.378 | 87.8 | 80.8 | 40.6 | 8.8 | 97.8 | 99.7 |
| optimal) | | | | | | | |
| 35 | 0.366 | 87.8 | 80.2 | 39.8 | 8.6 | 97.8 | 99.7 |
| 36 | 0.342 | 87.8 | 78.7 | 38.1 | 8.0 | 97.7 | 99.7 |
| 37 | 0.336 | 89.8 | 78.7 | 38.6 | 8.2 | 98.1 | 99.7 |
| 38 | 0.182 | 89.8 | 70.7 | 31.4 | 6.1 | 97.9 | 99.7 |
| 39 | 0.18 | 89.8 | 70.1 | 31.0 | 6.0 | 97.9 | 99.7 |
| 40 | 0.179 | 91.8 | 69.8 | 31.2 | 6.1 | 98.3 | 99.8 |
| 41 | 0.124 | 91.8 | 67.7 | 29.8 | 5.7 | 98.2 | 99.7 |
| 42 | 0.119 | 91.8 | 67.1 | 29.4 | 5.6 | 98.2 | 99.7 |
| 43 | 0.097 | 91.8 | 66.5 | 29.0 | 5.5 | 98.2 | 99.7 |
| 44 | 0.094 | 91.8 | 65.9 | 28.7 | 5.4 | 98.2 | 99.7 |
| 45 | 0.093 | 91.8 | 65.5 | 28.5 | 5.4 | 98.2 | 99.7 |
| 46 | 0.092 | 93.9 | 65.5 | 28.9 | 5.5 | 98.6 | 99.8 |
| 47 | 0.089 | 93.9 | 65.2 | 28.7 | 5.4 | 98.6 | 99.8 |
| 48 | 0.088 | 93.9 | 64.3 | 28.2 | 5.3 | 98.6 | 99.8 |
| 49 | 0.055 | 93.9 | 61.3 | 26.6 | 4.9 | 98.5 | 99.8 |
| 50 | 0.046 | 93.9 | 60.7 | 26.3 | 4.8 | 98.5 | 99.8 |
| 51 | 0.036 | 93.9 | 59.5 | 25.7 | 4.7 | 98.5 | 99.8 |
| 52 | 0.033 | 93.9 | 58.8 | 25.4 | 4.6 | 98.5 | 99.8 |
| 53 | 0.032 | 93.9 | 58.5 | 25.3 | 4.6 | 98.5 | 99.8 |
| 54 | 0.028 | 93.9 | 56.7 | 24.5 | 4.4 | 98.4 | 99.8 |
| 55 | 0.023 | 93.9 | 56.4 | 24.3 | 4.4 | 98.4 | 99.8 |
| 56 | 0.021 | 93.9 | 55.8 | 24.1 | 4.3 | 98.4 | 99.8 |
| 57 | 0.02 | 93.9 | 55.5 | 24.0 | 4.3 | 98.4 | 99.8 |
| 58 | 0.019 | 93.9 | 54.9 | 23.7 | 4.2 | 98.4 | 99.8 |
| 59 | 0.014 | 93.9 | 54.0 | 23.4 | 4.2 | 98.3 | 99.8 |
| 60 | 0.013 | 93.9 | 53.4 | 23.1 | 4.1 | 98.3 | 99.8 |
| 61 | 0.011 | 93.9 | 53.0 | 23.0 | 4.1 | 98.3 | 99.8 |
| 62 | 0.01 | 95.9 | 52.4 | 23.2 | 4.1 | 98.9 | 99.8 |
| 63 | 0.009 | 95.9 | 51.5 | 22.8 | 4.0 | 98.8 | 99.8 |
| 64 | 0.007 | 95.9 | 50.9 | 22.6 | 4.0 | 98.8 | 99.8 |
| 65 | 0.005 | 96.0 | 48.0 | 22.0 | 99.0 | 4.0 | 99.83 |
| 66 | 0.004 | 96.0 | 46.0 | 21.0 | 99.0 | 4.0 | 99.82 |
| 67 | 0.003 | 96.0 | 44.0 | 20.0 | 99.0 | 4.0 | 99.81 |
| 68 | 0.002 | 96.0 | 41.0 | 19.0 | 99.0 | 3.0 | 99.8 |
| 69 | 0.001 | 96.0 | 38.0 | 19.0 | 98.0 | 3.0 | 99.79 |
| 70 | 0.0 | 100.0 | 0.0 | 13.0 | 100.0 | 2.0 | 99.77 |

${NPV}_{C} = \frac{\frac{{π}_{study} ( 1 - {π}_{population} )}{{π}_{population} ( 1 - {π}_{study} )} TN}{FN + \frac{{π}_{study} ( 1 - {π}_{population} )}{{π}_{population} ( 1 - {π}_{study} )} TN} .$

### 10. Exhaustive Performances for all Operating Points

Table S1 provides the performances of the XGBoost trained to differentiate autistic from neurotypical participants using all app variables, for all the cut-off thresholds defining the operating points of the associated ROC.

### 11. Model Interpretability Using SHAP Values Analysis

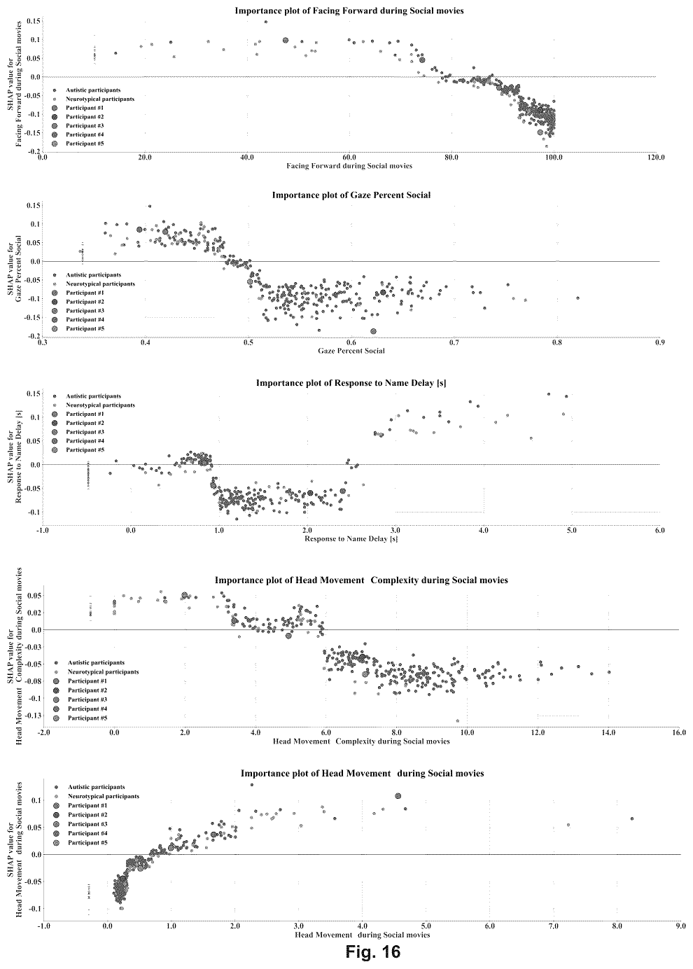

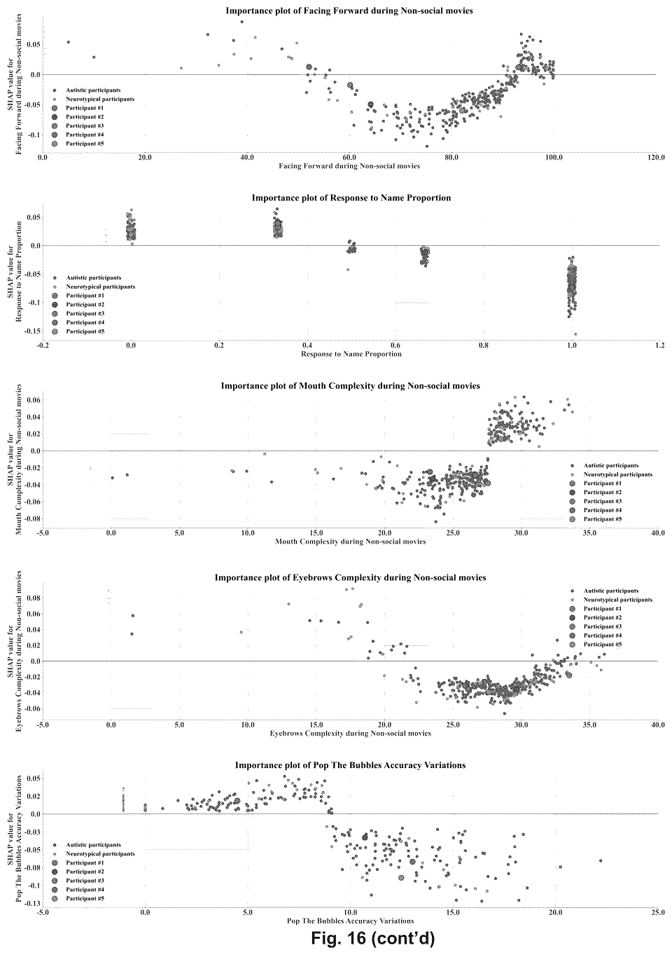

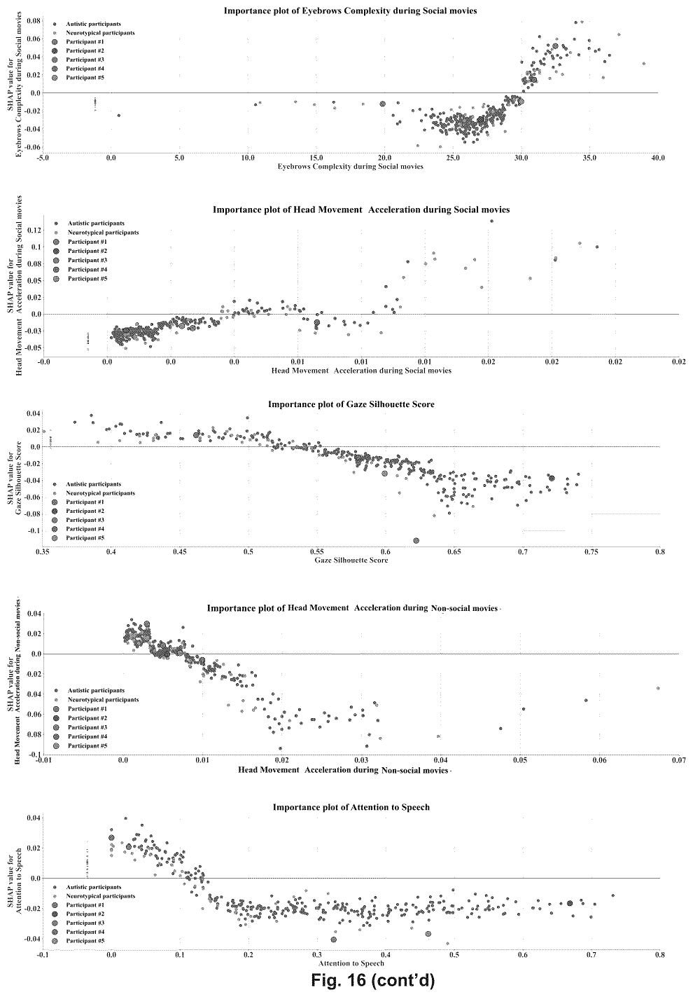

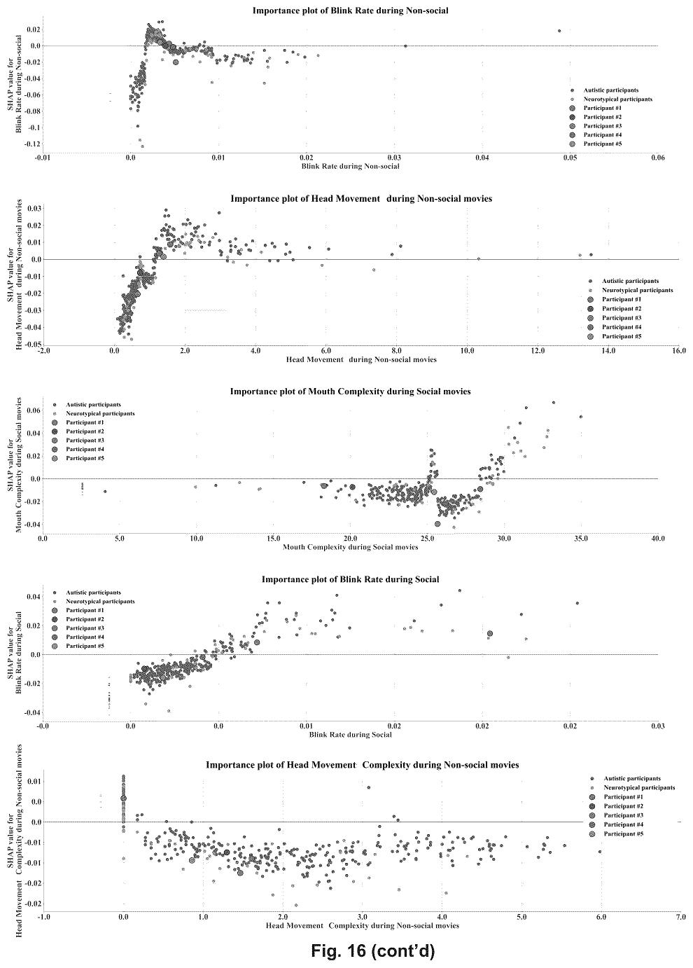

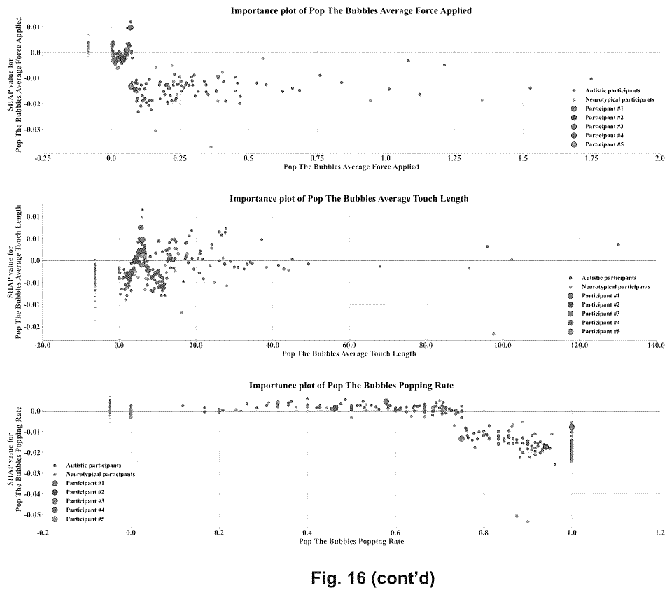

We provide the visualization of the dependence between the app variables values and their contribution to the model. This allows us to understand which ranges of the variables' values correspond to an increase or decrease of the model's prediction towards the autistic or neurotypical groups. The app variables are ordered by their global importance to the model; see FIG. 16.

### 12. Additional Illustrative Examples

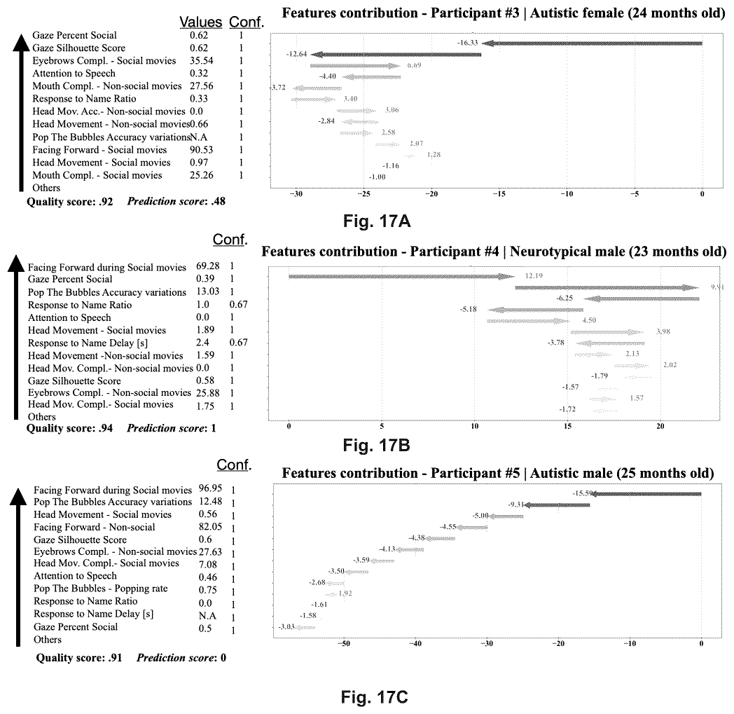

FIG. 17 illustrates three additional illustrative cases, as follows: (Participant #3) an autistic child who did not receive an M-CHAT-R/F administration; (Participant #4) a neurotypical child incorrectly predicted as autistic; and (Participant #5) an autistic participant incorrectly predicted as neurotypical. The proposed framework enables us to provide explanations on the misclassified cases.

### Example 3

### A Tablet-Based Game for the Assessment of Visual Motor Skills in Autistic Children

Increasing evidence suggests that early motor impairments are a common feature of autism. Thus, scalable, quantitative methods for measuring motor behavior in young autistic children are needed. This work presents an engaging and scalable assessment of visual-motor abilities based on a bubble-popping game administered on a tablet. Participants are 233 children ranging from 1.5 to 10 years of age (147 neurotypical children and 86 children diagnosed with autism spectrum disorder [autistic], of which 32 are also diagnosed with co-occurring attention-deficit/hyperactivity disorder [autistic+ADHD]). Computer vision analyses are used to extract several game-based touch features, which are compared across autistic, autistic+ADHD, and neurotypical participants. Results show that younger (1.5-3 years) autistic children pop the bubbles at a lower rate, and their ability to touch the bubble's center is less accurate compared to neurotypical children. When they pop a bubble, their finger lingers for a longer period, and they show more variability in their performance. In older children (3-10-years), consistent with previous research, the presence of co-occurring ADHD is associated with greater motor impairment, reflected in lower accuracy and more variable performance. Several motor features are correlated with standardized assessments of fine motor and cognitive abilities, as evaluated by an independent clinical assessment.

These results highlight the potential of touch-based games as an efficient and scalable approach for assessing children's visual motor skills, which can be part of a broader screening tool for identifying early signs associated with autism.

### Introduction

Early detection of autism provides an opportunity for early intervention, which can improve developmental trajectories and strengthen social, language, cognitive, and motor competencies during a period of heightened brain plasticity¹⁻⁴. The current standard of care for autism screening most often relies on a caregiver questionnaire, such as the Modified Checklist for Autism in Toddlers-Revised (MCHAT-R/F), which is used for neurodevelopmental screening in children between 16-30 months of age^{5,6}.

Although useful, the MCHAT-R/F has lower accuracy when administered in real-world settings, such as primary care^{7,8}. Furthermore, the MCHAT-R/F's performance is influenced by the family's socioeconomic status, maternal education level, and the child's sex, race, and ethnicity⁷⁻¹⁰. Thus, new objective screening and assessment tools based on direct assessment of the child's behavior are needed that can complement screening approaches based on caregiver questionnaires.

While autism is fundamentally characterized by qualitative differences in social and communication domains, impairments in motor abilities have also been documented in autistic children¹¹⁻¹⁵. The prevalence estimates of motor impairments in autism range from 50-85%,^{14,16-19} these estimates could potentially represent lower bounds since they are limited by the sensitivity of current assessment methods¹⁵. Motor impairments often are one of the earliest reported signs associated with autism²⁰⁻²², and have been documented in autistic children without cognitive impairment¹⁹. Thus, early assessment of motor skills could be a useful component of an early screening battery for autism. Several aspects of motor skills have been studied in autism, including gait and balance stability, coordination, movement accuracy, reaction time, manual dexterity, tone, hyperkinesis, and praxis^{15,21}. Various methods have been used to assess such skills using non-gamified paradigms, such as quantifying horizontal arm swings²³, variations in reaching to grasp²⁴ or touch²⁵, handwriting²⁶, and gait²⁷.

Research suggests that differences in motor skills associated with autism emerge during infancy. LeBarton and Landa examined motor skills in 6-month-old infants with and without an older sibling with autism. Motor skills at 6 months predicted both an autism diagnosis and level of expressive language acquisition by 30-36 months²⁸. These findings are consistent with other studies that have reported that the early development of motor skills is associated with expressive language outcomes among autistic children^{29,30}. A recent study of patterns of health care utilization in infants who were later diagnosed with autism found a higher rate of physical therapy visits below age 1, underscoring the early manifestation of motor impairments in autism³¹.

Studies that have sought to characterize the nature of motor impairments in autism have found that autistic children are particularly challenged by tasks that require efficient visual-motor integration³². Visual-motor integration ability affects many domains of functioning, including imitation, which is fundamental for developing social skills. There is some evidence supporting a bias toward proprioceptive feedback over visual feedback in autism^{33,34}. The tablet-based bubble-popping game developed for this study requires the temporal coordination of a dynamic visual stimulus with a motor response involving touch. As such, it is well suited to assess this aspect of early motor development.

The development of miniaturized inertial sensors, wearable sensors, and the ubiquity of mobile devices such as tablets and smartphones have allowed unprecedented access to massive multimodal data acquisition that has been used to characterize motor behavior. These data have been used to derive predictors of Parkinson's severity³⁵, identify and quantify an autism motor signature and characterize the nature of motor impairments in autism³⁶⁻⁴⁵. These studies demonstrate the usefulness of tablet-based assessments and games for assessing motor skills.

In the present EXAMPLE, we sought to extend current research findings in three ways. First, we sought to evaluate a tablet-based, gamified visual motor assessment in toddlers at the age when autism screening is typically conducted. Second, intellectual abilities have been found to be correlated with motor impairment in autistic children;^{24,46} thus, we accounted for the contribution of co-occurring cognitive impairment to motor ability in our analyses.

Third, as ADHD has also been associated with motor impairment, we sought to examine the combined contribution of autism and ADHD to the level and nature of motor impairment⁴⁷. Previous studies have found that the prevalence of motor impairment among autistic individuals increases when there is co-occurring cognitive impairment and/or psychiatric conditions, including ADHD. One study found that the proportion of autistic children with motor impairment increased by 4.4% if the child had cooccurring ADHD. This study found that the nature of motor impairment in autism versus ADHD may differ, however⁴⁸. Research suggests that, while autism has been associated with impairment in visual-proprioceptive integration, motor difficulties in ADHD tend to be associated with variability in the accuracy and speed of movement³⁴.

The bubble popping game examined in this study is one part of a mobile application (app) developed by our team that displays developmentally appropriate and strategically designed movies while recording the child's behavioral responses to the stimuli⁴⁹. The app is administered on smartphones and tablets and does not require spoken language or literacy. Direct observation offers a unique opportunity for capturing and objectively quantifying various aspects of child behavior. We have previously reported results from children's behavioral responses to the movies, which have been found to differentiate autistic from neurotypical toddlers⁵⁰⁻⁵⁶. In the current work, we focused on the bubble popping game, which utilizes inertial and touch features. Based on previous studies, we predicted that autistic children would have a distinct performance on the bubble-popping game, and this pattern would differ between autistic children with versus without co-occurring ADHD. Additionally, we examined whether the motor digital phenotypes derived from the game correlated with standardized measures of cognitive, language, and motor abilities, as well as level of autism-related behaviors, to better understand the relationship between children's motor behavior and their clinical profiles.

In summary, our goals were to: (i) assess motor behavior in children as young as 18 months using a tablet-based game to distinguish autism and neurotypical development at the age at which autism screening is typically conducted, (ii) control for the effects of cognitive ability in our analyses, (iii) evaluate the impact of co-occurring ADHD on motor function in young autistic children, and (iv) evaluate several novel visual-motor features derived from a simple, scalable game and their relationships with children's clinical profiles.

### Results

Correlations between motor performance and age. We first examined whether age of the participants was correlated with performance on the game. Combining samples from studies 1 and 2, results indicate that there was a strong correlation between the participant's age and their game performance. Age has a significant positive association with the number of touches (rho=0.62, p<1e-25, N=233) and the bubble popping rate (rho=0.50, p<1e-17, N=233); and a significant negative association with the median distance to the center (rho=−0.48, p<1e-16, N=233), the average touch duration (rho=−0.70, p<1e-36, N=233) and the average touch length (rho=−0.63, p<1e-28, N=233). Given these associations, age was added as a covariate for all group comparisons and correlations in both studies.

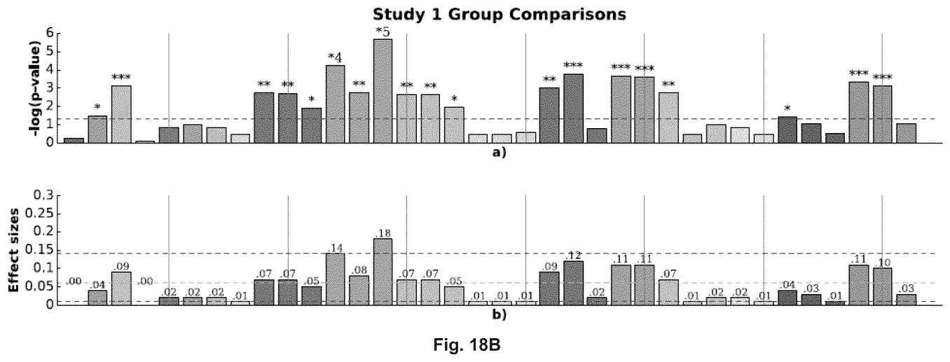

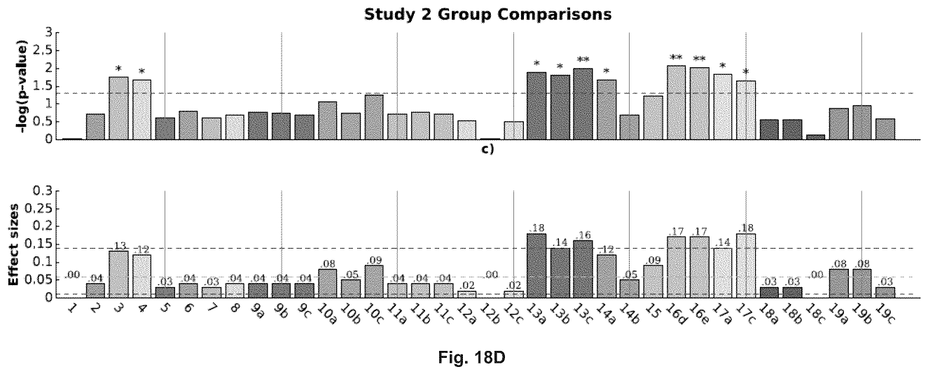

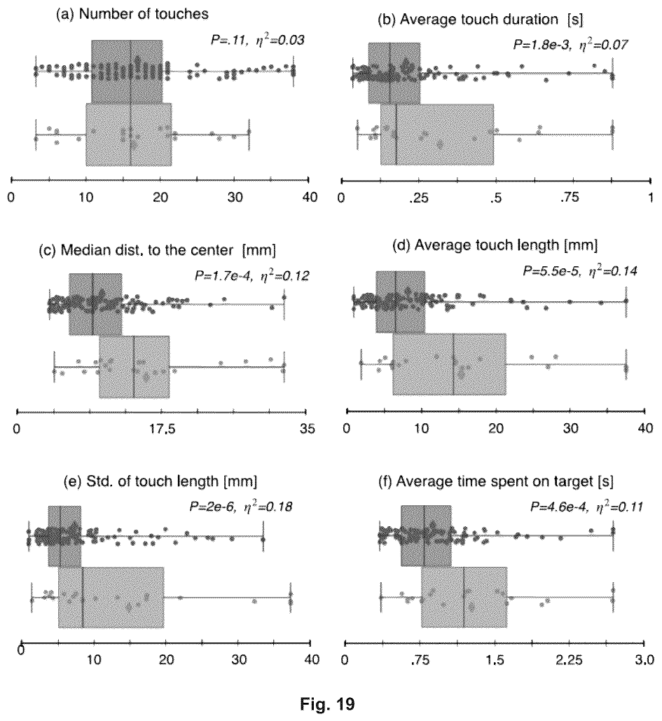

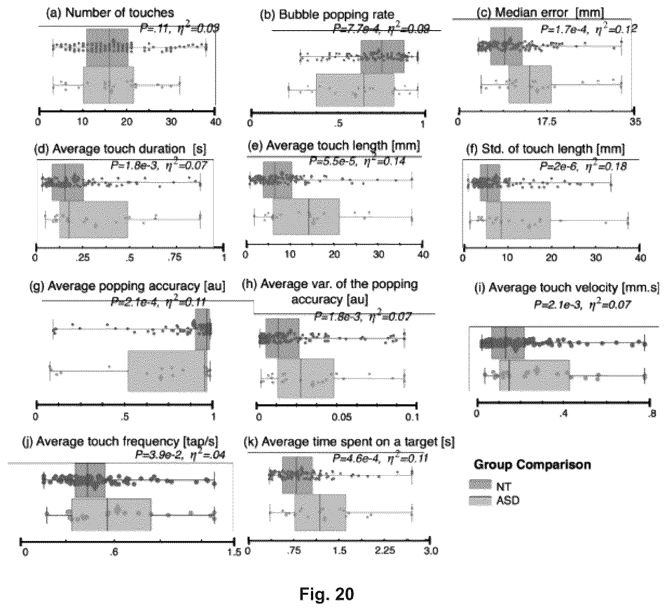

Study 1: Comparisons of younger autistic versus neurotypical children Autistic and neurotypical participants in study 1 did not statistically differ in terms of their previous experience playing tablet-based games (Z=0.96, p=0.33; proportion Z-test). The level of engagement/compliance was not a significant factor, indicated by the high completion rate, higher than 95% for both groups. The age distribution comparison between the age-matched neurotypical group (N=128) and autistic group was statistically non-significant (p=0.07, r=0.23; two-sided Mann-Whitney-U test). The two groups did not differ in terms of the mean number of touches, indicating similar levels of overall engagement with the game. However, the two groups were found to statistically differ in terms of several other motor variables. FIGS. 18A-18D shows the p-values and effect sizes when comparing autistic and neurotypical toddlers on these touch-related features, and the data distribution of a subset of features are shown in FIGS. 19A-19F and FIGS. 20A-20K. Results showed that the autistic participants exhibited a lower bubble popping rate (FIG. 20B, F(1, 148)=15.14, p=7.7e-4, η2=0.09), and their median distance to the center (mm) was larger (FIG. 19C, F(1,148)=20.14, p=1.7e-4, η2=0.12). Additionally, we observed that autistic participants had a longer average touch length (FIG. 19D, F(1, 148)=23.56, p=5.5e-5, η2=0.14), and showed greater variability in their touch length (FIG. 19E, F(1, 148)=32,70, p=2e-6, η2=0.18). We also found that the neurotypical participants took less time, on average, to pop a targeted bubble than autistic participants, as represented by the average time spent to pop a bubble (FIG. 19F, F(1, 148)=18.56, p=4.6e-4, η2=0.11). F-statistics and associated p-values were computed using a one-way ANCOVA.

Study 2: Comparisons of older autistic versus neurotypical children. We first compared the autistic group (including those with cooccurring ADHD) and the neurotypical group in terms of their game performance. The two groups were found to differ in level of cognitive ability (p=2e-5, r=0.64; two-sided Mann-Whitney-U test) but not age (p=0.15, r=0.21; two-sided Mann-Whitney-U test); thus, we included both age and IQ, as reflected in their General Conceptual Ability (GCA) score, as covariates in these analyses. The level of engagement, as reflected in the mean number of touches, did not differ between autistic and neurotypical children (F (1,78)=0.428, p=0.77, η2=0.01; one-way ANCOVA). However, autistic children showed a significantly lower average touch frequency (F (1,57)=14.77, p=1.1e-2, η2=0.21), and a lower median time spent targeting a bubble (F (1,57)=10.79, p=2.0e-2, η2=0.16).

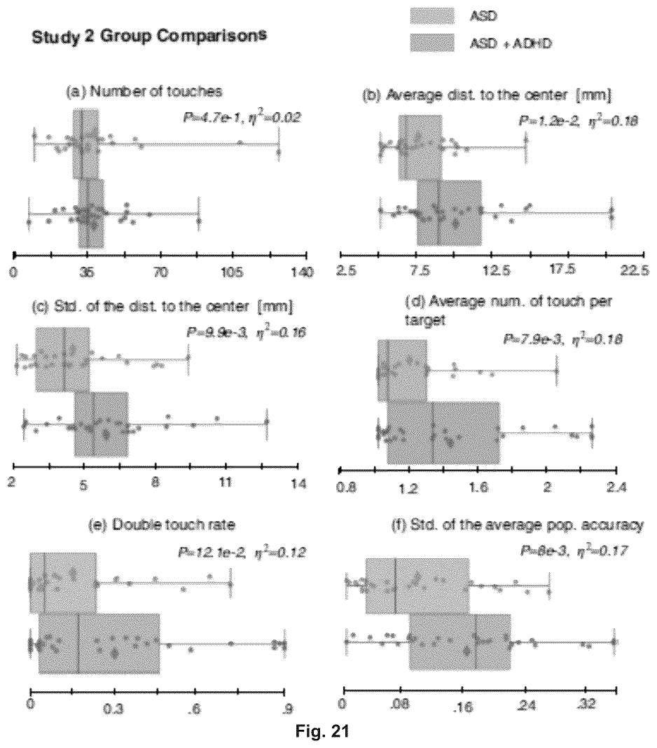

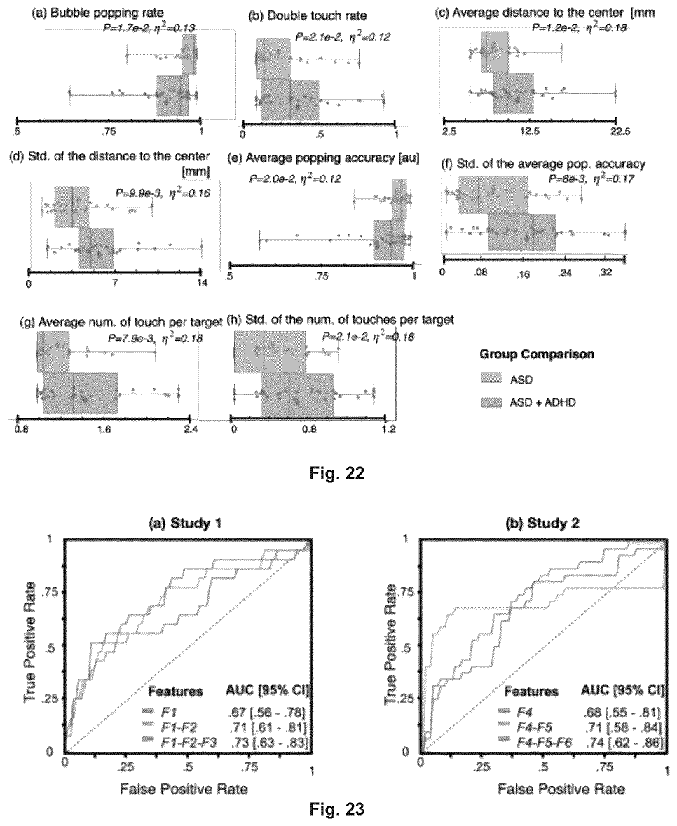

Study 2: Comparisons of older autistic children with and without ADHD Children with and without ADHD did not differ in terms of age (p=0.052, r=0.28), previous experience playing video games (Z=−1.08, p=0.28; proportion Z-test), or their cognitive ability (IQ) based on their GCA on the DAS (p=0.68, r=0.06; two-sided Mann-Whitney-U test). FIG. 21 shows the distribution of a subset of touch-related features for the autistic participants with and without co-occurring ADHD. Fatigue/noncompliance was not a significant factor as the dropout rate for both groups was <0.5%. Although the engagement in the task did not differ significantly between the autistic participants with and without ADHD, as indicated by the number of touches (FIG. 21A, F(1, 60)=0.02, p=0.90, h2=0.00; one-way ANCOVA), significant differences were observed in other motor features. FIG. 18 shows the p values and effect sizes when comparing children with and without ADHD on the touch-related features. Autistic participants with ADHD were, on average, less accurate as indicated by their average distance to the center (FIG. 21B, F(1, 60)=12.76, p=1.2e-2, η2=0.12), and consequently had a lower bubble popping rate (FIG. 22A, F(1, 60)=8.98, p=1.7e-2, η2=0.13). Although the total number of touches did not differ, the group with ADHD showed higher number of touches per target (FIG. 21D, F (1, 60)=10.0, p=1.4e-2, η2=0.14). In addition, the group with ADHD showed more variability in their movement and accuracy. Specifically, they showed a higher variability (std) in their number of touches per target (FIG. 22G, F(1,60)=13.10, p=2.1e-2, η2=0.18), the distance to the center (FIG. 21C, F(1,60)=11.26, p=9.9e-3, η2=0.16), and the average popping accuracy (FIG. 21F, F(1,60)=12.71, p=8.6e-3, η2=0.18). Additional results are presented in Supplementary FIG. 2.

Combining features for group discrimination. For study 1, we hypothesized that combining multiple features would improve discrimination of autistic and neurotypical toddlers. To this end, we trained logistic regression models to infer from the touch-based features the participant's clinical diagnosis and performed leave-one-out cross-validation to assess the generalization performances of these models. We compared the performance of individual features and a combination of them to assess their complementariness. FIG. 23 presents the receiver operating characteristic (ROC) curves and area under the curve (AUC) obtained for models trained by successively adding a single motor feature at a time. For study 1, the ROC shows the proportion of autistic participants correctly classified correctly vs. the proportion of autistic toddlers incorrectly classified by the model. Results showed that logistic regression trained on multiple game-based features improved the classification power; the AUCs using one, two, or three-motor features were 0.67 (95% CI, 0.56-0.78; average length), 0.71 (95% CI, 0.61-0.81; adding the average touch duration), and 0.73 (95% CI, 0.63-0.83; adding the average time spent), respectively.

For study 2, we also hypothesized that combining the motor related features would improve group discrimination. The same previously described feature selection procedure was used. The ROC curve in FIG. 23B shows the proportion of Autistic+ADH participants correctly classified vs. the proportion of autistic children incorrectly classified by the model. The AUCs using one, two or three motor features were 0.68 (95% CI, 0.55-0.81; average distance to the center), 0.74 (95% CI, 0.58-0.84; adding the number of targets), and 0.74 (95% CI, 0.62-0.86; adding the screen exploratory percentage), respectively. Supplementary Table 1 (below) provides the AUCs obtained using three motor features by sex and racial/ethnic background. The AUC values remained relatively consistent for these subgroups; however, Cis were larger owing to smaller sample sizes.

Study 1. Correlations between motor performance and clinical characteristics

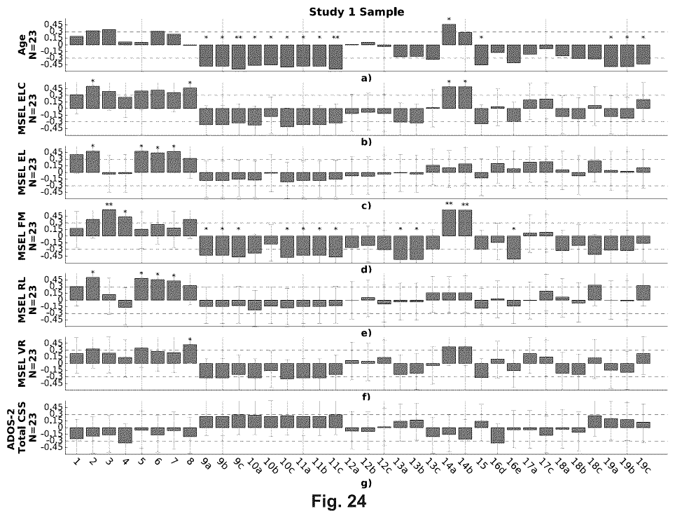

Spearman's rho correlation was used to assess the relationship between motor features and clinical variables, with statistical significance computed using a Student's t-distribution. We first examined the partial correlations between motor performance and the clinical characteristics based on clinician-administered measures, controlling for age, for the autistic children in study 1, including their performance on the Mullen Scales of Early Learning (MSEL) and the Autism Diagnostic Observation Schedule (ADOS total calibrated severity score). Partial correlations are illustrated in FIGS. 24A-24G for the autistic toddlers of the study 1 sample. The fine motor T-score of the MSEL was found to be positively correlated with the pop rate (rho=0.59, p=3.2e-3; Student's t-distribution from now on), the double touch rate (rho=0.43, p=4.8e-2), and the average popping accuracy (rho=0.62, p=2.0e-3), and negatively correlated with the average touch velocity (rho=−0.43, p=4.5e-2), the average and std touch duration (rho=−0.43, p=4.5e-5 and rho=−0.47, p=2.5e-2 respectively), and the variability of the maximum popping accuracy (rho=−0.52, p=1.2e-2). The early learning composite score of the MSEL was found to be positively associated with the number of pops (rho=0.51, p=1.5e-2) and the average popping accuracy (rho=0.49, p=1.9e-2). The expressive language T-score of the MSEL was found to be positively correlated with the screen exploratory percentage (rho=0.47, p=2.5e-2) and the total number of targets (rho=0.43, p=2.1e-2). The receptive language T-score was positively associated with the screen exploratory percentage (rho=0.48, p=2.1e-2), and the visual reception T-score was positively correlated with the repeat percentage variable (rho=0.42, p=4.8e-2). No significant correlations were found between the motor features and the total calibrated severity score of the ADOS.

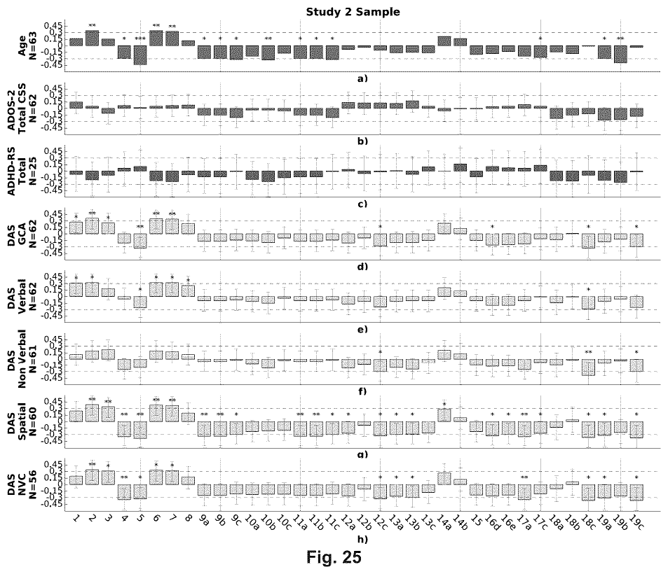

Study 2. Correlations between motor performance and clinical characteristics Spearman's rho correlation was again used to assess the relationship between motor features and clinical variables, with statistical significance computed using a Student's t-distribution. We examined the partial correlations between motor performance and the clinical characteristics, controlling for age, for the autistic children in study 2, including their performance on the ADOS total calibrated severity score, ADHD rating-scale total score, and the DAS. These analyses included children with and without cooccurring ADHD. Partial correlations are shown in FIGS. 25A-25H for the autistic children in the study 2 sample. We found that IQ was positively correlated with the number of pops (rho=0.35, p=4.9e-3; Student's t-distribution from now on) and negatively correlated with the screen exploratory percentage (rho=−0.34, p=6e-3) and variability of the touch frequency (rho=−0.32, p=0.3e-2). The verbal standard score of the DAS was positivity correlated with the number of touches (rho=0.31, p=1.4e-2). The spatial standard score of the DAS was positively correlated with the number of pops (rho=0.39, p=2.1e-3) and negatively correlated with the screen exploratory percentage (rho=−0.38, p=3e-3), the average touch duration (rho=−0.33, p=9.1e-3), the average touch velocity (rho=−0.33, p=9.1e-3), the variation of the force applied (rho=−0.32, p=1.2e-2), and the average time spent targeting a bubble (rho=−0.31, p=3.1e-2). The non-verbal composite score of the DAS was positively correlated with the number of pops (rho=0.34, p=9.4e-3) and negatively correlated with the double touches rate (rho=−0.34, p=9.4e-3), the screen exploratory percentage (rho=−0.32, p=1.5e-2), and the average time spent targeting a bubble (rho=−0.30, p=4.5e-2). No significant correlations were found between the motor features and the total calibrated severity score of the ADOS

### Discussion of Example 2

Given increasing evidence of the role of motor impairments in autism, objective and accurate evaluation of fine motor skills is an important component of a comprehensive behavioral assessment of autism. We found that an easy-to-administer and engaging bubble popping game can collect meaningful, quantitative, and objective measures of early motor skills in children ranging from 18 months to 10 years of age. Data were feasibly collected in both clinical research settings and pediatric primary care clinics with minimal instructions, using a tablet and without special equipment and training. Therefore, this simple yet informative tool has the potential of being deployed at scale to enhance detection and assessment of early autism signs and obtain objective and quantitative measures of toddler and school age children's visual motor skills. Our results suggest that toddlers as young as 18 months old and children up to 10 years old showed a significant level of engagement with the game. Importantly, autistic and neurotypical children were equally likely to complete the game and touched the screen with similar frequency. In addition to a simple and engaging game that children of a wide age range can readily use, we engineered a set of touch and sensory-based features from the information recorded by the device. Features to evaluate the participants' performance (e.g., number of touches, popping accuracy), their fine motor skills (e.g., popping accuracy, touch duration, applied force), and their preference for repetitive behaviors (e.g., repeat percentage, screen exploration) were measured.

We observed in both groups that several motor variables, including number of touches, bubble popping rate, median distance to the center, average touch duration, and average touch length, were correlated with age, suggesting that these features are promising as means to assess children's developmental trajectories in visual motor skills. Even after controlling for age by matching groups on this variable and using age as a covariate, several differences in visual motor skills between autistic and neurotypical children emerged. In the younger toddler sample, autistic children popped the bubbles at a lower rate despite an equal number of touches, and their ability to touch the center of the bubble was less accurate. When they popped a bubble, their finger lingered for a longer period, consistent with previous findings⁵⁷, and they showed more variability in their performance. In the older sample, compared to neurotypical children, the autistic children spent a longer period of time on a targeted bubble rather than moving quickly from one bubble to another.

Consistent with previous research⁴⁷, the presence of co-occurring ADHD was associated with lower visual motor skills. We found that autistic children with ADHD had lower accuracy (average distance from the center), lower number of pops despite an equal number of touches, higher number of touches per target, and overall, more variability in their motor behavior. These results are consistent with previous research showing that ADHD is associated with reduced visual motor accuracy and greater variability³⁴. Finally, we proposed several game-based features and demonstrated that they can be aggregated in simple machine learning algorithms, trained to combine behavioral measurements to discover patterns that distinguish diagnostic groups, offering a potential to use such algorithms based on motor performance to differentiate toddlers and children with neurotypical development, autism, and those with or without co-occurring ADHD.

We also examined whether the motor features derived from the game showed meaningful correlations with independent clinical assessments of the autistic children. In autistic toddlers, several motor features were found to be correlated with the fine motor T-score on the Mullen scales, including pop rate and accuracy, double-touching, touch velocity and duration, and variability in touch popping accuracy (rho=−0.52). Overall IQ was found to be correlated with the number of pops and popping accuracy.

Previous studies of infants who are later diagnosed with autism have found that early motor skills are associated with language acquisition²⁸⁻³⁰. We found that the number of different bubbles targeted during the game and the proportion of the screen explored by touch were positively associated with the expressive language T-score of the Mullen Scales. Interestingly, repetitive behavior during the game, reflected in the repeated popping of the same bubble, was positively associated with the Mullen visual reception T-score. It is possible that children with stronger visual perception skills were more likely to notice that the same bubble would appear after they popped it rather than quickly exploring other bubbles. Thus, the bubble-popping game might be able to identify visual perceptual strengths in autistic children. Finally, no associations between the motor features and level of autism related behaviors on the ADOS were found in the toddler group.

In the older group, children with higher overall IQ, as well as those with higher spatial skills and nonverbal reasoning skills, tended to show stronger visual motor skills, as reflected in a greater number of bubbles popped as well as other features. Spatial skills measured on the Differential Abilities Scales, in particular, were consistently correlated with strong visual motor skills, as reflected in a higher number of bubbles popped, average touch duration and velocity, lower variation in the force applied, and average time spent targeting a bubble. Unlike in the younger sample of children, fewer correlations between motor features and language ability were found. Higher verbal skills were correlated only with the number of touches. Gaming patterns hold promise for assessing children's motor skills and potentially detecting early differences in motor behaviors associated with autism and ADHD. In the present study, we examined the distributions of the touch-based features and observed that many of the motor features differentiated autistic and neurotypical toddlers and autistic children with and without co-occurring ADHD. When comparing neurotypical and autistic participants, we observed that on average, neurotypical children exhibited greater visual motor control and accuracy. Both groups showed a similar level of engagement with the game (touching the screen a similar number of times). Still, neurotypical participants played the game with quicker and more accurate touches. Autistic children with co-occurring ADHD touched more of the screen and were less accurate and more variable in their motor responses. These findings underscore the role of cooccurring ADHD in accounting for variability in motor skills in autistic children.

Limitations of this work include the relatively limited number of participants to perform analysis per-demographic and per-sex groups. The relatively small sample size in autistic participants also limits the evaluation of the generalization ability of machine learning algorithms. Studies 1 and 2 had different clinical measures, limiting the possibility of comparing their relationship with motor variables on a broader sample. Longer games beyond 20 seconds might provide information about learning, focus, and anticipation. For study 1 of younger children, although it is possible that a child in the neurotypical group had an autism diagnosis, developmental or language delay, or both, it was not feasible to administer diagnostic and cognitive testing to all children. Children in the neurotypical group did not have a positive score on the M-CHAT-R/F and their parents and providers did not express a developmental concern.

This work and the informative data presented here are important steps towards characterizing the heterogeneity of motor functions in autism. Further work is needed to understand, differentiate, and disentangle motor differences associated with co-occurring psychiatric conditions. Additionally, leveraging ecological tools for the longitudinal quantification of motor function could be beneficial for the development of evidence-based interventions targeting visual motor impairments.

The tools proposed here are designed in the context of a broader effort to develop objective, digital behavioral phenotyping tools. Because children's developmental trajectories are variable, it will be of interest to use digital phenotyping to longitudinally track a wider range of behaviors that can be captured with computer vision analysis, including gaze patterns/social attention⁵², facial expressions/dynamics^{51,55}, postural control⁵⁸, and fine motor control. The present study is a step in that direction. Future work includes evaluating the features proposed here in combination with others, advancing toward a multi-modal solution that objectively describes the rich and diverse realm of developmental variation precisely and quantitatively.

### Methods

Participants. Study 1 was comprised of 151 children between 18 and 36 months of age, 23 of whom were subsequently diagnosed with autism spectrum disorder (ASD) based on DSM-5 criteria (see below). Children were recruited and assessed during their well-child visit at one of four Duke pediatric primary care clinics. Inclusion criteria were age of 16-38 months, not ill, and caregiver language was English or Spanish. Exclusion criteria were sensory or motor impairment that precluded sitting or viewing the app, parent not interested or did not have time to participate, child was too upset following doctor appointment, caregiver popped bubbles, or insufficient clinical information. From a larger group of neurotypical participants recruited for the study, neurotypical participants were selected randomly within the age range that matched the autistic group to limit any potential effects of age on analyses of group differences.

Study 2 was comprised of an independent sample of 82 children between 36 and 120 months of age. Based on a diagnostic evaluation (see below), of the 82 children, 63 had a DSM-5 diagnosis of ASD, of which 32 had co-occurring ADHD, and 19 were neurotypical (NT). Children were recruited from the community through flyers and brochures, emails, social media posts, and the research center's registry. Inclusion criteria were aged 36-120 months, not ill, and caregiver language was English or Spanish. Exclusion criteria included a known genetic (e.g., fragile X) or neurological syndrome or condition with an established link to autism, history of epilepsy or seizure disorder (except for history of simple febrile seizures or if the child is seizure-free for the past year), motor or sensory impairment that would interfere with the valid completion of study measures, and history of neonatal brain damage (e.g., with diagnoses hypoxic or ischemic event).

In both studies, participants were excluded if the child did not understand the game (18 participants; NT=13, Autistic=5, Autistic+ADHD=0; none of the study 2 participants failed to understand the game) or if caregivers popped the bubbles when the child was supposed to pop the bubbles by themselves (5 participants), as reported by the trained research assistant administering the app. Children who did not engage sufficiently in the game, defined as having touched the screen fewer than three times, were also excluded from the analysis (NT=29, Autistic=3, Autistic+ADHD=0).

Table 1 below describes the participants' age, sex, and other demographic characteristics. Caregivers/legal guardians provided written informed consent, and the study was approved by the Duke University Health System Institutional Review Board (Pro00085434, Pro00085435, Pro00085156).

Clinical assessments. In study 1, at the time of app administration, caregivers also completed the Modified Checklist for Toddlers Revised with Follow-up (M-CHAT-R/F)⁶ during the well-child visit when the game was administered. M-CHAT-R/F is a caregiver-report screening questionnaire that asks about autism-related behaviors.

Children who failed the M-CHAT-R/F and/or children for whom the caregiver or physician expressed a developmental concern were referred for a diagnostic evaluation conducted by a licensed and research-reliable psychologist. The average time between referral for evaluation and completing an evaluation was 3.5 months. The diagnostic evaluation included the Autism Diagnostic Observation Schedule—2 (ADOS-2)⁵⁹ and the Mullen Scales of Early Learning (MSEL)⁶⁰, the latter of which yielded an Early Learning Composite Score (ELC) and the following subscale scores: (a) fine motor, (b) visual reception, (c) receptive language, and (d) expressive language. Children in study 1 were not evaluated for co-occurring ADHD because such a diagnosis is not considered reliable before age 3 years. Children were considered neurotypical if they did not fail the M-CHAT-R/F, and neither the caregiver nor their provider expressed a developmental concern. Neurotypical children did not receive a diagnostic or cognitive evaluation.

In study 2, an autism spectrum disorder diagnosis was established by a research-reliable clinical psychologist based on the ADOS-2 and the Autism Diagnostic Interview—Revised (ADIR)⁶¹. Cognitive ability was assessed via the Differential Abilities Scale (DAS)⁶². Co-occurring DSM-5 ADHD diagnosis was established by a licensed clinical psychologist with expertise in ADHD (Davis) via the Mini-International Neuropsychiatric Interview for Children and Adolescents (MINI-Kid) with supplementary questions for assessing ADHD in children⁶³, brief clinical child interview when appropriate, review of the parent-completed ADHD-Rating Scale (ADHD-RS)⁶⁴, reviews of teacher-completed ADHD-RS when available, and clinical consensus based on clinical observations and these instruments. The ADHD-RS yielded an overall ADHD-RS score and Hyperactivity and Impulsivity subscale scores. For Study 2, neurotypical children were defined as having an IQ>70, Vineland Adaptive Behavior Scale scores in the average range⁶⁵, and no clinical elevations on a set of parent-completed rating scales, including the Child Behavior Checklist⁶⁶, ADHD-RS, and the Social Responsiveness Scale⁶⁷. Clinical data were collected using REDCap software.

Pop the bubbles game. The bubble-popping game was delivered at the clinic directly following the well-child visit with the pediatrician. During the app, two types of stimuli are presented. First, a set of brief movies (in total, <10 min) with social and non-social content were displayed using the device's screen. While the child watched the movies, the device's frontal facing camera was used to capture their facial expressions, gaze, and postural/facial dynamics. Next, the bubble popping game was presented. Caregivers were asked to hold their child on their lap and the child was positioned such that they could independently and comfortably touch the Pad's screen and play the game. The iPad was placed on a tripod, around 50 cm from the participant, allowing a sufficient dynamical response of the tripod when the touchscreen is touched while preserving the stability of the device. To minimize distractions during the app administration, other family members and the research staff were asked to stay behind both the caregiver and the child. First, the caregiver was encouraged to pop a few bubbles as a demonstration. Once the child had popped two bubbles independently, the training session ended, and the analyzed data began to be recorded for 20 seconds. By design, a bubble popped when the starting location of a touch was within 18.5 mm of its center. Furthermore, when the child popped a bubble, an identical bubble (i.e., same color) began to ascend from the bottom of the screen and came to the same location. This component of the game allowed an assessment of repetitive versus exploratory behavior (popping a different bubble than last popped). During the data collection, caregivers were instructed not to touch the screen nor provide any further instructions to the child. We used 7th and 8th generation Pads, both 10.2″ inches. With a sampling rate of 60 Hz, on-device high precision inertial and gyroscopic sensors recorded the acceleration and orientation of the device, and screen-based features such as bubbles popping and screen touches. Inertial data were used to compute a proxy for the pressure applied on the screen. At the end of the game, caregivers were asked how frequently their child used tablets or smartphones; among those who responded (244/274, 89.1%), 94.3% of caregivers reported their child had previous experience watching or playing games on a tablet or smartphone (43% frequently, 33% occasionally, and 24% rarely).

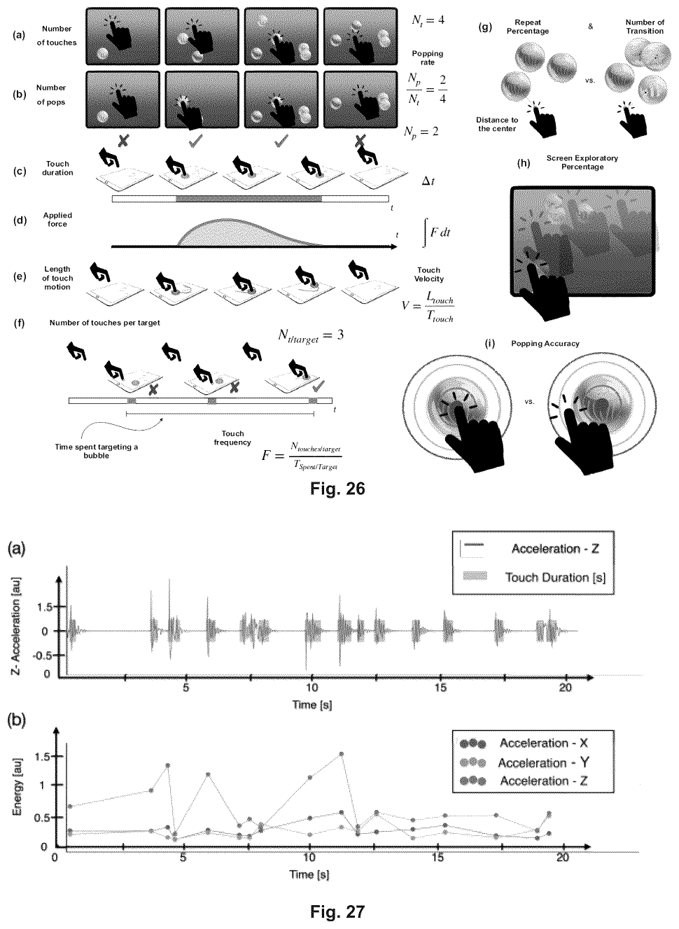

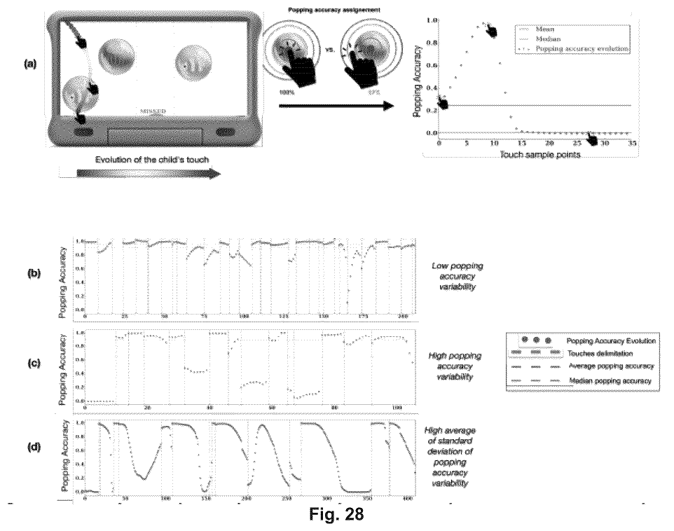

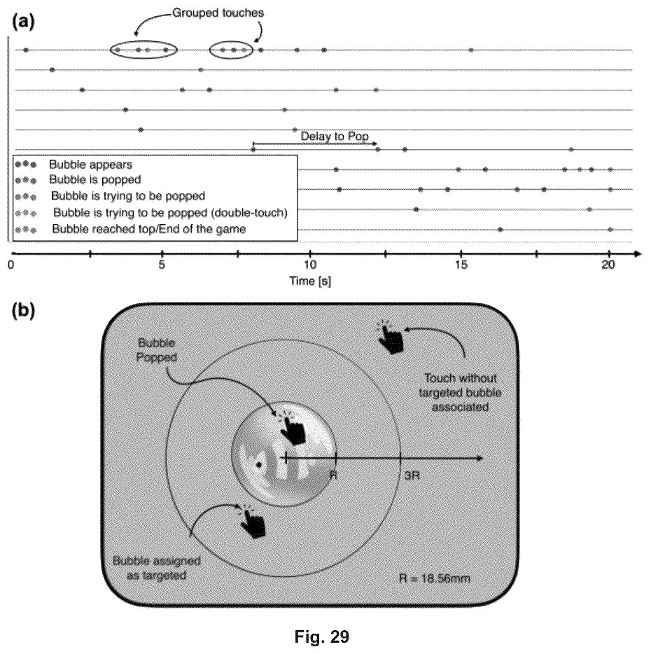

Feature extraction. Using the touch data collected and the tablet kinetic information provided by the device sensors, we computed a set of features representing the participants' motor behavior. More precisely we defined: (1) number of touches, representing the total number of unique times the participant touched the screen, see FIG. 26A; (2) number of pops, the number of bubbles successfully popped, FIG. 26B; (3) bubble popping rate, the ratio of popped bubbles over the number of touches, FIG. 26B; (4) double touch rate, number of times the child tried to double touch the screen over the total number of touches; (5) screen exploratory percentage, proportion of area of the screen that was explored by the child's touches, FIG. 26H; (6) number of targeted bubbles, representing the total number of bubbles that were targeted during the game, with a target defined as a bubble that is close enough to the location of a child's touch; (7) number of transitions, number of times a different type of bubble (different lane) was popped; (8) repeat percentage, percentage of repeated bubbles (same lane and animal character) consecutively popped, FIG. 26G; (9) average/median/std touch duration, mean/median/standard deviation of the touches, that is, time the finger is on the screen during a touch, FIG. 26C; (10) average/median/std touch length of the touch motion, mean/median/standard deviation of the spatial length of the touches, FIG. 26E; (11) average/median/std touch velocity, mean/median/standard deviation of the ratio the touch length and the touch duration, FIG. 26E; (12) average/median/std applied force, approximated by computing the integral of the square of the acceleration of the Pad over the touch duration, retrieved from the built-in device accelerometers (see FIG. 26D; Supplementary Algorithms 1 and 2, and FIGS. 27A and 27B for additional details); (13) average/median/std distance to the center, mean/average/standard deviation of the distance between the finger impact location and the center of the popped bubble, FIG. 26G; (14) average/median/std popping accuracy, for a touch motion, a measure of spatial accuracy. Specifically, for each sample of a touch motion, we measured how far it was from the bubble area, with 100% accuracy defined as located on the bubble area and decreasing accuracy reflecting distances farther from the bubble edges. We then computed the mean/median/standard deviation of this measure across touches; (15) the average variation of the popping accuracy represents the mean standard deviation of the popping accuracy, across all touches, and the variability of the average; (16d) popping accuracy represents the standard deviation of the average popping accuracy, across all touches (maximum; 16e). See additional information on the popping accuracy in FIG. 26I and on FIG. 28; (17) number of touches per target, representing the total number of time the participant hit near or on a bubble before it disappeared, FIG. 26F; (18) average/median/std touch frequency (touch/s), representing the number of touches per second while targeting a bubble, FIG. 26F; (19) average/median/std time spent on a targeted bubble, mean/median/standard deviation of the time a targeted bubble was touched, FIG. 26F. See additional illustrations of the extracted features in FIG. 26 and FIG. 29.

Statistical analysis. Differences in previous experience with electronic games were assessed using a proportion Z-test. Group differences in age and IQ were assessed using a two-sided Mann-Whitney-U test. Effect size, denoted as ‘r’, was evaluated with the rank-biserial correlation algorithm⁶⁸. Spearman's rho correlation was used to assess the relationship between motor features and clinical variables, with statistical significance computed using a Student's t-distribution⁶⁹. Group comparisons were made using one-way ANCOVA for motor-related variables, with the diagnostic group as the categorical predictor (autistic/NT and autistic/ADHD+autistic). We used age as a covariate for study 1 sample, and age and IQ as covariates for study 2. Eta-squared, denoted as η2, was calculated to quantify effect sizes.

Benjamini-Hochberg correction was applied to p-values to control for False Discovery Rate (FDR)⁶⁸. Significance was set at the 0.05 level. Logistic regression was used to assess performance for individual motor features and their combination. We started by using the features that most strongly differentiated the two groups, then selected the feature leading to the best AUC performances. This commonly used type of greedy approach helped address the statistical challenges of high dimensional data. Leave-one-out cross-validation was used to evaluate the generalization performance of models, as recommended in the case of relatively small sample size⁷⁰. Scikit-learn⁷¹ implementations LogisticRegression and GridSearchCV were used to define models and find optimal parameters for each set of motor features. Span of evaluated hyperparameters include: “C” in [0.01, 100], “penalty” in [I1, I2, none], “dual” in [True, False], “fit_intercept” in [True, False], and “solver” in [liblinear, lbfgs].

During the training process, we addressed class imbalance by up-sampling the minority group. Models used for prediction were evaluated using receiver operator curve characteristic (ROC) area under the curve (AUC) with 95% confidence intervals computed by the Hanley McNeil method⁷². Statistics were calculated in Python using SciPy low-level functions V.1.4.1, Statsmodels V.0.10.1, and Pingouin V.0.3.4⁷³⁻⁷⁵. Spearman's rho correlation was used to assess the relationship between motor features and clinical variables, with statistical significance computed using a Student's t-distribution.

TABLE 1
Demographic characteristics of study sample.

| Characteristics | Study 3 (N = 151) | Study 2 (N = 82) |
|---|---|---|
| Age (in months) - Mean (SD) | 23.9 (3.20) | 79.6 (15.65) |
| Sex - Total (55) | | |
| Male | 94 (625) | 57 (69.5) |
| Female | 57 (375) | 25 (30.5) |
| Ethnicity -- Total (%) | | |
| Hispanic/Latino | 16 (10.6) | 9 (10.9) |
| Not Hispanic/Latino | 135 (89.4) | 73 (89.1) |
| Race -Total (%) | | |
| American Indian/Alaskan Native | 3 (1.9) | 0 (0.0) |
| Asian | 1 (0.6) | 4 (4.8) |
| Black or African American | 14 (9.3) | 5 (6.0) |
| WhiteiCaucasian | 110 (72.8) | 64 (78.0) |
| More than one race | 17 (11.4) | 7 (8.5) |
| Other | 6 (4.0) | 2 (2.7) |
| Highest level of education - | | |
| Total (96) | | |
| Without high school diploma | 3 (2.0) | 0 (041 |
| High school diploma or | 8 (5.3) | 2 (2.5) |
| equivalent | | |
| Some college education 4-year | 22 (14.6) | 12 (14.6) |
| college degree or more | 118 (78.1) | 68 (82.9) |
| Familiarity playing game - Tato! | | |
| (95) Unknown/Not reported | 1 (OM | 13 (15.8) |
| Not at at | 13 (8.6) | 1 (12) |
| Rarely | 89 (58.9) | 11 (13.4) |
| Occasionally | 22 (14.6) | 15 (18.3) |
| Frequently | 26 (173) | 42 (51.3) |
| ADDS calibrated total severity | | |
| score | | |
| Unknown/not reported - | 128 (84.7) | 20 (24.4) |
| Total (%) | | |
| Restricted and repetitive | 8.39 (1.53) | 9.1 (0.97) |
| behavior C55 | | |
| Social affect CSS | 7.17 (1.82) | 7.31 (1.50) |
| Total CSS | 7.78 (1.90) | 8.11 (1.36) |
| Mullen Scales of Early Learning | | |
| Unknown/Not reported - | 123 (81.4) | 82 (100.0) |
| Total MI | | |
| Early learning composite score | 65.12 (11.79) | |
| Expressive language T-score | 2930 (8.55) | |
| Receptive language T-score | 23.90 (5.61) | |
| Fine motor T-score | 34.00 (11.85) | |
| Visual reception T-score | 35.65 (11.99) | |
| ADHD-rating scale | | |
| Unknown/Not reported - | 151 (100.0) | 47 (57.3) |
| Total (%) | | |
| Inattentive score | | 11.45 (8.10) |
| Hyperactive - impulsive score | | 11.77 (7.31) |
| Total score | | 23.22 (14.64) |
| Differential Abilities Scales | | |
| Unknown/Not reported - | 151 (100.0) | 9 (0.0) |
| total (%) | | |
| General conceptual ability | | 94.10 (23.89) |
| Verbal standard score | | 95.93 (25.78) |
| Non-verbal standard score | | 93.97 (18.31) |
| Spatial standard score | | 95.71 (22.63) |
| Special non-verbal composite | | 94.02 (2134) |
| standard score | | |

.41)OS-2 Autism Diagnostic Observation Schedule - Second Edition, C55 Calibrated Severity Score.

### REFERENCES FOR EXAMPLE 3

The disclosure of each of the following references is incorporated herein by reference in its entirety to the extent that it is not inconsistent herewith and to the extent that it supplements, explains, provides a background for, or teaches methods, techniques, and/or systems employed herein. The numbers below correspond to the superscripted numbers in EXAMPLE 3.
1. Dawson et al. Randomized, controlled trial of an intervention for toddlers with autism: The early start Denver model. Pediatrics 125, e17-e23 (2010).
2. Estes et al. Long-term outcomes of early intervention in 6-year-old children with autism spectrum disorder. J. Am. Acad. Child Adolesc. Psychiatry 54, 580-587 (2015).
3. Rogers et al. A multisite randomized controlled trial comparing the effects of intervention intensity and intervention style on outcomes for young children with autism. J. Am. Acad. Child Adolesc. Psychiatry 60, 710-722 (2021).
4. Franz et al. Early intervention for very young children with or at high likelihood for autism spectrum disorder: An overview of reviews. Dev. Med. Child Neurol. ://doi.org/10.1111/DMCN.15258 (2022).
5. Robins et al. Validation of the modified checklist for autism in toddlers, revised with follow-up (M-CHAT-R/F). Pediatrics 133, 37-45 (2014).
6. Chlebowsk et al. Large-scale use of the modified checklist for autism in low-risk toddlers. Pediatrics 131, e1121-e1127 (2013).
7. Donohue et al. Race influences parent report of concerns about symptoms of autism spectrum disorder. Autism 23, 100-111 (2019).
8. Guthrie et al. Accuracy of autism screening in a large pediatric network. Pediatrics 144, e20183963 (2019).
9. Scarpa et al. The modified checklist for autism in toddlers: Reliability in a diverse rural American sample. J. Autism Dev. Disord. 43, 2269-2279 (2013).
10. Dickerson et al. Autism spectrum disorder reporting in lower socioeconomic neighborhoods. Autism 21, 470-480 (2017).
11. Healy et al. Fundamental motor skill interventions in children with autism spectrum disorder: a systematic review of the literature including a methodological quality assessment. Res. Autism Spectr. Disord. 81, 101717 (2021).
12. Lloyd et al. Motor skills of toddlers with autism spectrum disorders. Autism 17, 133-146 (2013).
13. Carsone et al. Systematic review of visual motor integration in children with developmental disabilities. Occup. Ther. Int 2021, U.S. Pat. No. 1,801,196 (2021).
14. Wilson et al. Motor development and delay: Advances in assessment of motor skills in autism spectrum disorders. Curr. Opin. Neurol. 31, 134-139 (2018).
15. Wilson et al. What's missing in autism spectrum disorder motor assessments? J. Neurodev. Disord. 10, 33 (2018).
16. Bhat. Is motor impairment in autism spectrum disorder distinct from developmental coordination disorder a report from the SPARK study. Phys. Ther. 100, 633-644 (2020).
17. Green et al. Impairment in movement skills of children with autistic spectrum disorders. Dev. Med Child Neurol. 51, 311-316 (2009).
18. Miyahara et al. Brief report: Motor incoordination in children with Asperger syndrome and learning disabilities. J. Autism Dev. Disord. 27, 595-603 (1997).
19. Jansiewicz et al. Motor signs distinguish children with high functioning autism and Asperger's syndrome from controls. J. Autism Dev. Disord. 36, 613-621 (2006).
20. Esposito et al. An exploration of symmetry in early autism spectrum disorders: Analysis of lying. Brain Dev. 31, 131-138 (2009).
21. Fournier et al. Motor coordination in autism spectrum disorders: A synthesis and meta-analysis. J. Autism Dev. Disord. 40, 1227-1240 (2010).
22. Guinchat et al. Very early signs of autism reported by parents include many concerns not specific to autism criteria. Res Autism Spectr. Disord. 6, 589-601 (2012).
23. Cook et al. Atypical basic movement kinematics in autism spectrum conditions. Brain 136, 2816-2824 (2013).
24. Sacrey et al. Reaching and grasping in autism spectrum disorder: A review of recent literature. Front Neurol. 5, 6 (2014). Jan.
25. Dowd et al. Do planning and visual integration difficulties underpin motor dysfunction in autism? A kinematic study of young children with autism. J. Autism Dev. Disord. 42, 1539-1548 (2012).
26. Verma & Lahiri. Deficits in handwriting of individuals with autism: a review on identification and intervention approaches. Rev. J. Autism Dev. Disord. 9, 70-90 (2022).
27. Gong et al. Abnormal gait patterns in autism spectrum disorder and their correlations with social impairments. Autism Res. 13, 1215-1226 (2020).
28. LeBarton et al. Infant motor skill predicts later expressive language and autism spectrum disorder diagnosis. Infant Behav. Dev. 54, 37-47 (2019).
29. Choi et al. Development of fine motor skills is associated with expressive language outcomes in infants at high and low risk for autism spectrum disorder. J. Neurodev. Disord. 10, 14 (2018).
30. Garrido et al. Language and motor skills in siblings of children with autism spectrum disorder: A metanalytic review. Autism Res 10, 1737-1750 (2017).
31. Engelhard et al. Health system utilization before age 1 among children later diagnosed with autism or ADHD. Sci. Rep. 10, 17677 (2020).
32. Lidstone & Mostofsky. Moving toward understanding autism: visual motor integration, imitation, and social skill development. Pediatr. Neurol. 122, 98-105 (2021).
33. Glazebrook et al. The role of vision for online control of manual aiming movements in persons with autism spectrum disorders. Autism 13, 411-433 (2009).
34. Izawa et al. Motor learning relies on integrated sensory inputs in ADHD, but over-selectively on proprioception in autism spectrum conditions. Autism Res. 5, 124-136 (2012).
35. Torres. The rates of change of the stochastic trajectories of acceleration variability are a good predictor of normal aging and of the stage of Parkinson's disease. Front Integr. Neurosci. 0, 50 (2013).
36. Anzulewicz et al. Toward the Autism Motor Signature: Gesture patterns during smart tablet gameplay identify children with autism. Nat. Publishing Group 1-14 (2016) https://doi.orq/10.1038/srep31107.
37. Pitchford & Outhwaite. Can touch screen tablets be used to assess cognitive and motor skills in early years primary school children? A cross-cultural study. Front. Psychol. 7, 1-14 (2016).
38. Coutinho et al. Effectiveness of iPad apps on visual-motor skills among children with special needs between 4y0m-7y11 m. Disabil. Rehabil. Assist Technol. 12, 402-410 (2017).
39. Simeoli et al. Using technology to identify children with autism through motor abnormalities. Front Psychol. 12, 1-11 (2021).
40. Lu et al. Swipe kinematic differences in young children with autism spectrum disorders are task- and age-dependent: A smart tablet game approach. Brain Disord. 5, 100032 (2022).
41. Chua et al. Developmental differences in the prospective organisation of goal-directed movement between children with autism and typically developing children: A smart tablet serious game study. Dev. Sci. https://doi.org/10.1111/desc. 13195 (2021).
42. Anzulewicz et al. Toward the Autism Motor Signature: Gesture patterns during smart tablet gameplay identify children with autism. Sci. Rep. 6, 1-13 (2016). 2016 6:1.
43. Pitchford & Outhwaite. Can touch screen tablets be used to assess cognitive and motor skills in early years primary school children?A cross-cultural study. Front. Psychol. 7, 1666 (2016).
44. Intarasirisawat et al. Exploring the touch and motion features in game-based cognitive assessments. Proc. ACM Interact. Mob. Wearable Ubiquitous Technol. 3, 1-25 (2019).
45. Millar et al. Phase 3 diagnostic evaluation of a smart tablet serious game to identify autism in 760 children 3-5 years old in Sweden and the United Kingdom. BMJ Open 9, e026226 (2019).
46. Gowen & Miall. The cerebellum and motor dysfunction in neuropsychiatric disorders. Cerebellum 6, 268-279 (2007). 2007 6:3.
47. Goulardins et al. Attention deficit hyperactivity disorder and motor impairment. Percept. Mot. Skills 124, 425-440 (2017).
48. Bhat. Motor impairment increases in children with autism spectrum disorder as a function of social communication, cognitive and functional impairment, repetitive behavior severity, and comorbid diagnoses: a SPARK study report. Autism Res. 14, 202-219 (2021).
49. Dawson & Sapiro. Potential for digital behavioral measurement tools to transform the detection and diagnosis of autism spectrum disorder. JAMA Pediatr. 173, 305 (2019).
50. Dawson et al. Atypical postural control can be detected via computer vision analysis in toddlers with autism spectrum disorder. Sci. Rep. 8, 1-7 (2018).
51. Campbell et al. Computer vision analysis captures atypical attention in toddlers with autism. Autism 23, 619-628 (2019).
52. Chang et al. Computational methods to measure patterns of gaze in toddlers with autism spectrum disorder. JAMA Pediatr. 175, 827-836 (2021).
53. Perochon et al. A scalable computational approach to assessing response to name in toddlers with autism. J Child Psychol Psychiatry. https://doi.org/10.1111/jcpp.13381(2021).
54. Egger et al. Automatic emotion and attention analysis of young children at home: a ResearchKit autism feasibility study. NPJ Digit Med. 1, 1-10 (2018).
55. Krishnappababu et al. Exploring complexity of facial dynamics in autism spectrum disorder. IEEE Trans. Affect Comput. 01, 1-10 (2021).
56. Carpenter et al. Digital Behavioular phenotyping detects atypical pattern of facial expression in toddlers with autism. Autism Res. 14, 488-499 (2020).
57. Anzulewicz et al. Toward the Autism Motor Signature: Gesture patterns during smart tablet gameplay identify children with autism. Sci. Rep. 6, 1-13 (2016).
58. Dawson et al. Atypical postural control can be detected via computer vision analysis in toddlers with autism spectrum disorder. Sci. Rep. 8, 17008 (2018).
59. Gotham et al. The autism diagnostic observation schedule: Revised algorithms for improved diagnostic validity. J. Autism Dev Disord. 37, 613-627 (2007).
60. Braaten. Mullen scales of early learning. The SAGE encyclopedia of intellectual and developmental disorders (Western Psychological Services, 2018). https://doi.orq/10.4135/9781483392271.n327.
61. Lord et al. Autism Diagnostic Interview-Revised: A revised version of a diagnostic interview for caregivers of individuals with possible pervasive developmental disorders. J. Autism Dev. Disord. 24, 659-685 (1994). 1994 24:5.
62. Elliott et al. The differential ability scales (The Psychological Corporation, 2007).
63. Vitiello et al. Pharmacotherapy of the preschool ADHD treatment study (PATS) children growing up. J. Am. Acad. Child Adolesc. Psychiatry 54, 550-556 (2015).
64. DuPaul et al. ADHD Rating Scale-IV: Checklists, norms, and clinical interpretation (Guilford Press, 1998).
65. Sparrow et al. Vineland adaptive behavior scales (Pearson, 1984).
66. Achenbach & Rescorla. Manual for the ASEBA school-age forms & profiles: an integrated system of multi-informant assessment (Burlington: University of Vermont, Research Center for Children, Youth & Families., 2001).
67. Constantino & Gruber. Social Responsiveness Scale, Second Edition (SRS-2) (Western Psychological Services, 2012).
68. Benjamini & Hochberg. Controlling the false discovery rate: a practical and powerful approach to multiple testing. J. R. Stat. Soc. Ser. B Methodol. 57, 289-300 (1995).
69. Cureton. Rank-biserial correlation. Psychometrika 21, 287-290 (1956).
70. Vabalasid et al. Machine learning algorithm validation with a limited sample size. PLoS One. 2019; 14:e0224365. https://doi.org/10.1371/journal.pone.0224365.
71. Pedregosa et al. Scikit-learn: machine learning in python. J. Mach. Learn. Res. 12, 2825-2830 (2011).
72. Hanley & McNeil. The meaning and use of the area under a receiver operating characteristic (ROC) curve. Radiology 143, 29-36 (1982).
73. Virtanen et al. SciPy 1.0: fundamental algorithms for scientific computing in Python. Nat. Methods 17, 261-272 (2020).
74. Seabold & Perktold. Statsmodels: econometric and statistical modeling with python. In: Proceedings of the 9th Python in Science Conference 92-96 (2010). https://doi.org/10.25080/majora-92bf1922-011.
75. Vallat. Pingouin: statistics in Python. J. Open Source Softw. 3, 1026 (2018).

```
  Applied force computation - Algorithms
  Algorithm 1: Computation of a proxy for the force applied.
  Input: = X(t), Y(t) and Z(t) time series of the acceleration of the iPad. Child's
touchscreen information
  (Ti)i[1,4
  Output: Energy (Ei)i[I ,Isl] associated with each child touch.
  For each touch Ti of the child:
  # Find beginning and ending timestamps of the dynamical response of the iPad ti,
tf = retrieve...touch..timestamps(Ti, X(t), Y(t), Z(t)) # See Alg.
  # Compute the energy of the iPad associated to this touch
  Ei =titf(X2-1-Y2A-Z2)dt
```

```
  Algorithm 2: retrieve_touch_timestamps
  Input: = Ti single child's touch information, and X(t), Y(t), Z(t) the accelerations of
the iPad. Output: ti, tf beginning and ending timestamps of the dynamical response of the
iPad.
  # Initialize ti and tf to be the touch timestamps
  ti = Thirst timestamps
  tf = Tilast timestamps
  # Compute Z(t) standard deviation during the touch (orthogonal direction of the
screen) Z(t) = STD(Z(t), ti, tf)
  # Looking for the final timestamps tf as the ending of the device's dynamical
relaxation, by finding when
  Z(t) stays less than 0.512(t)
  tf = retrieve...final timestamps(Z(t)[ti,tf], Z(t))
```

SUPPLEMENTARY TABLE 1
AUCs obtained by the model when using three motor
features, by identified sex, race, and ethnicity

| | AUG [95% | |
|---|---|---|
| Subgroups | CO Study 1 (N = 151) | Study 2 [symbol] |
| All | 0/3 [0.63, 0.83] | 0.74 [0.62, 0.86] |
| Sex | | |
| Male | 0.72 [0.60 0.84] | 0.78 [0.66, 0.92] |
| Female | 00.76 [0.60 0.92] | 0.66 [0.41, 0.91] |
| Ethnicity | | |
| Not Hispanic/Latino | 0.75 [0.64 0.86] | 0.72 [0.59, 0.85] |
| Hispanic/Latino | 0.71 [0.45 0.97] | 1.00 [1.0, 1.0] |
| Race | | |
| Black or African American | 0.71 [0.55 0.87] | 1.0 [1.0, 1.0] |
| White/Caucasian | 0.77 [0.65 0.89] | 0.79 [0.65, 0.93] |
| All Other Races | 0.72 [0.52 0.92] | 0.59 [0.24, 0.94] |

[symbol]

indicates data missing or illegible when filed

The AUG values were relatively consistent across groups; however, confidence intervals were larger due to the smaller sample sizes. Leave-one out cross-validation approach was used. Features used to fit the model was the average length, the average touch duration, and the average time spent for the Study 1 sample, and the average distance to the center, the number of targets, and the screen exploratory percentage for the study 2 sample,

### Example 4

### Exploring Complexity of Facial Dynamics in Autism Spectrum Disorder

Atypical facial expression is one of the early symptoms of autism spectrum disorder (ASD) characterized by reduced regularity and lack of coordination of facial movements. Automatic quantification of these behaviors can offer novel biomarkers for screening, diagnosis, and treatment monitoring of ASD. in this work, 40 toddlers with ASD and 396 typically developing toddlers were shown developmentally-appropriate and engaging movies presented on a smart tablet during a well-child pediatric visit, The movies included social and non-social dynamic scenes designed to evoke certain behavioral and affective responses. The front-facing camera of the tablet was used to capture the toddlers' face. Facial landmarks' dynamics were then automatically computed using computer vision algorithms, Subsequently, the complexity of the landmarks' dynamics was estimated for the eyebrows and mouth regions using multiscale entropy. Compared to typically developing toddlers, toddlers with ASD showed higher complexity (i.e., less predictability) in these landmarks' dynamics. This complexity in facial dynamics contained novel information not captured by traditional facial affect analyses. These results suggest that computer vision analysis of facial landmark movements is a promising approach for detecting and quantifying early behavioral symptoms associated with ASD.

Facial expressions are often used as a mode of communication to initiate social interaction with others^{1,2}, and are one of the key social behaviors used by infants during early development³. Individuals with autism spectrum disorder (ASD) often experience challenges in establishing social communication coupled with difficulties in recognizing facial expressions and using them to communicate with others⁴. Reduced sharing of affect and differences in use of facial expressions for communication are core symptoms of ASD and are assessed as part of standard diagnostic evaluations, such as the Autism Diagnostic Observation Schedule (ADOS)⁵. Children with ASD more often display neutral affect and ambiguous expressions compared to children with other developmental delays and typically developing (TD) children⁶.

Standardized observational assessments of ASD symptoms require highly trained and experienced clinicians⁷. Research on facial expressions usually involves manually coding of observations of facial expressions from recorded videos, based on complex and time-intensive facial action coding systems. These methods are difficult to deploy at scale and universally. Therefore, researchers have been employing technological advancements to capture facial expressions using motion capture and computer vision (CV)⁸,9. The application of CV can help to quantify the intensity of emotional expression and the atypicality of facial expressions⁷. Prior work in CV shows that quantification of the differential ability in producing facial expressions can distinguish children with typical development versus ASD¹⁰. CV can also help in understanding the lag in developmental stages of facial expression production, offering cues to understand the emotional competence faced by the individuals with ASD¹¹.

Exploiting CV, it was shown that the facial expressions of children with ASD were often ambiguous⁹, a result in agreement with a study using a non-CV approach⁶. A recent study¹² extracted the dynamics of facial landmarks to estimate the group differences across various emotional expressions. Individuals with ASD exhibited a higher range of facial landmarks' dynamics compared to TD individuals across all emotions assessed. To quantify the complexity of facial landmarks' dynamics, researchers have started to explore computational tools such as autoregressive models¹³ and entropy measures¹⁴. These studies found that individuals with ASD exhibit distinctive complexity in their facial dynamics, compared to TD individuals when they were asked to mimic given emotions. One of the standard measures used to analyze the complexity of physiological signals (e.g., facial dynamics) is the multiscale entropy OVLSE)¹⁴⁻¹⁸, discussed and extended in this work.

The present Example focuses on analyzing the complexity of spontaneous facial dynamics of toddlers with and without ASD. Toddlers watched developmentally—appropriate and. engaging movies presented on a smart tablet. Simultaneously, the frontal camera of the tablet was used to record the toddlers' faces, providing the opportunity for the automatic analysis via CV. Specifically, we studied the facial landmarks' dynamics of the toddlers with ASD versus TD), quantified in terms of a complexity estimate derived from MSE analysis.

We hypothesized that the complexity in landmarks' dynamics would differentiate toddlers with and without ASD, offering a distinctive biomarker. We hypothesized that the toddlers with ASD would exhibit higher complexity (i.e., less predictability) in their landmarks' dynamics associated with regions such as the eyebrows—representing their uniqueness in raising eyebrows¹⁹, and mouth-potentially related to atypical vocalization patterns²⁰⁻²². Furthermore, we were interested in exploring whether our findings would support previous work showing atypical eyebrow¹⁹ and mouth²² movements in the ASD population. Lastly, we also examined whether the complexity in landmarks' dynamics provides complementary and nonredundant information to the estimated affective expressions that the toddlers manifested in response to the presented movies, or if they provide redundant information. In one of our previous studies¹⁹, we examined affect (i.e., emotional expressions) variation over a period of time while the toddlers were engaged and watched the presented movies. Though the work¹⁹ presented the feasibility of distinguishing between the ASD and TD groups based on patterns of affective expression, in our current work, we investigate the possibility of using the complexity of the raw facial landmarks dynamics without considering any variation in affect. This is motivated in part by the fact that individuals with ASD are prone to elicit a mixture of emotions at the same time [6], therefore using the complexity of the raw landmarks' dynamics can offer further confidence and add additional information beyond affect. Thus, for a much larger dataset and re-designed stimuli, we replicate the affect-related analysis similar to that of Carpenter et al., “Digital behavioral phenotyping detects atypical pattern of facial expression in toddlers with autism,” Autism Res., vol. 14, no. 3, pp. 488-499, 2020, and show that the complexity in facial dynamics are an independent and more powerful measure.

In this EXAMPLE, we demonstrate the following: (a) the MSE, as here extended to handle time-series with partially missing data and to compare across subjects, can characterize complexity in facial landmarks' dynamics; (11) the complexity in landmarks' dynamics can distinguish between ASD and TD groups; and (c) this complexity information is complementary to information about affective expressions estimated from computer vision-based algorithms.

TABLE 1
Demographics of the Study Participants

| | TD | ASD |
|---|---|---|
| Number of Participants | 396 | 40 |

N (%)

| | | |
|---|---|---|
| Males | 194 (48.9%) | 31 (77.5%) |
| Females | 202 (51.1%) | 9 (22.5%) |
| Race White/Caucasian | 308 (77.7%) | 19 (47.5%) |
| African American | 34 (8.6%) | 6 (15.0%) |
| Others | 54 (13.6%) | 15 (37.5%) |
| Hispanic/Latino | 29 (7.3%) | 13 02.5%) |
| M-CHAT-R/F (Positive) | 1 (0.25%) | 35 (87.5%) |

M ± SD

| | | |
|---|---|---|
| ADOS-T Total | 3.0 + 0.0 | 7.64 + 1.69 |
| Age in months | 20.62. ± 3.2.1 | 24.21 ± 4.72 |

Note:
One participant in the TD group screened positive on M-CHAT-R/F, was evaluated with ADOS-T and not diagnosed with ASD.

### 2. Data Collection and Stimuli

Participants and Study Procedures. Toddlers between 17-36 months of age were recruited at four pediatric primary care clinics during their well-child visit. Toddlers received a commonly-used, caregiver-completed autism screening questionnaire, Modified Checklist for Autism in Toddler—Revised with Follow-up (M-CHAT-R % F)²³ as part of routine clinical care. If a child screened positive on the M-CHAT-R/F, or a caregiver; clinician expressed any developmental concern, the child was evaluated by a child psychologist based on the Autism Diagnostic Observation Schedule—Toddler (ADOS-T) module²⁴ The exclusion criteria were: (1) known hearing or vision impairments; (ii) child too upset; (iii) caregiver expressed no interest, not enough time, needed to take care of siblings, or unable to give consent in English or Spanish; (iv) child did not complete study procedures; and (v) clinical information missing. A total of 436 children (ID er: 396 and ASD eri 40) participated; 82.8% of caregivers had a college degree, 155% had more than high school education, and 1.8% did not have a high school education; for additional demographics please see Table 1 above. Caregivers/legal guardians provided written informed consent, and the study was approved by the Duke University Health System Institutional Review Board (Pro00085434, Pro00085435).

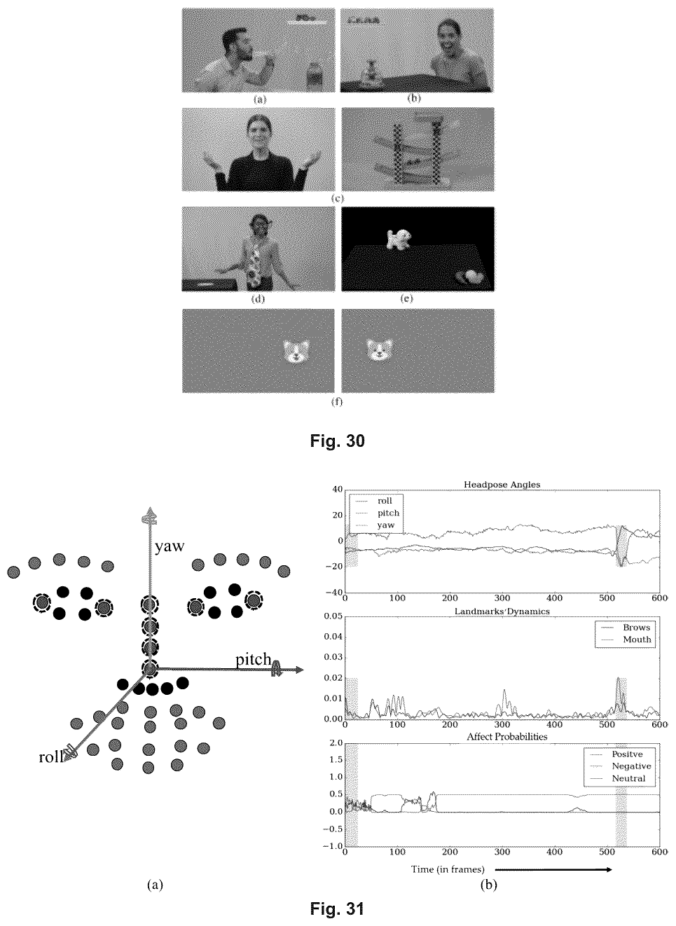

Caregivers were asked to hold their child on their lap and an iPad was placed at a distance of about 60 cm from the child. The tablet's frontal camera recorded the child's behavior while short movies (each less than a minute and a half long) were presented on the tablet's screen. The movies consisted of both social and non-social components; see FIG. 30 for illustrative snapshots. To minimize any distractions during the study, all other family members and the practitioners who administered the study were asked to stay behind both the caregiver and the child.

Movies. The movies were presented through an application (app) on a tablet. These movies were strategically designed to elicit autism—relevant behaviors. For our current analysis, we categorized the movies according to whether they contained primarily social versus non-social elements (FIG. 30). During the social movies, human actors demonstrated engaging actions, such as making eye contact, smiling, making gestures, acting silly, and narrating rhymes. The non-social movies were dynamic toys or animations. We studied the participants' response to 6 movies briefly described next.

Blowing Bubbles (˜44 secs): A man held a bubble wand and blew the bubbles, with some attempts successful and some failing, with eye contact, smiling and frowning. The movie included limited talking from the actor.

Spinning Top (˜53 secs): An actress played with a spinning top with both successful and unsuccessful attempts along with eye contact, smiling and frowning. The movie included limited talking from the actress.

Rhymes and Toys (˜49 secs): An actress recited nursery rhymes, such as Itsy-Bitsy Spider, while smiling and gesturing, followed by a series of dynamic, noise-making toys which were shown without the presence of the actress on the scene.

Make Me Laugh (˜56 sees): An actress engaged in silly, funny actions while smiling and making eye contact.

Mechanical Puppy (˜25 secs): A mechanical toy puppy barked and walked towards vegetable toys.

Dog in Grass Right-Right-Left (RRL) (˜40 secs): A barking puppy was shown in different parts of the screen, followed by a series of appearances in a right-right-left OW pattern.

### 3. Methods and Analysis

Facial Landmark Detection and Preprocessing. A face detection algorithm was used first to identify the number of faces detected in each of the recorded video frames²⁵. Using a low-dimensional facial embedding^{26,27}, we ensured that we tracked only the participant's face throughout the video, ignoring other detected faces associated with the caregiver, siblings (if any), and clinical practitioners. Once the child's facial image was detected and tracked, we extracted 49 facial landmark points²⁸. These 2D positional coordinates of 49 facial landmarks were time synchronized with the presented movies.

The facial landmarks were then preprocessed in two steps for our further analysis, namely (1) first compensating for the effects due to global head motions via global shape alignment, and (2) removing the time segments when the participants were not attending to the stimuli. For step 1, we utilized the points from the corners of the eyes and the nose (FIG. 31A) to align and normalize the data to a canonical face model through an affine transformation (refer to reference [29] for more details). During this process, we also estimated the head pose angles uroll, upitch, and uyaw (see upper plot in FIG. 31B) as angular coordinates relative to the camera. Step 1 of preprocessing was crucial since we did not want our landmarks' dynamics to be contaminated with additional noise caused due to head motions, rather only to study the actual facial expressions. In addition, the videos were recorded at 30 frames per second, and we removed the high frequency components of the landmark signals (generally associated with noise). To this end, we filtered the signal components above 15 Hz, this frequency value was chosen based on the facts that we require at least 260 ms to capture a facial expression³⁰, and 150-250 ms to exhibit a valid gaze fixation duration^{31,32}.

For the step 2 of preprocessing, we were interested in studying the dynamics of the facial landmarks as a spontaneous response to the presented movies, we focus our analysis on time segments in which the participants were considered to be engaged with the presented movies. To this end, we filtered the segments considering two criteria: (1) extreme non-frontal head pose, and (2) rapid head movement (this in part can render computation of landmarks very unstable). A non-frontal head pose was defined as the frames where the head pose angles lied outside the ranges ±20° for the upitch and uyaw, and _45_ for the uroll. Frames containing rapid head movement were removed by analyzing the angular speed of the head pose. We calculated a one second moving average (θ′) of the time-series data for θᵣₒₗₗ, θ_{pitch}, and θ_{yaw}. Using these smooth versions of the head pose coordinates, we excluded frames where the difference (θ_{diff}) between the current frame i and the previous frame i−1 was more than 5°, estimated using:

$${θ}_{diff} = {θ}_{i}^{′} - {θ}_{i - 1}^{′} .$$

Computation of Facial Landmarks' Dynamics. After the above described preprocessing, and considering only the valid/attending frames, we extracted the landmarks' dynamics, concentrating on the eyebrows and the mouth regions (FIG. 31A). To do so, we estimated each landmark's Euclidean displacements from the current frame i to the previous frame i−1, for all the points belonging to the eyebrows and mouth regions (represented as blue and green points in FIG. 31A). If any of the i or i−1 frames was missing, the respective Euclidean displacement was considered missing. Finally, an average value was computed by combining all the landmark points belonging to either of the eyebrows or mouth regions to form a one-dimensional time-series data for each of the two regions (middle plot in FIG. 31B).

Multiscale Entropy for Measuring the Complexity of Landmarks' Dynamics. Multiscale entropy (MSE) is used as a measure of dynamic complexity, quantifying the randomness or unpredictability of a time-series physiological signal operating at multiple temporal scales^{15,16}, including facial dynamics^{14,17}. Briefly, entropy helps in quantifying the unpredictability or randomness in a sequence of numbers; the higher the entropy, the higher its unpredictability. The sample entropy is a modified version widely used to assess the complexity of time-series^{33,34}. These concepts are formalized next.

The MSE is estimated by calculating the sample entropy on a time-series data

$$X = \{ \text{?} , … , {x}_{i} , \text{?} … , {x}_{N} \}$$

$$\text{?} \text{indicates text missing or illegible when filed}$$

with length N at multiple timescales t. To this end, the time-series X is represented at multiple resolutions ({yⱼ^{(τ)}}) by coarse-graining X as

$${y}_{j}^{( τ )} = \frac{1}{τ} \text{?} {x}_{i} , where 1 \leq j \leq \frac{N}{τ} . \qquad ( 2 )$$

$$\text{?} \text{indicates text missing or illegible when filed}$$

Here, we downsampled the landmarks' dynamics timeseries data across the frames for 0 to 30 scales. During this downsampling process, a downsampled data point yᵢ was filled with the average values of xᵢ−s, only when a minimum of 50% of the xᵢ−s were not missing (see FIG. 32A, where dotted circles represent the missing data points). Once we coarse grained the landmarks' dynamics for the eyebrows and mouth regions, the sample entropy was computed for each scale to obtain the MSE, further detailed next.

As mentioned before, the sample entropy is a measure of the irregularity of a signal; at a given embedding dimension m and a positive scalar tolerance r, the sample entropy is given by the negative logarithm of the conditional probability that if the sets of simultaneous data points having length m repeats within the distance r, then the sets of simultaneous data points having length m+1 also repeats within the distance r. Consider a time-series (landmarks' dynamics) of length N as X ¼ fx1; . . . ; xi; . . . ; xNg, from which the m-dimensional vector Xm i ¼{xmi, xm i [symbol] 1, xmi [symbol] 2, . . . , xm i [symbol] m_1} is formed. The distance d [symbol] xmi; xmj [symbol] between any two vectors is defined as

$$d xmi ; xmj\_\_ \frac{1}{4} max xm ipk\_ 1 \_xmj Þ k\_ {1}_{— —} n o ; k \frac{1}{4} 1 ; 2 ; … ; m : \qquad ( 3 )$$

Consider C^{m}(r) to be the cumulative sum of the number of repeating vectors in the m-dimension (see FIG. 32B) under the condition dδxmi; xmj [symbol] _r, with i 6¼ j, and analogously C^{m+}(r) be the cumulative sum of repeating vectors in m [symbol] 1-dimension. Then, the sample entropy (SampEn) is defined as

$$SampEn \frac{1}{4} \_ln Cm Þ 1 ð r Þ \_Cmð r Þ\_\_ : \qquad ( 4 )$$

When estimating C^{m} and C^{m+}, the choice of the dimension m, the tolerance value r³⁴, and the missing data in the landmarks' dynamics^{34,35Z}35] play a vital role. This is discussed next.

For our study, we set m=2 because it was the most commonly used value in previous similar studies, e.g., references [14], [15], [16], [17]. However, the choice of different m values did not greatly affect our findings (see Appendix I, which can be found on the Computer Society Digital Library at http://doi. ieeecomputersociety.org/10.1109/TAFFC.2021.3113876). We have selected r ¼0.15 s, where s is considered to be a standard deviation of the time-series. The value 0.15 was chosen as suggested in other studies, e.g., references [16], [35], [36]. It was also evident that a time-series having noisy spikes can increase the tolerance r because it is a function of s, which can be vulnerable to noise³⁴. As a result, even if the time-series data was complex (irregular), as r increases, we allow a higher degree of tolerance to match repeating vector sequences in both m an m [symbol] 1 dimensions, resulting in a lower SampEn value. This can be potentially mitigated by removing the noisy spikes; we have already taken care of this for our landmarks' dynamics via the previously described preprocessing steps. Another challenge arises when we compare the SampEn computed on the landmarks' dynamics that came from two different participants having two different values of s influencing the tolerance r. There is a persisting misconception in the literature in using s calculated on a time-series at the per-participant level, and comparing the resulting SampEn across different participants, causing severe bias in the final outcome. To overcome this spurious effect and compare signals consistently across participants, for the definition of r, we used the population standard deviation rather than the standard deviation associated with each participant.1 Finally, it could be possible that individuals with ASD tend to exhibit large amounts of head movements, causing a large amount of missing data in the time-series, challenging the SampEn estimation. To handle any such missing data and consistently estimate the SampEn, we selected the segment in m [symbol] 1 dimension only if the respective vector was embedded with no missing data (see FIG. 3s). Only those data points belonging to these segments were considered during the computation of the coefficients C^{m}(r) and C^{m+}(r) (similar to reference [35]). Additionally, at least 40% of the data was necessary to perform effective complexity analysis³⁴⁻³⁶.

Below this threshold the estimation of the SampEn may not be reliable. For our analysis, the 40% depended on the length of the movies. Participants having less than 40% data were removed from the analysis for a specific movie.

Estimation of Affective States. In addition to studying the complexity of facial dynamics, we also considered the more standard approach of investigating affective expressions, and explicitly show that these two measurements are not redundant. For consistency, we used the pose-invariant affect method from our previous work³⁷, while other approaches could be used for this secondary analysis. We estimated the probability of a participant's three different categories of affective expression using four facial expressions: positive (happy expression), neutral (neutral expression) and negative (angry and sad expression). FIG. 31B illustrates the time-series of the probabilities associated with positive, neutral, and negative affect. These quantities were estimated for those frames during which the participant was considered to be engaged (Section 3.1) and at a rate of 30 frames per second. To analyze the evolution of the displayed affect, we considered the first-derivative of each of these affect-based time-series. Finally, we computed the rate of change for each affect, defined as a moving average over 10 frames (⅓ second), followed by cumulative sum of these values to obtain energy of the signal. Previous work²⁹, presented validation results between human coding and the computer vision based automatic coding for ‘positive’ and ‘other’ (neutral and negative) affective expressions on a frame-by-frame basis for 136450 frames (belonging to different participants). The validation results showed excellent intra-class correlation coefficient (ICC) with 0.9 inter-rater reliability performance.

Statistical Analysis. Statistically significant difference between the groups' distribution was tested using Mann-Whitney U test, in particular, the python function pingouin.mwu was used. Within group comparisons (e.g., to compare between the eyebrows and mouth regions) were performed using Wilcoxon signed-rank test with python function pingouin.wilcoxon. Effect sizes were estimated with the standard r value for both significant tests.

Since, there could be possible confounds due to covariates such as (1) ‘percentage of missing data’ that was removed during preprocessing, and (2) ‘the variation in the landmark movements’ on our complexity measures; we have performed additional statistics, e.g., analysis of covariance (ANCOVA) using pingouin.ancova. Additionally, for cross-correlation analysis, we computed Spearman's r using scipy.stats.spearmanr in python. A decision tree-based classifier [38] was used to assess the possible separation between the ASD and TD groups using our complexity analysis and affect-related measures while considering each individual movies. ‘Gini impurity’ was used as automatic splitting criteria. The differences between Area Under the Curves (AUCs) of the Receiver Operating Characteristic (ROC) based on the different features and movies were compared using the DeLong method³⁹. Additionally, logistic regression (with a python function, sklearn.linear_model.LogisticRegression) was used to estimate odds ratio to predict the risk for ASDin toddlers. For the logistic regression, we have again used both the proposed landmarks' complexity and the affect-related measures.

### Results

The two main questions explored in this section are: (1) whether the estimation of complexity in landmarks' dynamics can be used as a distinctive biomarker to distinguish between ASD and TD participants, and (2) whether the estimated complexity measure adds value beyond traditional measures of facial emotional expressions.

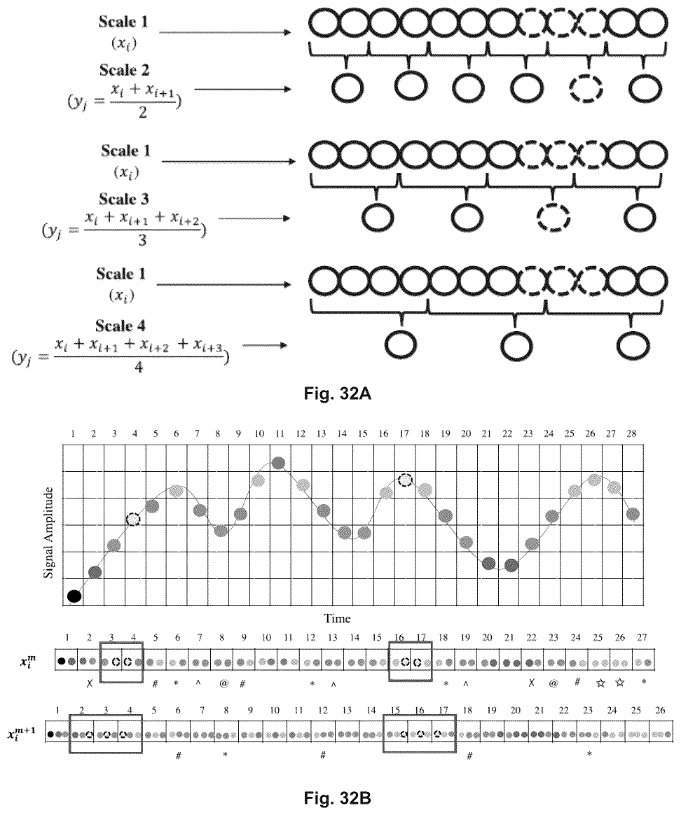

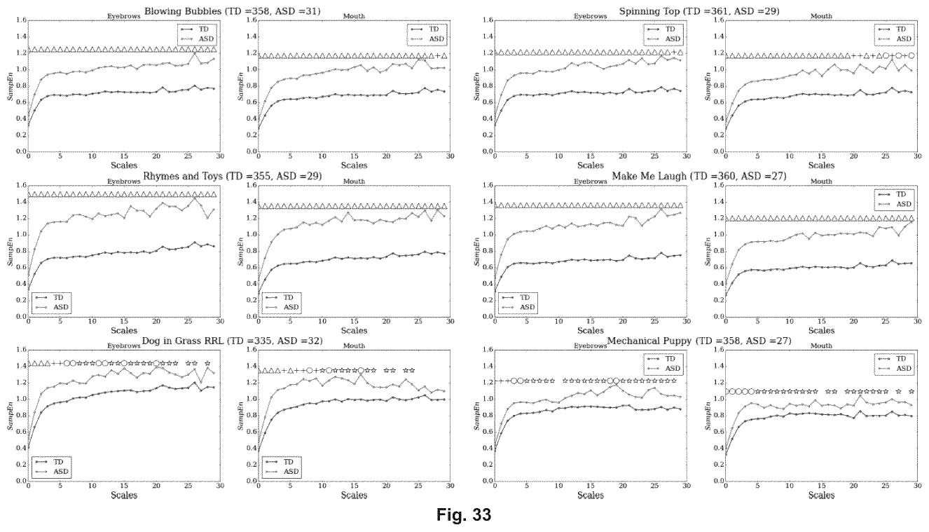

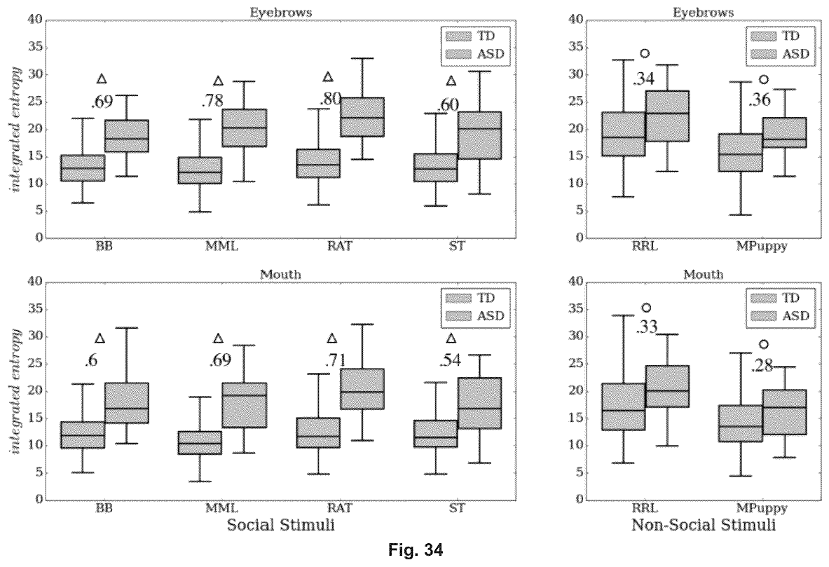

Complexity of Facial Landmarks' Dynamics. To address our first research question, we estimated the MSE for the eyebrows and mouth regions of the participants in both the ASD and TD groups (FIG. 33). Irrespective of the movies, whether the movie contained social or non-social content, the participants in the ASD group exhibited higher complexity (higher MSE) in their landmarks' dynamics, reflecting a higher level of ambiguous movements in such facial landmarks. The SampEn values were significantly different across the first 20 resolutions of the time-series for 4 out of the 6 presented movies (FIG. 33). Though the values were still significantly different between the ASD and TD groups, the mean difference was less pronounced across the multiple scales during the non-social movies (such as Dog in Grass RRL and Mechanical Puppy with p<0.01/0.05 and the effect sizes (r) were medium, in the 0.32-0.4 range: see FIG. 33) compared to the social movies (p<0.0001 with effect sizes (r) being large to very large, in the 0.51-0.82 range). Additionally, we computed the cumulative sum of SampEn values to compare between social and non-social movies. To do so, we aggregated the SampEn values associated with the first 20 scales for all the participants (integrated entropy: see FIG. 34). The results indicated that the integrated entropy was highly significantly different between the ASD and TD groups for all the social movies, having p<0.00001 with large or very large effect sizes (r) for both the eyebrows and mouth regions (FIG. 34). In contrast, the effect sizes (r) were smaller or medium for the non-social movies, though still significantly different (p<0.01). In short, the integrated entropy computed from the facial dynamics offered more confidence in distinguishing the ASD and TD groups, particularly while they watched the social movies. It is interesting to note that in spite of the relatively popular use of MSE in health research, this problem has never been addressed before, and often authors report lower MSE for data that is clearly more complex and vice-versa. Without taking this into consideration, it is challenging to use MSE to compare the complexity among signals that come from different participants.

Though the integrated entropy was significantly different between the ASD and TD groups, we still wanted to check if possible confounds such as (1) ‘percentage of missing data’ and the (2) ‘variation in the landmark dynamics’ had any influence on our findings. To do so, we performed a cross-correlation analysis. The results indicated a weak correlation (ranged 0.2-0.42) between the integrated entropy and ‘percentage of missing data’ as well as ‘variation in the landmark dynamics’. The lower values in the correlation analysis states that the (1) integrated entropy is not affected by the ‘percentage of missing data,’ and (2) integrated entropy measures are not just capturing the ‘variation in the landmarks' dynamics.’ Though we have considered only the participants that had a minimum of 40% of landmarks' data for our analysis after the preprocessing, we still wanted to see if the ‘percentage of missing data’ are statistically different between the groups. The results indicate that the two groups are not significantly different with regards to the ‘percentage of missing data’. Additionally, we have used these possible two confounds as a covariate in ANCOVA to further ensure the statistical difference between the ASD and TD groups. Even after adjusting for the two confounds, either individually or together, the significant difference was maintained between the ASD and TD groups, with p<0.0001 for social tasks and p<0.001 for non-social movies, indicating that the integrated entropy was not influenced by these confounds.

Additionally, to understand whether the two regions of interest (eyebrows and mouth) had different levels of complexity among themselves, we compared the integrated entropy values between these two regions within the ASD and TD groups. The results indicated that the integrated entropy for these two regions were not significantly different from each other (p>0.05 with effect size (r)<0.1), irrespective of the movies, for both the ASD and TD groups. Additionally, in order to understand individualized range of change in the integrated entropy across the tasks, we have estimated the range defined as,

$$integrated {entropy}_{RANGE} = integrated {entropy}_{MIN \_} integrated entropy MAX$$

for each participant across the six presented movies. A statistical analysis revealed that the ASD group had significantly larger change in their integrated entropyRANGE values than the TD group across the tasks in both the eyebrows (p=0.001, r=0.33) and mouth (p=0.0002, r=0.39) regions, with medium effect sizes. Such an observation again reflected that the participants with ASD had complex landmarks' dynamics that varied across the different movies presented.

Landmarks' Complexity and Affective State. This section addresses our second research question. A comparative analysis on the calculated energy upon the first-derivative of the time-series data related to neutral, positive, and negative affect indicated that the participants expressed a higher probability of neutral affect in response to all the movies (Table 2). This finding is consistent with previous work from our group¹⁹. Our movies were not designed to elicit any negative affect, as expected, we did not observe any significant negative facial emotions. We now consider the energy calculated from the first derivative of positive affect's time-series data (PositiveEnergy0) for further analysis. To understand if the observed complexity in facial landmarks' dynamics was simply an outcome of expressing positive affect in response to the movies, we extracted the correlation coefficient between the integrated entropy and PositiveEnergy0, for all the movies (combining both the ASD and TD groups). The results (Table 3) indicated that though some dependency existed for certain movies, such as Blowing Bubbles (BB) and Mechanical Puppy (MPuppy), it was not the case for the other movies such as Spinning Top (ST), Rhymes and Toys (RAT), Make Me Laugh (MML), Dog in Grass RRL (RRL). The results were similar even when the analysis was done separately for ASD and TD groups. Thus, the landmarks' dynamics were possibly a combination of affect and other manifestations such as atypical mouth movements²⁰⁻²², and frequently raised eyebrows and open mouth potentially reflecting level of attentional engagement¹⁹. Furthermore, the correlation coefficient (r) was comparatively more pronounced between the integrated entropy of the mouth region and the PositiveEnergy0, where the positive affect (smile) can be more prominent than the eyebrows. It was evident from these results that although a partial correlation existed with the affect data, the complexity of the landmarks' dynamics can offer a complementary measure with additional unique information.

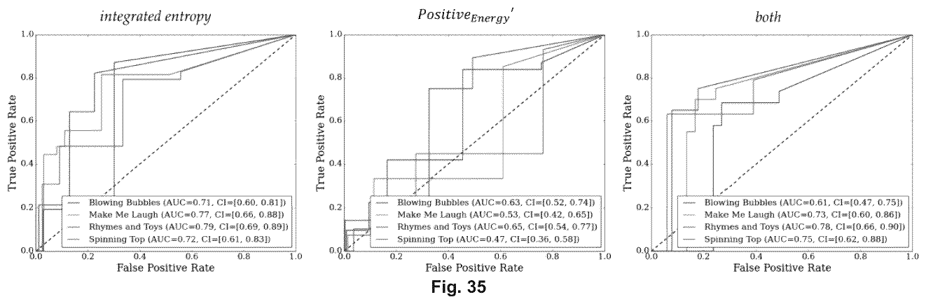

Classification Approach Using Landmarks' Complexity. To understand the feasibility of using the proposed complexity measure to automatically classify the individuals with ASD and TD, we used the integrated entropy values of the eyebrows and mouth regions as an input to a decision tree-based model. Since the statistical tests showed that the significant difference in integrated entropy between the groups had larger effect sizes during social movies, we have considered only those movies for this analysis. FIG. 35 shows the ROC curves and Table 4 shows other performance measures such as accuracy, precision, and recall using Leave-One-Out (LOO) cross-validation. Overall, the results indicated that the integrated entropy can serve as a promising biomarkers to classify the ASD and TD groups. In addition, we have also tested whether the affect data, e.g., PositiveEnergy0 could contribute to improved performance when included as input in such classification. The classification performance remained similar, indicating that the PositiveEnergy0 was not powerful enough to add additional value to the proposed integrated entropy. In fact, while using the affect data as the only input to the classifier, the performance was not up to the mark as it was while using only the integrated entropy as an input.

TABLE 2
Energy of the First-Derivative of Affect-Based Time-Series Data

| | Group | BB | ST | RAT | MML | RRL | Mpuppy |
|---|---|---|---|---|---|---|---|
| Nu | TD | 1.0 ± | 0.8 ± | 1.0 ± | 0.0 ± | 1.1 ± | 0.5 ± |
| | | 0.8 | 0.7 | 0.7 | 0.8 | 0.8 | 0.4 |
| | ASD | 1.8 ± | 1.9 ± | 2.1 ± | 2.1 ± | 0.5 ± | 0.7 ± |
| | | 1.0 | 2.2 | 1.0 | 1.4 | 0.8 | 0.5 |
| P | TD | 0.5 ± | 0.3 ± | 0.3 ± | 0.2 ± | 0.6 ± | 0.3 ± |
| | | 0.7 | 0.5 | 0.5 | 0.4 | 0.8 | 0.5 |
| | ASD | 1.2 ± | 0.7 ± | 0.9 ± | 0.6 ± | 1.2 ± | 0.4 ± |
| | | 1.4 | 0.7 | 0.9 | 0.7 | 1.0 | 0.5 |
| N | TD | 0.0 ± | 0.0 ± | 0.04 ± | 0.1 ± | 0.1 ± | 0.0 ± |
| | | 0.0 | 0.1 | 0.0 | 0.0 | 0.1 | 0.0 |
| | ASD | 0.1 ± | 0.1 ± | 0.07 ± | 0.1 ± | 0.1 ± | 0.0 ± |
| | | 0.0 | 0.1 | 0.1 | 0.0 | 0.1 | 0.0 |

Note:
No = neutral affect,
P = positive affect,
N negative affect,
BB = Blowing Bubbles,
ST = Spinning Top,
RAT = Rhymes and Tugs,
MAIM = Make Me laugh,
RR I. = Dog in Grass RIM, and
Mpuppy = Mechanical Puppy

TABLE 3
Correlation Coefficient Between Integrated
Entropy and Positive_{Energy}

| | BB | ST | RAT | MML | RRL | Mpuppy |
|---|---|---|---|---|---|---|
| Eyebrows | .49 | .08 | .05 | .11 | .01 | .41 |
| Mouth | .60 | .13 | .05 | .15 | .06 | .49 |

Note:
The values represent Sperman's ρ

Furthermore, the odds ratio calculated from the linear logistic regression (Table 5) indicates that the higher values of integrated entropy increase the chance to predict the risk of ASD by up to 1.8 times while considering either of the social movies. On the other hand, the changes in the affective expression (i.e., PositiveEnergy0) did not offered any positive odds ratio (that is, 0.8, which is less than 1). However, both the integrated entropy and PositiveEnergy0 had a significant contribution (p<0.05) in fitting the logistic regression model. Again, using only the integrated entropy has offered same results than when combined with PositiveEnergy0, indicating that the integrated entropy was independent from the affect measure and powerful enough to distinguish ASD and TD groups.

TABLE 4
Cross-Validation Results for the Decision Tree Model

| | Accuracy | | | Precision | | | Recall | | |
|---|---|---|---|---|---|---|---|---|---|
| Movies | A | B | C | A | B | C | A | B | C |
| Blowing Bubbles | 78.3% | 75.0% | 79.1% | 72.4% | 75.0% | 75.7% | 93.5% | 77.0% | 77.4% |
| Make Me Laugh | 78.3% © | 65.0% | 79.5% | 70.5% | 56% | 75.1% | 88.8% | 82.5% | 85.1% |
| Rhymes and Toys | 76.6% | 70.0% | 75.0% | 70.0% | 69.1% | 72.4% | 89.3% | 82.1% | 75.0% |
| Spinning Top | 76.7% | 61.6% | 63.3% | 70.3% | 62.5% | 61.3% | 89.6% | 51.7% | 65.5% |

Note:
A = integrated entropy from eyebrows and mouth regions, B = Positive and C features from both A and B.
The performances measures of the classifiers while using only the integrated entropy as input have showed stable and higher performance while comparing for all the social movies.
In contrast, the PositiveEnergy0 as only input has showed the lowest performance.
Combining both the integrated entropy and PositiveEnergy0 as an input to the classifier showed a slight increase in accuracy and precision only for Blowing Bubbles and Make Me Laugh (not otherwise), while the recall rate is reduced.
Thus, we can say that combining PositiveEnergy0 with integrated entropy is not adding additional information during the classification between the ASD and TD groups.

### Discussion and Conclusion

We designed an iPad-based application (app) that displayed strategically designed, developmentally appropriate short movies involving social and non-social components. Children diagnosed with ASD and children with typical development (TD) took part in our study, watching the movies during their well-child visit to pediatric clinics. The device's front-facing camera was used to record the children's behavior and capture ASD-related features. Our current work was focused on exploring biomarkers related to facial dynamics. We exploited the children's facial dynamics from the eyebrows and mouth regions using multiscale entropy (MSE) analysis to study the complexity of such facial landmarks' dynamics. The complexity analysis may give insights about the level of irregularity (or ambiguity) in the facial dynamics that can potentially be used as a biomarker to distinguish between children with ASD and those with typical development (TD). Basically, the complexity estimates using entropy offer information about how easy is to predict facial landmark dynamics rather than just their variation. Specifically, a time-series with higher variations that are highly periodic will result in extremely low values of the entropy measure. Similar is the case when the time-series data is almost stable with very low variations, the entropy value will still be low (which was the case for our TD participants). In contrast, for a time-series with higher variability, irregular and non-periodic movements, the entropy value will be high, indicating higher complexity in facial dynamics (which was the case for the ASI) participants). We speculate that the presence of greater predictability with minor facial movements in the TD toddlers reflects a higher level and more consistent understanding of the shared social meaning of content of the movies (e.g., rhymes, a conversation). If so, the TD toddlers might be expected to respond more predictably to the stimuli, whereas the responses of the children with autism may be more idiosyncratic. It has been previously documented (e.g., reference [9]) that children with autism make more atypical and ambiguous facial expressions and that these vary across children with autism.

As expected, the results of our modified approach to MSE analysis captured distinctive landmarks' dynamics in children with ASD, characterized by a significantly higher level of complexity in both the eyebrows and mouth regions when compared to typically-developing children. This measure can be robust and complementary to other measures such as affective state¹⁹. The observation from the integrated entropy supports recent work indicating that individuals with ASD often exhibit a higher probability of neutral expression¹⁹, Neutral expressions might be interpreted by others as more ambiguous in terms of the affective state they convey. The results here reported are also in agreement with other works related to atypical speech and mouth movements^{2,22}, offering a scope and directions for further exploration. Also, it was shown that the individuals with ASD have difficulties in affect coordination during interpersonal social interaction⁴⁰, it would be also interesting to study the potential of complexity/coordination in facial dynamics in such context in future. Finally, we observed that the proposed integrated entropy (the sum of SarnpEn from 1-20 scales of the MSE) might not only hold promise in distinguishing children with ASD versus TD, but also from other developmental disorders (e.g., developmental delay, /language delay. Additionally, the integrated entropy has offered better performance in classifying the ASD and TD groups of participants while using machine learning based classifiers, e.g., a decision tree-based model, offering avenue to build an automated decision—making pipeline for conveying a probability of risk in. children with ASD while using the app in home-based settings. Notwithstanding the fact that in addition to the complexity measure it would be necessary to combine other features such as gaze-related indices, name call response, signatures of motor deficits, and more, as mentioned in [7] before deploying such automated decision-making tool. Complementary to the facial landmarks' dynamics, the known deficits in motor control in children with ASD can be manifested in the form of poor postural control WI which can be captured easily with our data. Exploring complexity estimates in these head motions can be an interesting future work.

Limitations of this study include: landmarks and affect detection were based on algorithms trained primarily on adults, although our previous work showed it was still rah, able for toddlers; other measures of complexity might be more robust than the MSE in their ability to discriminate children with and without ASD; and the study sample, while relatively large, still has a limited number of ASD participants and did not have sufficient power to determine the impact of demographic characteristics on the results.

To conclude, in this work, we introduced a newly normalized measure of MSE more suited for across-subject comparison and demonstrated that the complexity of facial dynamics has potential as an ASI) biomarker beyond more traditional. measures of affective expression. Our findings were consistent with the previous work on patterns of affective expression in children with ASD, while adding new discoveries underscoring the value of dynamic facial primitives. Considering that autism is a very heterogeneous condition, the combination of the novel biomarkers described here with additional biomarkers has the potential to improve the development of scalable screening, diagnosis, and treatment monitoring tools.

TABLE 5
Odds Ration Results From Linear Logistic Regression

| | Integrated entropy | | | | | |
|---|---|---|---|---|---|---|
| | Eyebrows | | Mouth | | Positive_{Energy} | |
| Movies | Odds | P-value | Odds | P-value | Odds | P-value |
| Blowing Bubbles | 1.49 | .004 | 1.49 | .005 | 0.80 | .018 |
| Make Me Laugh | 1.54 | .004 | 1.51 | .004 | 0.82 | .030 |
| Rhymes and Toys | 1.52 | .004 | 1.80 | .004 | 0.81 | .026 |
| Spinning Top | 1.48 | .005 | 1.52 | .004 | 0.85 | .026 |

### REFERENCES

[1]P. Ekman, “Emotional and conversational nonverbal signals,” in Proc. Lang. Knowl. Representation, 2004, pp. 39-50.
[2]P. Ekman and W. V. Friesen, “Constants across cultures in the face and emotion,” J. Pers. Soc. Psychol., vol. 17, no. 2, pp. 124-129, 1971.
[3]C. E. Izard, R. R. Huebner, D. Risser, and L. Dougherty, “The young infant's ability to produce discrete emotion expressions,” Develop. Psychol., vol. 16, no. 2, pp. 132-140, 1980.
[4] American Psychiatric Association, “DSM-5 diagnostic classification,” in Diagnostic and Statistical Manual of Mental Disorders, Washington, D.C., USA: Amer. Psychiatr. Assoc. 2013.

[5]C. Lord et al., “The autism diagnostic observation schedule generic: A standard measure of social and communication deficits associated with the spectrum of autism,” J. Autism Develop. Disord., vol. 30, no. 3, pp. 205-223, 2000.

[6]N. Yirmiya, C. Kasari, M. Sigman, and P. Mundy, “Facial expressions of affect in autistic, mentally retarded and normal children,” J. Child Psychol. Psychiatry, vol. 30, no. 5, pp. 725-735, 1989.
[7]G. Dawson and G. Sapiro, “Potential for digital behavioral measurement tools to transform the detection and diagnosis of autism spectrum disorder,” JAMA Pediatrics, vol. 173, no. 4, pp. 305-306, 2019.
[8]A. Metallinou, R. B. Grossman, and S. Narayanan, “Quantifying atypicality in affective facial expressions of children with autism spectrum disorders,” in Proc. IEEE Int. Conf. Multimedia Expo, 2013, pp. 1-6.
[9]C. Grossard et al., “Children with autism spectrum disorder produce more ambiguous and less socially meaningful facial expressions: An experimental study using random forest classifiers,” Mol. Autism, vol. 11, no. 1, 2020, Art. no. 5. [10]J. Manfredonia et al., “Automatic recognition of posed facial expression of emotion in individuals with autism spectrum disorder,” J. Autism Develop. Disord., vol. 49, no. 1, pp. 279-293, 2019. [11]M. Leo et al., “Computational analysis of deep visual data for quantifying facial expression production,” Appl. Sci., vol. 9, no. 21, 2019, Art. no. 4542. [12]E. Zane, Z. Yang, L. Pozzan, T. Guha, S. Narayanan, and R. B. Grossman, “Motion-capture patterns of voluntarily mimicked dynamic facial expressions in children and adolescents with and without ASD,” J. Autism Develop. Disord., vol. 49, no. 3, pp. 1062-1079, 2019. [13]T. Guha et al., “On quantifying facial expression-related atypicality of children with Autism spectrum disorder,” in Proc. IEEE Int. Conf. Acoust. Speech Signal Process., 2015, pp. 803-807.
[14]T. Guha, Z. Yang, R. B. Grossman, and S. S. Narayanan, “A computational study of expressive facial dynamics in children with autism,” IEEE Trans. Affect. Comput., vol. 9, no. 1, pp. 14-20, First Quarter 2018.
[15]M. Costa, A. L. Goldberger, and C. K. Peng, “Multiscale entropy analysis of complex physiologic time series,” Phys. Rev. Lett., vol. 89, no. 6, 2002, Art. no. 068102.
[16]M. Costa, A. L. Goldberger, and C. K. Peng, “Multiscale entropy analysis of biological signals,” Phys. Rev. E-Stat. Nonlinear, Soft Matter Phys., vol. 71, no. 2, 2005, Art. no. 021906.
[17]S. Harati, A. Crowell, H. Mayberg, J. Kong, and S. Nemati, “Discriminating clinical phases of recovery from major depressive disorder using the dynamics of facial expression,” in Proc. Annu. Int. Conf. IEEE Eng. Med. Biolo. Soc., 2016, pp. 2254-2257.
[18]Z. Zhao et al., “Atypical head movement during face-to-face interaction in children with autism spectrum disorder,” Autism Res., vol. 14, no. 6, pp. 1197-1208, 2021.
[19]K. L. H. Carpenter et al., “Digital behavioral phenotyping detects atypical pattern of facial expression in toddlers with autism,” Autism Res., vol. 14, no. 3, pp. 488-499, 2020.
[20]J. R. Green, C. A. Moore, and K. J. Reilly, “The sequential development of jaw and lip control for speech,” J. Speech, Lang. Hear. Res., vol. 45, no. 1, pp. 66-79, 2002.
[21]L. D. Shriberg, J. R. Green, T. F. Campbell, J. L. McSweeny, and A. R. Scheer, “A diagnostic marker for childhood apraxia of speech: The coefficient of variation ratio,” Clin. Linguistics Phonetics, vol. 17, no. 7. pp. 575-595, 2003. [22]E. J. Tenenbaum et al., “A six-minute measure of vocalizations in toddlers with autism spectrum disorder, Autism Res., vol. 13, no. 8, pp. 1373-1382, 2020. [23]D. L. Robins, K. Casagrande, M. Barton, C. M. A. Chen, T. Dumont-Mathieu, and D. Fein, “Validation of the modified checklist for autism in toddlers, revised with follow-up (MCHAT-R/F),” Pediatrics, vol. 133, no. 1, pp. 37-45, 2014.
[24]R. Luyster et al., “The autism diagnostic observation schedule—Toddler module: A new module of a standardized diagnostic measure for autism spectrum disorders,” J. Autism Dev. Disord., vol. 39, no. 9, pp. 1305-1320, 2009. [25]D. E. King, “Dlib-ml: A machine learning toolkit,” J. Mach. Learn. Res., vol. 10, pp. 1755-1758, 2009.
[26]Z. Chang et al., “Computational methods to measure patterns of gaze in toddlers with autism spectrum disorder,” JAMA Pediatrics, vol. 175, no. 8, pp. 827-836, 2021.
[27]S. Perochon et al., “A scalable computational approach to assessing response to name in toddlers with autism,” J. Child Psychol. Psychiatry Allied Discip., vol. 62, no. 9, pp. 1120-1131, 2021.
[28]T. Baltrusaitis, A. Zadeh, Y. C. Lim, and L. P. Morency, “OpenFace 2.0: facial behavior analysis toolkit,” in Proc. 13th IEEE Int. Conf. Automat. Face Gesture Recognit., 2018, pp. 59-66.
[29]J. Hashemi et al., “Computer vision analysis for quantification of autism risk behaviors,” IEEE Trans. Affect. Comput., vol. 12, no. 1, pp. 215-226, First Quarter 2021. [30]W. J. Yan, Q. Wu, J. Liang, Y. H. Chen, and X. Fu, “How fast are the leaked facial expressions: The duration of micro-expressions,” J. Nonverbal Behav., vol. 37, no. 4, pp. 217-230, 2013.
[31]T. A. Salthouse and C. L. Ellis, “Determinants of eye-fixation duration,” Amer. J. Psychol., vol. 93, no. 2, pp. 207-234, 1980.
[32]N. Galley, D. Betz, and C. Biniossek, “Fixation durations—Why are they so highly variable?,” in Proc. Adv. Vis. Perception Res., 2015, pp. 83-106.
[33]J. S. Richman and J. R. Moorman, “Physiological time-series analysis using approximate and sample entropy,” Amer. J. Physiol. Hear. Circ Physiol., vol. 278, no. 6, pp. H2039-H2049, 2000.
[34]D. E. Lake, J. S. Richman, M. Pamela Griffin, and J. Randall Moorman, “Sample entropy analysis of neonatal heart rate variability,” Am. J. Physiol. Regul. Integr. Comput. Physiol., vol. 283, no. 3, pp. R789-R797, 2002.
[35]X. Dong et al., “An improved method of handling missing values in the analysis of sample entropy for continuous monitoring of physiological signals,” Entropy, vol. 21, no. 3, 2019, Art. no. 274. [36]E. Cirugeda-Roldan, D. Cuesta-Frau, P. Miro-Martinez, and S. Oltra-Crespo, “Comparative study of entropy sensitivity to missing biosignal data,” Entropy, vol. 16, no. 11, pp. 5901-5918, 2014.
[37]J. Hashemi, Q. Qiu, and G. Sapiro, “Cross-modality pose invariant facial expression,” in Proc. Int. Conf. Image Process., 2015, pp. 4007-4011.
[38]L. Breiman, J. H. Friedman, R. A. Olshen, and C. J. Stone, Classification and Regression Trees. London, U.K.: Routledge, 2017.
[39]E. R. DeLong, D. M. DeLong, and D. L. Clarke-Pearson, “Comparing the areas under two or more correlated receiver operating characteristic curves: A nonparametric approach,” Biometrics, vol. 44, no. 3, 1988, Art. no. 837.
[40]C. J. Zampella, L. Bennetto, and J. D. Herrington, “Computer vision analysis of reduced interpersonal affect coordination in youth with autism spectrum disorder,” Autism Res., vol. 13, no. 12, pp. 2133-2142, 2020. [41]G. Dawson et al., “Atypical postural control can be detected via computer vision analysis in toddlers with autism spectrum disorder,” Sci. Rep., vol. 8, no. 9, pp. 1-7, 2018.

### Example 5

Complexity Analysis of Head Movements in Autistic Toddlers Early differences in sensorimotor functioning have been documented in young autistic children and infants who are later diagnosed with autism. Previous research has demonstrated that autistic toddlers exhibit more frequent head movement when viewing dynamic audiovisual stimuli, compared to neurotypical toddlers. To further explore this behavioral characteristic, in this study, computer vision (CV) analysis was used to measure several aspects of head movement dynamics of autistic and neurotypical toddlers while they watched a set of brief movies with social and nonsocial content presented on a tablet. Methods: Data. were collected from 457 toddlers, 17 to 36 months old, during their well-child visit to four pediatric primary care clinics. Forty-one toddlers were subsequently diagnosed with autism. An application (app) displayed several brief movies on a tablet, and the toddlers watched these movies while sitting on their caregiver's lap. The front-facing camera in the tablet recorded the toddlers' behavioral responses. CV was used to measure the participants' head movement rate, movement acceleration, and complexity using multiscale entropy. Results: Autistic toddlers exhibited significantly higher rate, acceleration, and complexity in their head movements while watching the movies compared to neurotypical toddlers, regardless of the type of movie content (social vs. nonsocial). The combined features of head movement acceleration and complexity reliably distinguished the autistic and neurotypical toddlers. Conclusions: Autistic toddlers exhibit differences in their head movement dynamics when viewing audiovisual stimuli. Higher complexity of their head movements suggests that their movements were less predictable and less stable compared to neurotypical toddlers. CV offers a scalable means of detecting subtle differences in head movement dynamics, which may be helpful in identifying early behaviors associated with autism and providing insight into the nature of sensorimotor differences associated with autism.

Autism is characterized by differences in social communication and the presence of restrictive and repetitive behaviors (American Psychiatric Association, 2014). In addition to the presence of motor stereotypies, other motor differences often associated with autism include impairments in fine and gross motor skills, motor planning, and motor coordination (Bhat, 2021; Flanagan et al., 2012; Fournier et al., 2010; Melo et al., 2020). Studies based on home videos of infants who were later diagnosed with autism reported asymmetry in body movements (Baranek, 1999; Esposito & Venuti, 2009; Teitelbaum et al., 1098). Detailed motor assessments have documented difficulties in gait and balance stability, postural control, movement accuracy, manual dexterity, and praxis among autistic individuals (Chang et al., 20.10′; Minshew et al., 2004; Molloy et al., 2003; Wilson, Enticott, & Rinehart, 2018; Wilson, McCracken, et al., VMS).

Recent research utilizing computer vision analysis to measure differences in movement patterns has documented differences in patterns of head movement dynamics while watching dynamic audiovisual stimuli among autistic children. Martin and colleagues (Martin et al., 2018) examined differences in head movement displacement and velocity in 2.5- to 6.5-year-old children with and without a diagnosis of autism while they watched a video of social arid nonsocial stimuli. Head movement differences between the autistic and neurotypical children were found in the lateral (yaw and roll) but not vertical (pitch) movement and were specific to periods when children were watching social videos. These authors suggested that the autistic children may use head movements to modulate their perception of social scenes. Zhao et al. quantified three-dimensional head movements in 6- to 13-year-old children with and without autism while they were engaged in a conversation with an adult (Zhao et al., 2021). They found that the autistic children showed differences in their head movement dynamics not explained by whether they were fixating on the adult. Dawson et al., 2018 found that toddlers who were later diagnosed with autism exhibited a significantly higher rate of head movement while watching brief movies as compared to neurotypical toddlers.

In the current study, we extended our earlier work on head movements in autistic toddlers (Dawson et al., 2018) in two ways: First, we sought to replicate our earlier findings related to the head movement rate with a significantly larger sample using similar but re-designed novel movies with social versus nonsocial content. Second, we expanded our analysis by not only computing head movement rate, but also the acceleration and the complexity of the time-series associated with the head movements. The acceleration provided an estimate of changes in head movement rate (i.e., velocity), while the complexity estimate reflected the level of predictability and stability of head movements (Costa et al., 2002). We used multiscale entropy (MSE) to quantitatively assess the complexity or predictability of head movement dynamics (Costa et al., 2002). This metric quantified the regularity of the one-dimensional time-series on multiple scales (Costa et al., 2002; Zhao et al., 2021).

We hypothesized that, compared to neurotypical toddlers, autistic toddlers would exhibit higher head movement rate, acceleration, and increased complexity (less predictability). We also examined whether differences in head movement measures were more pronounced when autistic children watched movies with high levels of social content. Finally, we used machine learning classification analyses to determine whether these measures can be integrated to distinguish autistic and neurotypical toddlers.

### Methods for Example 5

Participants. Participants were 457 toddlers, 17-36 months of age, who were recruited from four pediatric primary care clinics during a well-child checkup. Forty-one toddlers were subsequently diagnosed with autism spectrum disorder (ASD) based on DSM-5 criteria. Inclusion criteria were: (a) age 16-38 months; (b) not ill at the time of visit; and (c) caregiver's primary, language at home was English or Spanish. Exclusion criteria were: (a) known hearing or vision impairments; (b) the child was too upset during the visit; (c) the caregiver expressed no interest, not enough time; (d) the child was not able to complete the study procedures (e.g., if child would not stay in their caregiver's lap, or the app or device failed to upload data), or the clinical information was missing; and/or (e) presence of a significant sensory or motor impairment that precluded the child from seeing the movies and/or sitting uptight. Table 1 shows the participants' demographic characteristics.

Ethical considerations. Caregivers provided written informed consent, and the study protocols were approved by the Duke University Health System Institutional Review Board (Pro00085434, Pro00085435).

Clinical measures Modified checklist for toddlers revised with follow-up (M-CHAT-R/F) As a part of routine clinical care, all participants were assessed with a commonly used autism screening questionnaire, the M-CHAT-R/F (Robins et al., 2014). The M-CHAT-R/F consists of 20 questions answered by the caregiver to evaluate the presence/absences of autism-related symptoms.

Diagnostic and cognitive assessments. Toddlers with a total M-CHAT-R/F score≥3 initially and those for whom the total score was 2 after the follow-up questions, and/or those for whom the pediatrician and/or parent expressed developmental concerns, were referred for diagnostic evaluation by the study team psychologist. The Autism Diagnostic Observation Schedule—Toddler Module (ADOS-2) was administered by research-reliable licensed psychologists who determined whether the child met DSM-5 criteria for ASD (Luvater et al., 2009). Cognitive and language abilities were assessed using the Mullen Scales for Early Learning (Mullen, 0.1995).

Group definitions. Group definitions were: (1) Autistic (Pia 41), defined as having a positive M-CHAT-R/F score or caregiver/physician raised concerns and subsequently meeting DSM-5 diagnostic criteria for autism spectrum disorder (ASD) with or without developmental delay based on both the ADOS-2 and clinical judgment by licensed psychologist; and (2) Neurotypical (N=416), defined as (a) having a high likelihood of typical development with a negative score on the M-CHAT-R/F and no developmental concerns raised by caregiver/physician or (b) having a positive M-CHAT-R/F score and/or care-giver/physician raised concerns, but then determined based on the ADOS-2 and clinical judgment of the psychologist as not having developmental or autism-related concerns. There was another group of participants (N=12) who had a positive M-CHAT-R/F score and received a diagnosis other than autism (e.g., language delay without autism). Given the small sample size, we have excluded these participants from our current analyses.

Application (app) administration and stimuli. In each of the four clinics, a quiet room with few distractions was identified in which the app could be administered. Although it was quiet and without a lot of distraction, it was not otherwise controlled as in a laboratory setting. The rooms for the four clinics were similar in size, lighting, and presence of distractions (e.g., table in the room).

App administration. We designed an app compatible with iOS devices which displayed developmentally appropriate movies. The front-facing camera was used to record the toddlers' behavioral responses while watching the movies. Caregivers were requested to hold their child on their lap while a tablet was placed on a tripod at about 60 cm in front of the child. No special instructions were given regarding how to hold the child on their lap. The parent sat quietly throughout the app administration and was asked not to guide the child's behavior or give instructions. Other family members (e.g., siblings) and the assistant who administered the app stayed behind both the caregiver and the child to reduce distractions during the experiment. We computed and analyzed the participants' head movements that were recorded while they watched four movies with high social content, and three movies with low. social content (nonsocial), described next.

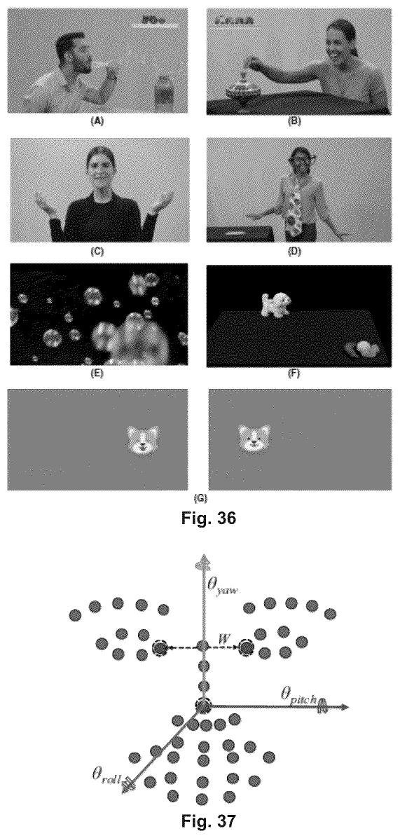

Social movies containing human actors in the scene. Blowing-Bubbles (˜64 s): An actor with a bubble wand blew the bubbles, along with smiling and frowning and limited verbal expressions (FIG. 36A). Spinning-Top (˜53 s): An actress played with a spinning top, smiled, and frowned with limited verbal expressions (FIG. 36B). Rhymes (˜30 5): An actress said nursery rhymes while smiling and gesturing (FIG. 36C). Make-Me-Laugh (˜56 s): An actress demonstrated funny actions while smiling (FIG. 36D).

Nonsocial movies containing dynamic objects. Floating-Bubbles (˜35 s): Bubbles were presented at random and moved throughout the frame (FIG. 36E). Mechanical-Puppy (˜25 s): A mechanical toy puppy barked and walked towards vegetable toys (FIG. 36F). Dog-in-Grass-Right-Right-Left (RRL) (˜40 s): A puppy appeared at parts of the screen, randomly in the right/left part of the screen followed by a constant right-right-left pattern (FIG. 36G).

Capturing facial landmarks and head orientation using computer vision. A computer vision algorithm was used to detect the faces in each frame of the recorded video (King, 2009). Similar to (Chang et al., 2021; Perochon et al., 2021), scarce human supervision was triggered by a face-tracking algorithm to ensure that we tracked only the participant's face. Then, we extracted 49 facial landmarks consisting of 2D-positional coordinates (Baltrusaitis et al., 2018) (FIG. 37) that were time-synchronized with the movies. Since we were interested in measuring the participants' head movements while watching the movies, we focused our analysis on time-segments for which the participants are looking towards the tablet's screen. To this end, for each frame we computed the child's head pose angles relative to the tablet (FIG. 37), θ_{yaw} (left-right), θ_{pitch} (up-down), and θᵣₒₗₗ (tilting left-right) (Hashemi et al., 2021). A criterion |θ_{yaw}|<˜20° of head pose angle was used as proxy for attention (Dawson et al., 2018; Hashemi et al., 2021), supported by ‘central bias’ theory for gaze estimation (Li et al., 2013; Mannan et al., 1995). In addition, we also ensured that the participants were looking towards the tablet's screen using their gaze information extracted using the automatic gaze estimation algorithm (Chang et al., 2021). The resulting time-segments were used to estimate the head movement timeseries data for our further analysis.

TABLE 1
Demographic characteristics

| | N(%) | |
|---|---|---|
| Groups | Neurotypical (N = 416) | Autistic (N - 41) |
| Age in months | | |
| Mean (Si), range) | 20.59 (3.18, 17.2-32.3)′ | 24.38 (4.73, 17.9-36.8r |
| Sex | | |
| Female | 207 (49.76%)″ | 12 (29.26%)″ |
| Male | 209 (50.24%)″ | 29 (70.73%)″ |
| Race | | |
| American Indian/Alaskan Native | 1 (0.24%) | 3 (7.32%) |
| Asian | 6 (1.44%) | 1 (2.44%) |
| Black or African American | 43 (10.33%) | 6 (14.63%) |
| Native Hawaiian or other Pacific Islander | 0 (0.00%) | 0 (0.00%) |
| White/Caucasian | 316 (75.96%) | 21(51.22%) |
| More than one race | 41 (9.85%) | 6 (14.63%) |
| Other | 9 (2.16%) | 4 (9.75%) |
| Ethnicity | | |
| Hispanic/Latino | 31 (7.45%)″ | 12 (29.26%)″ |
| Not Hispanic/Latino | 385 (92.54%)^{b} | 29 (70.73%)″ |
| Caregivers' highest level of education | | |
| Without high school diploma | 2 (0.49%)″ | 4 (9.76%)″ |
| High school diploma or equivalent | 14 (3.36%)″ | 5 (12.20%)″ |
| Some college education | 40 (9.61%)″ | 10 (24.39%)″ |
| 4-Year college degree or more | 356 (85.57%)″ | 21 (53.65%)″ |
| tiriknown/not reported | 4 (0.96%) | 0 (0.00%) |
| Clinical variables | Mean (SD, range) | |
| ADDS -.2 toddler module | | |
| Calibrated severity score | N/A | 7.56 (1.67, 2-70) |
| Mullen Scales of Early Learning | | |
| Early Learning Composite Score | N/A | 63.64 (10.17, 49-87) |
| Expressive Language T-Score | N/A | 28.08 (7.41, 20-50) |
| Receptive Language T-Score | N/A | 23.14 (4.93, 20-37) |
| Fine Motor 7-Score | N/A | 34.11 (10.76, 20-56) |
| Visual Reception 7-Score | N/A | 33.94 (10.63, 20-50) |

ADOS-2: Autism Diagnostic Observation Schedule - Second Edition. Age of diagnosis: Mean = 23.9, SD = 4.5, Range = 18-37. Time between the ages at diagnosis and app administration: Mean = 0.7, SO - 1.2, Range = −0.1-5.9.
″Significant difference between the. two groups based on ANOVA test.
″Significant difference between the two groups based on Chi-Square test.

### Computational Estimates of the Head Movement.

Rate. We computed the average Euclidean displacements of three central landmarks (represented in red colors in FIG. 37) between two consecutive frames. To reduce the effect of changes in the distance between the child and the camera, we normalized the landmarks' displacement, dividing by a 1 s window average of the distance between the eyes (W in Figure #7). We regularized the time-series corresponding to the normalized landmark's displacement by considering an average of the signals over a moving window of 10 frames (1/3 s) (Dawson et al., 2018). For further analysis, we estimated mean head movement rate (Mean_rateHM) from this time-series.

Acceleration. We estimated the child's acceleration from the rate of head movements. Intuitively, acceleration is associated with the child's head movement rate (i.e., their physical velocity), and therefore the first derivative of this quantity can be interpreted as the second derivative of the head positions. This second-order derivate is of particular interest since it relates to the magnitude of the instantaneous forces involved in head movement. We estimated the absolute mean of the acceleration (Mean_accelHM) as the difference between the head movement between two consecutive frames, averaged over a 1/3 s window. We have also estimated total energy of the head movements, which was not as powerful as Mean_accelHM in detecting a statistical difference between the two groups.

Complexity. To estimate the complexity of head movement rate (time-series) at multiple time-resolutions using MSE (Costa et al., 2002, 2005), a time-series X ¼fx1, . . . , xi, . . . , xN g was down-sampled to 30 different scales (T=1 to 30) and represented as

$$y ð τ Þ j \frac{1}{4} 1 τ \sum j τ i \frac{1}{4} ð j\_ 1 Þ τÞ1 xi , where 1 \leq j \leq N T ( 1 )$$

Subsequently, sample entropy (SampEn) was calculated on each of these resolutions of the time-series. SampEn can be defined as an estimate of irregularity in time-series: given an embedding dimension m and a positive scalar tolerance r, the SampEn is the negative logarithm of the conditional probability that if the sets of simultaneous data points of length m repeat within the distance r, then the sets of data points having length m+1 also repeat within the distance r (Richman & Moorman, 2000). If the repeatability is low, the SampEn will be high, and the time-series is considered more complex. Considering the m-dimensional embedding vector Xmi ¼xmi, xmi [symbol] 1, xmi [symbol] 2, . . . , xmi [symbol] m_1_ _from the time-series X ¼fx1, . . . , xi, . . . , xN g of length N, the distance d between two vectors xmi and xmj was defined as d

$$max \{ 1 , k = 1 , 2 , .$$

$$( 2 ) and {IC}^{′} 41 ( r ) .$$

$$SampEn — ( 3 ) C$$

Equation (3) defines the sample entropy, where Cmδr [symbol] and Cm [symbol] 1δr [symbol] denote the cumulative sum of the number of repeating vectors in the m and m+1 embedding spaces, respectively. Two vectors xmi and xmj were defined as repeating if they met the condition d xmi, xmj_ _s≤r, where i≠j. To handle any bias due to missing data while computing the SampEn, we only considered the segments where the data were available in the m+1 dimensional space (Dong et al., 2019). The parameter m as set to 2, similar to (Costa et al., 2002; Harati et al., 2016), and r=0.15*σ, where 0.15 is a scaling factor chosen, similar to (Costa et al., 2002; Dong et al., 2019; Lake, Richman, Pamela Griffin, & Randall Moorman, 2002). Σ denotes the signal's standard deviation that characterizes the time-series. Since a can vary across different participants, we used the population-wise standard deviation; this choice defined a distance threshold r consistent across participants (see Krishnappababu et al., 2021 for a detailed discussion). Finally, a global complexity estimation (across multiple scales) was obtained integrating the SampEn across the first 10 scales (Intergrated-entropyHM). At least 40% of the data were necessary to perform effective complexity analysis (Cirugeda-Roldan et al., 2014; Lake et al., 2002) after handling the missing segments similar to Krishnappababu et al. (2021). Below this threshold the estimation of the SampEn may not be reliable. Participants having <40% data were removed from the analysis for each specific movie.

Statistical analysis. Mann-Whitney U-test was used to estimate the statistical significance between the groups, using python (pingouin.mwu). Within group comparisons (e.g., to compare between the social and nonsocial movies) were performed using Wilcoxon signedrank test using python (pingouin.wilcoxon). The statistical power was calculated using the effect size, ‘r’ provided by pingouin.mwu and pingouin.wilcoxon. A 2×2 mixed ANOVA was used to estimate the main effects due to (a) group and (b) movie type (social and nonsocial) and their interaction effect with python function, pingouin.mixed_anova. For mixed ANOVA analysis, we have estimated the mean values of the Mean_rateHM, Mean_accelHM and Integrated_entropyHM for the social and non-social movies across all the participants. Additionally, analysis of covariance (ANCOVA) using pingouin. ancova was performed to determine the influence of covariates such as participants' age and percentage of missing data. A support vector machine (SVM)-based classifier with radial basis function (RBF) kernel (Cortes & Vapnik, 1995) was used to assess the classification power of the proposed features. Classification performance was compared using the area under the curve (AUC) of the receiver operating characteristic (ROC) with Leave-one-out cross-validation (Elisseeff & Pontil, 2003). 95% confidence intervals were computed with the Hanley and McNeil method (Hanley & McNeil, 1982). We chose SVM since it is popular when used on relatively smaller datasets, and also a cross-validation was done to minimize the risk of overoptimistic classification (Vabalas et al., 2019). Classification performance of the different models were compared based on their true difference (dt), estimated using observed difference in the error (d) and sum of variance of the error across the two models (ad), at a significant threshold of p<0.05: dt=d_ 1.96*σd (Tan et al., 2006). If the dt spans over zero, then the two models are considered not significantly different.

### Results

Engagement with the app administration. The percentages of assessments to which the child attended for the majority of the app administration were 95% and 93% for the neurotypical and autistic groups, respectively. Differences in rate, acceleration, and complexity of head movements

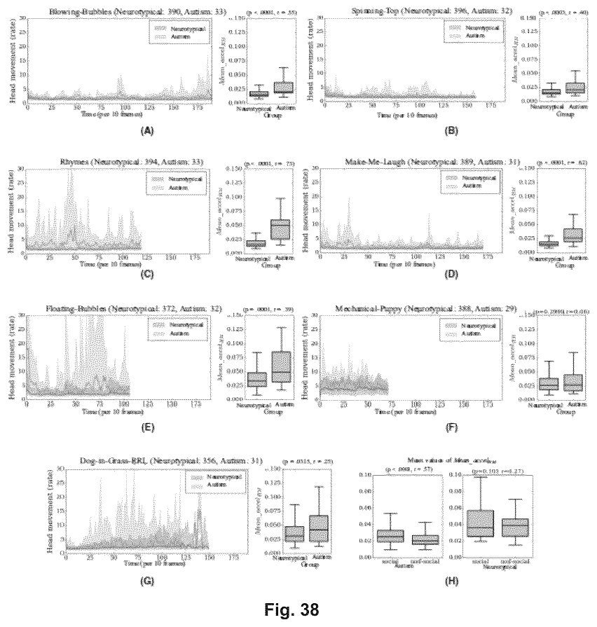

Rate. FIG. 38 displays the time-series plots of the head movement rate per 1/3 s for each of the movies. To replicate our findings in (Dawson et al., 2018), we used the mean head movement rate (Mean_rateHM) to examine between the two groups. A 2×2 mixed ANOVA was used to estimate the main effects of (a) group and (b) movie type (social and nonsocial) and the interaction between group * movie type. There was a significant effect of group, with autistic toddlers exhibiting a higher rate of movement compared to the neurotypical toddlers (F(1, 427)=42.48, p<0.0001), and a significant effect of movie type, with social movies eliciting more movement than nonsocial movies (F(1, 427)=35.22 p<0.0001), but no significant interaction effect (F(1, 427)=0.77, p=0.09). Analyzed by type of movie separately, the Mean_rateHM was significantly higher for the autistic group compared to neurotypical group for social movies (Blowing-Bubbles (p<0.0001, r=0.56), Spinning-Top (p=0.0001, r=0.39), Rhymes (p<0.0001, r=0.72), and Make-Me-Laugh (p<0.0001, r=0.62)), with medium to large effect sizes, as well as for the nonsocial movies except for Mechanical-Puppy (Floating-Bubbles (p<0.001, r=0.35), Dog-in-Grass-RRL (p=0.005, r=0.27)), with small to medium effect sizes.

Acceleration. FIG. 38 displays the mean acceleration values per group. A 2×2 mixed ANOVA results showed, again there was a significant effect of group, with autistic toddlers exhibiting higher acceleration than neurotypical toddlers (F(1, 427)=38.65, p<0.0001), and a significant effect of movie type, with social movies eliciting higher acceleration than nonsocial movies (F(1, 427)=70.14, p<0.0001), but no significant interaction effect (F(1, 427)=0.007, p=0.92). Analyzed by type of movie separately, across all the social movies, the Mean_accelHM of the autistic group was significantly higher than neurotypical group, with medium to large effect sizes (FIG. 38A-38F), as well as for the nonsocial movies (FIG. 38E-38G), except for Mechanical Puppy. Notably, the effect sizes comparing the two groups were smaller for the nonsocial movies compared with the effect sizes for the social movies.

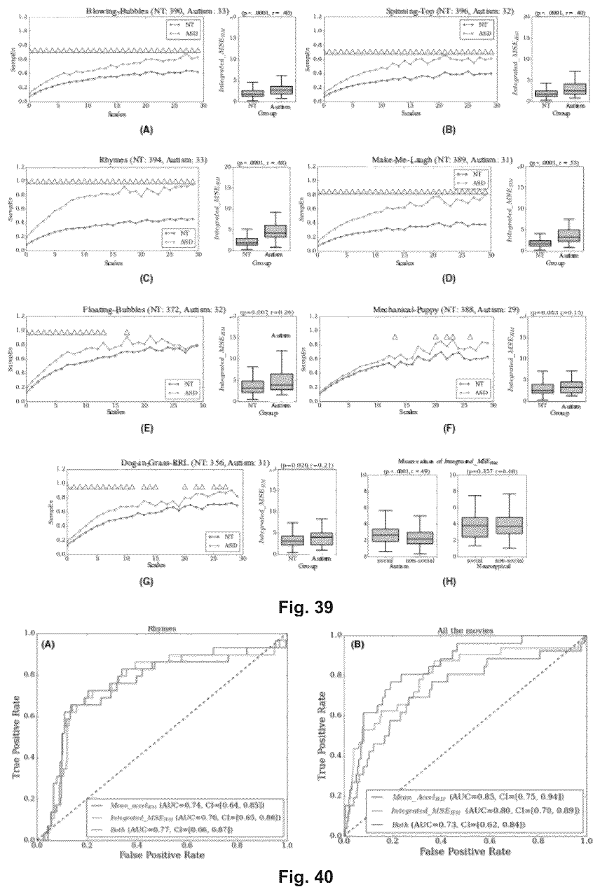

Complexity (entropy). FIG. 39A-39G displays the MSE of the head movements across 1-30 scales. With 2×2 mixed ANOVA a significant effect of group was found indicating a greater level of complexity for the autistic compared to the neurotypical group (F(1, 427)=29.68, p<0.0001). A significant effect of movie type was also found indicating greater complexity of movement during social compared to nonsocial movies (F(1, 427)=42.94, p<0.0001), but there was not a significant interaction effect (F (1, 427)=1.68, p=0.06). Analyzed by type of movie separately, the SampEn was significantly higher for the autistic group compared to the neurotypical group during social movies, especially over the first 10 scales, with effect size being small to medium (ranging from 0.25 to 0.48; p-values are provided per scale in FIG. 39A-39G). The Integrated_entropyHM during social movies (FIG. 39A-39D) was also significantly higher for the autistic group, with effect sizes ranging from medium (r=0.4) to large (r=0.68). Similar results were found for the nonsocial movies (FIG. 39E-39G), with significant group differences for the SampEn at some of the resolutions, but the effect sizes were smaller (0.15-0.2). The Integrated_entropyHM was also significantly different between the two groups during the nonsocial movies (with small to large effect sizes; see FIG. 39), except Mechanical Puppy.

Within-qroup differences. Further, we have analyzed the differences within each of the autistic and neurotypical groups for Mean_rateHM, Mean_accelHM, and Integrated_entropyHM in response to the social and nonsocial movies. The Wilcoxon signed rank test indicated that the neurotypical group exhibited significantly higher Mean_rateHM (p<0.0001, r=0.47), Mean_accelHM (p<0.0001, r=0.62; FIG. 38G), and Integrated_entropyHM (p<0.0001, r=0.51; FIG. 39G) during social movies than the nonsocial movies. In contrast, the autistic group did not exhibit differences in Mean_rateHM, Mean_accelHM, and Integrated_entropyHM during the social versus nonsocial movies. Influence of varying time segments and age. We repeated our analyses using the number of time segments and the participant's age as covariates in ANCOVA and found these two covariates did not affect our between-group analyses.

Relationship between head movement variables and cognitive ability. Mullen Scale scores were available for the autistic group. There was a positive correlation between the Mullen Early Learning Composite Score and the head movement measures, Mean_rateHM, Mean_accelHM, and Integrated_entropyHM, during the social movies (r's=0.36, 0.39 and 0.34, respectively; p's<0.05) but not during the nonsocial movies (all nonsignificant).

Combining acceleration and complexity via a classification framework. The Mean_accelHM and the Integrated_entropyHM were moderately correlated (r=0.4-0.5). We used these two measures from the four social movies for classification analysis since effect sizes for group differences in head movements were larger during the social movies. We trained an SVM-based classifier using these two input features and group as the classification target to evaluate how these measures can be used to discriminate groups. We evaluated the performance using information collected during a single movie and combination of all four social movies. For the latter analysis, we included the data from participants who had data from these two features from all four movies, resulting in N=31 for the autistic group and N=389 for the neurotypical group. Testing for individual movies, Mean_accelHM and Integrated_entropyHM distinguished the autistic and neurotypical groups (see FIG. 40), both when used alone and in combination. Combining either of the two features across all the movies (resulting in a 4-dimensional feature space), the AUC of the ROC increased to 0.85 for the Mean_accelHM and 0.80 for the Integrated_entropyHM. Combining either of the features across movies performed better than combining two features extracted during each of the movies, though the classifiers are not statistically significantly different. Combining all the features for all the movies (8-dimensional space) resulted in a lower performance (0.73 AUC compared to 0.85 and 0.80 AUC achieved with individual features combined across movies). For datasets of moderate size, a decrease in performance with the increase in number of features to a certain number (datadependent) is a common phenomenon.

### Discussion of Example 5

We demonstrated that a scalable app delivered to toddlers on an Pad during a well-child visit can be used to detect early head movement differences in toddlers diagnosed with autism. Similar to our previously published findings, autistic toddlers exhibited a higher rate of head movements while watching dynamic audiovisual movies, regardless of whether the content was social or nonsocial in nature. Furthermore, we found that the autistic toddlers also showed greater acceleration and complexity of their head movements compared to neurotypical toddlers. Our findings suggest that this sensorimotor behavior, which is exhibited while watching complex, dynamic stimuli and characterized by more frequent head movements that have higher acceleration and more complexity, is an early feature of autism. Moreover, in an analysis combining measures of head movement acceleration and complexity for each movie and across all movies with social content, we demonstrated that an SVM-based classifier based on head movement dynamics differentiated the autistic and neurotypical groups in a data-driven fashion.

The nature of these differences in head movement dynamics is not fully understood. Such differences do not appear to reflect the degree of attention to the movies, as the measures were only taken during the time frames when children were attending to the movies (facing forward and gazing towards the screen). Moreover, including the amount of attention to the movies as a covariate in our analyses did not affect our results. Similarly, the head movements do not appear to reflect degree of social engagement because the autistic children also showed differences in head movement dynamics while watching the nonsocial movies. Martin and colleagues found that autistic children exhibited higher levels of head movements only while viewing social stimuli and interpreted the movements as a mechanism for modulating their perception of the social stimuli (Martin et al., 2018). In contrast, we found that, whereas the neurotypical toddlers showed increased head movements during social as compared to nonsocial movies, the autistic toddlers nevertheless showed high levels of head movements during both types of movies. Thus, our data do not support the hypothesis that the head movements of the autistic toddlers were used to modulate the perception of social stimuli, per se. It is still possible, however, that the movements were more generally used to modulate sensory information across the different types of movies. Interestingly, autistic children with lower cognitive abilities showed higher levels of head movement rate, acceleration, and complexity specifically during viewing of the social movies. It is possible that children with lower cognitive abilities found the social movies more difficult to interpret, as these movies did involve the use of simple speech, facial expressions, and gestures by the actor.

Another possibility is that the head movements reflect differences in postural control. Previous studies of postural sway in autistic individuals have found that postural control difficulties increase when sensory demands are increased, such as when viewing stimuli requiring multisensory integration (Cham et al., 2021; Minshew et al., 2004). Examining the videos of toddlers with high levels of head movements in the present study revealed that the movement involves not just the head but also the upper body including trunk and shoulders, which were not captured by our computer vision algorithm, as we focused solely on the face in this study. Maintaining stability of posture and midline head control relies on complex sensorimotor processes which are challenged when viewing complex multisensory information. Difficulties in multisensory integration have been documented in autistic individuals (Donohue et al., 2012). Like some forms of repetitive behavior, head movements might also serve a regulatory function, especially if children found the stimuli arousing, similar to findings in studies of postural control (Cham et al., 2021).

Future research is needed to further explore the developmental course of differences in head movement dynamics in autism and elucidate their nature and neurobiological basis. Limitations of this study include the sample size, which was relatively large but included a smaller number of autistic children and did not offer sufficient power to determine the influence of biological and demographic characteristics, such as sex. Data from all participants were not used in some of the analyses because we used data from participants who attended for at least 40% of the movie length. The autistic and neurotypical group differed in cognitive ability and, thus, it is not clear the degree to which differences in cognition contributed to our findings. In summary, results of this study confirm that a difference in head movement dynamics is one of the early sensorimotor signs associated with autism. Combining this feature with other behavioral biomarkers such as gaze, facial dynamics, and response to name will allow us to develop a multimodal computer vision-based digital phenotyping tool capable of offering a quantitative and objective characterization of early behaviors associated with autism.

### Representative Points

Autistic children exhibit more frequent head movements while watching dynamic stimuli compared to neurotypical children.

Earlier research suggested that computer vision can automatically measure these head movement patterns.

This larger study confirmed that computer vision can be used to objectively and automatically measure head movement dynamics in toddlers from videos recorded via a digital app during a well-child checkup in primary care.

Rate, acceleration, and complexity of head movements were found to be significantly higher in autistic toddlers compared to neurotypical toddlers.

Combining head movements with other behavioral biomarkers, a multimodal computer vision and machine learning-based digital autism screening tool can be developed, offering quantitative and objective characterization of early autism-related behaviors.

### REFERENCES FOR EXAMPLE 5

The disclosure of each of the following references is incorporated herein by reference in its entirety to the extent that it is not inconsistent herewith and to the extent that it supplements, explains, provides a background for, or teaches methods, techniques, and/or systems employed herein. The numbers below correspond to the superscripted numbers in EXAMPLE 5.
American Psychiatric Association. (2014). Diagnostic and statistical manual of mental disorders: DSM-5. Washington, DC: Author.
Baltrusaitis, T., Zadeh, A., Lim, Y. C., & Morency, L. P. (2018). OpenFace 2.0: Facial behavior analysis toolkit. In Proceedings—13th IEEE International conference on automatic face and gesture recognition, FG 2018 (pp. 59-66).
Baranek, G. T. (1999). Autism during infancy: A retrospective video analysis of sensory-motor and social behaviors at 9-12 months of age. Journal of Autism and Developmental Disorders, 29, 213-224.
Bhat, A. N. (2021). Motor impairment increases in children with autism spectrum disorder as a function of social communication, cognitive and functional impairment, repetitive behavior severity, and comorbid diagnoses: A SPARK study report. Autism Research, 14, 202-219.
Cham, R., Iverson, J. M., Bailes, A. H., Jennings, J. R., Eack, S. M., & Redfern, M. S. (2021). Attention and sensory integration for postural control in young adults with autism spectrum disorders. Experimental Brain Research, 239, 1417-1426.
Chang, C. H., Wade, M. G., Stoffregen, T. A., Hsu, C. Y., & Pan, C. Y. (2010). Visual tasks and postural sway in children with and without autism spectrum disorders. Research in Developmental Disabilities, 31, 1536-1542.
Chang, Z., Di Martino, J. M., Aiello, R., Baker, J., Carpenter, K., Compton, S., . . . & Sapiro, G. (2021). Computational methods to measure patterns of gaze in toddlers with autism Spectrum disorder. JAMA Pediatrics, 175, 827-836.
Cirugeda-Roldan, E., Cuesta-Frau, D., Miro-Martinez, P., & Oltra-Crespo, S. (2014). Comparative study of entropy sensitivity to missing biosignal data. Entropy, 16, 5901-5918.
Cortes, C., & Vapnik, V. (1995). Support-vector networks. Machine Learning, 20, 273-297.
Costa, M., Goldberger, A. L., & Peng, C. K. (2002). Multiscale entropy analysis of complex physiologic time series. Physical Review Letters, 89, 68102.
Costa, M., Goldberger, A. L., & Peng, C. K. (2005). Multiscale entropy analysis of biological signals. Physical Review E-Statistical, Nonlinear, and Soft Matter Physics, 71, 21906.
Dawson, G., Campbell, K., Hashemi, J., Lippmann, S. J., Smith, V., Carpenter, K., . . . & Sapiro, G. (2018). Atypical postural control can be detected via computer vision analysis in toddlers with autism spectrum disorder. Scientific Reports, 8, 1-7.
Dong, X., Chen, C., Geng, Q., Cao, Z., Chen, X., Lin, J., . . . & Zhang, X. D. (2019). An improved method of handling missing values in the analysis of sample entropy for continuous monitoring of physiological signals. Entropy, 21, 274.
Donohue, S. E., Darling, E. F., & Mitroff, S. R. (2012). Links between multisensory processing and autism. Experimental Brain Research, 222, 377-387.
Elisseeff, A., & Pontil, M. (2003). Leave-one-out error and stability of learning algorithms with applications. Advances in Learning Theory: Methods, Models and Applications, NATO Science Series III: Computer & Systems Sciences, 190, 111-130.
Esposito, G., & Venuti, P. (2009). Symmetry in infancy: Analysis of motor development in autism spectrum disorders. Symmetry, 1, 215-225.
Flanagan, J. E., Landa, R., Bhat, A., & Bauman, M. (2012). Head lag in infants at risk for autism: A preliminary study. American Journal of Occupational Therapy, 66, 577-585.
Fournier, K. A., Hass, C. J., Naik, S. K., Lodha, N., & Cauraugh, J. H. (2010). Motor coordination in autism spectrum disorders: A synthesis and meta-analysis. Journal of Autism and Developmental Disorders, 40, 1227-1240.
Hanley, J. A., & McNeil, B. J. (1982). The meaning and use of the area under a receiver operating characteristic (ROC) curve. Radiology, 143, 29-36.
Harati, S., Crowell, A., Mayberg, H., Kong, J., & Nemati, S. (2016). Discriminating clinical phases of recovery from major depressive disorder using the dynamics of facial expression. In Proceedings of the Annual International conference of the IEEE Engineering in Medicine and Biology Society, EMBS, 2016-October (pp. 2254-2257).
Hashemi, J., Dawson, G., Carpenter, K. L. H., Campbell, K., Qiu, Q., Espinosa, S., . . . & Sapiro, G. (2021). Computer vision analysis for quantification of autism risk behaviors. IEEE Transactions on Affective Computing, 12, 215-226.
King, D. E. (2009). Dlib-ml: A machine learning toolkit. Journal of Machine Learning Research, 10, 1755-1758.
Krishnappababu, P. R., Di Martino, M., Chang, Z., Perochon, S. P., Carpenter, K. L. H., Compton, S., . . . & Sapiro, G. (2021). Exploring complexity of facial dynamics in autism Spectrum disorder. IEEE Transactions on Affective Computing, 1-12. https://doi.orq/10.1109/taffc.2021.3113876 Lake, D. E., Richman, J. S., Pamela Griffin, M., & Randall Moorman, J. (2002). Sample entropy analysis of neonatal heart rate variability. American Journal of Physiology—Regulatory Integrative and Comparative Physiology, 283, R789-R797.
Li, Y., Fathi, A., & Rehg, J. M. (2013). Learning to predict gaze in egocentric video. In Proceedings of the IEEE International conference on computer vision (pp. 3216-3223).
Luyster, R., Gotham, K., Guthrie, W., Coffing, M., Petrak, R., Pierce, K., . . . & Lord, C. (2009). The autism diagnostic observation schedule—toddler module: A new module of a standardized diagnostic measure for autism spectrum disorders. Journal of Autism and Developmental Disorders, 39, 1305-1320.
Mannan, S., Ruddock, K. H., & Wooding, D. S. (1995). Automatic control of saccadic eye movements made in visual inspection of briefly presented 2-D images. Spatial Vision, 9, 363-386.
Martin, K. B., Hammal, Z., Ren, G., Cohn, J. F., Cassell, J., Ogihara, M., . . . & Messinger, D. S. (2018). Objective measurement of head movement differences in children with and without autism spectrum disorder. Molecular Autism, 9, 1-10.
Melo, C., Ruano, L., Jorge, J., Pinto Ribeiro, T., Oliveira, G., Azevedo, L., & Temudo, T. (2020). Prevalence and determinants of motor stereotypies in autism spectrum disorder: A systematic review and meta-analysis. Autism, 24, 569-590.
Minshew, N. J., Sung, K. B., Jones, B. L., & Furman, J. M. (2004). Underdevelopment of the postural control system in autism. Neurology, 63, 2056-2061.
Molloy, C. A., Dietrich, K. N., &Bhattacharya, A. (2003). Postural stability in children with autism Spectrum disorder. Journal of Autism and Developmental Disorders, 33, 643-652.
Mullen, E. M. (1995). Mullen scales of early learning (pp. 58-64). Circle Pines, MN: American Guidance Service.
Perochon, S., Di Martino, M., Aiello, R., Baker, J., Carpenter, K., Chang, Z., . . . & Dawson, G. (2021). A scalable computational approach to assessing response to name in toddlers with autism. Journal of Child Psychology and Psychiatry, 62, 1120-1131.
Richman, J. S., & Moorman, J. R. (2000). Physiological timeseries analysis using approximate and sample entropy. American Journal of Physiology—Heart and Circulatory Physiology, 278, H2039-H2049.
Robins, D. L., Casagrande, K., Barton, M., Chen, C. M. A., Dumont-Mathieu, T., & Fein, D. (2014). Validation of the modified checklist for autism in toddlers, revised with follow-up (M-CHAT-R/F). Pediatrics, 133, 37-45.
Tan, P.-N., Steinbach, M., & Kumar, V. (2006). Classification: Basic concepts, decision trees, an model evaluation. Introduction to Data Mining, 1, 145-205.
Teitelbaum, P., Teitelbaum, O., Nye, J., Fryman, J., & Maurer, R. G. (1998). Movement analysis in infancy may be useful for early diagnosis of autism. Proceedings of the National Academy of Sciences of the United States of America, 95, 13982-13987.
Vabalas, A., Gowen, E., Poliakoff, E., & Casson, A. J. (2019). Machine learning algorithm validation with a limited sample size. PLoS One, 14, e0224365.
Wilson, R. B., Enticott, P. G., & Rinehart, N. J. (2018). Motor development and delay: Advances in assessment of motor skills in autism spectrum disorders. Current Opinion in Neurology, 31, 134-139.
Wilson, R. B., McCracken, J. T., Rinehart, N. J., & Jeste, S. S. (2018). What's missing in autism spectrum disorder motor assessments?Journal of Neurodevelopmental Disorders, 10, 1-13.
Zhao, Z., Zhu, Z., Zhang, X., Tang, H., Xing, J., Hu, X., . . . & Qu, X. (2021). Atypical head movement during face-to-face interaction in children with autism spectrum disorder. Autism Research, 14, 1197-1208.

### Example 6

### Blink Rate and Facial Orientation Reveal Distinctive Patterns of Attentional Engagement in Autistic Toddlers: A Digital Phenotyping Approach

Differences in social attention are well-documented in autistic individuals, representing one of the earliest signs of autism. Spontaneous blink rate has been used to index attentional engagement, with lower blink rates reflecting increased engagement. We evaluated novel methods using computer vision analysis (CVA) for automatically quantifying patterns of attentional engagement in young autistic children, based on facial orientation and blink rate, which were captured via mobile devices. Participants were 474 children (17-36 months old), 43 of whom were diagnosed with autism. Movies containing social or nonsocial content were presented via an iPad app, and simultaneously, the device's camera recorded the children's behavior while they watched the movies. CVA was used to extract the duration of time the child oriented towards the screen and their blink rate as indices of attentional engagement. Overall, autistic children spent less time facing the screen and had a higher mean blink rate compared to neurotypical children. Neurotypical children faced the screen more often and blinked at a lower rate during the social movies compared to the nonsocial movies. In contrast, autistic children faced the screen less often during social movies than during nonsocial movies and showed no differential blink rate to social versus nonsocial movies.

A large body of literature has utilized eye tracking to document differences in gaze patterns to social versus nonsocial stimuli in autistic individuals across the lifespan¹⁻³. While the majority of studies of attention in autism have focused on gaze patterns, spontaneous eye blink rate has also been used to assess attention⁴. Studies have demonstrated task-related modulation of blink rate, with rate of blinking inversely related to level of encoding of information in working memory and attentional engagement⁵-7. The evolutionary basis of varying blink rate stems from the idea that real-time assessments of the salience and value of information unconsciously change blink rate to increase or decrease the amount of visual information that is processed⁸. Evidence suggests a connection between spontaneous blink rate and striatal dopamine activity, with decreased blink rate found in persons with Parkinson's disease, attention-deficit/hyperactivity disorder (ADHD), and fragile X syndrome⁹⁻¹¹. Hornung et al.¹² found that, compared to neurotypical children, blink rate and theta spectral EEG power, another measure of attentional engagement, were both reduced in autistic children. Another study using eye tracking found that neurotypical children exhibited lower blinking when watching scenes with high affective content, whereas autistic children blinked less frequently when looking at physical objects¹³. These results are consistent with findings that autism is associated with reduced social attention’, which is evident as early as 2-6 months of age^{14,15}.

Traditionally, eye tracking has been used to measure gaze and blink rate patterns. We explored whether it was possible to detect meaningful patterns of attention via blink rate in toddlers using computer vision analysis (CVA) based on data collected via an application (app) on a smart tablet without the use of additional equipment. In a previous study, we demonstrated that it was possible to reliably measure atypical patterns of gaze, characterized by reduced attention to social stimuli, via CVA in young autistic toddlers compared to their neurotypical peers¹⁶.

The current analysis extends previous work by studying blink rate as an additional method for capturing patterns of attentional engagement in toddlers while they watched a series of strategically-designed social and nonsocial movies on a smart tablet. Along with blink rate, we also estimated the duration of the child orienting towards the tablet's screen, denoted as total time facing forward (TFF). We predicted that neurotypical toddlers would reduce their blinking and thus exhibit lower blink rate when viewing movies with high social content, as compared to those without social content. In contrast, we predicted that autistic toddlers would either fail to exhibit a differential blink rate to movies with social versus nonsocial content or show lower blink rates when viewing movies with nonsocial content, suggesting higher attentional engagement when viewing nonsocial stimuli.

### Results

Effects of group and stimulus type on facing forward and blink rate variables. To estimate the main effects of group and stimulus type (social versus nonsocial movies) and their interaction effects for total time facing forward (TFF) and blink rate, a 2×2 mixed ANOVA was conducted. This analysis was based on the movies that had primarily social or nonsocial content (refer to the “Methods and materials” section along with FIG. 1 for details of the movies presented in the app). Mean TFF and mean blink rate were estimated for both the social and nonsocial movies. “Blowing Bubbles” and “Spinning Top” were excluded during this analysis since they contain both social and nonsocial content (see FIG. 1). FIG. 1 depicts the mean with 5th and 95^{th} percentile of the time-series associated with the ‘facing forward’ variable per one second window (see “Methods and materials” for details on the computation of ‘facing forward’). The distributions associated with the neurotypical/autistic groups are shown in blue/orange. Moments of presentation of social and nonsocial movies are highlighted with blue and green (respectively) semitransparent boxes.

A main effect of group was found for mean TFF (F (1, 440)=40.76, P<0.0001, ηp 2=0.086) and mean blink rate (F (1, 440)=17.63, P<0.0001, ηp 2=0.04). On average, autistic children had lower mean TFF and higher mean blink rate compared to neurotypical children. A main effect of stimulus type was also found for TFF (F (1, 440)=98.17, P<0.0001, ηp 2=0.18) and blink rate (F (1, 440)=54.30, P<0.0001, ηp 2=0.12), indicating that, on average, participants exhibited higher TFF and lower blink rate during the social movies compared to nonsocial ones.

Interaction effects between group and stimulus type were found for both mean TFF (F (1, 440)=28.27, P<0.0001, ηp 2=0.06) and mean blink rate (F (1, 440)=7.78, P=0.005, ηp 2=0.02). Comparisons of the mean TFF and blink rate values within the neurotypical and autistic groups during social versus nonsocial movies are shown in FIG. 2. Within-group statistical analysis using Wilcoxon signed-rank test was performed for each of the two groups while comparing the social versus nonsocial movies. The results indicate that the neurotypical children exhibited significantly higher mean TFF (P<0.0001, r=0.68; FIG. 2a) and lower mean blink rate (P<0.0001, r=0.55; FIG. 2b), both with large effect sizes, during social movies compared to nonsocial. This potentially indicates higher levels of attentional engagement during the social than the nonsocial movies in the neurotypical group. In contrast, the autistic group had lower mean TFF (P=0.043, r=0.33; FIG. 2a) during social compared to nonsocial movies with medium effect size and showed no difference in mean blink rate for social versus nonsocial movies (P=0.21, r=0.17; FIG. 2b). Examining the differences between the groups using the Mann-Whitney U test for movies of a specific type (social or nonsocial), on average, the neurotypical children exhibited higher mean TFF during the social movies than autistic children (P<0.0001, r=0.61; FIG. 2), whereas the two groups did not differ in their mean TFF during the nonsocial movies (P=0.1, r=0.12; FIG. 2) (see also FIG. 1 for line plot of ‘facing forward’ during the task progression). In terms of the mean blink rate, the autistic group exhibited significantly higher mean blink rate than the neurotypical group both during social (P<0.001, r=0.60; FIG. 2) and nonsocial (P=0.011, r=0.25; FIG. 2) movies.

To ensure that the overall group difference in TFF was not driving results, we repeated these analyses using only the participants having TFF>0.80 and found that the pattern of results remained consistent along with statistical significance (see supplementary materials FIGS. S1 and S2 for more details and statistics). The number of participants with TFF>0.80 for the mean TFF and mean blink rate of the social and nonsocial movies are: autistic group (N=20) and neurotypical group (N=394). The numbers of participants for each individual movie are presented in Figure S2 of the supplementary material. Furthermore, to test whether the participant's age had any effect on the measures, ANCOVA was conducted using ‘age’ as covariate. The pattern of results remained consistent after including the covariate.

It is possible that the autistic children were facing forward less during the social movies because, on average, the social movies were longer and tended to come toward the end of the app administration, as compared to the nonsocial movies. To address this, group differences in TFF were also examined separately for each individual movie (FIG. 3). For each social movie, even those that were shorter and presented earlier in the sequence rather than toward the end (e.g., “Rhymes”), the difference in TFF between the two groups was significantly different with medium to large effect size (P-values and the effect size are presented in FIG. 3), with the autistic group having a reduced TFF. For each nonsocial movie, except for “Toys,” there were no significant differences between the two groups. Thus, even for the nonsocial movie that was of comparable length to the social movies (“Dog in the Grass”=56 s), the groups did not differ. Additionally, while considering “Toys,” a nonsocial movie, which was presented right after the “Rhymes,” a social movie, the autistic group exhibited a large increase in their ‘facing forward’ (FIG. 1) towards “Toys,” potentially indicating increased attention to dynamic toys, which was not seen for the neurotypical group since they were already ‘facing forward’ during the social movie, “Rhymes”. Group differences in blink rate were also examined separately for each individual movie (FIG. 3). During each of the social movies, the blink rate was significantly different between the two groups with medium effect size (P-values and the effect sizes are presented in FIG. 3); the neurotypical group exhibited lower blink rate than the autistic group during the social movies. For the nonsocial movies, the autistic group showed significantly higher blink rates than the neurotypical for “Floating Bubbles” (medium effect size) and “Toys” (small effect size), but no significant differences were observed during “Dog in Grass Right-Right-Left (RRL)” and “Mechanical Puppy”.

In addition to the estimation of the blink rate (see “Methods and materials”), in the supplementary material we present the (i) valid number of frames (Table S1) and (ii) raw blinks quantity without normalizing with respect to the valid number of frames (Table S2) for both the groups. The blink rate is a normalized representation of the ratio of raw blink quantity and valid number of frames for each participant during a movie since we wanted to have an estimate of blinking only when the participants are ‘facing forward’ towards the movie. However, to ensure that the valid number of frames are not inflating the blink rate, we present a similar statistical analysis for the valid number of frames and the raw blinks quantity (see Tables S1 and S2). The statistically significant differences between the two groups remained the same for the raw blinks quantity. Furthermore, we observed only a moderate correlation (Pearson correlation coefficient, r=−0.45) between the mean TFF and mean blink rate. This level of correlation indicates that the TFF and blink rate are two different measures that are complementing each other to quantify the participant's engagement towards the movies.

Distinguishing groups based on three CVA-based attention measures. We next examined how well the attention measures, mean TFF and mean blink rate, along with mean gaze percent social (MGPS; social attention variable) distinguished the two groups using a classification tool. MGPS was based on the percentage of time the child gazed at the social elements during “Blowing Bubbles” and “Spinning Top” which displayed both social and nonsocial elements separately either on the right or left side of the screen (see “Methods and materials” for details about the movies, and FIG. 1). The MGPS variable was available from a previously published analysis¹⁶.

We have included the MGPS for classification analysis because we excluded the movies “Blowing Bubbles” and “Spinning Top” in the estimation of mean TFF and mean blink rate. Since MGPS gives an estimate of the child's percentage of look duration towards the social part (left/right) of the screen, we explored its importance in complementing the mean TFF and mean blink rate for classification.

We considered mean values during social movies (mean TFFsocial and mean blink ratesocial) for this analysis. These two measures were moderately correlated (negative) with each other (r=−0.45), when analyzed using the Pearson correlation coefficient. The mean TFFsocial (r=0.13) was positively correlated and mean blink ratesocial (r=−0.13) was negatively correlated with MGPS. We trained the logistic regression-based classifier using these three attention features and the participant diagnostic group as the classification target to assess how these measures can potentially be used to identify behaviors linked to autism (FIG. 4). Combining the three features achieved a higher area under the curve (AUC) of the receiver operating characteristic (ROC) curve compared to when these features were used individually, indicating that these features complement each other. The confidence intervals of the ROC curves indicate there was an overlap between the individual features and their combination, though the combination still achieved a higher performance.

Relationship between attention variables and clinical characteristics. For the autistic group, we examined the relationship between the mean TFF and blink rate during the social and nonsocial movies and several clinical variables, including Mullen Early Learning Composite Score and Visual Reception Score, and Autism Diagnostic Observation Schedule (ADOS) Calibrated Severity Scores (ADOS CSS total, restricted/repetitive behavior, social affect). As shown in Table 1, total time facing forward during the social movies was negatively correlated with ADOS total and social affect scores. Autistic children with higher total and social affect ADOS CSS spent less time facing forward during the social movies. Mean total time facing forward (TFF) during the nonsocial, but not the social, movies was negatively correlated with cognitive abilities (Mullen Early Learning Composite Score and Visual Reception Score). Children with higher cognitive abilities spent less time facing forward during the nonsocial movies. We did not find any relationships between the mean blink rate and the clinical variables (Table 1).

### Discussion of Example 6

Research has consistently documented differences in attentional patterns in autistic individuals, characterized by reduced visual social engagement¹. Such differences are apparent during infancy and offer a means of detecting early signs of autism^{14,15,17}.

Thus, developing scalable, objective, and quantitative methods for measuring patterns of attentional engagement in infants and toddlers is an important goal. We have previously shown that CVA can be used to detect distinct patterns of gaze in autistic toddlers, characterized by reduced social attentional engagement, using relatively low-cost, scalable devices without any special set-up, equipment, or calibration¹⁶.

In the present study, we extend this work by demonstrating that using the same app shown on a tablet, we can use CVA to capture distinctive patterns of attentional engagement to social and nonsocial stimuli in autistic toddlers, based on facial orientation and blink rate. This offers an additional quantitative, objective approach to assessing early attention in toddlers. Overall, autistic toddlers spent less time with their face oriented forward to the movies and exhibited higher blink rates compared to neurotypical toddlers. Our finding of reduced attentional engagement, regardless of stimulus type, is consistent with past work¹⁸, performed with consumer-grade eye-tracking tools, indicating that reduced visual engagement in autistic toddlers is not limited to social stimuli, but also extends to nonsocial stimuli. This finding is also consistent with eye tracking studies that reported that autistic toddlers exhibit lower overall sustained attention to any dynamic stimuli¹⁹.

A recent review of studies using functional brain imaging to assess social and nonsocial reward processing in autistic individuals suggested that autism is associated with general differences in reward anticipation that are not specific to social stimuli²⁰. Considering previous findings linking blink rate to reward circuitry mediated by dopaminergic activity^{11,12}, it is possible that differences in blink rate in autistic children found in the present study are associated with alterations in brain circuitry related to reward anticipation while watching the movies.

Clinical measures. Mullen scales of early learning ADOS calibrated severity score Early learning composite score Visual reception Restricted repetitive behavior Social affect Total Mean total facing forward

TABLE 1
Relationships between attention variables and
clinical characteristics for autistic group.

Social − 0.23 0.08 − 0.16 − 0.57* − 0.5*
Nonsocial − 0.38* − 0.37* 0.03 − 0.01 − 0.01
Mean blink rate
Social − 0.14 − 0.09 0.11 0.27 0.26
Nonsocial 0.1 0.11 0.06 − 0.04 0.01
* P < 0.05, the r values are based on Pearson correlation coefficient.

In addition to overall differences in attentional engagement, autistic and neurotypical toddlers displayed distinctive patterns of attentional engagement when viewing social compared to the nonsocial movies. These results align with previous findings indicating that toddlers later diagnosed with autism tend to exhibit reduced attention to social scenes in free-viewing eye tracking tasks¹⁴, evident as early as 6 months of age²¹.

Neurotypical children faced the screen more often and blinked at a lower rate during social than nonsocial movies, with large effect sizes, suggesting that the social stimuli had higher salience. In contrast, autistic children faced the screen less often during social than nonsocial movies and did not exhibit a differential blink rate to social versus nonsocial movies. This is consistent with a previous study of blink rate which found reduced blink rate in neurotypical children during viewing of social stimuli, possibly due to their increased engagement with the stimuli¹³.

Group comparisons showed that, on average, the neurotypical children faced toward the screen more often during the social movies than autistic children, whereas the two groups did not differ in their tendency to face toward the screen during the nonsocial movies. The combination of three different measures of attentional engagement (facing the screen and blink rate during social movies and percent time gazing at social stimuli) distinguished between autistic and neurotypical children with an AUC=0.82.

Limitations of this study include the sample size, which despite being relatively large, did not offer sufficient power to determine the influence of sex and other demographic characteristics, such as race and ethnicity. Future studies are planned to assess the generalizability of these findings to diverse populations. Such studies are particularly important in light of previous findings linking differences in gaze patterns to face stimuli of same—versus different-race^{22,23}. Moreover, future studies will be needed to examine the specificity of the findings to autism by directly comparing blink rate and facial orientation during viewing of social and nonsocial stimuli in autistic children to that of children with other neurodevelopmental disorders, such as ADHD and language or developmental delay.

By combining these novel indices of attention with other digital phenotypic features, such as facial dynamics^{24,25}, orienting²⁶, and head movements^{27,28}, in the future, it may be possible to develop a scalable robust phenotyping tool to detect autism in toddlers, as well as monitor longitudinal development and response to early intervention.

### Methods and Materials for Example 6

Participants. Participants were 474 toddler age children recruited during their well-child checkup at four pediatric primary care clinics. Based on DSM-5 criteria, 43 toddlers were subsequently diagnosed with autism spectrum disorder. Further, 15 toddlers were diagnosed with language delay/developmental delay, and the remaining 416 participants were neurotypical (NT). Inclusion criteria were: (i) age 16-38 months and (ii) caregiver's primary language was English or Spanish. Exclusion criteria were: (i) hearing or vision impairments; (ii) the child was too upset or ill during the visit; (iii) the caregiver expressed they had no interest or did not have enough time; (iv) the child would not stay in their caregiver's lap, or the app or device failed to upload data, or the clinical information was missing; and (v) presence of a significant sensory or motor impairment that precluded the child from watching the movies and/or sitting upright.

Ethical considerations. The study protocols were reviewed and approved by the Duke University Health System Institutional Review Board (Pro00085434, Pro00085435). All the methods used in this study were performed in accordance with all relevant guidelines and regulations. Informed consent was obtained from all participants' parents or their legal guardians. Informed consent was obtained from actors shown in FIG. 1 to publish identifying information/images in an online open-access publication.

Clinical measures. Modified checklist for autism in toddlers: revised with follow-up (M-CHAT-R/F). A commonly used screening questionnaire, M-CHAT-R/F²⁹ was administered to all the participants. The caregiver completed M-CHAT-R/F (20 questions) was used to evaluate the presence/absence of autism-related symptoms.

Diagnostic and cognitive assessments. Participants whose M-CHAT-R/F score was ≥3 initially or had a total score 2 after the follow-up questions, or whose pediatrician or caregiver expressed developmental concerns, were referred for diagnostic evaluation. The Autism Diagnostic Observation Schedule-Toddler Module (ADOS-2) was administered by a research-reliable licensed psychologist from the study team who determined whether the child met DSM-5 criteria for autism³⁰.

The Mullen Scales of Early Learning³¹ was used to assess the participant's cognitive and language abilities.

### Group Definitions

Autistic (N=43). This group included toddlers with an M-CHAT-R/F positive score and/or with developmental concerns raised by the pediatrician/caregiver who subsequently met DSM-5 diagnostic criteria for autism spectrum disorder with or without developmental delay based on both the ADOS-2, Mullen Scales, and clinical judgment by a research reliable psychologist.

Neurotypical (N=416). This group included toddlers having a high likelihood of typical development with an M-CHAT-R/F score ≤1 and no developmental concerns raised by the pediatrician/caregiver, or those who had a positive M-CHAT-R/F score and/or the pediatrician/caregiver raised concerns but then were determined to not have developmental or autism-related concerns by the psychologist based on the ADOS-2, cognitive testing via Mullen Scales, and clinical judgment. Table 2 shows the participants' demographic characteristics for the autistic and neurotypical groups, consisting of 459 participants. There was another group of participants (N=15) who had a positive M-CHAT-R/F score and received a diagnosis of language delay/developmental delay (LD-DD) without autism. Children included in the LD-DD group were those who had failed the M-CHAT-R/F or had provider or caregiver developmental concerns, were referred for evaluation and administered the ADOS-2 and Mullen Scales and were then determined by a licensed psychologist not to meet DSM-5 criteria for autism. All children in the LD-DD group scored ≥9 points below the mean on at least one Mullen Early Learning Subscale (1 SD=10 points). Given the small sample size, we present data for the LD-DD group only in the supplementary materials (refer to Table S1, FIGS. S3 and S4). The demographic characteristics of 474 participants, including the LD-DD participants, are presented in Table S3.

Application (app) administration and stimuli. The app was administered on a tablet (iPad) that displayed developmentally appropriate, short social and nonsocial movies during the child's well-child visit. The tablet was mounted on a tripod placed at ˜60 cm from the child while the caregiver was holding the child on their lap. Any other family members (e.g., siblings) and the research staff who administered the app stayed behind both the caregiver and the child. The tablet's frontal camera recorded the video of the child at 30 fps which was further used for CVA to automatically capture their behavioral responses. The social and nonsocial movies were presented in the same order for all participants, as described next. The total duration of the movies was about 8 min. All movies contained both visual and auditory stimuli, described below. In both the social and nonsocial movies, visual and auditory stimuli were sometimes synchronized (e.g., “Dog in the Grass” and “Rhymes”) and sometimes non-synchronized (e.g., “Floating Bubbles and “Make Me Laugh”). Nonsocial movies contained dynamic objects with sound, unlike the social movies that had higher social content with ethnically and racially diverse human actors in the scenes. All the social movies depicted human actors. The language used by the actors was provided in English or Spanish depending on the child's primary language at home. FIG. 1 shows a snapshot of the movies.

TABLE 2
Demographic characteristics for neurotypical and autistic groups

Groups
N (%)
Neurotypical (N = 416; 90.63%) Autistic (N = 43; 9.37%)
Age in months
Mean (SD) 20.59 (3.18)a 24.32 (4.64)a
Sex
Boy 209 (50.24%)b 32 (74.42%)b
Girl 207 (49.76%)b 11 (25.58%)b
Race
American Indian/Alaskan Native 1 (0.24%) 3 (6.97%)
Asian 6 (1.44%) 1 (2.32%)
Black or African American 43 (10.33%) 6 (13.95%)
Native Hawaiian or Other Pacific Islander 0 (0.00%) 0 (0.00%)
White/Caucasian 316 (75.96%) 22 (51.16%)
More than one race 41 (9.85%) 7 (16.28%)
Other 9 (2.16%) 4 (9.30%)
Ethnicity
Hispanic/Latino 31 (7.45%)b 13 (30.23%)b
Not Hispanic/Latino 385 (92.54%)b 30 (69.77%)b
Caregiver's highest level of education
Without high school diploma 2 (0.49%)b 4 (9.30%)b
High school diploma or equivalent 14 (3.36%)b 6 (13.95%)b
Some college education 40 (9.61%)b 10 (23.25%)b
4-year college degree or more 356 (85.57%)b 23 (53.48%)b
Unknown/not reported 4 (0.96%) 0 (0.00%)
Clinical variables Mean (SD)
ADOS-2 Toddler Module
Calibrated Severity Score - 7.60 (1.67)
Mullen Scales of Early Learning
Early Learning Composite Score - 63.15 (9.94)
Expressive Language T-score - 28.02 (7.25)
Receptive Language T-score - 22.90 (4.81)
Fine Motor T-score - 33.97 (10.40)
Visual Reception T-score - 33.22 (10.67)

Demographic characteristics for neurotypical and autistic groups. The age (in months) at which participants received their diagnosis (ADOS-2): M=23.9, SD=4.5. The interval (in months) between the age at diagnosis and the app administration: M=0.7, SD=1.2. ADOS-2 Autism Diagnostic Observation Schedule-Second Edition. a Significant difference between the two groups based on ANOVA test. b Significant difference between the two groups based on Chi-Square test.
(1) Floating Bubbles (35 s; nonsocial). Bubbles move randomly throughout the frame of the screen with a gurgling sound.
(2) Dog in Grass (16 s; nonsocial). In the first part of this movie, a cartoon barking puppy appears at the center and the four corners of the screen.
(3) Dog in Grass Right-Right-Left (RRL) (40 s; nonsocial). In the second part of this movie, the barking puppy appears randomly in the right/left side of the screen at first, followed by a constant right-right-left (RRL) pattern. Total length of Dog in Grass=56 s.
(4) Spinning Top (53 s; social). An actress plays with a spinning top with successful and unsuccessful attempts at spinning, looks towards the screen to convey eye contact, smiles, frowns, and makes a few verbal expressions in English or Spanish.
(5) Mechanical Puppy (25 s; nonsocial). A mechanical toy puppy barks, jumps, and walks towards a group of toys.
(6) Blowing Bubbles (64 s; social). An actor with a bubble wand blows bubbles with successful and unsuccessful attempts blowing, along with smiling and frowning, and looks towards the screen to convey eye contact with a few verbal expressions in English or Spanish.
(7) Rhymes (30 s; social). An actress says nursery rhymes such as Itsy-Bitsy Spider in English or Spanish with smiles and gestures.
(8) Toys (19 s; nonsocial). Dynamic toys with sound are shown.
(9) Make Me Laugh (56 s; social). An actress demonstrates silly, funny actions with smiling and eye contact.
(10) Playing with Blocks (71 s; social). Two child actors, a boy and a girl, interact and play with toys with occasional verbalizations in English or Spanish.
(11) Fun at the Park (51 s; social). Two actresses stand at each side of the frame, having a turn-taking conversation in English or Spanish with no gestures.

Estimation of ‘facing forward’ and blink rate variables. We first used CVA to determine the amount of time the child's face was oriented toward the screen of the device (‘facing forward’). A face detection algorithm³² was used to capture the child's face in each frame of the recorded video. In order to track only the participant's face and ignore all other faces in the frame, we performed a semi-supervised face detection algorithm (for details, see Refs. 16,26). Subsequently, we extracted 49 facial landmark points comprising 2D-positional coordinates³³ that were time-synchronized with the movies. Using the facial landmarks, for each frame, we computed the child's head pose angles relative to the tablet's frontal camera such as θyaw (left-right), θpitch (up-down), and θroll (tilting left-right) (as described in Ref.34).

Facing forward. A child's orientation towards the screen, i.e. ‘facing forward’ during any given frame was defined using their (i) head pose angle, (ii) eye gaze, and (iii) rapidity in head movement. The child's head pose |θyaw|<25° was used, acting as a proxy for attentional focus on the screen, consistent with our previous work^{27,34}, which is supported by the central bias theory for gaze estimation^{35,36}. Then, for each frame, we checked if the estimated gaze of the participant was on the tablet's screen and if their eyes were open. The participant's gaze information was extracted using an automatic gaze estimation algorithm based on a pre-trained deep neural network^{16,37}. Finally, we excluded the frames where the head was moving rapidly (this can lead to errors in the CVA). To this end, we first performed smoothing of the head pose signal θyaw, obtaining θyaw′. The head was considered to be moving rapidly if at any point θyaw′ of the current frame was >150% of the previous frame. Finally, the total facing forward variable (TFF) was estimated as a percentage of frames ‘facing forward’ out of the number of frames for each movie (ranging between 0 and 100). Details on the algorithm are presented in the supplementary materials, Algorithm S1.

Blink rate. We estimated the participant's number of blinks while they were watching each of the presented movies, as described next. OpenFace, a facial analysis toolkit³⁸ that offers facial action units on a frame-by-frame basis, was used. These action units are based on the standard facial action coding system³⁹. For the blinking action, we used action unit 45 (AU45) to estimate the participant's blinks. A smoothing of the AU45 time-series signal was performed, followed by detecting the number of peaks, which are associated with blink actions (see supplementary materials, Algorithm S2). To obtain the blink rate (blink rate), we normalized the number of blinks with respect to the number of valid frames. The valid frames were defined as frames during which the participant was (i) ‘facing forward’ (see above) and (ii) the confidence outcome of the OpenFace was at or above the recommended threshold (i.e. 0.75)³⁸.

Social attention variable using eye gaze estimation. The “Spinning Top” and “Blowing Bubbles” stimuli had equally spatially halved representations of social (actor/actress) and nonsocial (toys/bubbles) components on the right or left side of the screen (see FIG. 1). For these two movies, we computed the percentage the social portion across the two movies was referred to as mean gaze percent social (MGPS). Previous work by our team based on this app¹⁶ showed that autistic toddlers looked significantly less to the side of the screen that displayed the social elements compared to neurotypical toddlers.

Statistical analysis. A 2×2 mixed ANOVA was used to estimate the main effects due to (i) participant group and (ii) movie type (social and nonsocial) and their interaction effects via the Python method pinguouin. mixed_anova from Pingouin package version 0.5.2⁴⁰. The Mann-Whitney U test was used to estimate the statistical significance between the groups, using Python method pingouin.mwu. Within group comparisons were performed using the Wilcoxon signed-rank test using pingouin.wilcoxon. The statistical power was presented with effect size, ‘r’ for pingouin.mwu and pingouin.wilcoxon, and ‘ηp 2’ for ANOVA. Additionally, analysis of covariance (ANCOVA) using pingouin.ancova was performed to determine the influence of covariates. To assess the contribution of the three attention features (TFF, blink rate, and MGPS) either individually or in combination to distinguish the autistic and neurotypical groups, we used a linear logistic regression from sklearn Python package version 0.23.2⁴¹. The classification performance was compared using the area under the curve of the receiver operating characteristic considering leave-one-out cross-validation⁴². Using the Hanley and McNeil method⁴³, we have presented the 95% confidence interval (CI).

Analysis of participants who ‘faced forward’ for 80% of the movie duration. An analysis of blink rate was conducted to determine whether similar results are obtained when we only include participants who faced the screen in Figure (total facing forward, TFF) 80% of the time. As shown in Figure S1 and Figure S2, results were consistent with those in FIGS. 2 and 3, respectively. The statistically significant differences between and within the groups are presented S1 and within the table of Figure S2.

### Tables S1 and S2

### Statistics on Valid Number of Frames and Raw Blink Quantity

TABLE S1
Percentage of valid frames used for blink rate computation

| | Autistic | Neurotypical |
|---|---|---|
| Movies | Mean (SD)% | Mean (SD)% |
| Social | | |
| Spinning Top** | 77.2 (24.4)% | 95.3 (10.5)% |
| Blowing Bubbles** | 81.3 (24.4)% | 95.6 (10.9)% |
| Rhymes** | 45.8 (14.8)% | 58.1 (5.7)% |
| Make Me Laugh** | 79.8 (22.8)% | 95.6 (10.9)% |
| Playing with Blocks** | 73.6 (27.8)% | 92.1 (12.5)% |
| Fun at the Park** | 62.1 (19.7)% | 77.6 (21.7)% |
| Nonsocial | | |
| Floating Bubbles** | 68.2 (21.1)% | 71.7 (19.9)% |
| Dog in Grass RRL* | 68.1 (25.2)% | 76.6 (19.4)% |
| Mechanical Puppy | 81.3 (24.4)% | 83.1 (16.1)% |
| Toys** | 32.5 (6.1)% | 36.2 (4.7)% |

**= P < 0.0001 and
*= P < 0.5

TABLE S2
Number of raw blinks quantity without normalization
with respect to valid frames

| | Autistic | Neurotypical |
|---|---|---|
| Movies | Mean (SD)% | Mean (SD)% |
| Social | | |
| Spinning Top** | 4.9 (5.3)% | 3.2 (4.5)% |
| Blowing Bubbles** | 5.8 (4.5)% | 4.5 (5.5)% |
| Rhymes** | 3.8 (2.5)% | 2.6 (3.0)% |
| Make Me Laugh** | 6.6 (5.3)% | 5.1 (5.2)% |
| Playing with Blocks** | 6.5 (4.4)% | 4.9 (4.9)% |
| Fun at the Park** | 7.9 (5.5)% | 4.8 (3.7)% |
| Nonsocial | | |
| Floating Bubbles** | 4.5 (3.6)% | 2.7 (2.5)% |
| Dog in Grass RRL | 5.7 (3.8)% | 5.6 (4.4)% |
| Mechanical Puppy | 2.2 (2.2)% | 2.2 (2.18)% |
| Toys* | 2.6 (1.6)% | 2.3 (2.3)% |

**= P < 0.0001 and
*= P < 0.5

Table S1 presents the mean and standard deviation of the percentage of valid frames used for the blink rate computation for each of the groups. Similarly, Table S2 indicates the raw blinks quantities for both the groups. When comparing the two groups for each different movie using a statistical test (Mann-Whitney U test), both the (i) percentage of valid frames and (ii) raw blink quantity had similar statistical significance as blink rate.

Results for children with language delay/development delay (LD-DD). Table S3 shows the details of all the participants (neurotypical, autistic, and LD-DD). Figure S3 shows the mean total facing forward (TFF) and mean blink rate for the social and nonsocial stimuli. The distribution of the LD-DD group appears to have similar attentional patterns as that of neurotypical group, unlike the autistic group, indicating the potential specificity of the proposed CVA-based measures for autism. Results for the TFF and blink rate for individual movies are presented in Figure S4. The statistical results for the individual movies in Figure S4 (P-value and effect sizes) are presented by comparing the autistic and LD-DD groups only. Overall, the distribution of the LD-DD group was observed to be different from the autistic and similar to the neurotypical group.

TABLE S3
Participant demographic characteristics

| | N (%) | | |
|---|---|---|---|
| | Neurotypical | Autistic | LD-DD |
| Groups | (N = 416; 87.76%) | (N = 43; 9.07%) | (N = 15; 3.17%) |
| Age in months | | | |
| Mean (SD) | 20.59 (3.18)^{a} | 24.32 (4.64)^{a} | 22.62 (3.48) |
| Sex | | | |
| Male | 209 (50.24%)^{b} | 32 (74.42%)^{b} | 12 (74.42%)^{b} |
| Female | 207 (49.76%)^{b} | 11 (25.58%)^{b} | 3 (25.58%)^{b} |
| Race | | | |
| American Indian/Alaskan Native | 1 (0.24%) | 3 (6.97%) | 0 (0.00%) |
| Asian | 6 (1.44%) | 1 (2.32%) | 0 (0.00%) |
| Black or African American | 43 (10.33%) | 6 (13.95%) | 5 (33.33%) |
| Native Hawaiian or Other Pacific | 0 (0.00%) | 0 (0.00%) | 0 (0.00%) |
| Islander | | | |
| White/Caucasian | 316 (75.96%) | 22 (51.16%) | 7 (46.67%) |
| More Than One Race | 41 (9.85%) | 7 (16.28%) | 0 (0.00%) |
| Other | 9 (2.16%) | 4 (9.30%) | 2 (13.33%) |
| Unknown/declined | 0 (0.00%) | 0 (0.00) | 1 (6.67) |
| Ethnicity | | | |
| Hispanic/Latino | 31 (7.45%)^{b} | 13 (30.23%)^{b} | 5 (33.33%)^{b} |
| Not Hispanic/Latino | 385 (92.54%)^{b} | 30 (69.77%)^{b} | 10 (66.67%)^{b} |
| Caregivers' Highest Level of | 2 (0.49%)^{b} | 4 (9.30%)^{b} | 4 (26.67%)^{b} |
| Education | | | |
| Without High School Diploma | 14 (3.36%)^{b} | 6 (13.95%)^{b} | 5 (33.33%)^{b} |
| High School Diploma or Equivalent | 40 (9.61%)^{b} | 10 (23.25%)^{b} | 0 (0.00%)^{b} |
| Some College Education | 356 (85.57%)^{b} | 23 (53.48%)^{b} | 6 (40.00%)^{b} |
| 4-Year College Degree or More | 4 (0.96%) | 0 (0.00%) | 0 (0.00%) |
| Unknown/Not Reported | | | |
| Clinical Variables | Mean (SD) | | |
| ADOS-2 Toddler Module | | | |
| Calibrated Severity Score | — | 7.60 (1.67) | 4.06 (1.38) |
| Mullen Scales of Early Learning | | | |
| Early Learning Composite Score | — | 63.15 (9.94) | 72.53 (14.93) |
| Expressive Language T-Score | — | 28.02 (7.25) | 35.06 (10.28) |
| Receptive Language T-Score | — | 22.90 (4.81) | 31.13 (12.41) |
| Fine Motor T-Score | — | 33.97 (10.40) | 38.46 (6.18) |
| Visual Reception T-Score | — | 33.22 (10.67) | 35.80 (11.79) |

ADOS-2: Autism Diagnostic Observation Schedule - Second Edition
^{a}Significant difference between the two groups based on ANOVA test.
^{b}Significant difference between the two groups based on Chi-Square test.

### Pseudocode for Estimating the Features Via Computer Vision Analysis

Algorithm S1: Facing forward

```
CalculateFacingForward (partcipant's video, θ_{yaw}, gaze):
    for a_frame in participant's video:
      if “| θ_{yaw} | < 25°” and
        “gaze lied within the screen” and
        “θ_{yaw}′ in the current frame <150% of θ_{yaw}′ in the previous
        frame”:
          then facing_forward[a_frame] = 1
      else:
          facing_forward[a_frame] = 0
    return the array of facing_forward
CalculateTotalFacingForward (facing_forward):
  (sum of the 1's in the facing_forward / duration of the movie stimuli in
number of frames) * 100
```

Algorithm S2: Blink rate

```
CalculateBlinkRate (AU45 time series, OpenFace confidence value,
facing_forward):
  # Filter Step
    valid_frames = 0
    for a_frame in video:
      if “facing_forward[a_frame] == 1” and
        “OpenFace confidence value < .75”:
            then valid_frames += 1
        else:
            do nothing
  # Peak Detection Step
  run python scipy peak detection on filtered AU45 time series:
      return number of peaks
  blink_rate = number of peaks / valid_frames
  return blink rate
```

### REFERENCES FOR EXAMPLE 6

The disclosure of each of the following references is incorporated herein by reference in its entirety to the extent that it is not inconsistent herewith and to the extent that it supplements, explains, provides a background for, or teaches methods, techniques, and/or systems employed herein. The numbers below correspond to the superscripted numbers in EXAMPLE 6.
1. Klin, A., Shultz, S. & Jones, W. Social visual engagement in infants and toddlers with autism: Early developmental transitions and a model of pathogenesis. Neurosci. Biobehav. Rev. 50, 189-203 (2015).
2. Chita-Tegmark, M. Social attention in ASD: A review and meta-analysis of eye-tracking studies. Res. Dev. Disabil. 48, 79-93 (2016).
3. Setien-Ramos, I. et al. Eye-tracking studies in adults with autism spectrum disorder: A systematic review and meta-analysis. J. Autism Dev. Disord. https://doi. org/10. 1007/s10803-022-05524-z (2022).
4. Ortega, J., Plaska, C. R., Gomes, B. A. & Ellmore, T. M. Spontaneous eye blink rate during the working memory delay period predicts task accuracy. Front. Psychol. 13, 169 (2022).
5. Oh, J., Jeong, S. Y. & Jeong, J. The timing and temporal patterns of eye blinking are dynamically modulated by attention. Hum. Mov. Sci. 31, 1353-1365 (2012).
6. Rac-Lubashevsky, R., Slagter, H. A. & Kessler, Y. Tracking real-time changes in working memory updating and gating with the event-based eye-blink rate. Sci. Rep. 7, 1-9 (2017).
7. Ranti, C., Jones, W., Klin, A. & Shultz, S. Blink rate patterns provide a reliable measure of individual engagement with scene content. Sci. Rep. 10, 1-10 (2020).
8. Hoppe, D., Helfmann, S. & Rothkopf, C. A. Humans quickly learn to blink strategically in response to environmental task demands. Proc. Natl. Acad. Sci. U.S.A 115, 2246-2251 (2018).
9. Groen, Y., Börger, N. A., Koerts, J., Thome, J. & Tucha, O. Blink rate and blink timing in children with ADHD and the influence of stimulant medication. J. Neural Transm. 124, 27-38 (2017).
10. Reddy, V. C., Patel, S. V., Hodge, D. O. & Leavitt, J. A. Corneal sensitivity, blink rate, and corneal nerve density in progressive supranuclear palsy and Parkinson disease. Cornea 32, 631-635 (2013).
11. Roberts, J. E., Symons, F. J., Johnson, A. M., Hatton, D. D. & Boccia, M. L. Blink rate in boys with fragile X syndrome: Preliminary evidence for altered dopamine function. J. Intellect. Disabil. Res. 49, 647-656 (2005).
12. Hornung, T., Chan, W. H., Müller, R. A., Townsend, J. & Keehn, B. Dopaminergic hypo-activity and reduced theta-band power in autism spectrum disorder: A resting-state EEG study. Int. J. Psychophysiol. 146, 101-106 (2019).
13. Shultz, S., Klin, A. & Jones, W. Inhibition of eye blinking reveals subjective perceptions of stimulus salience. Proc. Natl. Acad. Sci. U.S.A 108, 21270-21275 (2011).
14. Chawarska, K., Macari, S. & Shic, F. Decreased spontaneous attention to social scenes in 6-month-old infants later diagnosed with autism spectrum disorders. Biol. Psychiatry 74, 195-203 (2013).
15. Jones, W. & Klin, A. Attention to eyes is present but in decline in 2-6-month-old infants later diagnosed with autism. Nature 504, 427-431 (2013).
16. Chang, Z. et al. Computational methods to measure patterns of gaze in toddlers with autism spectrum disorder. JAMA Pediatr. 175, 827-836 (2021).
17. Jones, E. J. H. et al. Reduced engagement with social stimuli in 6-month-old infants with later autism spectrum disorder: A longitudinal prospective study of infants at high familial risk. J. Neurodev. Disord. 8, 1-20 (2016).
18. McLaughlin, C. S. et al. Reduced engagement of visual attention in children with autism spectrum disorder. Autism 25, 2064-2073 (2021).
19. Chawarska, K., Ye, S., Shic, F. & Chen, L. Multilevel differences in spontaneous social attention in toddlers with autism spectrum disorder. Child Dev. 87, 543-557 (2016).
20. Keifer, C. M., Day, T. C., Hauschild, K. M. & Lerner, M. D. Social and nonsocial reward anticipation in typical development and autism spectrum disorders: Current status and future directions. Curr. Psychiatry Rep. 23, 1-6 (2021).
21. Shic, F., Macari, S. & Chawarska, K. Speech disturbs face scanning in 6-month-old infants who develop autism spectrum disorder. Biol. Psychiatry 75, 231-237 (2014).
22. Krasotkina, A., Götz, A., Höhle, B. & Schwarzer, G. Infants' gaze patterns for same-race and other-race faces, and the other-race effect. Brain Sci. 10, 331 (2020).
23. Pickron, C. B., Fava, E. & Scott, L. S. Follow my gaze: Face race and sex influence gaze-cued attention in infancy. Infancy 22, 626-644 (2017).
24. Carpenter, K. L. H. et al. Digital behavioral phenotyping detects atypical pattern of facial expression in toddlers with autism. Autism Res. 14, 488-499 (2021).
25. Krishnappa Babu, P. R. et al. Exploring complexity of facial dynamics in autism spectrum disorder. IEEE Trans. Affect. Comput. https://doi. org/10. 1109/taffc. 2021. 31138 76 (2021).
26. Perochon, S. et al. A scalable computational approach to assessing response to name in toddlers with autism. J. Child Psychol. Psychiatry Allied Discip. 62, 1120-1131 (2021).
27. Dawson, G. et al. Atypical postural control can be detected via computer vision analysis in toddlers with autism spectrum disorder. Sci. Rep. 8, 1-7 (2018).
28. Krishnappa Babu, P. R. et al. Complexity analysis of head movements in autistic toddlers. J. Child Psychol. Psychiatry 64, 156-166 (2023).
29. Robins, D. L. et al. Validation of the modified checklist for autism in toddlers, revised with follow-up (M-CHAT-R/F). Pediatrics 133, 37-45 (2014).
30. Luyster, R. et al. The autism diagnostic observation schedule—Toddler module: A new module of a standardized diagnostic measure for autism spectrum disorders. J. Autism Dev. Disord. 39, 1305-1320 (2009).
31. Mullen, E. M. Mullen scales of early learning. Circ. Pines MN Am. Guid. Serv. https://doi. org/10. 1002/97811 18660 584. ese16 02 (1995).
32. King, D. E. Dlib-ml: A machine learning toolkit. J. Mach. Learn. Res. 10, 1755-1758 (2009).
33. De La Torre, F. et al. IntraFace. in 2015 11th IEEE International Conference and Workshops on Automatic Face and Gesture Recognition, F G 2015 1-8 (2015). doi:https://doi. org/10. 1109/FG. 2015. 71630 82.
34. Hashemi, J. et al. Computer vision analysis for quantification of autism risk behaviors. IEEE Trans. Affect. Comput. 12, 215-226 (2021).
35. Li, Y., Fathi, A. & Rehg, J. M. Learning to predict gaze in egocentric video. in Proceedings of the IEEE International Conference on Computer Vision 3216-3223 (2013). doi:https://doi. org/10. 1109/ICCV. 2013. 399.
36. Mannan, S., Ruddock, K. H. & Wooding, D. S. Automatic control of saccadic eye movements made in visual inspection of briefly presented 2-D images. Spat. Vis. 9, 363-386 (1995).
37. Krafka, K. et al. Eye tracking for everyone. In Proceedings of the IEEE Computer Society Conference on Computer Vision and Pattern Recognition (ed. Krafka, K.) 2176-2184 (IEEE Computer Society, 2016). https://doi. org/10. 1109/CVPR. 2016. 239.
38. Baltrusaitis, T., Zadeh, A., Lim, Y. C. & Morency, L. P. OpenFace 2.0: Facial behavior analysis toolkit. in Proceedings—13th IEEE International Conference on Automatic Face and Gesture Recognition, F G 2018 59-66 (2018). doi:https://doi. org/10. 1109/FG. 2018. 00019.
39. Ekman, P. & Wallace, V. F. Facial Action Coding System (FACS). APA PsycTests. https://doi. org/10. 1037/t27734-000 (1978).
40. Vallat, R. Pingouin: Statistics in Python. J. Open Source Softw. 3, 1026 (2018).
41. Pedregosa, F. et al. Scikit-learn: Machine learning in Python. J. Mach. Learn. Res. 12, 2825-2830 (2011).
42. Elisseeff, A. & Pontil, M. Leave-one-out error and stability of learning algorithms with applications. NATO Sci. Ser. III Comput. Syst. Sci. 190, 111-130 (2003).
43. Hanley, J. A. & McNeil, B. J. The meaning and use of the area under a receiver operating characteristic (ROC) curve. Radiology 143, 29-36 (1982).

### REFERENCES

All references listed below, as well as all references cited in the instant disclosure, including but not limited to all patents, patent applications and publications thereof, scientific journal articles, and database entries (e.g., GENBANK® and UniProt biosequence database entries and all annotations available therein) are incorporated herein by reference in their entireties to the extent that they supplement, explain, provide a background for, or teach methodology, techniques, and/or compositions employed herein.
1. Robins D L, Casagrande K, Barton M, Chen C M, Dumont-Mathieu T, Fein D. Validation of the modified checklist for Autism in toddlers, revised with follow-up (M-CHAT-R/F). Pediatrics 2014; 133(1): 37-45.
2. Lord C, Rutter M, Goode S, et al. Autism diagnostic observation schedule: a standardized observation of communicative and social behavior. J Autism Dev Disord 1989; 19(2): 185-212.
3. Bishop S L, Guthrie W, Coffing M, Lord C. Convergent validity of the Mullen Scales of Early Learning and the differential ability scales in children with autism spectrum disorders. Am J Intellect Dev Disabil 2011; 116(5): 331-43.
4. Guthrie W, Wallis K, Bennett A, et al. Accuracy of Autism Screening in a Large Pediatric Network. Pediatrics 2019; 144(4).
5. Chang Z, Di Martino J M, Aiello R, et al. Computational Methods to Measure Patterns of Gaze in Toddlers With Autism Spectrum Disorder. JAMA Pediatr 2021; 175(8): 827-36.
6. Krishnappa Babu P R, Di Martino M, Chang Z, et al. Exploring complexity of facial dynamics in autism spectrum disorder. IEEE Trans Affect Comput 2021;. 14(2):919-930.
7. Krishnappa Babu P R, Di Martino J M, Chang Z, et al. Complexity analysis of head movements in autistic toddlers. J Child Psychol Psychiatry 2023; 64(1): 156-66.
8. Perochon S, Di Martino M, Aiello R, et al. A scalable computational approach to assessing response to name in toddlers with autism. J Child Psychol Psychiatry 2021; 62(9): 1120-31.
9. Perochon S, Di Martino J M, Carpenter K L H, et al. A tablet-based game for the assessment of visual motor skills in autistic children. NPJ Digit Med 2023; 6(1): 17.
10. Chen T, Guestrin C. XGBoost: A Scalable Tree Boosting System. Proceedings of 22nd ACM SIGKDD International Conference on Knowledge Discovery and Data Mining 2016: 785-94.
11. Scott M L, Su-In L. A unified approach to interpreting model predictions. Proceedings of 31st International Conference on Neural Information Processing Systems 2017: 4768-77.
12. Hanley J A, McNeil B J. The meaning and use of the area under a receiver operating characteristic (ROC) curve. Radiology 1982; 143(1): 29-36.
13. Perkins N J, Schisterman E F. The Youden Index and the optimal cut-point corrected for measurement error. Biom J 2005; 47(4): 428-41.
14. Krishnappa Babu, P. R., Aikat, V., Di Martino, J. M., Chang, Z., Perochon, S., Espinosa, S., Aiello, R., Carpenter, K. L. H., Compton, S., Davis, N., Eichner, B., Flowers, J., Franz, L., Dawson, G. & Sapiro, G. (2023). Blink rate and facial orientation reveal distinctive patterns of attentional engagement in autistic toddlers: a digital phenotyping approach. Scientific Reports, 13(1): 7158. PMID: 37137954.

It will be understood that various details of the subject matter described herein may be changed without departing from the scope of the subject matter described herein. Furthermore, the foregoing description is for the purpose of illustration only, and not for the purpose of limitation, as the subject matter described herein is defined by the claims as set forth hereinafter.

What is claimed is:

1. A method for detection of a neurodevelopmental or psychiatric disorder using scalable computational behavioral phenotyping, the method comprising:
at a computing platform including at least one processor and memory:
obtaining user related information, wherein the user related information includes metrics derived from a user interacting with one or more applications executing on at least one user device;
generating, using the user related information and a machine learning based model, a user assessment report including a prediction value indicating a likelihood that the user has a neurodevelopmental or psychiatric (neurodevelopmental/psychiatric) disorder or risk for such a disorder and a prediction confidence value computed using relative contributions of the metrics to the prediction value generated using the machine learning based model; and
providing the user assessment report to a display or a data store.

2. The method of claim 1 comprising:
administering to the user a therapy for treating the neurodevelopmental/psychiatric disorder.

3. The method of claim 1 wherein the user assessment report includes an assessment administration quality value, wherein the assessment administration quality value indicates whether a user assessment should be readministered or wherein the assessment administration quality value is computed based on the metrics weighted by their relative contributions to the prediction value.

4. The method of claim 1 wherein computing the prediction confidence value includes performing a model interpretability analysis involving the metrics and the machine learning based model.

5. The method of claim 4 wherein performing the model interpretability analysis includes generating normalized Shapley additive explanations (SHAP) interaction values for the metrics and using the normalized SHAP interaction values for the metrics to generate an individualized summary report indicating how the metrics affected the prediction value.

6. The method of claim 1 wherein the machine learning based model includes a multiple tree-based extreme gradient-boosting (XGBoost) algorithm.

7. The method of claim 1 wherein obtaining the user related information includes providing a survey and/or stimuli to the user via a display, capturing user survey data and/or user interaction data using one or more input devices, and generating the metrics, wherein the metrics relate to facial orientation, attention, social attention, facial expressions, head movements, eye movements, gaze, eyebrow movements, mouth movements, user responses to name, hand motor skills, visual motor skills, or any combinations thereof.

8. The method of claim 1 wherein the neurodevelopmental/psychiatric disorder or risk for such a disorder comprises autism spectrum disorder (ASD), language or developmental delay, an attention deficient and hyperactivity disorder (ADHD), an anxiety disorder diagnosis, or any combination thereof.

9. The method of claim 1 wherein the computing platform includes a mobile device, a smartphone, a tablet computer, a laptop computer, a computer, a user assessment device, or a medical device.

10. A system for detection of a neurodevelopmental or psychiatric disorder using scalable computational behavioral phenotyping, the system comprising:
a computing platform including at least one processor and memory, the computing platform configured for:
obtaining user related information, wherein the user related information includes metrics derived from a user interacting with one or more applications executing on at least one user device;
generating, using the user related information and a machine learning based model, a user assessment report including a prediction value indicating a likelihood that the user has a neurodevelopmental or psychiatric (neurodevelopmental/psychiatric) disorder or risk for such a disorder and a prediction confidence value computed using relative contributions of the metrics to the prediction value generated using the machine learning based model; and
providing the user assessment report to a display or a data store.

11. The system of claim 10 wherein the computing platform or another entity administers to the user a therapy for treating the neurodevelopmental/psychiatric disorder.

12. The system of claim 10 wherein the user assessment report includes an assessment administration quality value, wherein the assessment administration quality value indicates whether a user assessment should be readministered or wherein the assessment administration quality value is computed based on the metrics weighted by their relative contributions to the prediction value.

13. The system of claim 10 wherein the computing platform is configured for performing a model interpretability analysis involving the metrics and the machine learning based model.

14. The system of claim 13 wherein performing the model interpretability analysis includes generating normalized Shapley additive explanations (SHAP) interaction values for the metrics and using the normalized SHAP interaction values for the metrics to generate an individualized summary report indicating how the metrics affected the prediction value.

15. The system of claim 10 wherein the machine learning based model includes a multiple tree-based extreme gradient-boosting (XGBoost) algorithm.

16. The system of claim 10 wherein the computing platform is configured for providing stimuli to the user via a display, capturing user interaction data using one or more input devices, and generating the metrics, wherein the metrics relate to facial orientation, attention, social attention, facial expressions, head movements, eye movements, gaze, eyebrow movements, mouth movements, user responses to name, hand motor skills, visual motor skills, or any combinations thereof.

17. The system of claim 10 wherein the neurodevelopmental/psychiatric disorder or risk for such a disorder comprises autism spectrum disorder (ASD), an attention deficient and hyperactivity disorder (ADHD), developmental or language delay, an anxiety disorder diagnosis, or any combination thereof.

18. The system of claim 10 wherein the computing platform includes a mobile device, a smartphone, a tablet computer, a laptop computer, a computer, a user assessment device, or a medical device.

19. A non-transitory computer readable medium comprising computer executable instructions embodied in a computer readable medium that when executed by at least one processor of a computer cause the computer to perform steps comprising:
obtaining user related information, wherein the user related information includes metrics derived from a user interacting with one or more applications executing on at least one user device;
generating, using the user related information and a machine learning based model, a user assessment report including a prediction value indicating a likelihood that the user has a neurodevelopmental or psychiatric (neurodevelopmental/psychiatric) disorder or risk for such a disorder and a prediction confidence value computed using relative contributions of the metrics to the prediction value generated using the machine learning based model; and
providing the user assessment report to a display or a data store.

20. The non-transitory computer readable medium of claim 19 comprising additional computer executable instructions embodied in the computer readable medium that when executed by the at least one processor of the computer cause the computer to perform steps comprising:
administering to the user a therapy for treating the neurodevelopmental/psychiatric disorder.

21. A method for automated motor skills assessment, the method comprising:
at a computing platform including at least one processor and memory:
obtaining touch input data associated with a user using a touchscreen while the user plays a video game involving touching visual elements that move;
analyzing the touch input data to generate motor skills assessment information associated with the user, wherein the motor skills assessment information indicates multiple touch related motor skills metrics;
determining, using the motor skills assessment information, that the user exhibits behavior indicative of a neurodevelopmental or psychiatric (neurodevelopmental/psychiatric) disorder or risk for such a disorder; and
providing, via a communications interface, the motor skills assessment information, a diagnosis, or related data.

22. The method of claim 21 comprising:
administering to the user a therapy for treating the neurodevelopmental/neuropsychiatric disorder, optionally wherein the therapy includes recommendations for improving motor skills or an interactive game or digital content for improving motor skills over time.

23. The method of claim 21 wherein the touch related motor skills metrics relate to number of touches, number of misses, number of pops, a popping rate, touch duration, applied force, length of touch motion, number of touch per target, time spent targeting a visual element, touch frequency, touch velocity, popping accuracy, repeat percentage, distance to the center of a visual element, or number of transitions.

24. The method of claim 21 wherein determining, using the motor skills assessment information, that the user exhibits behavior indicative of the neurodevelopmental/psychiatric disorder includes comparing the motor skills assessment information to information from a population having the neurodevelopmental/psychiatric disorder.

25. The method of claim 21 wherein determining, using the motor skills assessment information, that the user exhibits behavior indicative of the neurodevelopmental/psychiatric disorder includes using the motor skills assessment information as input for a trained machine learning algorithm or model that outputs diagnostic or predictive information regarding the likelihood of the user having the neurodevelopmental/psychiatric disorder.

26. The method of claim 25 wherein the trained machine learning algorithm or model also takes as input other metrics related to digital phenotyping involving the user.

27. The method of claim 26 wherein the other metrics relate to gaze patterns, social attention, facial expressions, facial dynamics, or postural control.

28. The method of claim 21 wherein the neurodevelopmental/psychiatric disorder or risk for such a disorder comprises autism spectrum disorder (ASD), an attention deficient and hyperactivity disorder (ADHD), language or developmental delay, an anxiety disorder diagnosis, or any combination thereof.

29. The method of claim 21 wherein the computing platform includes a mobile device, a smartphone, a tablet computer, a laptop computer, a computer, a motor skills assessment device, or a medical device.

30. A system for automated motor skills assessment, the system comprising:
a computing platform including at least one processor and memory, wherein the computing platform is configured for:
obtaining touch input data associated with a user using a touchscreen while the user plays a video game involving touching visual elements that move;
analyzing the touch input data to generate motor skills assessment information associated with the user, wherein the motor skills assessment information indicates multiple touch related motor skills metrics;
determining, using the motor skills assessment information, that the user exhibits behavior indicative of a neurodevelopmental or psychiatric (neurodevelopmental/psychiatric) disorder or risk for such a disorder; and
providing, via a communications interface, the motor skills assessment information, a diagnosis, or related data.

31. The system of claim 30 wherein the computing platform or another entity administers to the user a therapy for treating the neurodevelopmental/psychiatric disorder, optionally wherein the therapy includes recommendations for improving motor skills or an interactive game or digital content for improving motor skills over time.

32. The system of claim 30 wherein the touch related motor skills metrics relate to number of touches, number of misses, number of pops, a popping rate, touch duration, applied force, length of touch motion, number of touch per target, time spent targeting a visual element, touch frequency, touch velocity, popping accuracy, repeat percentage, distance to the center of a visual element, or number of transitions.

33. The system of claim 30 wherein determining, using the motor skills assessment information, that the user exhibits behavior indicative of the neurodevelopmental/psychiatric disorder includes comparing the motor skills assessment information to information from a population having the neurodevelopmental/psychiatric disorder.

34. The system of claim 30 wherein determining, using the motor skills assessment information, that the user exhibits behavior indicative of the neurodevelopmental/psychiatric disorder includes using the motor skills assessment information as input for a trained machine learning algorithm or model that outputs diagnostic or predictive information regarding the likelihood of the user having the neurodevelopmental/psychiatric disorder.

35. The system of claim 34 wherein the trained machine learning algorithm or model also takes as input other metrics related to digital phenotyping involving the user.

36. The system of claim 35 wherein the other metrics relate to gaze patterns, social attention, facial expressions, facial dynamics, or postural control.

37. The system of claim 30 wherein the neurodevelopmental/psychiatric disorder or risk for such a disorder comprises autism spectrum disorder (ASD), an attention deficient and hyperactivity disorder (ADHD), language or developmental delay, an anxiety disorder diagnosis, or any combination thereof.

38. The system of claim 30 wherein the computing platform includes a mobile device, a smartphone, a tablet computer, a laptop computer, a computer, a motor skills assessment device, or a medical device.

39. A non-transitory computer readable medium comprising computer executable instructions embodied in a computer readable medium that when executed by at least one processor of a computer cause the computer to perform steps comprising:
obtaining touch input data associated with a user using a touchscreen while the user plays a video game involving touching visual elements that move;
analyzing the touch input data to generate motor skills assessment information associated with the user, wherein the motor skills assessment information indicates multiple touch related motor skills metrics;
determining, using the motor skills assessment information, that the user exhibits behavior indicative of a neurodevelopmental or psychiatric (neurodevelopmental/psychiatric) disorder or risk for such a disorder; and
providing, via a communications interface, the motor skills assessment information, a diagnosis, or related data.

40. The non-transitory computer readable medium of claim 39 comprising additional computer executable instructions embodied in the computer readable medium that when executed by the at least one processor of the computer cause the computer to perform steps comprising:
administering to the user a therapy for treating the neurodevelopmental/psychiatric disorder, optionally wherein the therapy includes recommendations for improving motor skills or an interactive game or digital content for improving motor skills over time.
